SEQUENCE LISTING (826 sequences)

SEQ ID NO: 1 - DNA, 3629 bp, Cannabis sativa
  misc_feature (1)..(3629): THCAS gene  CM011610.122243181-22246809
tattaatttttgtagaacatatacaatattattatgtataaaaatacatgtactttttttttgttaattttaccattttaatatttatcacaaaatcactacttttatatattttaaaaatagcaaattatgtttctgatttcaatactattactttatttataataacacacatcaaaatgttatattttaaattatatcactcagtataagtacaatatatatatatatatatatatatatataaaatattacaaaagttggacaacaccgagcgaagcgcggcttagttccctagttgaataataaagtagatagtagaggagattagaactcataatttttcatttatatatttgaatttagtttactgttctttattattcttcatttaattttgctatatgttattttacttttcaaaaattctaattttgattaccaaataaaaaataaaatatatattggtacttcatatttagtttttatgggaatgataatgaatgatattgtaataaactataaaagtaattttgtatacttgcatagttcttaatttttacccaaaaaaatatatattaatgaaaaaaaaggtggaaggtgccataggcacctatatcccacaaactagctataagatattgaaagacaaatagatccatagataaatatttgcgtgtcctttcttttcatatctaatttcttgtttttttatttttttttcaatagccaatttcggcagcttaacttctttccatatataatgatgccaaactattcaatgtaaaatttagatttattttcattaagggcttcacctaacaaatgtgcctaatttttgtggatttttttaccatatgtcactatttaatgactaccaaattatagaaatatttaagtcaatttctcagtccccgctccaatataaagttatagaaaggacaattcttaattcatagtaattcaaaaatcattaagactaaaaaaaatgaagtactactcaacattctcctttaggtttgtttacaaaattatatttttctttctctcattcaatatcaaaatttcaatagctaatcctcaagaaaatttcctaaattgcttctcccaatatattcataacaatccagcaaatctaaaactcgtatacactcaacacgaccaattgtatatgtctgtcctgaatttgacaatacaaaatcttagatttacctctgatacaaccccaaaaccactcgttattgtcactccttcaaatgtctcccatatccaagccactattctatgctccaagaaagttggcttgcagattcgaactcgaagcggtggccatgatgctgagggtttgtcctacacatctcaagtcccatttgttatagtagacttgagaaacatgcattcggtgaaaatagatattcgtagccaaactgcgtgggttgaagccggagctacccttggagaagtttattattggattaatgagaagaatgagaatcttagttttcctggtgggtattgccctactgttggcgtaggtggacactttagtggaggaggctatggagcattaatgcgaaattatggcctcgcagctgataatatcattgatgcacacttagtcaatgttgatggaaaagttctagatcgaaaatccatgggggaagatctattttgggctatacgtggtggtggaggtgaaaactttggaatcattgcagcgtggaaaattagactggttgctgtcccatcaagggctactatattcagtgttaaaaggaatatggagatacatgggcttgtcaagttatttaacaaatggcaaaatattgcttacaagtatgacaaagatttattactcatgactcacttcataaccaggaatattatagataatcaaggaaagaataagactacagtacacggttacttctcttgcattttccatggtggagtggatagtctagtcaacttgatgaacaagagctttcctgagttgggtattaaaaaaactgattgcaaagaattgagctggattgatactaccatcttctacagtggtgttgtaaattataacactactaattttcaaaaggaaattttgcttgatagatcagctgggcagaaagtagctttctcaattaagttagactacgttaagaaaccaattccagaaactgcaattgtcaaaattttggagaaattgtatgaagaagatgtaggagttggagtgtatgtattgtacccttacggtggtataatggacaagatctcagaatcaacaattcctttccctcatcgagctggaatcatgtacgaagtttggtacgcagctacctgggagaagcaagaagataatgaaaagcatataaactgggttcgaagtgtttataatttcatgacgccttatgtgtcccaaaatccaagaatggcgtatctcaattatagggaccttgatttaggaaaaactgatcccaagagtcctaataattacacccaagcacgtatctggggtgaaaagtactttggtaaaaactttgacaagttagttaaggtgaaaaccaaagttgatcccaataatttttttagaaacgagcaaagcatcccacctcttccgccacgacgtcattaattatttttaaatatatctatttctcttatacataagttaatgcttataccatacatgtatctgttatatatataatttgtttacctgtactatgtatgctcctaagtatgaacatctacagtacaactgtgtagacatcataagatatatggaataaaataaattatctttcttatttaatggagaataaattaatattcttttaacttacatgtttatggaatagttttattttaatgcattaaaattataatatattcattaataaaatattgggtacttgttattaaaatgataactgaagttttatagtaaaagagtgttgtgtagtgacttcctttaattataatctagagttacttttagaattttgggtatatgtagttaagttataatcttattttactttcttaaagaatccttggttcaattagattaatcatacaaaagattgagatccacatatccctccctgtatgaaagctccaatatgaacttaaattttgaagggaaaatatcaccaccaccaaatggccttatttaaatgatccacccaccaacattagagaaaaagttgaaattatcaaatccaactataaactgtcacaactaacttacagcacaagaacttaaaatatctcaggcccaaaggcttgcctcaccaaactctcaaatagacactattacaaaaatgtcctttcatggagatcaaagttggatggaaaaactattttttgtcacttagttgtttgacatgatatctagcaccataaaatgggtatttttatgttcagcaatgttgaacatgaaaaaaacatcatgttcaatattattggacctgataatgggatccacaacaatgctcatttcatgtccaacaatgtgataataatttatatttatatttcacatcaggcaaagttggacagacataatgttttttttttttaaaaaataaaataataattaaaatagttcaaagaactaaccatgacaaaca

SEQ ID NO: 2 - DNA, 1641 bp, Cannabis sativa
  CDS (1)..(1641)
atgaagtactactcaacattctcctttaggtttgtttacaaaattatatttttctttctctcattcaatatcaaaatttcaatagctaatcctcaagaaaatttcctaaattgcttctcccaatatattcataacaatccagcaaatctaaaactcgtatacactcaacacgaccaattgtatatgtctgtcctgaatttgacaatacaaaatcttagatttacctctgatacaaccccaaaaccactcgttattgtcactccttcaaatgtctcccatatccaagccactattctatgctccaagaaagttggcttgcagattcgaactcgaagcggtggccatgatgctgagggtttgtcctacacatctcaagtcccatttgttatagtagacttgagaaacatgcattcggtgaaaatagatattcgtagccaaactgcgtgggttgaagccggagctacccttggagaagtttattattggattaatgagaagaatgagaatcttagttttcctggtgggtattgccctactgttggcgtaggtggacactttagtggaggaggctatggagcattaatgcgaaattatggcctcgcagctgataatatcattgatgcacacttagtcaatgttgatggaaaagttctagatcgaaaatccatgggggaagatctattttgggctatacgtggtggtggaggtgaaaactttggaatcattgcagcgtggaaaattagactggttgctgtcccatcaagggctactatattcagtgttaaaaggaatatggagatacatgggcttgtcaagttatttaacaaatggcaaaatattgcttacaagtatgacaaagatttattactcatgactcacttcataaccaggaatattatagataatcaaggaaagaataagactacagtacacggttacttctcttgcattttccatggtggagtggatagtctagtcaacttgatgaacaagagctttcctgagttgggtattaaaaaaactgattgcaaagaattgagctggattgatactaccatcttctacagtggtgttgtaaattataacactactaattttcaaaaggaaattttgcttgatagatcagctgggcagaaagtagctttctcaattaagttagactacgttaagaaaccaattccagaaactgcaattgtcaaaattttggagaaattgtatgaagaagatgtaggagttggagtgtatgtattgtacccttacggtggtataatggacaagatctcagaatcaacaattcctttccctcatcgagctggaatcatgtacgaagtttggtacgcagctacctgggagaagcaagaagataatgaaaagcatataaactgggttcgaagtgtttataatttcatgacgccttatgtgtcccaaaatccaagaatggcgtatctcaattatagggaccttgatttaggaaaaactgatcccaagagtcctaataattacacccaagcacgtatctggggtgaaaagtactttggtaaaaactttgacaagttagttaaggtgaaaaccaaagttgatcccaataatttttttagaaacgagcaaagcatcccacctcttccgccacgacgtcattaa

SEQ ID NO: 3 - protein, 546 aa, Cannabis sativa
MKYYSTFSFRFVYKIIFFFLSFNIKISIANPQENFLNCFSQYIHNNPANLKLVYTQHDQLYMSVLNLTIQNLRFTSDTTPKPLVIVTPSNVSHIQATILCSKKVGLQIRTRSGGHDAEGLSYTSQVPFVIVDLRNMHSVKIDIRSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIRLVAVPSRATIFSVKRNMEIHGLVKLFNKWQNIAYKYDKDLLLMTHFITRNIIDNQGKNKTTVHGYFSCIFHGGVDSLVNLMNKSFPELGIKKTDCKELSWIDTTIFYSGVVNYNTTNFQKEILLDRSAGQKVAFSIKLDYVKKPIPETAIVKILEKLYEEDVGVGVYVLYPYGGIMDKISESTIPFPHRAGIMYEVWYAATWEKQEDNEKHINWVRSVYNFMTPYVSQNPRMAYLNYRDLDLGKTDPKSPNNYTQARIWGEKYFGKNFDKLVKVKTKVDPNNFFRNEQSIPPLPPRRH

SEQ ID NO: 4 - DNA, 3635 bp, Cannabis sativa
  misc_feature: CBDAS gene CM011610.121836038-21839672
atgattaagagaagggttttgttttgtagtcttgggttcgacaaacgacttttcaaatatgatatataagtatttcaagtcttgatattataatatagattattttacaatctttattcacattacctatttttttttattaaattgtgtagtaatagagttttatttttaattattttgtaagtaatttttatttaggtgatgtaatagagctattttttttttttttgtaggtcggttttgttaagggtctacgttatactgtatttgttgataagtttatatttttacatataatttatttgagagtttttatcttcttactattgataatttcttttattagtttttaatataattgttaggggataattcattatttttatcttatttttttgtagatattttgtggaccatttgttatatgagttggtgtgtagtttttcagtgttacaagttgaatttctagctcttttagtgtgttgtttgaagatgtgtagctttattttctaaatttttagtggtagttttgtcccatgttgcttgcttactttagcaattcatagtttgacaaagcatgctattggttagacgatgatctatcctagttcgaggaagtttctgcaatatttgtggatatttacatcgtaaattcatgagtgattcttatcacttcgtaaaaaaaagatagaaagatacataaataaatatttactagtcctttctttccatagtcaacttcaatatggcatcttagcttctttccatcaacaaatgatgctcaactacttaatgtacaatttatatttatttttaataagggatgcacctaacaatacaatgatgcctaatttttgtgaattttttttaccacatgacttaatgatatcaaattatgaaatatttagttaatttatttgcccctgctccaatatataaagctataaataggatagttcttaatccatagtaattcaaaattcattagaactaaagaaaaatgaagtactcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcatcattaatgatcttaaatagatctttttctcttattaattagtccttataatatacatatattgataatatatataaaaaagtttgtccggatgtactgtgtatgctccaatgtaagaacatctacagtacagctatgtagacaatcataagatatatgtaataaaataaattgtctttcttattttaatggagaatgaaataatattattttagcttactgaataattttattttacatgtttatagattagaacaatataatatattcattattaaaatgtttggtacgtgttattaaaaagataactgaagttttatacttaaaagagtgttttgcagtgtcttcttttaattataatctagagatacttttagaatttcaggtacatatttagataagttataatcttatattaattttgtaaagaacctatggttcaattaaattaataatacaaaaaaggcctatcttaacagacccagctccccattattgatctatcttggtgggtccgacccgccataaatttcaatgtcgagatttgttgtgagtcgatctcggcattcccatcctgcccttatcggaggcaacctcggcgggctttgtccaccgttaatgatattgtaatctcagaggtctatacttgccgagataggtatattttaagtctcaatttagtagttttcgacacctatcacgatggacttggcccttagagattggtcaatctcggcagtcttaacgtcgagatagtttacaagtggaaaaaaaattaatttccccttctattacattgattgtatacaaaaatttacagttcattaaaaactagacaattattttctctctttctaacatgttgcaactctcgggccagtgctatggtgaaacaatagctaagacgcaaggaaaattgagatttatggtaaagaaacgcaagaagaatggccatgcttaaacttttgatgttcagaaaataaagtctatggatgagattcttggaa

SEQ ID NO: 5 - DNA, 1635 bp, Cannabis sativa
  CDS (1)..(1635)
atgaagtactcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcatcattaa

SEQ ID NO: 6 - protein, 544 aa, Cannabis sativa
MKYSTFSFWFVCKIIFFFFSFNIQTSIANPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHHH

SEQ ID NO: 7 - DNA, 10729 bp, Cannabis sativa
  misc_feature (2)..(10729): Genomic Sequence PT
catgctatcatgaaaaagaggagagttgagaataaagtgtgttcgtttactgccaatggagttgttgttgatgagtatgacaaagtggttgagcattttttaaaccactttcgcaacttcatggggaggcaaagctcggcttctagacgaatggatgaggatgcctcgagtttgggcaaaagcttactctcgaacatcaagtgagtctaattcgtcctttcagcaaaaaggatgtgaaagaagctttgtttgggattcactcgaccaaaagtccggggccggatgggtttggttcgggttttttcagagagctttggaatgatattggggatgacttctcttgtgcggtgttgggattttttcaaactggtaagcttcctcaggaagtgagtgcaacaaccatatctctcataccgaagattgataaccctcaaggagctactgactacagaccaatagcctgctgcaccactacttacaaatgcatttcaaaaatgatttgtaccagactctcggaagttctcccggtccttattcatgaaaaccaaggtgcttttgtcaagaagagacttttagctcataacgtcttgattttgcaggatcttctaaaaggatacactaggaagaccatttcttctagatgtcttatgaagattgatttgagcaaggcttatgatacggtagactggtgctttgtggctgatattttaaaagctcttcgcttcccttctaaatttatcacctggatcttaagctgccttcacggagcttcttatgcccttctgcttaatggaagaatccaagggtcgtttaaaggtgaaaaaggccttcgtcaaggggacccgatgtccccgcttctctttgttattataatggaatacttaaccagacttcttattcaatgttctaagaagaaagggtttggttttcacccgttgtgcaaatccttaggccttgtcaacctgtgttttgcagacgatctcattattttctgcaagggcaatgacaagtctgttcagatggtgaaaaatgccttcaatcggtttagtgaagcaacgggcctggttgctaacaaagacaagtcaacggtgttttttgggggtattccaaatgccagcaaacagagacttgccaacatcttacaaatggaagagggttctttccctatgaagtacctcggggtgaatcttagaccaaccaagtggcgatctgctgattgtggtatcattttagacaaactgcacaagaatttacacacttgggctagtaggaatctctcgtttgctggaagggctcaattgattcattcgattttgttggggatcaggaattattggatgggcttatttttgcttcctcaaaaaattacggcggctattgacaaatgttgcagagattttctctggggttctgtgggaaacagaagcaaacttcatattccttcttgggaaaaagtttgtctccccaagaatgcaggaggccttggctttagagaaggtaaggcttggaatatagcgcttatggcaaagtatatctgggctatatctagtaaacaggataacctttgggttagatggattgatgcaatctatctgagagggctgagcttttggacagttgaattcaagcaagatgcgagctggtacttcaaaaaattacttcgtcttcgttcaactgtcaatgaaacaatagtcactgctgctattaaaagaggtaagtttagtgctaaactcttttatctctcttttattcaagctcaaaagattgattatgcaaggaacatttggaatagaatgattgttccaaagcataggttcataggatggcaaatagtgaataatcagctgctaaccagggacaatctaagccgagttatgccaatctcttctcctctgtgcccggtctgctgtagggaaaatgaatcgcaccagcatctttttgtaacctgctgctttactcgcaatctggttgatgagattaccaaatggtttggccaatcgacttcagctgttggttctcaaaggctgctgtcaacttgcagggaagaatcaccaatgctgttattttggctaccctttacatggtttggacaaacagaaatagttgcatatttgagttgagttgtaagacagctagcaccttaagtagagagatcaaatcttgtgtaaaatatagatgtttaattggttgtaatgggggaaaagggaagctagataatcacatttttaatgtaatttccagctggttttaatggcagatttcggtctgcagctggtgttgtttgcttgtttgttttgtctgtttgaataaagatcttcttcttgttcaaaaaaaaaattcaaggttaagatattgtcgggtcattacaactctagatgtggaaatatgaaagtgaaattttaaataaaatttagtgtcttacactatattattatatatttttgctgtaaacaaataatgcatacatttaaatttaaataggtaagatccaccactaaattatacgaatatttagctataaataatataatgtgttggacacctaatgtgtcttaattatatgaaaacatatactaaatattcagcataataaagcttccacatgaactcgtaaaatcttctgcctgcttcctgcattgtacaacccaatataaaatataatcagatgtaaaattaattcattagaaaattagtattatttttgtatgtaccaataaagaggataataacacaattaatggtagtcattttgagtattaccgggtcgtaatttgttaacgcaaaatctcgagtctgtaaagtacaattaaacaattaaaaatatttagaatttttttttaaatataatgatatatttgttttttttaaatagataatttaattcaaaatggcaaacctggaggattaaccaaaatgctaagattgcatgaagtaacattacgttactgttgaaagcctgtaattaaataagaaatgatgagaagaaatgatggcaaacctggttttttaataaattcttaaagctaagggccaccattgacatagaggctttgattcgagttggcaggtatggaaagtttaaagtcaaagagttttatctctctctaatcaaagcaaaaaaagttgaatatgctccaaaagtttggcacaagctcaatattatacctaagcaccggcccctttatttactgacaagctattaatgattaactattaactagggatcacataagcaacattatgcctattccttctaatttgtgtcctgtttgtgaagcttatagagaaacccaccatcacttgtttgtgaattgttgttttaccaagaaagttataactgatataggaagatggcttggtctgtatcaatggcctcaagacttcaaattggaaggattggtactttcaagcctctcactaatttgcaaatagtgtttaaactctgtcatgacaacaaccatttattttttgtggttgaacaggaacaattgcatttttgactttacttgtaatactaccaatcatgttagttggcttcaaaattggtagtttggctttctttgagctgctgcatttattgcagtaggctttggtttggtttggtttgaataaaaagttttcttttgtttaaataaataaataaatgaaagttataattgaatgagactaataaagagtattattcctatatggtaaatgtaacttgatttgtgtttaaattttcacttaaattagagtatattaattctgatattaaactttattaattgtctcacaatatatattgaaatacaaataatatagatgagatgagatacctggggccagataatcccagcaagtatagcagccacataggataggagaacaattccagaacaaaataatgtcaagtttctggaaccatatttacttgccaacgttgatatgccaaatcttgaaagaaatatataaaaattatttcatcaataataataattatttaaactaattataaggtttatatatagaatttaacatacttagtgtcgccttcaacgtctgaagcatctttgattaaagccaaagctgcacccattgatttaataaatgctagcaggaaagtaaaagaaggcctgaaaataatcaaattgaaattcaaacatataatacaatcaaattaaaatacagggaaatcgtttatgttattggaaaagtttacaaagtttaagtacactccaatctctaaaatacgtgtttcagtatttatcagaatttttagacatgtaaatttttaaaaaaatttaaatagtttatagtgttgaaaataatatttaattaaatatattattactgatttttatttttttatatactgtttgaatcttattttttgaatattttaaattattcaatttttttgttttgaaattttacagagaattatatatgaaaaaattgaactaaaacttaattactctcgaatgccaaaagaaaattcttaagtggtgtattataaacttatatatcctatattatatagtatatcttgggatagtattaagttttcaattaaatatactattacccacctcaactcaaatggtaggacaagagctgctctgctggcatgataaaatgtgaaatttgtaataatatgggcctgtgcaagcagattaaaaaaattagaaaattataaattttataagtttttacattttgtaaaaatatagtttttttttaatatgagaaatttgctaattagatactcttatagtaaccagagaggtaactaattactataataaataactaagtatcttatgttattttttatatatatattttttaacttttttttttaagatagttttttacaaatttatgctttagaaatcataaaaataaaattccaacgaatatatttttatataaatatgggagtgtacaatcttactttaataaattttttgatagaaatcttactttaataattatctcatctaactaaatggtgatcttaataaaattaagaaattagtaacagaaaaatatgatagttataattaaagagaacatattttgtgtgaagatatgattattgttagtaaagtaaaataccaggaaattgagaagaaatgcagtggaaggattttgcttccatctaaatggtggaacagaatagacaattccaccaaaaataccaaaacagtagccaaatatatagagtggtccacccttcatttttatagttattatcaatccaaacagtgccacaattatgctcataatccaagctgtgtttactgatatttcccctgaagctagtggtagatcaggcttgtttattctgaaaattattactcatcaataacttaaaattcatctcaatttttttttttttgttacaaaaaattgttgtagctagggagattagtagtgcacttatttttataaatctaaaaaaaatcttacacattcttttaataaacaaataacccaactttatttatgacccacaaaaattataattgttatcctactagaagttcaatagcataacaagtatattaaatattcctaaaattattaactcaaattaataattaaaaacacagtcaaaaaaaaaaaaaagtcataaaatgcatataatttcttctttcttttttttttttgaaatctattcaaatttcttttaataagtagaatttagattgttatataaaaaattgaaattagaattatgtgatacctgtcaatgtgaagatcgtaaatctgattgatggtagttgtaaaagaagcaatgcataatacagccaccaaaaaaaagaatgccttgaacatcagagaccaacttattaaatttgtgttatgcaacaactctttcccaaacaatccactgctctcccataccaaaattaaaacaaatatatattaattatttgtaacaatgttgttttattcaaattatacaacttaatatttatgtattgccacatttaactctcaaatctaaatttttggggacaaaatatgttaatcttttttgttatttaaggtggccttttctttcttatgttaaatgccaaatagattgacattgtacacgtggatgtatgtgtttttatatgttcgaataagtttagttatttaaatattataaaattgatttaaaattaattttgaaatttgaaaaaataataacaaataaaaaatattatataaaaaaataaatatatcttcttcttcttccttctgtaacgagattttctttcttttttctctcacgagatctctctttgtattatatgaaggtccaacttaagcatagacggactaggcatgtgcctagggcccacctagctccaaggtataattttatttttttttttaaaaaaataaatactcaaaaataattatttttctatggcccactaaatcactgcttatataaagtaaaaccaataacttatgagggtttgggtttgtaagaataagaaagttaatctatgtttgctatgatctctccaatttttttggttagtaaataatgtacaattagagctcgtttggtcatataaaaattgtattgtataaataataagtaacattacgtttagataaaatattatattatattaattaaaaattaaaactaggaatgtacctacgtgcagatgagggtgagcgccacccctttaaaataaaattataacatatatttttaaattaattacatcatatagtataattaaaactagcaaaaaaaattaacaaagggtttgttagagtaaaattagtactttacatatgtgtgttcaaataccaaagttattttatttttcattttatgaaaatgctcatgatagaaattaaaactcctaccattaagaattttattaaagagttatccattgacaccaaatctatatcattatgttttagctttaaattatattaaatacttaaaaatagggcccttaaaaatacaattttttagcatatttatattatttaaaatgatacttatctatagctcagctcgctactgccatgcccctaccaacttaaatgtcaacaggagggcggggcaaggacagagattctttcctatttccatacatactttctaaatttactcttaacctcaatacttatttaaattaaaacaaagactggacgagaattacccatcaagaaaaagattcctattaagtacccattacatatatatatatttttaaaaaataatgtctaaccagaatttaaaagaaaaacaagcataaaaactcattactacaataaacataagtgaaatagtgtgtatagtaaatagaattaataaatacaaaattggtaatcgtaaataaatggttaagtgtcatttttcttttaaagcagttaaatgtgattattaatatgcatcaccccgaacatttttatgaaaattgaaaggggttttttcattagtgtacaacaaaaacattatattacaaaaaatgtgcaaaaaaaacacaatttcaaaaatgtaacatttgaaaatatatttacaaaaattcatacatatttttaattaaattataatagaatataatgattcaactttctatatttagatttgtataactattttttatgactaattgatatggtattaatgagtaagcctaataattaacaaaatatatatattaataattaaaaaacataataagattctaattataaacaataatatttaaaatatacagatgtaatattttaaatataagacaaaaaaaaaattaacgattaaaataataaacagttttaaattaataataaaaagtgaaaaaatacattaaaaaaataaacaaaaacaacccatcaagtttaacaaaaacatatataaattaaaaaaaaaatacaaaaacaaataaacaatattaaaaaaatattacaacacgatacacaacaaaacataataaaaaaacccaaaacaacaaaagaaaaaaaaacaactaacaaagataaaaataccaaaaaaaggacatttatagatggagcatgaataattcttccaccaaaaaaatacattaaaaaaataaacaaaaacaacccatcaagattaacaaaaacatatataaattaaaaaaaaaacactaaaaacaaataaacaatattaaaaaaatattacaatgcgatacacaacaaaacataataagaaaaaaaaaaaaaaaaaaaaaaaagaaaaaaaacaactaacaaagataaaaataccaaaaaaaaaaggacctttatagatggagcatgaataattcttccaccaaaaaaatacattaaaaaaataaacaaaaacaacccatcaagtttaacaaaaacatatataaatttaaaaaaaaaacactaaaaacatataagcaatattaaagaaatattacaataccatacacaacaaaacataataagaaaatagttatacaaatagttatacaaatctaaatatagaaagttgaatcattaatatatctattatattaattaaagaatttctatgaaattttgtaaatatgttttcaaatatgtgcattttcaaaattgtgtttttttgcacgttttttgtaattatttaaaaaaatgtatacatttatgtaaaatgtccaaattgaaatatctcattagtgtaatcagtaaaaaaaagcatgaagttattttaattgtagtattatgattttaaaacataaatatatacatatatcatatcggaacgagttcggggtgggtatttattcccatgcccctttattatttttgactcgagtatgaatatttaaactcatacctgttcccacccccgaaagtccgcccctaccccattaggagcaggtacccgcatgtataattgcgatccgtaatttttatttttatagtttttaagatataaatttgtaagtatataatatatagttttttttttttttttaaaaaaaaaaaaaagttattttatggaaaattttaaaataaaaaagcttaaaaaatatgaaaaaaataatataaagtaactactcatttctctagtaactctaagggtattcttatttgattttttcatatttgaaaaaaataccatatttttgcacagaaagtataaaaactcataaaatttgtaattttctcaatttgtaaattaaatattaaaaataaaattatgagaatatatacttaccaagcgcatgaagtaaatgctatgattgtatatggtctttgaagcttccaacatgccttcccaaagtttaaaattttagttgctactgaatgattatcagattctggaggctcagtttgatttgtagtagctgcctgttaacacattattattattattattattattgttgttgttgttgtgtatttttttcttttaggttgaagaatagaaagcacaattctaattaccctctaattaaatataatattaattaaatattatgtatcattaagaagaaaaaaaaatatacaaatatatagaagtaagagacacacacataccctaatggaattttttgcgattgataatgattctgagcatttgttttgtagatgaaaactcttggtggagcaatgtttagagggaaaattattgtaagagtatttaattggtgttttggggtgtcgatataataatgaggttttgggattattattgtgaggatttaataaagtatggtaattagtttgaaatgaaaaggtacaaactgatgagagtcccatgaagattattattgattgaatattaaaaagaaaaaatgagggaaagaaagggttagaataacatagtgatatcaataatgaaaaattaacaagatttatagtaaaacaatttttgcttggtttcaaaatttaaagtcatccatgttttagaagtcatgcatggtaggcacatctatctttcttgcctaattaattaattatggctatacaaaccgtgcatgcatgtagagctctacactacgatgagattaaatttatacaatcatatcaaaattaatattttgatttagtgttgaaaatttatgaattattgtaactattaaaacatgttcggaaatccctcatctcgaccaccaaataccatctagaatcttcctacaggaccctcctacccggagcctcacatctagaacttccaatccgggacctcccaactagagccttctcaaaataaatccttaaccacacacacacatttttgaaactatcaatttggaccccatctaaaaactttgatccagaatataccatccaaacctcactcatcacctattcgggatcccttgataaggatactaacaaccaggtcacaccaatccaagcaaccaaaactctaggctcttaactctagattcaccaaccccaaacctaatcattgaaaacactgtctaggactacccttccgaaccttaccactcaaatcacatatgacaagtacaactcattcgaaaccattttgggcagaaaagagttcaaaactactcaactttcagaccttcaaaattaagtcattccttgaccaaaggctatcaaacttaccaaaaagagggtaatctaaacaaaacatacacttcaaaagcatctaaaaccacctaaaactaatttcaccatattgttgagtggactttgagttcaaaaactcgaactcctccaacacgaactacatcagctaaaatcttacaaaccaacacagaattcaaccttaaatcaaaccaacaac

SEQ ID NO: 8 - DNA, 1182 bp, Cannabis sativa
  CDS (1)..(1182)
  misc_feature (1)..(1182): PT CDS
atgggactctcatcagtttgtaccttttcatttcaaactaattaccatactttattaaatcctcacaataataatcccaaaacctcattattatatcgacaccccaaaacaccaattaaatactcttacaataattttccctctaaacattgctccaccaagagttttcatctacaaaacaaatgctcagaatcattatcaatcgcaaaaaattccattagggcagctactacaaatcaaactgagcctccagaatctgataatcattcagtagcaactaaaattttaaactttgggaaggcatgttggaagcttcaaagaccatatacaatcatagcatttacttcatgcgcttgcggattgtttgggaaagagttgttgcataacacaaatttaataagttggtctctgatgttcaaggcattcttttttttggtggctgtattatgcattgcttcttttacaactaccatcaatcagatttacgatcttcacattgacaggataaacaagcctgatctaccactagcttcaggggaaatatcagtaaacacagcttggattatgagcataattgtggcactgtttggattgataataactataaaaatgaagggtggaccactctatatatttggctactgttttggtatttttggtggaattgtctattctgttccaccatttagatggaagcaaaatccttccactgcatttcttctcaatttccaggcccatattattacaaatttcacattttatcatgccagcagagcagctcttgtcctaccatttgagttgaggccttcttttactttcctgctagcatttattaaatcaatgggtgcagctttggctttaatcaaagatgcttcagacgttgaaggcgacactaagtttggcatatcaacgttggcaagtaaatatggttccagaaacttgacattattttgttctggaattgttctcctatcctatgtggctgctatacttgctgggattatctggccccaggctttcaacagtaacgtaatgttacttcatgcaatcttagcattttggttaatcttacagactcgagattttgcgttaacaaattacgacccggaagcaggcagaagattttacgagttcatgtggaagctttattatgctgaatatttagtatatgttttcatataa

SEQ ID NO: 9 - protein, 393 aa, Cannabis sativa
MGLSSVCTFSFQTNYHTLLNPHNNNPKTSLLYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAVLCIASFTTTINQIYDLHIDRINKPDLPLASGEISVNTAWIMSIIVALFGLIITIKMKGGPLYIFGYCFGIFGGIVYSVPPFRWKQNPSTAFLLNFQAHIITNFTFYHASRAALVLPFELRPSFTFLLAFIKSMGAALALIKDASDVEGDTKFGISTLASKYGSRNLTLFCSGIVLLSYVAAILAGIIWPQAFNSNVMLLHAILAFWLILQTRDFALTNYDPEAGRRFYEFMWKLYYAEYLVYVFI

SEQ ID NO: 10 - DNA, 3002 bp, Cannabis sativa
  misc_feature: Genomic Sequence OLS CM011613.12335391- 2338392
ttgaagcttttaattgtcctttttataatgtcgtgatgtttttttttttaataactaaactcctgtacctttttactttttgttttttctaatttcacttaacaaaaattttagtttctttttagttcttaattaactaaattaataaaaagtaattatctaattgttaccaacaagcaagcactaaattttgttatttaaataaaaagaaacaaaaaataatctccctaattcttttctttaataacaatagaaaaagttttatcaattttggttattttacatgactataaatataagtacaccttaaaaaataattacatatattagaagcaaaattacatatatatatatatttatttatatgtagaatctattattatatctaataattaacaaatattaacaaatcattttgaaaaattatattaaaaaaaaaacttgaaaagtcaaagattaaccatcaatttgccaaatcaaattagtgagaaagtaggtattatatacctaacactcattttacatatgtctagacgtttacatgtatagcgttgttgtgtgtaataagttcacttgtagtatcttgtacatacataatatatatatataggtgtgtttgtgaacaattatattatcacacatacacaactcattattatcataataataataataatatgaatcatcttcgtgctgagggtccggcctccgttctcgccatcggcaccgccaatccggagaacattttaatacaagatgagtttcctgactactactttcgggtcaccaaaagtgaacacatgactcaactcaaagaaaagtttcgaaaaatatgtatgtattatgaatatatatatatatatatatttccaactaataatacttttccatgtataagcattatctatttatataataaaggaatttttacatcatgtgttaagcatgaagaatttatgtttaatatcttgtatcattattacttactttatatgtatatgtaggtgacaaaagtatgataaggaaacgtaactgtttcttaaatgaagaacatctaaagcaaaacccaagattggcggagcacgagatgcaaactctggatgcacgtcaagacatgttggtagttgaggttccaaaacttgggaaggatgcttgtgcaaaggccatcaaagaatggggtcaacccaagtctaaaatcactcatttaatcttcactagcgcatcaaccactgacatgcccggtgcagactaccattgcgctaagcttctcggactcagtccctcagtgaagcgtgtgatgatgtatcaactaggctgttatggtggtggaaccgttctacgcattgccaaggacatagcagagaataacaaaggcgcacgagttctcgccgtgtgttgtgacataatggcttgcttgtttcgtgggccttcagagtctgacctcgaattactagtgggacaagctatctttggtgatggggctgctgctgtcattgttggagccgaacccgatgagtcagttggggaaaggccgatatttgagttagtgtcaactgggcaaacaatcttaccaaactcggaaggaactattgggggacatataagggaagcaggactgatatttgatttacataaggatgtgcctatgttgatctctaataatattgagaaatgtttgattgaggcatttactcctattgggattagtgattggaactccatattttggattacacacccaggtgggaaagctattttggacaaagtggaggagaagttggatctgaagaaggagaagtttgtggattcacgtcatgtgctgagtgagcatgggaatatgtctagctcaactgtcttgtttgttatggatgagttgaggaagaggtcgttggaggaagggaaatctaccactggagatggatttgagtggggtgttctttttgggtttggaccaggtttgactgtcgaaagagtggtcgtgcgtagtgttcccatcaaatattaataaaagataataataaatgataaaaagaacaataaagaataatgcccaatttaaatttaacttcaaatttaaatatatgttgcttattgtctaatcaaagttagctttatgtttttaattttcacgtggattcaattttcttgaattatgaacagttgtaataaagttgttggttaattaagtttttaattaaataatttacgagttgccaaattaacagaataattcaataatttatcatgattaaatcagtattttgttttatagtagattacttatttattgtactcataaaacaatacttattttacttttaaaattaaaattcataattatttcaaacaaatttaaaggtgaaaatgtatatttactatgtcccatttacataataaaaaaatttatcaccttaaaaaataaaagaaatatatatatatataagagaaatactagtagctagcttacttaaaaatatcatatcattattgatgtaattaagtattgaatttttatttaatttaatttaataattattttaaaatatcattaatatttataaaacgtcacctataattatttgtatttaaggaagaatatattgctaacttcttgcaaaattggctaagatatatgttgttgcactgtataatatgtattattcaatttacttttgggtccaggtaaaacttaaaatatgtagaggaaattacattgtatattcactttactttatatgttttaatttttacctttatttttatattttttaaaatatatctacttttatgatgtcccagttatacttttagattaatacaaattgtgtgttagacttaaataaaggttgaaagtgtattgggcaacccacttataaaaattggaaaattttaatgaaatataatttaaaagtatttttttatcaatggatacaaaaaacaatatttaacaacccaattaaattgggtttattttataaatatggcttttttgtaattggttt

SEQ ID NO: 11 - DNA, 1158 bp, Cannabis sativa
  CDS (1)..(1158)
  misc_feature (1)..(1158): OLS Coding Sequence
atgaatcatcttcgtgctgagggtccggcctccgttctcgccatcggcaccgccaatccggagaacattttaatacaagatgagtttcctgactactactttcgggtcaccaaaagtgaacacatgactcaactcaaagaaaagtttcgaaaaatatgtgacaaaagtatgataaggaaacgtaactgtttcttaaatgaagaacatctaaagcaaaacccaagattggcggagcacgagatgcaaactctggatgcacgtcaagacatgttggtagttgaggttccaaaacttgggaaggatgcttgtgcaaaggccatcaaagaatggggtcaacccaagtctaaaatcactcatttaatcttcactagcgcatcaaccactgacatgcccggtgcagactaccattgcgctaagcttctcggactcagtccctcagtgaagcgtgtgatgatgtatcaactaggctgttatggtggtggaaccgttctacgcattgccaaggacatagcagagaataacaaaggcgcacgagttctcgccgtgtgttgtgacataatggcttgcttgtttcgtgggccttcagagtctgacctcgaattactagtgggacaagctatctttggtgatggggctgctgctgtcattgttggagccgaacccgatgagtcagttggggaaaggccgatatttgagttagtgtcaactgggcaaacaatcttaccaaactcggaaggaactattgggggacatataagggaagcaggactgatatttgatttacataaggatgtgcctatgttgatctctaataatattgagaaatgtttgattgaggcatttactcctattgggattagtgattggaactccatattttggattacacacccaggtgggaaagctattttggacaaagtggaggagaagttggatctgaagaaggagaagtttgtggattcacgtcatgtgctgagtgagcatgggaatatgtctagctcaactgtcttgtttgttatggatgagttgaggaagaggtcgttggaggaagggaaatctaccactggagatggatttgagtggggtgttctttttgggtttggaccaggtttgactgtcgaaagagtggtcgtgcgtagtgttcccatcaaatattaa

SEQ ID NO: 12 - protein, 385 aa, Cannabis sativa
MNHLRAEGPASVLAIGTANPENILIQDEFPDYYFRVTKSEHMTQLKEKFRKICDKSMIRKRNCFLNEEHLKQNPRLAEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLDLKKEKFVDSRHVLSEHGNMSSSTVLFVMDELRKRSLEEGKSTTGDGFEWGVLFGFGPGLTVERVVVRSVPIKY

SEQ ID NO: 13 - DNA, 17257 bp, Cannabis sativa
  misc_feature: AAE1 Genomic Sequence CM011611.11210973- 1228229
tcttaaaattaattgggcataggaatatgctaattaattagagaatcaaaccttaaatactttagaaaaaggattatgacattttaaattcctagttgacattatcaatttacaacaatactcatttgcactattatatcaactttgtatttttaggccaatttaataattttagcttgtttttttttcatagtttgtgtttgttaaatttttacttattctcattactttattttattttgtttaaaccaatttgtgagtaattagttttataaattaatttttaattttcaatccctgtggagacgatactcgattatcactttattacttgttacgattgcatacacttacatatacgatttttcacaacattgatccttagagatcactctctataatctaagctactattgttaataaatgttggttacaaggtggtttcccttgggaattagatccatgcagaaaattgtttgagattgaatctcattaaatctgttgtctctcgttttttatgctacaatattctgttatagcatcaatctgttattttagttttttttttctaattttctttaattgtaattttttgttattctactttattattgaaactaatctatattaatagcgtgataaattataaaagtactacattaagtatttattatttcagtttttttttttctaaaagtaactattcttattctaaatcacaaatattatattgaagggatatgatgtagtggactatacagtgtagtcagtatttattaaattcaataaaaataatgtcaaatgcactaaaaatgtgttgaatgaaaatttatggttcaatatgactatcttaatcaaatctataatcctgccataagatttaacacatattttagagcatttaacttattattagtgcatttgataatgcaaactgctctatgtaatcaactacactttattcaaagcatatatgtatgtattatattataacacttcattcaaagtgagaaaattgttggcgcaaaacccttctcatgatcttgttggttgctgttctcggaagtgatgaaagaggcacaacacgagtgacctatttaataattaaccaaacaccaaattagaattagtatacaattataagatcatcatcatagtgtggaagtgtataggataagaatagtctaattttttactaaatatacacccttgagagtcaagactaaaaattaacaaacaaaaaagttaataatgatgaaattggtaccttgaacagaggatttagtttcttctgtaaacccaagttgaaagataacctcaattgatttaagtcaattgttgtatcatttgaatcttttaatacaaagaaaattactaattgctcaggtccaccccgcccaaaggtggcaccccaatagcagttgtctcgaaaactctgtcatcaacttcattacaaactcgttcaatctctatagaactaatctgtttacataaacaaaatcatagtcaattcaaatctacagttagaaatatatatgtagagttataatcctatcaaattatcaaaagtaacaagagatatagtagatttagttacatttgttattatccaagatcgatttagctgacctttcttaatttagctacttactcagttgcctatataagacaagtcttattcattatttagtgaatattttttagatcaccatagaagcacgaatctagagaaagagagtgcaagaagatcagataatgagagaatactgtgaaagagtttggggattcggtttttgtaatgagctacattgacgtacgaatttgtatgctcggatgagagtgtgaaagtgaaaaacttgtttgaacccaagattgagaatccttactcaactgtgatgtactcgatttgttgctaattaataaagaaatctgagatcgtattccaagctgaagtaggactaagtttacatcgaacttaggttcttaaccaggatatttcctactttgtctattgttagaatattagctgtatattaggtgtaaaagaattagggtaacttgtatttagtagtttcctattttgttggtagtttcctatttcctagtctctctagctttgtatataaatatgtactattctatgaaatatacacacaattcgaatcactattttctacatggtatcagagcattgtgattcaaaattcgaaattcgaaattagggttctgaaaaaaaaaaaaaaaaaaaaaattagggtttgagtttagggttgttttttcgaaaatcctatttggagtgaacattgtttctcaccgcccacataggaggactgcccagccgccgatctacaacccccgaagtccggtcgccggattcctcgcgcgtggggctcacgcgccgcctgaagcttctgcgcgtggggctcacgcgccggcgcgtggaggcgcgtgtgacggcgccttcgacggcgttctgttgccgattcttcactgggttcttctaagcccactcgcatctgttctaggttgtaattcggtatttgtttgtcttcttcgtgtttgggtgttcattctttggtgttcttttttctttttctctgtctttgtgtttgcttttgagattatggcagagataagatcagattcaaaatcagttgttgtttctgatgttgttcccgtatgtctaaaatcacggatcataagttgattggttcgaattatttggaatggagtaagacgattcgactgtatttgaggagtattgacaaagatgatcacttgactgacgatcctcctgaagaaacaaacgattcaaggaaaatatggctccgtgaggatgctcgcttgttccttcaaattcgaaattctagataatgaggtaattagtttgattaatcattgtgaattggttaaagaactaatgggttacttggagtttttgtattctggcaaagacaacatatctcgcatatatgatgtttgcaaagctttttatcgcgcggagaaacaagataagtctcttatgaattattttatggctttcaagaaaacatatgaagaacttaatgtgctattaccgtttagtcctgatgtaaaagttcaacagcgccaacgagaacagatggctattatgagttttcttgcaggactctcatctgaatttgactctgcaaagtcacaagttctctctggttctgatgtctcctctttacaagatgtgtttactcgtgttcttcgcacagagactacctcttcaactcctctgaatagcgccttggtgagtcgtaataattctgcaccggaaagaaactctactccaagtggatttaaaggaggtaattcacaaggacctgataaccgcacaccaaattctgggagcatcatgtgcaattattgtaagaaaccaggccacaccaagtttgagtgtaggaagttacaatttaagaatcgacaacaacactctgccaatattgcaagcactagtgatgcatctgacaaatcggtccttatttctgctgatgaatttgccaagttctcaaggtatcaggaaacacttaagtcttcatcttcttctgtcactgcgattgctgattcaggtaactctaatgcctgccttctttccaaatcctcaaaatgggtcattgattctggtgccacagatcatatgacaggtaattcccgtctcttttccacttttcagtctcacagtcccacttctgttgtcaccttagctgatgggtcaacagcttctgttcttggatctggcactattgttcctacacctatgctatctttatcatcagtcttacatttacctgatttgtcctttaatttgctttctgttagtcaactcactcgtaatctaaattgttgcatctcattctttcctgatcattgtttgtttcaggatcttatgacgaagaagattattggtaaaggacatgaatctggtggcttgtatgttcttgacccacttccgccaacctctattgcttgttcgagtgttgcttccgcttttgaggctcattgtcgtttaggtcatccatcccttcctttgctcaagaaactgtgtccccaatatagtagtttatcttcgttagaatgtgagtcatgtcagtttgccaaacatcatcgtgttagtttgagtccacgtgtcaataaacgtgctagtgttccttttgagttagttcattctgatgtttggggtccttctcctattttgtctcaacctggattcaggtattttgttacttttgtggatgatcattctcgtttaacttggttatatttaatgaaaaatcgttctgagttgttttctatattctgtgctttttgtgctgagattcaaactcaatttaatgtatctattcgtactttgagaagtgataatgccaaagagtacatgtctgctcaatttcaatcttatatgacctctaatggcatgcttcatgaaacttcttgtgttgatacggcacctcagaatggtgttgcagaacgtaaaaacagacatcttcttgaaactgcacgtgctctcttgtttcaaatgaaagtccctaaacctttttgggccgatgcagtttccacggcatgctttcttattaatcgcatgccctcttctgttcttaatggtgaaattccatttaaaattctttttcctaataagtcattatttccagttgctccgcgagtatttggtagtgtttgttttgctcgcgatgtccgtccatctgtcaccaaattagaccctaaggcactaaagtgtgtctttcttggttattctcgtcttcagaaagggtataggtgttattgtcctgatctcaacaaatatcttgtgtctattgatgttacttttgtagaaaatacaccttatttttcatcttccactacttctactagtcagggggaggatgatgatttgttggtttattctgtcacatcctatgcgcctgctccatgttctggcgggtctcttgttccttcacctgcctcggtcgtcacccccacaatgtctacacctccaccacctccctccaccacctccaccactccctcggcctcctatagtgtacaccaggcgcccccctcctccacctcctgtttcatgtcctgcacctgcatcttcgtcatcagatctggacatctcagatgatcttcccattgcactacgtaaaggtaaacgtcaatgtacttttcctattacttcttttgtgtcttataatcatctctcctcttcttcttgttcttttattgcttctcttgattctatcactattcctaaaactgttcgagaagcgatgtctcatcctggttggcttgctgcaatgatagaagagatgaatgctttggttgcgaatggtacttgggtcttggttgatttaccttccggaaaacaggccatcgggtgtaaatgggtgttcacggttaaattcaatccagacggcacagttgctcgcttaaaggcccgtcttgttgccaagggttatgctcaaacctatggagtggactattgtgatactttttctcctgttgctaaactcacatctgttcgactgttcatttccatggcagctactcatcattggcctttgcatcagcttgatattaaaaatgcttttcttcatggagatcttgaggaagaagtatatatggagcaacctcctgggtttgttgctcagggggagttagggcgagtttgtcatcttcgcaaatctttatatggattgaaacaaagccctcgtgcttggtttggtaaatttagtcaggctgttgagaagtttggtttgttgaaaagtaagtcagatcattctgtgttttataaaagatcaactgttggtatcattttgttagtggtgtatgttgatgacatcgttattactggaaatgatactgaaggcatctcatcccttaagtctttcattcacacccagtttaacacgaaagatttaggggaactcaagtatttcttggggattgaagttactcggagtaaacaaggtatttttctgtctcaaagaaagtatgtacttgatttgctaactgagacagggaaattgggatcaaaaccttgcaatgctccaatgaatacagctgtgcatcttacacgtgatgggaaaccatttgaagatcctgagaaatatcgcaggttagttggaaagttgaattatttaactgtgactcgtccagatattgcatttccagttagtgttatcagtcagttcatgtcttccccaacaattcatcattgggcagcattagaacaaattttatgttacttaaaaggagcaccaggacgaggtatagtttacaaggatcatggacatacccagattgagtgtttttctgatgcagattgggcaggctccaaggtagatagacgatccacttcaggatattgtgtatttgttgggggcaatctggtctcttggaagagtaagaaacaaaatgttgtatctagatccagtgctgaatcagaatatagagctatggcccagtccgtgtgtgaggtaatatggatttatcagcttttgactgaattcgggtttaagacttctattccagcaaaattatggtgtgataatcaagctgctcttcatattgcctcgaatcccgtattccacgagcgaactaagcacattgagatcgattgtcattttgttcgtgagaaaattcaacaaggtctgatctccacaggatatgtgaaaaccggagaacaactaggagatatttttacaaaagctttgaatggggtgcggatagactatctttgtaacaagctgggcatgattaacatctatgctccagcttgagggggagtgttagaatattagctgtatattaggtgtaaaagaattagggtaacttgtatttagtagtttcctattttgttggtagtttcctatttcctagtctcctagctttgtatataaatatgtactattctatgaaatatacgcacaattcgaatcactattttctacatctatgaactaagaatatgtgtcatatctaaaacctaattaagaattataaaaaatatatatgatcaaaatagtaaatttaaataaataaataaaaaaaattattggtgggcagggcaaacctaactaattaatagcacacataattcatttcataccttgatgcctccaatattcattgtatcatctgcacgaccatgtgcatgataataaccattagatgtaagctcaaaaatgtccccatgcctccttaaaacctctccattcaatgtaggcattcccttaaaataaacatcatggtgattaccattcaacagagtcttcgatgctccaaacatgactggaccgagcgctaattcaccaattcctggtttgtttttaggctgcatgtcataaagtatatatataatacaacaattcagtcaaatgtaacataattttatatgggattaatacaagaattgtgaatttaaattgatccatcaaatcttatttgaaaagtcttatccaatatttttaaattatattttataagtttattaattaccattggataaccattgttgtcaagtatgtataaagtgcaacccatacattgtgaactaaatgaagataatgattgagcttgtaagaaagagccagcagaaaatgcaccaccaatttctgtgccaccacacatttcgataacaggcttgtagtttgctctccccatcaaccataggtattcatctacattagatgcttcaccagaagaggaaaagcaactacaagaagggatataaaacttattttaactatccatataatacgtttaatataatctagattgatgttatgtgcatagataagaaatattggaaaatatattataattctttaccggatggtggaccaatcatagccactaacacaattgctacttttccatgatcgaacaatgctagggaccacacctagcattgttactttagcatcctacacacatattagaaaacattaaaataaagagaaataaagtgaaaataaaattatagtcaaaatgttttagctaaataccgaacttatcatgttttttattttaaaaaataataataataaaaatgatgtgattattggtgtactttcgttttcacccttctataatgaattagacatttacaaatataacttatctatagaaaagcacaaaacacataaataacataactaaaaagtttattagttaatggaagtttagtttacctgcacaaatttggcgaagctagaaacaagtggtgatccattatacaaggcaagagaagccccattaaggagtgaagcatagaccagccaaggacccatcatccaaccaagattagtgggccaaacaatgacatcacctttcctaatgtccaaatggctccacccatctgcagctgcttttaaaggagttgcttgggtccatggaattgcctttggctcccctataccaaaacaatattgtcttccatcaaaagtgtttagattgatgaacaaaactaattaaggatattaagggggctcatgagattttttaacggtctttgattttgagaatgtgttctttttcataccaagcatcattatttactgaattaccttttagtttgcgctcttaaaagtttagttgtagacatgttttgtttaaagataatagcgggcataagtactcaaaattttatattgtaagcggcataaactcattgtttattttagcttactcaatattgtaaaattgtaatttttctttaattttatccgtataaactctcttattgtcttaaacaggggatatataatgcctaaattcttgatcattaaatctacataaaaatatgtaatatcagttacttccaaatgctattttaataaattcaaaacatggcggtggcttcaattacctgttgttccagatgagaagaggatgtttgtataggcatcaacgggttgttctctagcagtaaattcacaatttctgcaggtaccactcaagaagatattagtcccacatatatgcttattattttggagataatacttttttttttcttttttttaataatattctagatataatacttaaaataatatagttctttttgtcagcatataactaatttgcctgtgttacttttaattaaaaaaaaataaattaataaaataaaaaataagatgaatttagttaagccccaaaataactcctaaattaataatgcccaacttgataaaacccctgataataagaaacgtgatacttatttccactaacattcacatgtattttagttcaaactccaatggatccaaatgttcatatatatagatatgtatatattcacctctactgtttatatattccacattaatctctatacgaattaaacaattgaaattgatgtaacaaaagaagtaaaataaattaacttacttaaactcttttgctctttctagaaagtaatcccaagaaatatcgccatcacgcaattctgcacaatattagagccactacaaggaataacaatggccatgggagacttggcttccacaactctactgcacaatttaacttattttgtgagatggatgaatgaataataataataataatttattattatttgtcaggaatgataaattttgaatgtgtactaattaatgatatttaaatgtcattttttaattaactaattcataaatgtaattaaaaatttaaaatcggataaagagaatgtgacctgtataagggaatacgcttcttccacgaataatagatcctgaaaacgaaggaattaattaagtaaaaaaaaaaaataactaaataggatatatatatatatatatatggtgactattcaatggttacatttttattgtaaccatatggttacatttttttttaacctgtaatagcaggttactaataggtaaactttccttttttagatttaattaattacaattaactattttcttaaaaataattaaatagacattctttttttagaattatttaatttaataaactattttcttaaaataattaattaaacatttaacaagacctctttagcaatataatatttatatttaaatccttacttgacttattttaataattaaaccatttaattataatattacaataaatatttttttttttacaaaaattatactcttttgacacaaattaaaattccttcttataaaatttcaaataattaaatattttaaatgatatttaattattttgttacaattatatgaactaagtatgaggcctcaagctaatcaatcgataacagtgcagccattttttttctaaaaaatccttcaacttatttcattttttactttttaaatatttaaataaaaaaattaaataattaagtgtaataatttaaaattaagattacaagtataataaaatttaaattatgaaattatatgtgtataattttaaattataaaaagttttgctataaaattttaaataatttaagtataaaaaaaataaattgctaaaagatccttatatagtaatgaattgtaaaaaattaattaataattataattaatttaattttaaaatataattaatcttaattaaagttttataaggaaataaatgattaggttactttcaggttacaagttatttattatttttttttaatttccaaataatcacaaccatttaatagtaatctaatggcttaaaaaaatgtaaccatgatggttacaataaaatgtaaccatgaaacctcttccatatatatatatatatatatatatatatattatattatatatatatatattaccttcttactaaataatataaagagaaatgacattatatattgttgggtaaatatcattttggaccctgtattttgcaaaagttacaaattggactttgtgttttgttaaatgacaaaatggaccctctattttctaaaatagtaaaaatagaaccctgagcttaatttttgacaacttttttttaatacaaccaacttgaagacaatgcttaatacgaacagatacaaaaatgtaaacattttgtcatagcacttttagatcggattataattaaattttattttgacaaaaaatcaattcagggtcctatttgtactattttagaaatacagggtctattttgtcatttaacaaacacagggtccaattaattttgtaaaacagaaggtccaaatggtatttacccctatatgttacatgttatattgtacaaattgatatcacacaacattactagctatcttggcaaaggaaacagcaaattaaaactaaattactagctattggcataaaatttggagaagtatactttatcgtcagtggccgagagagatttgcaaagtggagggcaaattgttaagacaaagatttggggtacttattattaataattattatataatttaattttcttacatatttaatatatagatgaattttacttttttgggaggactatagccccactaagtgcctccgtcaatgtatatcgtactagtttaatatcagcagtattcagtaaccattatatgtaggtgacatttaattggaggtaatgaaataaaatagaatgaaaaaaaaaataaaaaatatttgattcctggttgcatttaaactattagaataccattccaatagaatgacctttctaatatattttagtggaatgactattccattttgaaatagaaggaaagattattccaaagcaagatttgaaaaactttaataatttttttttatcaatttttttatgcattttaaattttattccattccattcctatttcaattcacattcttattcctattcctatatttcattactcccaaccaaacgtcacctaaatatttttaatttttattttgatagtatatattttaattgtataatttgtttttttttatgttattcaataaaaaaaattataacaaatatttaatttacaagttatttattataataataattttaatttaattagtgtaatacttttaatgttataatagttttcattgtttttgaaattttgtacactctttttaattttttttaaataataataataaaatatatttttttggtaaaaaaataataataaattttttttttttaaatatatgcaatgtgttataatttatatgcaatatcaaaaaaaaaaaattaacttatatagacacatttaatataaggtaaaaaataagagagatatgtatattgagtttttaaatttaaaattggaattttttagaaaaaaaaaaaataaaacttttgtcatatatctgcatttaatataacatagacatagatataattctttaaattaataatctaaaataaaatatacatatacatatgaagtaatggtcggtgaaatgtttttatttttaattttttacacttaatttcttatattttttgtggcaaaacctctttatgttttaaatttacttttctttgtaaatttgatgcgtcagttaaatcacaattaaatactagttggatgaactgtttacacatgtgtatatgtgaaagtaaacatcgaagtcaatacaataataatttagttggtattttagcagtgatattcagccgacaccttaaatttgcatagaaaagtaaacataaaaagattttgctacaatttagagattgatttttaattttcttttttatcttacacacatttaaattatgtttccttaagtattggaacatcaatttttaatgaattaacttaagaacatatttaaattatcaagctttcttaaacactaataattgcatccgatatttacttttaattgtcaaaattataattatattatatagatacacataataataataatctcacctaataactaatatttttttctttaattttaattatatcacttttaaataacataaaaaaaaaactctcataaaatttaatatacgtatctatcacgtaactttttgctagtacatgtctatataaggagtgagtatttatagaaattatatatatacctgtgtaaaaatggcttttgcttttgatattcgaagtcttgttgatatttcaggagcagaaaaactatcagcaatagaaacaactacatatcccgcaagaacaatagctagatagataaccacagcatccacatgcattggcatatcaattgcaattgcacaacccttctccaaacccatttcttcaagtgcataaccaactaaccttcaaattaattaccatacatagtttcaatatccaaataaaccatatcatattaactttatgaattcttgttatatgtaaaaacagaaagatttaaagtaaattcaatgtatatatatacccaacacgtttacgcaattggtcaagtgtcaatttgtttagaggcaaatcatcatttccttcatcacgccatacaatcattgtatcattcaatttcttgttactatttacattcaagcaattctttgctgagttaagataacctcctggaagccattcactaccccctggattattaatatcatcatctctacgcaatatacattctggatcctttgaaaaacttatcttcatctcatccattagtaatgttctccaatacacctaacattatataatattaaccattaattacaaataattaacataaaaaccaccctcaaataaaataataaatagttattattattacctcagggtttcttacagaaaattcttggaaatgagaaaagcttgaaatgggatccttatacttgactcccaaaaactcttttcctcgcttctccaaaagtgcgcccatattggtggactttactttctccctgaatcataataaaagaaaaacaaccattaatgattttggtgttcattcatcttgtgtgatcgatcgccttctaattaattactcttaattaggcatgccttctcggatatacaacatatattataagatttaaatttcaatgaacaccttataatatagagcaccatgctgccttgcacctctagatatatatgtcgccttgtgattgactaacgatactctttaaaaattattatattaaactatataggacccgatacttatttaaacaatagcaatattaacacataagaggatgctaaacactagtgccttttagtatttctctataatatcttcaatgattagtttacaaaactgcatttatcaacaaagttattatctcctaacacaaaaaaaaaaggacacccaaagttgttgttttttatgaacattttagtcacaacataaatttttaatgattaaatgtaatctttattcacaataaaaaattaattgtgattaaagcatataatattagatataagatatcactaatatacttttagttataatattgtgactaaaaatacattaattataataaattatggtggcgtttggtaacactttttaatcagttttctatttttaaaagtagaaaagtaaaaatatttttcaaaaacatgttctataaaactgttttttactttttcaattttataattagaaatcaaaattttaaaaacaaaaaaaaatcactttcaatatttttttaaatagtttttttttcttaatcaatcttttagattacgactagacccggatccaaatcccctccccacgccctggcgctgaacccggcttctgacttgaacccgaatctgaccccgacctagacccgacttaaataaaatcaaaaagtaaaaataaaaatgaactttacagaacacacttttgttttctgttattaaaatcaaaaaaataaagtggttacagacatatttttgtttttcaaaaataattttttaaaaacaaaaattttactttcattttgtgattaaaaaattaaaaaacaaaaatgttaccaaacggcacctatatttttttttataactaatatttttaactacgaattttttaatcaaaatataaaaataataatttataagtagctatcaattttttacttctaatcataaaattttattattattattaaaagtaaattttttatagtgctccttaattgtttaattgtttgacaaatgaaaagttggcatatcatttagcttaataattcaatttaacaatattgtttgtaattatagggaaatgtgaagtaagtgaatttggttggcacacaaaaatattatattaattaataagaaaagtgaatccatgaattttaattatattaaaaaacattaagagtgtgaccagaaaattaatgtgtaattagaattgagtgtaatttgaagtattataagatgatacatatttgtttaagtaattgcacggcaattataatataatggaaagtaattgaggagtcttaattatattctcaaattctcatagtcttactaggattattggatatgtataattatattgtgtaactataagatattatatttttttagctaattaatgttattaaactattattaactcatttgttaaacgtaaaaagaagaagtcatatgtaattgtactccacatcaaatacataaatgtattttaaaaaagagtgtaattacaagtaagatacagcaataacaacgaatttgaatgtagtgacgattataaatgagatacactactaatgataatagctttaaatatagtgatgactacaattgatgcagtcgtgataataactttaaatatagtgacgacaacgagtaaatacggactttaatttttttaacttttttttttcaaatttaaagtttgggtttctaaagtagtttctctggtagtttttacgtgtgttgctacacgatttattattgcgatttaagttgcatcgctagttgcaatagagatttctatagaattccgtaaaaaaaaaaaaaaaaaaaaaaaaaaaaactgtttttaaagtgtaaaaatgaaaaaatatatatatagtaaggaaagagagagattacgggtcgggtatccatgcaggaggggcgggtccaaagtctttatagcaaccatagtagagcatctggtgcagggagaaggggaggtcaggcgagagaatatggttggcaatattgatccatgtttggggagtggcagcgccataattacacacgatctcggccagtctaccatggagtgtctcagcaacttcaggggtgatacctagggctatgaagtcagaggccacaacagagtccagggacttgtaattcttacccattattacttctattaaactgtgctctgctattttagaagatagaaagagagagtttgaatattatatatatttatattggattttgggatgggttatttgatctaacaacatgcactttttttatcaaagacttgctcaataatggattaggctggaatttggtttgggtacgtactacacaacctatcacttgtgttgcctttgaacatgcatataaattaatatacttactcctacttatttatcatgagaaatgctaaaagctacttattttctttaattaattaatattgagggacaattattttacttttaataaaaattataaacttatataaaacgtcaccgtttagcatcttggtacacataacaatgcatatttattttattaacaaattggaatagaaaaaaaaatgtacgtatatagtgtagggtaggtagtgtagggttgttttatgcatggtgttaagtaactagggcggttcagccaccaaatttttatttcttccacaatcaattttccaatgaattagtcgtttcttttttttttaaatttaacaaacactatatgtaaatctaccatttcaattagtactacttgtttcttgtaaaatcaactttaattttgtacatattctcaactgaaatttatttatttgtttgccatattatctatcttacaatattaacttaattagtatcttattatttaaaattcgtccaataatacaattgcaatagattatatgggctcgtttagtatgtcgtataaaggtcatattgtattaaataataaataacattatatttggataaagtaatatattgtattaattattacgaccaaattttttaatacaatatatgttgtattatttaatacataataatggtctgtattagcttatattataatatttacaaagaatttaaaatcaacatcaatcttcaacccggacccgaacccagacccagacccaaacccatacccagacccgga

SEQ ID NO: 14 - DNA, 2166 bp, Cannabis sativa
  CDS (1)..(2166)
atgggtaagaattacaagtccctggactctgttgtggcctctgacttcatagccctaggtatcacccctgaagttgctgagacactccatggtagactggccgagatcgtgtgtaattatggcgctgccactccccaaacatggatcaatattgccaaccatattctctcgcctgacctccccttctccctgcaccagatgctctactatggttgctataaagactttggacccgcccctcctgcatggatacccgacccggagaaagtaaagtccaccaatatgggcgcacttttggagaagcgaggaaaagagtttttgggagtcaagtataaggatcccatttcaagcttttctcatttccaagaattttctgtaagaaaccctgaggtatattggagaacattactaatggatgagatgaagataagtttttcaaaggatccagaatgtatattgcgtagagatgatgatattaataatccagggggtagtgaatggcttccaggaggttatcttaactcagcaaagaattgcttgaatgtaaatagtaacaagaaattgaatgatacaatgattgtatggcgtgatgaaggaaatgatgatttgcctctaaacaaattgacacttgaccaattgcgtaaacgtgtttggttagttggttatgcacttgaagaaatgggtttggagaagggttgtgcaattgcaattgatatgccaatgcatgtggatgctgtggttatctatctagctattgttcttgcgggatatgtagttgtttctattgctgatagtttttctgctcctgaaatatcaacaagacttcgaatatcaaaagcaaaagccatttttacacaggtatataaaaaaagagttaaattgtgcagtagagttgtggaagccaagtctcccatggccattgttattccttgtagtggctctaatattggtgcagaattgcgtgatggcgatatttcttgggattactttctagaaagagcaaaagagtttaagattatcaggggtaattgtgaatttactgctagagaacaacccgttgatgcctatacaaacatcctcttctcatctggaacaacaggtgagccaaaggcaattccatggacccaagcaactcctttaaaagcagctgcagatgggtggagccatttggacattaggaaaggtgatgtcattgtttggcccactaatcttggttggatgatgggtccttggctggtctatgcttcactccttaatggggcttctcttgccttgtataatggatcaccacttgtttctagcttcgccaaatttgtgcaggatgctaaagtaacaatgctaggtgtggtccctagcattgttcgatcatggaaaagtagcaattgtgttagtggctatgattggtccaccatccggtgcttttcctcttctggtgaagcatctaatgtagatgaatacctatggttgatggggagagcaaactacaagcctgttatcgaaatgtgtggtggcacagaaattggtggtgcattttctgctggctctttcttacaagctcaatcattatcttcatttagttcacaatgtatgggttgcactttatacatacttgacaacaatggttatccaatgcctaaaaacaaaccaggaattggtgaattagcgctcggtccagtcatgtttggagcatcgaagactctgttgaatggtaatcaccatgatgtttattttaagggaatgcctacattgaatggagaggttttaaggaggcatggggacatttttgagcttacatctaatggttattatcatgcacatggtcgtgcagatgatacaatgaatattggaggcatcaagattagttctatagagattgaacgagtttgtaatgaagttgatgacagagttttcgagacaactgctattggggtgccacctttgggcggtggacctgagcaattagtaattttctttgtattaaaagattcaaatgatacaacaattgacttaaatcaattgaggttatctttcaacttgggtttacagaagaaactaaatcctctgttcaaggtcactcgtgttgtgcctctttcatcacttccgagaacagcaaccaacaagatcatgagaagggttttgcgccaacaattttctcactttgaatga

SEQ ID NO: 15 - protein, 721 aa, Cannabis sativa
MGKNYKSLDSVVASDFIALGITPEVAETLHGRLAEIVCNYGAATPQTWINIANHILSPDLPFSLHQMLYYGCYKDFGPAPPAWIPDPEKVKSTNMGALLEKRGKEFLGVKYKDPISSFSHFQEFSVRNPEVYWRTLLMDEMKISFSKDPECILRRDDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRISKAKAIFTQVYKKRVKLCSRVVEAKSPMAIVIPCSGSNIGAELRDGDISWDYFLERAKEFKIIRGNCEFTAREQPVDAYTNILFSSGTTGEPKAIPWTQATPLKAAADGWSHLDIRKGDVIVWPTNLGWMMGPWLVYASLLNGASLALYNGSPLVSSFAKFVQDAKVTMLGVVPSIVRSWKSSNCVSGYDWSTIRCFSSSGEASNVDEYLWLMGRANYKPVIEMCGGTEIGGAFSAGSFLQAQSLSSFSSQCMGCTLYILDNNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 16 - DNA, 20 bp, Cannabis sativa
  misc_feature (1)..(20): Complimentary to position 919 of SEQ. ID NO. 1 THCAS CM011610.122243181-22246809
tataactttatattggagcg

SEQ ID NO: 17 - DNA, 20 bp, Cannabis sativa
  misc_feature: Complimentary to position 920 of SEQ. ID NO. 1 THCAS CM011610.122243181-22246809
ctataactttatattggagc

SEQ ID NO: 18 - DNA, 20 bp, Cannabis sativa
  misc_feature: Complimentary to position 921 of SEQ. ID NO. 1 THCAS CM011610.122243181-22246809
tctataactttatattggag

SEQ ID NO: 19 - DNA, 20 bp, Cannabis sativa
  misc_feature: Complimentary to position 926 of SEQ. ID NO. 1 THCAS CM011610.122243181-22246809
tcctttctataactttatat

SEQ ID NO: 20 - DNA, 20 bp, Cannabis sativa
  misc_feature: Complimentary to position 936 of SEQ. ID NO. 1 THCAS CM011610.122243181-22246809
tccaatataaagttatagaa

SEQ ID NO: 21 - DNA, 20 bp, Cannabis sativa
  misc_feature: Complimentary to position 1013 of SEQ. ID NO. 1 THCAS CM011610.122243181-22246809
actactcaacattctccttt

SEQ ID NO: 22 - DNA, 20 bp, Cannabis sativa
  misc_feature: Complimentary to position 1017 of SEQ. ID NO. 1 THCAS CM011610.122243181-22246809
aattttgtaaacaaacctaa

SEQ ID NO: 23 - DNA, 20 bp, Cannabis sativa
  misc_feature: Complimentary to position 1085 of SEQ. ID NO. 1 THCAS CM011610.122243181-22246809
caatttaggaaattttcttg

SEQ ID NO: 24 - DNA, 20 bp, Cannabis sativa
  misc_feature: Complimentary to position 1099 of SEQ. ID NO. 1 THCAS CM011610.122243181-22246809
tatattgggagaagcaattt

SEQ ID NO: 25 - DNA, 20 bp, Cannabis sativa
  misc_feature: Complimentary to position 1113 of SEQ. ID NO. 1 THCAS CM011610.122243181-22246809
tggattgttatgaatatatt

SEQ ID NO: 26 - DNA, 20 bp, Cannabis sativa
  misc_feature: Complimentary to position 1114 of SEQ. ID NO. 1 THCAS CM011610.122243181-22246809
ctggattgttatgaatatat

SEQ ID NO: 27 - DNA, 20 bp, Cannabis sativa
  misc_feature: Complimentary to position 1133 of SEQ. ID NO. 1 THCAS CM011610.122243181-22246809
tatacgagttttagatttgc

SEQ ID NO: 28 - DNA, 20 bp, Cannabis sativa
  misc_feature: Complimentary to position 1168 of SEQ. ID NO. 1 THCAS CM011610.122243181-22246809
tcaggacagacatatacaat

SEQ ID NO: 29 - DNA, 20 bp, Cannabis sativa
  misc_feature: Complimentary to position 1186 of SEQ. ID NO. 1 THCAS CM011610.122243181-22246809
gattttgtattgtcaaattc

SEQ ID NO: 30 - DNA, 20 bp, Cannabis sativa
  misc_feature: Complimentary to position 1218 of SEQ. ID NO. 1 THCAS CM011610.122243181-22246809
tggttttggggttgtatcag

SEQ ID NO: 31 - DNA, 20 bp, Cannabis sativa
  misc_feature: Complimentary to position 1230 of SEQ. ID NO. 1 THCAS CM011610.122243181-22246809
gacaataacgagtggttttg

SEQ ID NO: 32 - DNA, 20 bp, Cannabis sativa
  misc_feature: Complimentary to position 1231 of SEQ. ID NO. 1 THCAS CM011610.122243181-22246809
tgacaataacgagtggtttt

SEQ ID NO: 33 - DNA, 20 bp, Cannabis sativa
  misc_feature: Complimentary to position 1232 of SEQ. ID NO. 1 THCAS CM011610.122243181-22246809
gtgacaataacgagtggttt

SEQ ID NO: 34 - DNA, 20 bp, Cannabis sativa
  misc_feature: Complimentary to position 1238 of SEQ. ID NO. 1 THCAS CM011610.122243181-22246809
gaaggagtgacaataacgag

SEQ ID NO: 35 - DNA, 20 bp, Cannabis sativa
  misc_feature: Complimentary to position 1256 of SEQ. ID NO. 1 THCAS CM011610.122243181-22246809
tggatatgggagacatttga

SEQ ID NO: 36 - DNA, 20 bp, Cannabis sativa
  misc_feature: Complimentary to position 1269 of SEQ. ID NO. 1 THCAS CM011610.122243181-22246809
tagaatagtggcttggatat

SEQ ID NO: 37 - DNA, 20 bp, Cannabis sativa
  misc_feature: Complimentary to position 1270 of SEQ. ID NO. 1 THCAS CM011610.122243181-22246809
atagaatagtggcttggata

SEQ ID NO: 38 - DNA, 20 bp, Cannabis sativa
  misc_feature: Complimentary to position 1276 of SEQ. ID NO. 1 THCAS CM011610.122243181-22246809
tggagcatagaatagtggct

SEQ ID NO: 39 - DNA, 20 bp, Cannabis sativa
  misc_feature: Complimentary to position 1281 of SEQ. ID NO. 1 THCAS CM011610.122243181-22246809
tttcttggagcatagaatag

SEQ ID NO: 40 - DNA, 20 bp, Cannabis sativa
  misc_feature: Complimentary to position 1296 of SEQ. ID NO. 1 THCAS CM011610.122243181-22246809
aatctgcaagccaactttct

SEQ ID NO: 41 - DNA, 20 bp, Cannabis sativa
  misc_feature: Complimentary to position 1297 of SEQ. ID NO. 1 THCAS CM011610.122243181-22246809
attctatgctccaagaaagt

SEQ ID NO: 42 - DNA, 20 bp, Cannabis sativa
  misc_feature: Complimentary to position 1321 of SEQ. ID NO. 1 THCAS CM011610.122243181-22246809
ttgcagattcgaactcgaag

SEQ ID NO: 43 - DNA, 20 bp, Cannabis sativa
  misc_feature: Complimentary to position 1324 of SEQ. ID NO. 1 THCAS CM011610.122243181-22246809
cagattcgaactcgaagcgg

SEQ ID NO: 44 - DNA, 20 bp, Cannabis sativa
  misc_feature: Complimentary to position 1336 of SEQ. ID NO. 1 THCAS CM011610.122243181-22246809
aggacaaaccctcagcatca

SEQ ID NO: 45 - DNA, 20 bp, Cannabis sativa
  misc_feature: Complimentary to position 1338 of SEQ. ID NO. 1 THCAS CM011610.122243181-22246809
aagcggtggccatgatgctg

SEQ ID NO: 46 - DNA, 20 bp, Cannabis sativa
  misc_feature: Complimentary to position 1339 of SEQ. ID NO. 1 THCAS CM011610.122243181-22246809
agcggtggccatgatgctga

SEQ ID NO: 47 - DNA, 20 bp, Cannabis sativa
  misc_feature: Complimentary to position 1356 of SEQ. ID NO. 1 THCAS CM011610.122243181-22246809
aaatgggacttgagatgtgt

SEQ ID NO: 48 - DNA, 20 bp, Cannabis sativa
  misc_feature: Complimentary to position 1372 of SEQ. ID NO. 1 THCAS CM011610.122243181-22246809
tcaagtctactataacaaat

SEQ ID NO: 49 - DNA, 20 bp, Cannabis sativa
  misc_feature: Complimentary to position 1373 of SEQ. ID NO. 1 THCAS CM011610.122243181-22246809
ctcaagtctactataacaaa

SEQ ID NO: 50 - DNA, 20 bp, Cannabis sativa
  misc_feature: Complimentary to position 1398 of SEQ. ID NO. 1 THCAS CM011610.122243181-22246809
agacttgagaaacatgcatt

SEQ ID NO: 51 - DNA, 20 bp, Cannabis sativa
  misc_feature: Complimentary to position 1429 of SEQ. ID NO. 1 THCAS CM011610.122243181-22246809
cggcttcaacccacgcagtt

SEQ ID NO: 52 - DNA, 20 bp, Cannabis sativa
  misc_feature: Complimentary to position 1430 of SEQ. ID NO. 1 THCAS CM011610.122243181-22246809
atattcgtagccaaactgcg

SEQ ID NO: 53 - DNA, 20 bp, Cannabis sativa
  misc_feature: Complimentary to position 1431 of SEQ. ID NO. 1 THCAS CM011610.122243181-22246809
tattcgtagccaaactgcgt

SEQ ID NO: 54 - DNA, 20 bp, Cannabis sativa
  misc_feature: Complimentary to position 1441 of SEQ. ID NO. 1 THCAS CM011610.122243181-22246809
caaactgcgtgggttgaagc

SEQ ID NO: 55 - DNA, 20 bp, Cannabis sativa
  misc_feature: Complimentary to position 1449 of SEQ. ID NO. 1 THCAS CM011610.122243181-22246809
aacttctccaagggtagctc

SEQ ID NO: 56 - DNA, 20 bp, Cannabis sativa
  misc_feature: Complimentary to position 1453 of SEQ. ID NO. 1 THCAS CM011610.122243181-22246809
gttgaagccggagctaccct

SEQ ID NO: 57 - DNA, 20 bp, Cannabis sativa
  misc_feature: Complimentary to position 1458 of SEQ. ID NO. 1 THCAS CM011610.122243181-22246809
ccaataataaacttctccaa

SEQ ID NO: 58 - DNA, 20 bp, Cannabis sativa
  misc_feature: Complimentary to position 1459 of SEQ. ID NO. 1 THCAS CM011610.122243181-22246809
tccaataataaacttctcca

SEQ ID NO: 59 - DNA, 20 bp, Cannabis sativa
  misc_feature: Complimentary to position 1469 of SEQ. ID NO. 1 THCAS CM011610.122243181-22246809
cccttggagaagtttattat

SEQ ID NO: 60 - DNA, 20 bp, Cannabis sativa
  misc_feature: Complimentary to position  1504 of SEQ. ID NO. 1 THCAS CM011610.122243181-22246809
aatgagaatcttagttttcc

SEQ ID NO: 61 - DNA, 20 bp, Cannabis sativa
  misc_feature: Complimentary to position 1507 of SEQ. ID NO. 1 THCAS CM011610.122243181-22246809
gagaatcttagttttcctgg

SEQ ID NO: 62 - DNA, 20 bp, Cannabis sativa
  misc_feature: Complimentary to position 1508 of SEQ. ID NO. 1 THCAS CM011610.122243181-22246809
agaatcttagttttcctggt

SEQ ID NO: 63 - DNA, 20 bp, Cannabis sativa
  misc_feature: Complimentary to position 1511 of SEQ. ID NO. 1 THCAS CM011610.122243181-22246809
acagtagggcaatacccacc

SEQ ID NO: 64 - DNA, 20 bp, Cannabis sativa
  misc_feature: Complimentary to position 1525 of SEQ. ID NO. 1 THCAS CM011610.122243181-22246809
ggtgggtattgccctactgt

SEQ ID NO: 65 - DNA, 20 bp, Cannabis sativa
  misc_feature: Complimentary to position 1525, -1, of SEQ. ID NO. 1 THCAS CM011610.122243181-22246809
gtccacctacgccaacagta

SEQ ID NO: 66 - DNA, 20 bp, Cannabis sativa
  misc_feature: Complimentary to position 1526 of SEQ. ID NO. 1 THCAS CM011610.122243181-22246809
tgtccacctacgccaacagt

SEQ ID NO: 67 - DNA, 20 bp, Cannabis sativa
  misc_feature: Complimentary to position 1531 of SEQ. ID NO. 1 THCAS CM011610.122243181-22246809
tattgccctactgttggcgt

SEQ ID NO: 68 - DNA, 20 bp, Cannabis sativa
  misc_feature: Complimentary to position 1534 of SEQ. ID NO. 1 THCAS CM011610.122243181-22246809
tgccctactgttggcgtagg

SEQ ID NO: 69 - DNA, 20 bp, Cannabis sativa
  misc_feature: Complimentary to position 1546 of SEQ. ID NO. 1 THCAS CM011610.122243181-22246809
ggcgtaggtggacactttag

SEQ ID NO: 70 - DNA, 20 bp, Cannabis sativa
  misc_feature: Complimentary to position 1549 of SEQ. ID NO. 1 THCAS CM011610.122243181-22246809
gtaggtggacactttagtgg

SEQ ID NO: 71 - DNA, 20 bp, Cannabis sativa
  misc_feature: Complimentary to position 1552 of SEQ. ID NO. 1 THCAS CM011610.122243181-22246809
ggtggacactttagtggagg

SEQ ID NO: 72 - DNA, 20 bp, Cannabis sativa
  misc_feature: Complimentary to position 1558 of SEQ. ID NO. 1 THCAS CM011610.122243181-22246809
cactttagtggaggaggcta

SEQ ID NO: 73 - DNA, 20 bp, Cannabis sativa
  misc_feature: Complimentary to position 1579 of SEQ. ID NO. 1 THCAS CM011610.122243181-22246809
ggagcattaatgcgaaatta

SEQ ID NO: 74 - DNA, 20 bp, Cannabis sativa
  misc_feature: Complimentary to position 1591 of SEQ. ID NO. 1 THCAS CM011610.122243181-22246809
caatgatattatcagctgcg

SEQ ID NO: 75 - DNA, 20 bp, Cannabis sativa
  misc_feature: Complimentary to position 1627 of SEQ. ID NO. 1 THCAS CM011610.122243181-22246809
gcacacttagtcaatgttga

SEQ ID NO: 76 - DNA, 20 bp, Cannabis sativa
  misc_feature: Complimentary to position 1653 of SEQ. ID NO. 1 THCAS CM011610.122243181-22246809
agttctagatcgaaaatcca

SEQ ID NO: 77 - DNA, 20 bp, Cannabis sativa
  misc_feature: Complimentary to position 1654 of SEQ. ID NO. 1 THCAS CM011610.122243181-22246809
gttctagatcgaaaatccat

SEQ ID NO: 78 - DNA, 20 bp, Cannabis sativa
  misc_feature: Complimentary to position 1655 of SEQ. ID NO. 1 THCAS CM011610.122243181-22246809
ttctagatcgaaaatccatg

SEQ ID NO: 79 - DNA, 20 bp, Cannabis sativa
  misc_feature: Complimentary to position 1656 of SEQ. ID NO. 1 THCAS CM011610.122243181-22246809
tctagatcgaaaatccatgg

SEQ ID NO: 80 - DNA, 20 bp, Cannabis sativa
  misc_feature: Complimentary to position 1659 of SEQ. ID NO. 1 THCAS CM011610.122243181-22246809
ccaaaatagatcttccccca

SEQ ID NO: 81 - DNA, 20 bp, Cannabis sativa
  misc_feature: Complimentary to position 1670 of SEQ. ID NO. 1 THCAS CM011610.122243181-22246809
ccatgggggaagatctattt

SEQ ID NO: 82 - DNA, 20 bp, Cannabis sativa
  misc_feature: Complimentary to position 1671 of SEQ. ID NO. 1 THCAS CM011610.122243181-22246809
catgggggaagatctatttt

SEQ ID NO: 83 - DNA, 20 bp, Cannabis sativa
  misc_feature: Complimentary to position 1681 of SEQ. ID NO. 1 THCAS CM011610.122243181-22246809
gatctattttgggctatacg

SEQ ID NO: 84 - DNA, 20 bp, Cannabis sativa
  misc_feature: Complimentary to position 1684 of SEQ. ID NO. 1 THCAS CM011610.122243181-22246809
ctattttgggctatacgtgg

SEQ ID NO: 85 - DNA, 20 bp, Cannabis sativa
  misc_feature: Complimentary to position 1687 of SEQ. ID NO. 1 THCAS CM011610.122243181-22246809
ttttgggctatacgtggtgg

SEQ ID NO: 86 - DNA, 20 bp, Cannabis sativa
  misc_feature: Complimentary to position 1690 of SEQ. ID NO. 1 THCAS CM011610.122243181-22246809
tgggctatacgtggtggtgg

SEQ ID NO: 87 - DNA, 20 bp, Cannabis sativa
  misc_feature: Complimentary to position 1702 of SEQ. ID NO. 1 THCAS CM011610.122243181-22246809
ggtggtggaggtgaaaactt

SEQ ID NO: 88 - DNA, 20 bp, Cannabis sativa
  misc_feature: Complimentary to position 1718 of SEQ. ID NO. 1 THCAS CM011610.122243181-22246809
actttggaatcattgcagcg

SEQ ID NO: 89 - DNA, 20 bp, Cannabis sativa
  misc_feature: Complimentary to position 1731 of SEQ. ID NO. 1 THCAS CM011610.122243181-22246809
tgcagcgtggaaaattagac

SEQ ID NO: 90 - DNA, 20 bp, Cannabis sativa
  misc_feature: Complimentary to position 1748 of SEQ. ID NO. 1 THCAS CM011610.122243181-22246809
gactggttgctgtcccatca

SEQ ID NO: 91 - DNA, 20 bp, Cannabis sativa
  misc_feature: Complimentary to position 1749 of SEQ. ID NO. 1 THCAS CM011610.122243181-22246809
actggttgctgtcccatcaa

SEQ ID NO: 92 - DNA, 20 bp, Cannabis sativa
  misc_feature: Complimentary to position 1750 of SEQ. ID NO. 1 THCAS CM011610.122243181-22246809
tgaatatagtagcccttgat

SEQ ID NO: 93 - DNA, 20 bp, Cannabis sativa
  misc_feature: Complimentary to position 1751 of SEQ. ID NO. 1 THCAS CM011610.122243181-22246809
ctactatattcagtgttaaa

SEQ ID NO: 94 - DNA, 20 bp, Cannabis sativa
  misc_feature: Complimentary to position 1772 of SEQ. ID NO. 1 THCAS CM011610.122243181-22246809
ctactatattcagtgttaaa

SEQ ID NO: 95 - DNA, 20 bp, Cannabis sativa
  misc_feature: Complimentary to position 1779 of SEQ. ID NO. 1 THCAS CM011610.122243181-22246809
attcagtgttaaaaggaata

SEQ ID NO: 96 - DNA, 20 bp, Cannabis sativa
  misc_feature: Complimentary to position 1789 of SEQ. ID NO. 1 THCAS CM011610.122243181-22246809
aaaaggaatatggagataca

SEQ ID NO: 97 - DNA, 20 bp, Cannabis sativa
  misc_feature: Complimentary to position 1790 of SEQ. ID NO. 1 THCAS CM011610.122243181-22246809
aaaggaatatggagatacat

SEQ ID NO: 98 - DNA, 20 bp, Cannabis sativa
  misc_feature: Complimentary to position 1814 of SEQ. ID NO. 1 THCAS CM011610.122243181-22246809
ttgtcaagttatttaacaaa

SEQ ID NO: 99 - DNA, 20 bp, Cannabis sativa
  misc_feature: Complimentary to position 1874 of SEQ. ID NO. 1 THCAS CM011610.122243181-22246809
tcatgactcacttcataacc

SEQ ID NO: 100 - DNA, 20 bp, Cannabis sativa
  misc_feature: Complimentary to position 1881 of SEQ. ID NO. 1 THCAS CM011610.122243181-22246809
ttgattatctataatattcc

SEQ ID NO: 101 - DNA, 20 bp, Cannabis sativa
  misc_feature: Complimentary to position 1894 of SEQ. ID NO. 1 THCAS CM011610.122243181-22246809
aggaatattatagataatca

SEQ ID NO: 102 - DNA, 20 bp, Cannabis sativa
  misc_feature: Complimentary to position 1918 of SEQ. ID NO. 1 THCAS CM011610.122243181-22246809
aagaataagactacagtaca

SEQ ID NO: 103 - DNA, 20 bp, Cannabis sativa
  misc_feature: Complimentary to position 1994 of SEQ. ID NO. 1 THCAS CM011610.122243181-22246809
  misc_feature: Complimentary to position 1942 of SEQ. ID NO. 1 THCAS CM011610.122243181-22246809
tacttctcttgcattttcca

SEQ ID NO: 104 - DNA, 20 bp, Cannabis sativa
  misc_feature: Complimentary to position 1945 of SEQ. ID NO. 1 THCAS CM011610.122243181-22246809
ttctcttgcattttccatgg

SEQ ID NO: 105 - DNA, 20 bp, Cannabis sativa
  misc_feature: Complimentary to position 1948 of SEQ. ID NO. 1 THCAS CM011610.122243181-22246809
ctagactatccactccacca

SEQ ID NO: 106 - DNA, 20 bp, Cannabis sativa
  misc_feature: Complimentary to position 1950 of SEQ. ID NO. 1 THCAS CM011610.122243181-22246809
ttgcattttccatggtggag

SEQ ID NO: 107 - DNA, 20 bp, Cannabis sativa
  misc_feature: Complimentary to position 1992 of SEQ. ID NO. 1 THCAS CM011610.122243181-22246809
gaacaagagctttcctgagt

SEQ ID NO: 108 - DNA, 20 bp, Cannabis sativa
  misc_feature: Complimentary to position 1993 of SEQ. ID NO. 1 THCAS CM011610.122243181-22246809
aacaagagctttcctgagtt

SEQ ID NO: 109 - DNA, 20 bp, Cannabis sativa
  misc_feature: Complimentary to position 1994 of SEQ. ID NO. 1 THCAS CM011610.122243181-22246809
gtttttttaatacccaactc

SEQ ID NO: 110 - DNA, 20 bp, Cannabis sativa
  misc_feature: Complimentary to position 2027 of SEQ. ID NO. 1 THCAS CM011610.122243181-22246809
ctgattgcaaagaattgagc

SEQ ID NO: 111 - DNA, 20 bp, Cannabis sativa
  misc_feature: Complimentary to position 2949 of SEQ. ID NO. 1 THCAS CM011610.122243181-22246809
tacaacaccactgtagaaga

SEQ ID NO: 112 - DNA, 20 bp, Cannabis sativa
  misc_feature: Complimentary to position 2053 of SEQ. ID NO. 1 THCAS CM011610.122243181-22246809
gatactaccatcttctacag

SEQ ID NO: 113 - DNA, 20 bp, Cannabis sativa
  misc_feature: Complimentary to position 2088 of SEQ. ID NO. 1 THCAS CM011610.122243181-22246809
taacactactaattttcaaa

SEQ ID NO: 114 - DNA, 20 bp, Cannabis sativa
  misc_feature: Complimentary to position 2113 of SEQ. ID NO. 1 THCAS CM011610.122243181-22246809
attttgcttgatagatcagc

SEQ ID NO: 115 - DNA, 20 bp, Cannabis sativa
  misc_feature: Complimentary to position 2114 of SEQ. ID NO. 1 THCAS CM011610.122243181-22246809
ttttgcttgatagatcagct

SEQ ID NO: 116 - DNA, 20 bp, Cannabis sativa
  misc_feature: Complimentary to position 2168 of SEQ. ID NO. 1 THCAS CM011610.122243181-22246809
acaattgcagtttctggaat

SEQ ID NO: 117 - DNA, 20 bp, Cannabis sativa
  misc_feature: Complimentary to position 2174 of SEQ. ID NO. 1 THCAS CM011610.122243181-22246809
attttgacaattgcagtttc

SEQ ID NO: 118 - DNA, 20 bp, Cannabis sativa
  misc_feature: Complimentary to position 2190 of SEQ. ID NO. 1 THCAS CM011610.122243181-22246809
aactgcaattgtcaaaattt

SEQ ID NO: 119 - DNA, 20 bp, Cannabis sativa
  misc_feature: Complimentary to position 2215 of SEQ. ID NO. 1 THCAS CM011610.122243181-22246809
aaattgtatgaagaagatgt

SEQ ID NO: 120 - DNA, 20 bp, Cannabis sativa
  misc_feature: Complimentary to position 2221 of SEQ. ID NO. 1 THCAS CM011610.122243181-22246809
tatgaagaagatgtaggagt

SEQ ID NO: 121 - DNA, 20 bp, Cannabis sativa
  misc_feature: Complimentary to position 2245 of SEQ. ID NO. 1 THCAS CM011610.122243181-22246809
gtgtatgtattgtaccctta

SEQ ID NO: 122 - DNA, 20 bp, Cannabis sativa
  misc_feature: Complimentary to position 2248 of SEQ. ID NO. 1 THCAS CM011610.122243181-22246809
tatgtattgtacccttacgg

SEQ ID NO: 123 - DNA, 20 bp, Cannabis sativa
  misc_feature: Complimentary to position 2248, -1, of SEQ. ID NO. 1 THCAS CM011610.122243181-22246809
tgtccattataccaccgtaa

SEQ ID NO: 124 - DNA, 20 bp, Cannabis sativa
  misc_feature: Complimentary to position 2249 of SEQ. ID NO. 1 THCAS CM011610.122243181-22246809
ttgtccattataccaccgta

SEQ ID NO: 125 - DNA, 20 bp, Cannabis sativa
  misc_feature: Complimentary to position 2256 of SEQ. ID NO. 1 THCAS CM011610.122243181-22246809
gtacccttacggtggtataa

SEQ ID NO: 126 - DNA, 20 bp, Cannabis sativa
  misc_feature: Complimentary to position 2291 of SEQ. ID NO. 1 THCAS CM011610.122243181-22246809
attccagctcgatgagggaa

SEQ ID NO: 127 - DNA, 20 bp, Cannabis sativa
  misc_feature: Complimentary to position 2296 of SEQ. ID NO. 1 THCAS CM011610.122243181-22246809
acatgattccagctcgatga

SEQ ID NO: 128 - DNA, 20 bp, Cannabis sativa
  misc_feature: Complimentary to position 2297 of SEQ. ID NO. 1 THCAS CM011610.122243181-22246809
tacatgattccagctcgatg

SEQ ID NO: 129 - DNA, 20 bp, Cannabis sativa
  misc_feature: Complimentary to position 2299 of SEQ. ID NO. 1 THCAS CM011610.122243181-22246809
attcctttccctcatcgagc

SEQ ID NO: 130 - DNA, 20 bp, Cannabis sativa
  misc_feature: Complimentary to position 2318 of SEQ. ID NO. 1 THCAS CM011610.122243181-22246809
ctggaatcatgtacgaagtt

SEQ ID NO: 131 - DNA, 20 bp, Cannabis sativa
  misc_feature: Complimentary to position 2333 of SEQ. ID NO. 1 THCAS CM011610.122243181-22246809
aagtttggtacgcagctacc

SEQ ID NO: 132 - DNA, 20 bp, Cannabis sativa
  misc_feature: Complimentary to position 2334 of SEQ. ID NO. 1 THCAS CM011610.122243181-22246809
agtttggtacgcagctacct

SEQ ID NO: 133 - DNA, 20 bp, Cannabis sativa
  misc_feature: Complimentary to position 2340 of SEQ. ID NO. 1 THCAS CM011610.122243181-22246809
attatcttcttgcttctccc

SEQ ID NO: 134 - DNA, 20 bp, Cannabis sativa
  misc_feature: Complimentary to position 2369 of SEQ. ID NO. 1 THCAS CM011610.122243181-22246809
ataatgaaaagcatataaac

SEQ ID NO: 135 - DNA, 20 bp, Cannabis sativa
  misc_feature: Complimentary to position 2370 of SEQ. ID NO. 1 THCAS CM011610.122243181-22246809
taatgaaaagcatataaact

SEQ ID NO: 136 - DNA, 20 bp, Cannabis sativa
  misc_feature: Complimentary to position 2408 of SEQ. ID NO. 1 THCAS CM011610.122243181-22246809
cttggattttgggacacata

SEQ ID NO: 137 - DNA, 20 bp, Cannabis sativa
  misc_feature: Complimentary to position 2418 of SEQ. ID NO. 1 THCAS CM011610.122243181-22246809
atacgccattcttggatttt

SEQ ID NO: 138 - DNA, 20 bp, Cannabis sativa
  misc_feature: Complimentary to position 2419 of SEQ. ID NO. 1 THCAS CM011610.122243181-22246809
gatacgccattcttggattt

SEQ ID NO: 139 - DNA, 20 bp, Cannabis sativa
  misc_feature: Complimentary to position 2424 of SEQ. ID NO. 1 THCAS CM011610.122243181-22246809
tgtgtcccaaaatccaagaa

SEQ ID NO: 140 - DNA, 20 bp, Cannabis sativa
  misc_feature: Complimentary to position 2426 of SEQ. ID NO. 1 THCAS CM011610.122243181-22246809
taattgagatacgccattct

SEQ ID NO: 141 - DNA, 20 bp, Cannabis sativa
  misc_feature: Complimentary to position 2441 of SEQ. ID NO. 1 THCAS CM011610.122243181-22246809
gaatggcgtatctcaattat

SEQ ID NO: 142 - DNA, 20 bp, Cannabis sativa
  misc_feature: Complimentary to position 2442 of SEQ. ID NO. 1 THCAS CM011610.122243181-22246809
aatggcgtatctcaattata

SEQ ID NO: 143 - DNA, 20 bp, Cannabis sativa
  misc_feature: Complimentary to position 2455 of SEQ. ID NO. 1 THCAS CM011610.122243181-22246809
aattatagggaccttgattt

SEQ ID NO: 144 - DNA, 20 bp, Cannabis sativa
  misc_feature: Complimentary to position 2455, -1, of SEQ. ID NO. 1 THCAS CM011610.122243181-22246809
gatcagtttttcctaaatca

SEQ ID NO: 145 - DNA, 20 bp, Cannabis sativa
  misc_feature: Complimentary to position 2477 of SEQ. ID NO. 1 THCAS CM011610.122243181-22246809
gtgtaattattaggactctt

SEQ ID NO: 146 - DNA, 20 bp, Cannabis sativa
  misc_feature: Complimentary to position 2478 of SEQ. ID NO. 1 THCAS CM011610.122243181-22246809
ggtgtaattattaggactct

SEQ ID NO: 147 - DNA, 20 bp, Cannabis sativa
  misc_feature: Complimentary to position 2486 of SEQ. ID NO. 1 THCAS CM011610.122243181-22246809
cgtgcttgggtgtaattatt

SEQ ID NO: 148 - DNA, 20 bp, Cannabis sativa
  misc_feature: Complimentary to position 2499 of SEQ. ID NO. 1 THCAS CM011610.122243181-22246809
ttcaccccagatacgtgctt

SEQ ID NO: 149 - DNA, 20 bp, Cannabis sativa
  misc_feature: Complimentary to position 2500 of SEQ. ID NO. 1 THCAS CM011610.122243181-22246809
tttcaccccagatacgtgct

SEQ ID NO: 150 - DNA, 20 bp, Cannabis sativa
  misc_feature: Complimentary to position 2504 of SEQ. ID NO. 1 THCAS CM011610.122243181-22246809
attacacccaagcacgtatc

SEQ ID NO: 151 - DNA, 20 bp, Cannabis sativa
  misc_feature: Complimentary to position 2505of SEQ. ID NO. 1 THCAS CM011610.122243181-22246809
ttacacccaagcacgtatct

SEQ ID NO: 152 - DNA, 20 bp, Cannabis sativa
  misc_feature: Complimentary to position 2506 of SEQ. ID NO. 1 THCAS CM011610.122243181-22246809
tacacccaagcacgtatctg

SEQ ID NO: 153 - DNA, 20 bp, Cannabis sativa
  misc_feature: Complimentary to position 2521 of SEQ. ID NO. 1 THCAS CM011610.122243181-22246809
atctggggtgaaaagtactt

SEQ ID NO: 154 - DNA, 20 bp, Cannabis sativa
  misc_feature: Complimentary to position 2547 of SEQ. ID NO. 1 THCAS CM011610.122243181-22246809
aaactttgacaagttagtta

SEQ ID NO: 155 - DNA, 20 bp, Cannabis sativa
  misc_feature: Complimentary to position 2565 of SEQ. ID NO. 1 THCAS CM011610.122243181-22246809
aaaattattgggatcaactt

SEQ ID NO: 156 - DNA, 20 bp, Cannabis sativa
  misc_feature: Complimentary to position 2576 of SEQ. ID NO. 1 THCAS CM011610.122243181-22246809
tcgtttctaaaaaaattatt

SEQ ID NO: 157 - DNA, 20 bp, Cannabis sativa
  misc_feature: Complimentary to position 2577 of SEQ. ID NO. 1 THCAS CM011610.122243181-22246809
ctcgtttctaaaaaaattat

SEQ ID NO: 158 - DNA, 20 bp, Cannabis sativa
  misc_feature: Complimentary to position 2608 of SEQ. ID NO. 1 THCAS CM011610.122243181-22246809
gacgtcgtggcggaagaggt

SEQ ID NO: 159 - DNA, 20 bp, Cannabis sativa
  misc_feature: Complimentary to position 2609  of SEQ. ID NO. 1 THCAS CM011610.122243181-22246809
tgacgtcgtggcggaagagg

SEQ ID NO: 160 - DNA, 20 bp, Cannabis sativa
  misc_feature: Complimentary to position 2612 of SEQ. ID NO. 1 THCAS CM011610.122243181-22246809
taatgacgtcgtggcggaag

SEQ ID NO: 161 - DNA, 20 bp, Cannabis sativa
  misc_feature: Complimentary to position 2618 of SEQ. ID NO. 1 THCAS CM011610.122243181-22246809
aataattaatgacgtcgtgg

SEQ ID NO: 162 - DNA, 20 bp, Cannabis sativa
  misc_feature: Complimentary to position 2621 of SEQ. ID NO. 1 THCAS CM011610.122243181-22246809
aaaaataattaatgacgtcg

SEQ ID NO: 163 - DNA, 20 bp, Cannabis sativa
  misc_feature: Complimentary to position 2684 of SEQ. ID NO. 1 THCAS CM011610.122243181-22246809
atatataacagatacatgta

SEQ ID NO: 164 - DNA, 20 bp, Cannabis sativa
  misc_feature: Complimentary to position 2719 of SEQ. ID NO. 1 THCAS CM011610.122243181-22246809
cttaggagcatacatagtac

SEQ ID NO: 165 - DNA, 20 bp, Cannabis sativa
  misc_feature: Complimentary to position 2736 of SEQ. ID NO. 1 THCAS CM011610.122243181-22246809
tactgtagatgttcatactt

SEQ ID NO: 166 - DNA, 20 bp, Cannabis sativa
  misc_feature: Complimentary to position 2776 of SEQ. ID NO. 1 THCAS CM011610.122243181-22246809
tgtagacatcataagatata

SEQ ID NO: 167 - DNA, 20 bp, Cannabis sativa
  misc_feature: Complimentary to position 2807 of SEQ. ID NO. 1 THCAS CM011610.122243181-22246809
aaattatctttcttatttaa

SEQ ID NO: 168 - DNA, 20 bp, Cannabis sativa
  misc_feature: Complimentary to position 927 of SEQ. ID NO. 4 CBDAS CM011610.121836038-21839672
agctttatatattggagcag

SEQ ID NO: 169 - DNA, 20 bp, Cannabis sativa
  misc_feature: Complimentary to position 928 of SEQ. ID NO. 4 CBDAS CM011610.121836038-21839672
tagctttatatattggagca

SEQ ID NO: 170 - DNA, 20 bp, Cannabis sativa
  misc_feature: Complimentary to position 929 of SEQ. ID NO. 4 CBDAS CM011610.121836038-21839672
atagctttatatattggagc

SEQ ID NO: 171 - DNA, 20 bp, Cannabis sativa
  misc_feature: Complimentary to position 935 of SEQ. ID NO. 4 CBDAS CM011610.121836038-21839672
ctatttatagctttatatat

SEQ ID NO: 172 - DNA, 20 bp, Cannabis sativa
  misc_feature: Complimentary to position 947 of SEQ. ID NO. 4 CBDAS CM011610.121836038-21839672
caatatataaagctataaat

SEQ ID NO: 173 - DNA, 20 bp, Cannabis sativa
  misc_feature: Complimentary to position 971 of SEQ. ID NO. 4 CBDAS CM011610.121836038-21839672
taatgaattttgaattacta

SEQ ID NO: 174 - DNA, 20 bp, Cannabis sativa
  misc_feature: Complimentary to position 1022 of SEQ. ID NO. 4 CBDAS CM011610.121836038-21839672
agtactcaacattctccttt

SEQ ID NO: 175 - DNA, 20 bp, Cannabis sativa
  misc_feature: Complimentary to position 1026 of SEQ. ID NO. 4 CBDAS CM011610.121836038-21839672
tatcttgcaaacaaaccaaa

SEQ ID NO: 176 - DNA, 20 bp, Cannabis sativa
  misc_feature: Complimentary to position 1026 of SEQ. ID NO. 4 CBDAS CM011610.121836038-21839672
gaggattagcaatggaagtt

SEQ ID NO: 177 - DNA, 20 bp, Cannabis sativa
  misc_feature: Complimentary to position 1083 of SEQ. ID NO. 4 CBDAS CM011610.121836038-21839672
gttttctcgaggattagcaa

SEQ ID NO: 178 - DNA, 20 bp, Cannabis sativa
  misc_feature: Complimentary to position 1094 of SEQ. ID NO. 4 CBDAS CM011610.121836038-21839672
catttaaggaagttttctcg

SEQ ID NO: 179 - DNA, 20 bp, Cannabis sativa
  misc_feature: Complimentary to position 1108 of SEQ. ID NO. 4 CBDAS CM011610.121836038-21839672
tatattgcgagaagcattta

SEQ ID NO: 180 - DNA, 20 bp, Cannabis sativa
  misc_feature: Complimentary to position 1133 of SEQ. ID NO. 4 CBDAS CM011610.121836038-21839672
tttagatttgttgcattatt

SEQ ID NO: 181 - DNA, 20 bp, Cannabis sativa
  misc_feature: Complimentary to position 1134 of SEQ. ID NO. 4 CBDAS CM011610.121836038-21839672
ttttagatttgttgcattat

SEQ ID NO: 182 - DNA, 20 bp, Cannabis sativa
  misc_feature: Complimentary to position 1177  of SEQ. ID NO. 4 CBDAS CM011610.121836038-21839672
ttaggacagacatatacaat

SEQ ID NO: 183 - DNA, 20 bp, Cannabis sativa
  misc_feature: Complimentary to position 1178  of SEQ. ID NO. 4 CBDAS CM011610.121836038-21839672
tttaggacagacatatacaa

SEQ ID NO: 184 - DNA, 20 bp, Cannabis sativa
  misc_feature: Complimentary to position 1195  of SEQ. ID NO. 4 CBDAS CM011610.121836038-21839672
gattgtgtattgtcgaattt

SEQ ID NO: 185 - DNA, 20 bp, Cannabis sativa
  misc_feature: Complimentary to position 1239 of SEQ. ID NO. 4 CBDAS CM011610.121836038-21839672
gacgataacaagtggttttg

SEQ ID NO: 186 - DNA, 20 bp, Cannabis sativa
  misc_feature: Complimentary to position 1240  of SEQ. ID NO. 4 CBDAS CM011610.121836038-21839672
tgacgataacaagtggtttt

SEQ ID NO: 187 - DNA, 20 bp, Cannabis sativa
  misc_feature: Complimentary to position 1241 of SEQ. ID NO. 4 CBDAS CM011610.121836038-21839672
gtgacgataacaagtggttt

SEQ ID NO: 188 - DNA, 20 bp, Cannabis sativa
  misc_feature: Complimentary to position 1247 of SEQ. ID NO. 4 CBDAS CM011610.121836038-21839672
gaaggagtgacgataacaag

SEQ ID NO: 189 - DNA, 20 bp, Cannabis sativa
  misc_feature: Complimentary to position 1256 of SEQ. ID NO. 4 CBDAS CM011610.121836038-21839672
tggatatgagagacatgtga

SEQ ID NO: 190 - DNA, 20 bp, Cannabis sativa
  misc_feature: Complimentary to position 1279 of SEQ. ID NO. 4 CBDAS CM011610.121836038-21839672
tcacatgtctctcatatcca

SEQ ID NO: 191 - DNA, 20 bp, Cannabis sativa
  misc_feature: Complimentary to position 1285 of SEQ. ID NO. 4 CBDAS CM011610.121836038-21839672
tggagcatagaatagtgcct

SEQ ID NO: 192 - DNA, 20 bp, Cannabis sativa
  misc_feature: Complimentary to position 1305 of SEQ. ID NO. 4 CBDAS CM011610.121836038-21839672
aatctgcaagccaactttct

SEQ ID NO: 193 - DNA, 20 bp, Cannabis sativa
  misc_feature: Complimentary to position 1306 of SEQ. ID NO. 4 CBDAS CM011610.121836038-21839672
attctatgctccaagaaagt

SEQ ID NO: 194 - DNA, 20 bp, Cannabis sativa
  misc_feature: Complimentary to position 1330 of SEQ. ID NO. 4 CBDAS CM011610.121836038-21839672
ttgcagattcgaactcgaag

SEQ ID NO: 195 - DNA, 20 bp, Cannabis sativa
  misc_feature: Complimentary to position 1333 of SEQ. ID NO. 4 CBDAS CM011610.121836038-21839672
cagattcgaactcgaagtgg

SEQ ID NO: 196 - DNA, 20 bp, Cannabis sativa
  misc_feature: Complimentary to position 1347 of SEQ. ID NO. 4 CBDAS CM011610.121836038-21839672
aagtggtggtcatgattctg

SEQ ID NO: 197 - DNA, 20 bp, Cannabis sativa
  misc_feature: Complimentary to position 1348 of SEQ. ID NO. 4 CBDAS CM011610.121836038-21839672
agtggtggtcatgattctga

SEQ ID NO: 198 - DNA, 20 bp, Cannabis sativa
  misc_feature: Complimentary to position 1365 of SEQ. ID NO. 4 CBDAS CM011610.121836038-21839672
aaatgggacttgagatatgt

SEQ ID NO: 199 - DNA, 20 bp, Cannabis sativa
  misc_feature: Complimentary to position 1381 of SEQ. ID NO. 4 CBDAS CM011610.121836038-21839672
tcaagtctactataacaaat

SEQ ID NO: 200 - DNA, 20 bp, Cannabis sativa
  misc_feature: Complimentary to position 1382 of SEQ. ID NO. 4 CBDAS CM011610.121836038-21839672
ctcaagtctactataacaaa

SEQ ID NO: 201 - DNA, 20 bp, Cannabis sativa
  misc_feature: Complimentary to position  1438 of SEQ. ID NO. 4 CBDAS CM011610.121836038-21839672
cggcttcaacccatgcagtt

SEQ ID NO: 202 - DNA, 20 bp, Cannabis sativa
  misc_feature: Complimentary to position 1439 of SEQ. ID NO. 4 CBDAS CM011610.121836038-21839672
atgttcatagccaaactgca

SEQ ID NO: 203 - DNA, 20 bp, Cannabis sativa
  misc_feature: Complimentary to position 1440 of SEQ. ID NO. 4 CBDAS CM011610.121836038-21839672
tgttcatagccaaactgcat

SEQ ID NO: 204 - DNA, 20 bp, Cannabis sativa
  misc_feature: Complimentary to position 1450 of SEQ. ID NO. 4 CBDAS CM011610.121836038-21839672
caaactgcatgggttgaagc

SEQ ID NO: 205 - DNA, 20 bp, Cannabis sativa
  misc_feature: Complimentary to position  1458 of SEQ. ID NO. 4 CBDAS CM011610.121836038-21839672
aacttctccaagggtagctc

SEQ ID NO: 206 - DNA, 20 bp, Cannabis sativa
  misc_feature: Complimentary to position 1462 of SEQ. ID NO. 4 CBDAS CM011610.121836038-21839672
gttgaagccggagctaccct

SEQ ID NO: 207 - DNA, 20 bp, Cannabis sativa
  misc_feature: Complimentary to position 1467 of SEQ. ID NO. 4 CBDAS CM011610.121836038-21839672
ccaataataaacttctccaa

SEQ ID NO: 208 - DNA, 20 bp, Cannabis sativa
  misc_feature: Complimentary to position 1468 of SEQ. ID NO. 4 CBDAS CM011610.121836038-21839672
cccaataataaacttctcca

SEQ ID NO: 209 - DNA, 20 bp, Cannabis sativa
  misc_feature: Complimentary to position 1478 of SEQ. ID NO. 4 CBDAS CM011610.121836038-21839672
cccttggagaagtttattat

SEQ ID NO: 210 - DNA, 20 bp, Cannabis sativa
  misc_feature: Complimentary to position 1479 of SEQ. ID NO. 4 CBDAS CM011610.121836038-21839672
ccttggagaagtttattatt

SEQ ID NO: 211 - DNA, 20 bp, Cannabis sativa
  misc_feature: Complimentary to position 1509 of SEQ. ID NO. 4 CBDAS CM011610.121836038-21839672
gaaaaatgagagtcttagtt

SEQ ID NO: 212 - DNA, 20 bp, Cannabis sativa
  misc_feature: Complimentary to position 1516 of SEQ. ID NO. 4 CBDAS CM011610.121836038-21839672
gagagtcttagtttggctgc

SEQ ID NO: 213 - DNA, 20 bp, Cannabis sativa
  misc_feature: Complimentary to position 1517 of SEQ. ID NO. 4 CBDAS CM011610.121836038-21839672
agagtcttagtttggctgct

SEQ ID NO: 214 - DNA, 20 bp, Cannabis sativa
  misc_feature: Complimentary to position 1534 of SEQ. ID NO. 4 CBDAS CM011610.121836038-21839672
gtccacctgcgcaaacagta

SEQ ID NO: 215 - DNA, 20 bp, Cannabis sativa
  misc_feature: Complimentary to position 1540 of SEQ. ID NO. 4 CBDAS CM011610.121836038-21839672
tattgccctactgtttgcgc

SEQ ID NO: 216 - DNA, 20 bp, Cannabis sativa
  misc_feature: Complimentary to position 1543 of SEQ. ID NO. 4 CBDAS CM011610.121836038-21839672
tgccctactgtttgcgcagg

SEQ ID NO: 217 - DNA, 20 bp, Cannabis sativa
  misc_feature: Complimentary to position 1552 of SEQ. ID NO. 4 CBDAS CM011610.121836038-21839672
gtttgcgcaggtggacactt

SEQ ID NO: 218 - DNA, 20 bp, Cannabis sativa
  misc_feature: Complimentary to position 1558 of SEQ. ID NO. 4 CBDAS CM011610.121836038-21839672
gcaggtggacactttggtgg

SEQ ID NO: 219 - DNA, 20 bp, Cannabis sativa
  misc_feature: Complimentary to position 1561of SEQ. ID NO. 4 CBDAS CM011610.121836038-21839672
ggtggacactttggtggagg

SEQ ID NO: 220 - DNA, 20 bp, Cannabis sativa
  misc_feature: Complimentary to position 1567 of SEQ. ID NO. 4 CBDAS CM011610.121836038-21839672
cactttggtggaggaggcta

SEQ ID NO: 221 - DNA, 20 bp, Cannabis sativa
  misc_feature: Complimentary to position 1580 of SEQ. ID NO. 4 CBDAS CM011610.121836038-21839672
aggccatagcttctcatcaa

SEQ ID NO: 222 - DNA, 20 bp, Cannabis sativa
  misc_feature: Complimentary to position 1588 of SEQ. ID NO. 4 CBDAS CM011610.121836038-21839672
ggaccattgatgagaagcta

SEQ ID NO: 223 - DNA, 20 bp, Cannabis sativa
  misc_feature: Complimentary to position 1596 of SEQ. ID NO. 4 CBDAS CM011610.121836038-21839672
gatgagaagctatggcctcg

SEQ ID NO: 224 - DNA, 20 bp, Cannabis sativa
  misc_feature: Complimentary to position 1600 of SEQ. ID NO. 4 CBDAS CM011610.121836038-21839672
caatgatattatcagccgcg

SEQ ID NO: 225 - DNA, 20 bp, Cannabis sativa
  misc_feature: Complimentary to position 1636 of SEQ. ID NO. 4 CBDAS CM011610.121836038-21839672
gcacacttagtcaacgttca

SEQ ID NO: 226 - DNA, 20 bp, Cannabis sativa
agtgctagatcgaaaatcta

SEQ ID NO: 227 - DNA, 20 bp, Cannabis sativa
  misc_feature: Complimentary to position 1663 of SEQ. ID NO. 4 CBDAS CM011610.121836038-21839672
gtgctagatcgaaaatctat

SEQ ID NO: 228 - DNA, 20 bp, Cannabis sativa
  misc_feature: Complimentary to position 1664 of SEQ. ID NO. 4 CBDAS CM011610.121836038-21839672
tgctagatcgaaaatctatg

SEQ ID NO: 229 - DNA, 20 bp, Cannabis sativa
  misc_feature: Complimentary to position 1665 of SEQ. ID NO. 4 CBDAS CM011610.121836038-21839672
gctagatcgaaaatctatgg

SEQ ID NO: 230 - DNA, 20 bp, Cannabis sativa
  misc_feature: Complimentary to position 1679 of SEQ. ID NO. 4 CBDAS CM011610.121836038-21839672
ctatgggggaagatctcttt

SEQ ID NO: 231 - DNA, 20 bp, Cannabis sativa
  misc_feature: Complimentary to position 1680  of SEQ. ID NO. 4 CBDAS CM011610.121836038-21839672
tatgggggaagatctctttt

SEQ ID NO: 232 - DNA, 20 bp, Cannabis sativa
  misc_feature: Complimentary to position 1690 of SEQ. ID NO. 4 CBDAS CM011610.121836038-21839672
gatctcttttgggctttacg

SEQ ID NO: 233 - DNA, 20 bp, Cannabis sativa
  misc_feature: Complimentary to position 1693 of SEQ. ID NO. 4 CBDAS CM011610.121836038-21839672
ctcttttgggctttacgtgg

SEQ ID NO: 234 - DNA, 20 bp, Cannabis sativa
  misc_feature: Complimentary to position 1669 of SEQ. ID NO. 4 CBDAS CM011610.121836038-21839672
ttttgggctttacgtggtgg

SEQ ID NO: 235 - DNA, 20 bp, Cannabis sativa
  misc_feature: Complimentary to position 1711 of SEQ. ID NO. 4 CBDAS CM011610.121836038-21839672
ggtggtggagcagaaagctt

SEQ ID NO: 236 - DNA, 20 bp, Cannabis sativa
  misc_feature: Complimentary to position 1727  of SEQ. ID NO. 4 CBDAS CM011610.121836038-21839672
gcttcggaatcattgtagca

SEQ ID NO: 237 - DNA, 20 bp, Cannabis sativa
  misc_feature: Complimentary to position 1740  of SEQ. ID NO. 4 CBDAS CM011610.121836038-21839672
tgtagcatggaaaattagac

SEQ ID NO: 238 - DNA, 20 bp, Cannabis sativa
  misc_feature: Complimentary to position 1759 of SEQ. ID NO. 4 CBDAS CM011610.121836038-21839672
cactaaacatagtagacttt

SEQ ID NO: 239 - DNA, 20 bp, Cannabis sativa
  misc_feature: Complimentary to position  1760 of SEQ. ID NO. 4 CBDAS CM011610.121836038-21839672
acactaaacatagtagactt

SEQ ID NO: 240 - DNA, 20 bp, Cannabis sativa
  misc_feature: Complimentary to position 1785 of SEQ. ID NO. 4 CBDAS CM011610.121836038-21839672
gtttagtgttaaaaagatca

SEQ ID NO: 241 - DNA, 20 bp, Cannabis sativa
  misc_feature: Complimentary to position 1820 of SEQ. ID NO. 4 CBDAS CM011610.121836038-21839672
ttgtcaagttagttaacaaa

SEQ ID NO: 242 - DNA, 20 bp, Cannabis sativa
  misc_feature: Complimentary to position 1880 of SEQ. ID NO. 4 CBDAS CM011610.121836038-21839672
tcatgactcacttcataact

SEQ ID NO: 243 - DNA, 20 bp, Cannabis sativa
  misc_feature: Complimentary to position 1900  of SEQ. ID NO. 4 CBDAS CM011610.121836038-21839672
aggaacattacagataatca

SEQ ID NO: 244 - DNA, 20 bp, Cannabis sativa
  misc_feature: Complimentary to position 1901 of SEQ. ID NO. 4 CBDAS CM011610.121836038-21839672
ggaacattacagataatcaa

SEQ ID NO: 245 - DNA, 20 bp, Cannabis sativa
  misc_feature: Complimentary to position 1948 of SEQ. ID NO. 4 CBDAS CM011610.121836038-21839672
tacttctcttcagttttcct

SEQ ID NO: 246 - DNA, 20 bp, Cannabis sativa
  misc_feature: Complimentary to position 1951 of SEQ. ID NO. 4 CBDAS CM011610.121836038-21839672
ttctcttcagttttccttgg

SEQ ID NO: 247 - DNA, 20 bp, Cannabis sativa
  misc_feature: Complimentary to position 1954 of SEQ. ID NO. 4 CBDAS CM011610.121836038-21839672
ctagactatccactccacca

SEQ ID NO: 248 - DNA, 20 bp, Cannabis sativa
  misc_feature: Complimentary to position 1956 of SEQ. ID NO. 4 CBDAS CM011610.121836038-21839672
ttcagttttccttggtggag

SEQ ID NO: 249 - DNA, 20 bp, Cannabis sativa
  misc_feature: Complimentary to position 1998 of SEQ. ID NO. 4 CBDAS CM011610.121836038-21839672
gaacaagagttttcctgagt

SEQ ID NO: 250 - DNA, 20 bp, Cannabis sativa
  misc_feature: Complimentary to position 1999 of SEQ. ID NO. 4 CBDAS CM011610.121836038-21839672
aacaagagttttcctgagtt

SEQ ID NO: 251 - DNA, 20 bp, Cannabis sativa
  misc_feature: Complimentary to position 2000 of SEQ. ID NO. 4 CBDAS CM011610.121836038-21839672
gtttttttaatacccaactc

SEQ ID NO: 252 - DNA, 20 bp, Cannabis sativa
  misc_feature: Complimentary to position 2013 of SEQ. ID NO. 4 CBDAS CM011610.121836038-21839672
tgagttgggtattaaaaaaa

SEQ ID NO: 253 - DNA, 20 bp, Cannabis sativa
  misc_feature: Complimentary to position 2033 of SEQ. ID NO. 4 CBDAS CM011610.121836038-21839672
cggattgcagacaattgagc

SEQ ID NO: 254 - DNA, 20 bp, Cannabis sativa
  misc_feature: Complimentary to position 2059   of SEQ. ID NO. 4 CBDAS CM011610.121836038-21839672
gatactatcatcttctatag

SEQ ID NO: 255 - DNA, 20 bp, Cannabis sativa
  misc_feature: Complimentary to position 2094 of SEQ. ID NO. 4 CBDAS CM011610.121836038-21839672
cgacactgataattttaaca

SEQ ID NO: 256 - DNA, 20 bp, Cannabis sativa
  misc_feature: Complimentary to position 2119 of SEQ. ID NO. 4 CBDAS CM011610.121836038-21839672
attttgcttgatagatccgc

SEQ ID NO: 257 - DNA, 20 bp, Cannabis sativa
  misc_feature: Complimentary to position 2120 of SEQ. ID NO. 4 CBDAS CM011610.121836038-21839672
ttttgcttgatagatccgct

SEQ ID NO: 258 - DNA, 20 bp, Cannabis sativa
  misc_feature: Complimentary to position 2124 of SEQ. ID NO. 4 CBDAS CM011610.121836038-21839672
gaaagcaccgttctgcccag

SEQ ID NO: 259 - DNA, 20 bp, Cannabis sativa
  misc_feature: Complimentary to position 2128 of SEQ. ID NO. 4 CBDAS CM011610.121836038-21839672
gatagatccgctgggcagaa

SEQ ID NO: 260 - DNA, 20 bp, Cannabis sativa
  misc_feature: Complimentary to position 2174   of SEQ. ID NO. 4 CBDAS CM011610.121836038-21839672
acaaatacagattctggaat

SEQ ID NO: 261 - DNA, 20 bp, Cannabis sativa
  misc_feature: Complimentary to position 2180 of SEQ. ID NO. 4 CBDAS CM011610.121836038-21839672
atttggacaaatacagattc

SEQ ID NO: 262 - DNA, 20 bp, Cannabis sativa
  misc_feature: Complimentary to position 2196 of SEQ. ID NO. 4 CBDAS CM011610.121836038-21839672
atctgtatttgtccaaattt

SEQ ID NO: 263 - DNA, 20 bp, Cannabis sativa
  misc_feature: Complimentary to position 2197 of SEQ. ID NO. 4 CBDAS CM011610.121836038-21839672
catataatttttccaaaatt

SEQ ID NO: 264 - DNA, 20 bp, Cannabis sativa
  misc_feature: Complimentary to position 2221 of SEQ. ID NO. 4 CBDAS CM011610.121836038-21839672
aaattatatgaagaagatat

SEQ ID NO: 265 - DNA, 20 bp, Cannabis sativa
  misc_feature: Complimentary to position 2227 of SEQ. ID NO. 4 CBDAS CM011610.121836038-21839672
tatgaagaagatataggagc

SEQ ID NO: 266 - DNA, 20 bp, Cannabis sativa
  misc_feature: Complimentary to position 2228 of SEQ. ID NO. 4 CBDAS CM011610.121836038-21839672
atgaagaagatataggagct

SEQ ID NO: 267 - DNA, 20 bp, Cannabis sativa
  misc_feature: Complimentary to position 2251 of SEQ. ID NO. 4 CBDAS CM011610.121836038-21839672
atgtatgcgttgtaccctta

SEQ ID NO: 268 - DNA, 20 bp, Cannabis sativa
  misc_feature: Complimentary to position 2254 of SEQ. ID NO. 4 CBDAS CM011610.121836038-21839672
tatgcgttgtacccttacgg

SEQ ID NO: 269 - DNA, 20 bp, Cannabis sativa
  misc_feature: Complimentary to position 2254, -1, of SEQ. ID NO. 4 CBDAS CM011610.121836038-21839672
catccattataccaccgtaa

SEQ ID NO: 270 - DNA, 20 bp, Cannabis sativa
  misc_feature: Complimentary to position 2255 of SEQ. ID NO. 4 CBDAS CM011610.121836038-21839672
tcatccattataccaccgta

SEQ ID NO: 271 - DNA, 20 bp, Cannabis sativa
  misc_feature: Complimentary to position 2262 of SEQ. ID NO. 4 CBDAS CM011610.121836038-21839672
gtacccttacggtggtataa

SEQ ID NO: 272 - DNA, 20 bp, Cannabis sativa
  misc_feature: Complimentary to position 2297 of SEQ. ID NO. 4 CBDAS CM011610.121836038-21839672
attccagctcgatgagggaa

SEQ ID NO: 273 - DNA, 20 bp, Cannabis sativa
  misc_feature: Complimentary to position 2302 of SEQ. ID NO. 4 CBDAS CM011610.121836038-21839672
acaagattccagctcgatga

SEQ ID NO: 274 - DNA, 20 bp, Cannabis sativa
  misc_feature: Complimentary to position 2303 of SEQ. ID NO. 4 CBDAS CM011610.121836038-21839672
tacaagattccagctcgatg

SEQ ID NO: 275 - DNA, 20 bp, Cannabis sativa
  misc_feature: Complimentary to position 2305 of SEQ. ID NO. 4 CBDAS CM011610.121836038-21839672
attccattccctcatcgagc

SEQ ID NO: 276 - DNA, 20 bp, Cannabis sativa
  misc_feature: Complimentary to position 2324of SEQ. ID NO. 4 CBDAS CM011610.121836038-21839672
ctggaatcttgtatgagtta

SEQ ID NO: 277 - DNA, 20 bp, Cannabis sativa
  misc_feature: Complimentary to position 2339 of SEQ. ID NO. 4 CBDAS CM011610.121836038-21839672
agttatggtacatatgtagc

SEQ ID NO: 278 - DNA, 20 bp, Cannabis sativa
  misc_feature: Complimentary to position 2340 of SEQ. ID NO. 4 CBDAS CM011610.121836038-21839672
gttatggtacatatgtagct

SEQ ID NO: 279 - DNA, 20 bp, Cannabis sativa
  misc_feature: Complimentary to position 2375 of SEQ. ID NO. 4 CBDAS CM011610.121836038-21839672
ataacgaaaagcatctaaac

SEQ ID NO: 280 - DNA, 20 bp, Cannabis sativa
  misc_feature: Complimentary to position 2414 of SEQ. ID NO. 4 CBDAS CM011610.121836038-21839672
cttggattttgggacacata

SEQ ID NO: 281 - DNA, 20 bp, Cannabis sativa
  misc_feature: Complimentary to position 2424 of SEQ. ID NO. 4 CBDAS CM011610.121836038-21839672
atatgccaatcttggatttt

SEQ ID NO: 282 - DNA, 20 bp, Cannabis sativa
  misc_feature: Complimentary to position 2425 of SEQ. ID NO. 4 CBDAS CM011610.121836038-21839672
gatatgccaatcttggattt

SEQ ID NO: 283 - DNA, 20 bp, Cannabis sativa
  misc_feature: Complimentary to position 2430 of SEQ. ID NO. 4 CBDAS CM011610.121836038-21839672
tgtgtcccaaaatccaagat

SEQ ID NO: 284 - DNA, 20 bp, Cannabis sativa
  misc_feature: Complimentary to position 2432 of SEQ. ID NO. 4 CBDAS CM011610.121836038-21839672
taattgagatatgccaatct

SEQ ID NO: 285 - DNA, 20 bp, Cannabis sativa
  misc_feature: Complimentary to position 2461 of SEQ. ID NO. 4 CBDAS CM011610.121836038-21839672
aattatagagaccttgatat

SEQ ID NO: 286 - DNA, 20 bp, Cannabis sativa
  misc_feature: Complimentary to position 2461, -1, of SEQ. ID NO. 4 CBDAS CM011610.121836038-21839672
gatcatttattcctatatca

SEQ ID NO: 287 - DNA, 20 bp, Cannabis sativa
  misc_feature: Complimentary to position 2483 of SEQ. ID NO. 4 CBDAS CM011610.121836038-21839672
gtgtaattatttggattctt

SEQ ID NO: 288 - DNA, 20 bp, Cannabis sativa
  misc_feature: Complimentary to position 2484 of SEQ. ID NO. 4 CBDAS CM011610.121836038-21839672
tgtgtaattatttggattct

SEQ ID NO: 289 - DNA, 20 bp, Cannabis sativa
  misc_feature: Complimentary to position 2492 of SEQ. ID NO. 4 CBDAS CM011610.121836038-21839672
cgtgcttgtgtgtaattatt

SEQ ID NO: 290 - DNA, 20 bp, Cannabis sativa
  misc_feature: Complimentary to position 2510 of SEQ. ID NO. 4 CBDAS CM011610.121836038-21839672
attacacacaagcacgtatt

SEQ ID NO: 291 - DNA, 20 bp, Cannabis sativa
  misc_feature: Complimentary to position 2511 of SEQ. ID NO. 4 CBDAS CM011610.121836038-21839672
ttacacacaagcacgtattt

SEQ ID NO: 292 - DNA, 20 bp, Cannabis sativa
  misc_feature: Complimentary to position 2512 of SEQ. ID NO. 4 CBDAS CM011610.121836038-21839672
tacacacaagcacgtatttg

SEQ ID NO: 293 - DNA, 20 bp, Cannabis sativa
  misc_feature: Complimentary to position 2527 of SEQ. ID NO. 4 CBDAS CM011610.121836038-21839672
atttggggtgagaagtattt

SEQ ID NO: 294 - DNA, 20 bp, Cannabis sativa
  misc_feature: Complimentary to position 2543 of SEQ. ID NO. 4 CBDAS CM011610.121836038-21839672
attttggtaaaaattttgac

SEQ ID NO: 295 - DNA, 20 bp, Cannabis sativa
  misc_feature: Complimentary to position 2565 of SEQ. ID NO. 4 CBDAS CM011610.121836038-21839672
gctagtaaaagtgaaaaccc

SEQ ID NO: 296 - DNA, 20 bp, Cannabis sativa
  misc_feature: Complimentary to position 2571  of SEQ. ID NO. 4 CBDAS CM011610.121836038-21839672
aaaattattgggatcaacca

SEQ ID NO: 297 - DNA, 20 bp, Cannabis sativa
  misc_feature: Complimentary to position 2572 of SEQ. ID NO. 4 CBDAS CM011610.121836038-21839672
aaaaattattgggatcaacc

SEQ ID NO: 298 - DNA, 20 bp, Cannabis sativa
  misc_feature: Complimentary to position 2582 of SEQ. ID NO. 4 CBDAS CM011610.121836038-21839672
tcgtttctaaaaaaattatt

SEQ ID NO: 299 - DNA, 20 bp, Cannabis sativa
  misc_feature: Complimentary to position 2583 of SEQ. ID NO. 4 CBDAS CM011610.121836038-21839672
ttcgtttctaaaaaaattat

SEQ ID NO: 300 - DNA, 20 bp, Cannabis sativa
  misc_feature: Complimentary to position 2614 of SEQ. ID NO. 4 CBDAS CM011610.121836038-21839672
gatgatgccgtggaagaggt

SEQ ID NO: 301 - DNA, 20 bp, Cannabis sativa
  misc_feature: Complimentary to position 2615n of SEQ. ID NO. 4 CBDAS CM011610.121836038-21839672
tgatgatgccgtggaagagg

SEQ ID NO: 302 - DNA, 20 bp, Cannabis sativa
  misc_feature: Complimentary to position 2618 of SEQ. ID NO. 4 CBDAS CM011610.121836038-21839672
aaagcatcccacctcttcca

SEQ ID NO: 303 - DNA, 20 bp, Cannabis sativa
  misc_feature: Complimentary to position 2618, -1, of SEQ. ID NO. 4 CBDAS CM011610.121836038-21839672
taatgatgatgccgtggaag

SEQ ID NO: 304 - DNA, 20 bp, Cannabis sativa
  misc_feature: Complimentary to position 2624 of SEQ. ID NO. 4 CBDAS CM011610.121836038-21839672
gatcattaatgatgatgccg

SEQ ID NO: 305 - DNA, 20 bp, Cannabis sativa
  misc_feature: Complimentary to position 2533 of SEQ. ID NO. 7  PT
ctataaataatataatgtgt

SEQ ID NO: 306 - DNA, 20 bp, Cannabis sativa
  misc_feature: Complimentary to position 2548 of SEQ. ID NO. 7  PT
tcatataattaagacacatt

SEQ ID NO: 307 - DNA, 20 bp, Cannabis sativa
  misc_feature: Complimentary to position 2605 of SEQ. ID NO. 7  PT
gaagattttacgagttcatg

SEQ ID NO: 308 - DNA, 20 bp, Cannabis sativa
  misc_feature: Complimentary to position 2630 of SEQ. ID NO. 7  PT
ggttgtacaatgcaggaagc

SEQ ID NO: 309 - DNA, 20 bp, Cannabis sativa
  misc_feature: Complimentary to position 2637 of SEQ. ID NO. 7  PT
tatattgggttgtacaatgc

SEQ ID NO: 310 - DNA, 20 bp, Cannabis sativa
  misc_feature: Complimentary to position 2651 of SEQ. ID NO. 7  PT
atctgattatattttatatt

SEQ ID NO: 311 - DNA, 20 bp, Cannabis sativa
  misc_feature: Complimentary to position 2652 of SEQ. ID NO. 7  PT
catctgattatattttatat

SEQ ID NO: 312 - DNA, 20 bp, Cannabis sativa
  misc_feature: Complimentary to position 2717 of SEQ. ID NO. 7  PT
ttttgtatgtaccaataaag

SEQ ID NO: 313 - DNA, 20 bp, Cannabis sativa
  misc_feature: Complimentary to position 2717, -1, of SEQ. ID NO. 7  PT
tgtgttattatcctctttat

SEQ ID NO: 314 - DNA, 20 bp, Cannabis sativa
  misc_feature: Complimentary to position 2736 of SEQ. ID NO. 7  PT
gaggataataacacaattaa

SEQ ID NO: 315 - DNA, 20 bp, Cannabis sativa
  misc_feature: Complimentary to position 2758 of SEQ. ID NO. 7  PT
gtagtcattttgagtattac

SEQ ID NO: 316 - DNA, 20 bp, Cannabis sativa
  misc_feature: Complimentary to position 2759 of SEQ. ID NO. 7  PT
tagtcattttgagtattacc

SEQ ID NO: 317 - DNA, 20 bp, Cannabis sativa
  misc_feature: Complimentary to position 2766 of SEQ. ID NO. 7  PT
tgcgttaacaaattacgacc

SEQ ID NO: 318 - DNA, 20 bp, Cannabis sativa
  misc_feature: Complimentary to position 2886 of SEQ. ID NO. 7  PT
atagataatttaattcaaaa

SEQ ID NO: 319 - DNA, 20 bp, Cannabis sativa
  misc_feature: Complimentary to position 2895 of SEQ. ID NO. 7  PT
ttaattcaaaatggcaaacc

SEQ ID NO: 320 - DNA, 20 bp, Cannabis sativa
  misc_feature: Complimentary to position 2898 of SEQ. ID NO. 7  PT
attcaaaatggcaaacctgg

SEQ ID NO: 321 - DNA, 20 bp, Cannabis sativa
  misc_feature: Complimentary to position 2902 of SEQ. ID NO. 7  PT
agcattttggttaatcctcc

SEQ ID NO: 322 - DNA, 20 bp, Cannabis sativa
  misc_feature: Complimentary to position 2915 of SEQ. ID NO. 7  PT
ttcatgcaatcttagcattt

SEQ ID NO: 323 - DNA, 20 bp, Cannabis sativa
  misc_feature: Complimentary to position 2962 of SEQ. ID NO. 7  PT
atcatttcttatttaattac

SEQ ID NO: 324 - DNA, 20 bp, Cannabis sativa
  misc_feature: Complimentary to position 2988 of SEQ. ID NO. 7  PT
gaaatgatgagaagaaatga

SEQ ID NO: 325 - DNA, 20 bp, Cannabis sativa
  misc_feature: Complimentary to position 2997 of SEQ. ID NO. 7  PT
agaagaaatgatggcaaacc

SEQ ID NO: 326 - DNA, 20 bp, Cannabis sativa
  misc_feature: Complimentary to position 3004 of SEQ. ID NO. 7  PT
taagaatttattaaaaaacc

SEQ ID NO: 327 - DNA, 20 bp, Cannabis sativa
  misc_feature: Complimentary to position 3817 of SEQ. ID NO. 7  PT
tatagatgagatgagatacc

SEQ ID NO: 328 - DNA, 20 bp, Cannabis sativa
  misc_feature: Complimentary to position 3818 of SEQ. ID NO. 7  PT
atagatgagatgagatacct

SEQ ID NO: 329 - DNA, 20 bp, Cannabis sativa
  misc_feature: Complimentary to position 3819 of SEQ. ID NO. 7  PT
tagatgagatgagatacctg

SEQ ID NO: 330 - DNA, 20 bp, Cannabis sativa
  misc_feature: Complimentary to position 3824 of SEQ. ID NO. 7  PT
tgctgggattatctggcccc

SEQ ID NO: 331 - DNA, 20 bp, Cannabis sativa
  misc_feature: Complimentary to position 3831 of SEQ. ID NO. 7  PT
ctatacttgctgggattatc

SEQ ID NO: 332 - DNA, 20 bp, Cannabis sativa
  misc_feature: Complimentary to position 3840 of SEQ. ID NO. 7  PT
atgtggctgctatacttgct

SEQ ID NO: 333 - DNA, 20 bp, Cannabis sativa
  misc_feature: Complimentary to position 3841 of SEQ. ID NO. 7  PT
tatgtggctgctatacttgc

SEQ ID NO: 334 - DNA, 20 bp, Cannabis sativa
  misc_feature: Complimentary to position 3854 of SEQ. ID NO. 7  PT
agcaagtatagcagccacat

SEQ ID NO: 335 - DNA, 20 bp, Cannabis sativa
  misc_feature: Complimentary to position 3857 of SEQ. ID NO. 7  PT
aattgttctcctatcctatg

SEQ ID NO: 336 - DNA, 20 bp, Cannabis sativa
  misc_feature: Complimentary to position 3859 of SEQ. ID NO. 7  PT
gtatagcagccacataggat

SEQ ID NO: 337 - DNA, 20 bp, Cannabis sativa
  misc_feature: Complimentary to position 3880 of SEQ. ID NO. 7  PT
aacttgacattattttgttc

SEQ ID NO: 338 - DNA, 20 bp, Cannabis sativa
  misc_feature: Complimentary to position 3896 of SEQ. ID NO. 7  PT
acaaaataatgtcaagtttc

SEQ ID NO: 339 - DNA, 20 bp, Cannabis sativa
  misc_feature: Complimentary to position 3910 of SEQ. ID NO. 7  PT
tcaacgttggcaagtaaata

SEQ ID NO: 340 - DNA, 20 bp, Cannabis sativa
  misc_feature: Complimentary to position 3923 of SEQ. ID NO. 7  PT
aagatttggcatatcaacgt

SEQ ID NO: 341 - DNA, 20 bp, Cannabis sativa
  misc_feature: Complimentary to position 3937 of SEQ. ID NO. 7  PT
tatatatttctttcaagatt

SEQ ID NO: 342 - DNA, 20 bp, Cannabis sativa
  misc_feature: Complimentary to position 3994 of SEQ. ID NO. 7  PT
aattatttaaactaattata

SEQ ID NO: 343 - DNA, 20 bp, Cannabis sativa
  misc_feature: Complimentary to position 4038 of SEQ. ID NO. 7  PT
aaagatgcttcagacgttga

SEQ ID NO: 344 - DNA, 20 bp, Cannabis sativa
  misc_feature: Complimentary to position 4069 of SEQ. ID NO. 7  PT
taaatcaatgggtgcagctt

SEQ ID NO: 345 - DNA, 20 bp, Cannabis sativa
  misc_feature: Complimentary to position 4080 of SEQ. ID NO. 7  PT
ctagcatttattaaatcaat

SEQ ID NO: 346 - DNA, 20 bp, Cannabis sativa
  misc_feature: Complimentary to position 4081 of SEQ. ID NO. 7  PT
gctagcatttattaaatcaa

SEQ ID NO: 347 - DNA, 20 bp, Cannabis sativa
  misc_feature: Complimentary to position 4095 of SEQ. ID NO. 7  PT
ttgatttaataaatgctagc

SEQ ID NO: 348 - DNA, 20 bp, Cannabis sativa
  misc_feature: Complimentary to position 4109 of SEQ. ID NO. 7  PT
gctagcaggaaagtaaaaga

SEQ ID NO: 349 - DNA, 20 bp, Cannabis sativa
  misc_feature: Complimentary to position 4121  of SEQ. ID NO. 7  PT
atttcaatttgattattttc

SEQ ID NO: 350 - DNA, 20 bp, Cannabis sativa
  misc_feature: Complimentary to position 4166 of SEQ. ID NO. 7  PT
atacaatcaaattaaaatac

SEQ ID NO: 351 - DNA, 20 bp, Cannabis sativa
  misc_feature: Complimentary to position 4167 of SEQ. ID NO. 7  PT
tacaatcaaattaaaataca

SEQ ID NO: 352 - DNA, 20 bp, Cannabis sativa
  misc_feature: Complimentary to position 4186 of SEQ. ID NO. 7  PT
agggaaatcgtttatgttat

SEQ ID NO: 353 - DNA, 20 bp, Cannabis sativa
  misc_feature: Complimentary to position 4224 of SEQ. ID NO. 7  PT
gaaacacgtattttagagat

SEQ ID NO: 354 - DNA, 20 bp, Cannabis sativa
  misc_feature: Complimentary to position 4473 of SEQ. ID NO. 7  PT
accacttaagaattttcttt

SEQ ID NO: 355 - DNA, 20 bp, Cannabis sativa
  misc_feature: Complimentary to position 4483 of SEQ. ID NO. 7  PT
gccaaaagaaaattcttaag

SEQ ID NO: 356 - DNA, 20 bp, Cannabis sativa
  misc_feature: Complimentary to position 4515 of SEQ. ID NO. 7  PT
caagatatactatataatat

SEQ ID NO: 357 - DNA, 20 bp, Cannabis sativa
  misc_feature: Complimentary to position 4527 of SEQ. ID NO. 7  PT
ctatattatatagtatatct

SEQ ID NO: 358 - DNA, 20 bp, Cannabis sativa
  misc_feature: Complimentary to position 4528 of SEQ. ID NO. 7  PT
tatattatatagtatatctt

SEQ ID NO: 359 - DNA, 20 bp, Cannabis sativa
  misc_feature: Complimentary to position 4573 of SEQ. ID NO. 7  PT
cctaccatttgagttgaggt

SEQ ID NO: 360 - DNA, 20 bp, Cannabis sativa
  misc_feature: Complimentary to position 4574 of SEQ. ID NO. 7  PT
tcctaccatttgagttgagg

SEQ ID NO: 361 - DNA, 20 bp, Cannabis sativa
  misc_feature: Complimentary to position 4577 of SEQ. ID NO. 7  PT
ttgtcctaccatttgagttg

SEQ ID NO: 362 - DNA, 20 bp, Cannabis sativa
  misc_feature: Complimentary to position 4580 of SEQ. ID NO. 7  PT
attacccacctcaactcaaa

SEQ ID NO: 363 - DNA, 20 bp, Cannabis sativa
  misc_feature: Complimentary to position 4584 of SEQ. ID NO. 7  PT
cccacctcaactcaaatggt

SEQ ID NO: 364 - DNA, 20 bp, Cannabis sativa
  misc_feature: Complimentary to position 4603 of SEQ. ID NO. 7  PT
taggacaagagctgctctgc

SEQ ID NO: 365 - DNA, 20 bp, Cannabis sativa
  misc_feature: Complimentary to position 4635 of SEQ. ID NO. 7  PT
atgtgaaatttgtaataata

SEQ ID NO: 366 - DNA, 20 bp, Cannabis sativa
  misc_feature: Complimentary to position 4636 of SEQ. ID NO. 7  PT
atgtgaaatttgtaataata

SEQ ID NO: 367 - DNA, 20 bp, Cannabis sativa
  misc_feature: Complimentary to position 4648 of SEQ. ID NO. 7  PT
tttttttaatctgcttgcac

SEQ ID NO: 368 - DNA, 20 bp, Cannabis sativa
  misc_feature: Complimentary to position 4763 of SEQ. ID NO. 7  PT
tactcttatagtaaccagag

SEQ ID NO: 369 - DNA, 20 bp, Cannabis sativa
  misc_feature: Complimentary to position 4766 of SEQ. ID NO. 7  PT
atagtaattagttacctctc

SEQ ID NO: 370 - DNA, 20 bp, Cannabis sativa
  misc_feature: Complimentary to position 4898 of SEQ. ID NO. 7  PT
ttatataaaaatatattcgt

SEQ ID NO: 371 - DNA, 20 bp, Cannabis sativa
  misc_feature: Complimentary to position 4915 of SEQ. ID NO. 7  PT
aatatatttttatataaata

SEQ ID NO: 372 - DNA, 20 bp, Cannabis sativa
  misc_feature: Complimentary to position 4916 of SEQ. ID NO. 7  PT
atatatttttatataaatat

SEQ ID NO: 373 - DNA, 20 bp, Cannabis sativa
  misc_feature: Complimentary to position 4989 of SEQ. ID NO. 7  PT
aattatctcatctaactaaa

SEQ ID NO: 374 - DNA, 20 bp, Cannabis sativa
  misc_feature: Complimentary to position 5099 of SEQ. ID NO. 7  PT
tgttagtaaagtaaaatacc

SEQ ID NO: 375 - DNA, 20 bp, Cannabis sativa
  misc_feature: Complimentary to position 5106 of SEQ. ID NO. 7  PT
tgcatttcttctcaatttcc

SEQ ID NO: 376 - DNA, 20 bp, Cannabis sativa
  misc_feature: Complimentary to position 5121 of SEQ. ID NO. 7  PT
gaaattgagaagaaatgcag

SEQ ID NO: 377 - DNA, 20 bp, Cannabis sativa
  misc_feature: Complimentary to position 5125 of SEQ. ID NO. 7  PT
ttgagaagaaatgcagtgga

SEQ ID NO: 378 - DNA, 20 bp, Cannabis sativa
  misc_feature: Complimentary to position 5146 of SEQ. ID NO. 7  PT
ggattttgcttccatctaaa

SEQ ID NO: 379 - DNA, 20 bp, Cannabis sativa
  misc_feature: Complimentary to position 5146, -1, of SEQ. ID NO. 7  PT
attctgttccaccatttaga

SEQ ID NO: 380 - DNA, 20 bp, Cannabis sativa
  misc_feature: Complimentary to position 5149 of SEQ. ID NO. 7  PT
ttttgcttccatctaaatgg

SEQ ID NO: 381 - DNA, 20 bp, Cannabis sativa
  misc_feature: Complimentary to position 5177 of SEQ. ID NO. 7  PT
tactgttttggtatttttgg

SEQ ID NO: 382 - DNA, 20 bp, Cannabis sativa
  misc_feature: Complimentary to position 5180 of SEQ. ID NO. 7  PT
ggctactgttttggtatttt

SEQ ID NO: 383 - DNA, 20 bp, Cannabis sativa
  misc_feature: Complimentary to position 5189 of SEQ. ID NO. 7  PT
tatatatttggctactgttt

SEQ ID NO: 384 - DNA, 20 bp, Cannabis sativa
  misc_feature: Complimentary to position 5201 of SEQ. ID NO. 7  PT
ggtggaccactctatatatt

SEQ ID NO: 385 - DNA, 20 bp, Cannabis sativa
  misc_feature: Complimentary to position 5206 of SEQ. ID NO. 7  PT
cagtagccaaatatatagag

SEQ ID NO: 386 - DNA, 20 bp, Cannabis sativa
  misc_feature: Complimentary to position 5219 of SEQ. ID NO. 7  PT
ataactataaaaatgaaggg

SEQ ID NO: 387 - DNA, 20 bp, Cannabis sativa
  misc_feature: Complimentary to position 5222  of SEQ. ID NO. 7  PT
ataataactataaaaatgaa

SEQ ID NO: 388 - DNA, 20 bp, Cannabis sativa
  misc_feature: Complimentary to position 5223 of SEQ. ID NO. 7  PT
gataataactataaaaatga

SEQ ID NO: 389 - DNA, 20 bp, Cannabis sativa
  misc_feature: Complimentary to position 5249 of SEQ. ID NO. 7  PT
agcataattgtggcactgtt

SEQ ID NO: 390 - DNA, 20 bp, Cannabis sativa
  misc_feature: Complimentary to position 5259 of SEQ. ID NO. 7  PT
ttggattatgagcataattg

SEQ ID NO: 391 - DNA, 20 bp, Cannabis sativa
  misc_feature: Complimentary to position 5278of SEQ. ID NO. 7  PT
aaatatcagtaaacacagct

SEQ ID NO: 392 - DNA, 20 bp, Cannabis sativa
  misc_feature: Complimentary to position 5301 of SEQ. ID NO. 7  PT
tgatctaccactagcttcag

SEQ ID NO: 393 - DNA, 20 bp, Cannabis sativa
  misc_feature: Complimentary to position 5302 of SEQ. ID NO. 7  PT
ctgatctaccactagcttca

SEQ ID NO: 394 - DNA, 20 bp, Cannabis sativa
  misc_feature: Complimentary to position 5303 of SEQ. ID NO. 7  PT
cctgatctaccactagcttc

SEQ ID NO: 395 - DNA, 20 bp, Cannabis sativa
  misc_feature: Complimentary to position 5305 of SEQ. ID NO. 7  PT
gatatttcccctgaagctag

SEQ ID NO: 396 - DNA, 20 bp, Cannabis sativa
  misc_feature: Complimentary to position 5314 of SEQ. ID NO. 7  PT
cctgaagctagtggtagatc

SEQ ID NO: 397 - DNA, 20 bp, Cannabis sativa
  misc_feature: Complimentary to position 5403 of SEQ. ID NO. 7  PT
tacaaaaaattgttgtagct

SEQ ID NO: 398 - DNA, 20 bp, Cannabis sativa
  misc_feature: Complimentary to position 5404 of SEQ. ID NO. 7  PT
acaaaaaattgttgtagcta

SEQ ID NO: 399 - DNA, 20 bp, Cannabis sativa
  misc_feature: Complimentary to position 5484 of SEQ. ID NO. 7  PT
tgtgggtcataaataaagtt

SEQ ID NO: 400 - DNA, 20 bp, Cannabis sativa
  misc_feature: Complimentary to position 5485 of SEQ. ID NO. 7  PT
ttgtgggtcataaataaagt

SEQ ID NO: 401 - DNA, 20 bp, Cannabis sativa
  misc_feature: Complimentary to position 5501 of SEQ. ID NO. 7  PT
ataacaattataatttttgt

SEQ ID NO: 402 - DNA, 20 bp, Cannabis sativa
  misc_feature: Complimentary to position 5502 of SEQ. ID NO. 7  PT
gataacaattataatttttg

SEQ ID NO: 403 - DNA, 20 bp, Cannabis sativa
  misc_feature: Complimentary to position 5524 of SEQ. ID NO. 7  PT
tatgctattgaacttctagt

SEQ ID NO: 404 - DNA, 20 bp, Cannabis sativa
  misc_feature: Complimentary to position 5565 of SEQ. ID NO. 7  PT
taatttgagttaataatttt

SEQ ID NO: 405 - DNA, 20 bp, Cannabis sativa
  misc_feature: Complimentary to position 5737 of SEQ. ID NO. 7  PT
tttacgatcttcacattgac

SEQ ID NO: 406 - DNA, 20 bp, Cannabis sativa
  misc_feature: Complimentary to position 5760 of SEQ. ID NO. 7  PT
aagatcgtaaatctgattga

SEQ ID NO: 407 - DNA, 20 bp, Cannabis sativa
  misc_feature: Complimentary to position 5802 of SEQ. ID NO. 7  PT
caaggcattcttttttttgg

SEQ ID NO: 408 - DNA, 20 bp, Cannabis sativa
  misc_feature: Complimentary to position 5805 of SEQ. ID NO. 7  PT
gttcaaggcattcttttttt

SEQ ID NO: 409 - DNA, 20 bp, Cannabis sativa
  misc_feature: Complimentary to position 5820 of SEQ. ID NO. 7  PT
aagttggtctctgatgttca

SEQ ID NO: 410 - DNA, 20 bp, Cannabis sativa
  misc_feature: Complimentary to position 5836 of SEQ. ID NO. 7  PT
ataacacaaatttaataagt

SEQ ID NO: 411 - DNA, 20 bp, Cannabis sativa
  misc_feature: Complimentary to position 5872 of SEQ. ID NO. 7  PT
gggagagcagtggattgttt

SEQ ID NO: 412 - DNA, 20 bp, Cannabis sativa
  misc_feature: Complimentary to position 5873 of SEQ. ID NO. 7  PT
tgggagagcagtggattgtt

SEQ ID NO: 413 - DNA, 20 bp, Cannabis sativa
  misc_feature: Complimentary to position 5882 of SEQ. ID NO. 7  PT
attttggtatgggagagcag

SEQ ID NO: 414 - DNA, 20 bp, Cannabis sativa
  misc_feature: Complimentary to position 5892 of SEQ. ID NO. 7  PT
atttgttttaattttggtat

SEQ ID NO: 415 - DNA, 20 bp, Cannabis sativa
  misc_feature: Complimentary to position 5893 of SEQ. ID NO. 7  PT
tatttgttttaattttggta

SEQ ID NO: 416 - DNA, 20 bp, Cannabis sativa
  misc_feature: Complimentary to position 5898 of SEQ. ID NO. 7  PT
atatatatttgttttaattt

SEQ ID NO: 417 - DNA, 20 bp, Cannabis sativa
  misc_feature: Complimentary to position 5979 of SEQ. ID NO. 7  PT
ttagatttgagagttaaatg

SEQ ID NO: 418 - DNA, 20 bp, Cannabis sativa
  misc_feature: Complimentary to position 5998 of SEQ. ID NO. 7  PT
aactctcaaatctaaatttt

SEQ ID NO: 419 - DNA, 20 bp, Cannabis sativa
  misc_feature: Complimentary to position 5999 of SEQ. ID NO. 7  PT
actctcaaatctaaattttt

SEQ ID NO: 420 - DNA, 20 bp, Cannabis sativa
  misc_feature: Complimentary to position 6000 of SEQ. ID NO. 7  PT
ctctcaaatctaaatttttg

SEQ ID NO: 421 - DNA, 20 bp, Cannabis sativa
  misc_feature: Complimentary to position 6033 of SEQ. ID NO. 7  PT
ttaatcttttttgttattta

SEQ ID NO: 422 - DNA, 20 bp, Cannabis sativa
  misc_feature: Complimentary to position 6036 of SEQ. ID NO. 7  PT
atcttttttgttatttaagg

SEQ ID NO: 423 - DNA, 20 bp, Cannabis sativa
  misc_feature: Complimentary to position 9091 of SEQ. ID NO. 7  PT
atactttctgtgcaaaaata

SEQ ID NO: 424 - DNA, 20 bp, Cannabis sativa
  misc_feature: Complimentary to position 9190 of SEQ. ID NO. 7  PT
tagcatttacttcatgcgct

SEQ ID NO: 425 - DNA, 20 bp, Cannabis sativa
  misc_feature: Complimentary to position 9214 of SEQ. ID NO. 7  PT
gtaaatgctatgattgtata

SEQ ID NO: 426 - DNA, 20 bp, Cannabis sativa
  misc_feature: Complimentary to position 9238 of SEQ. ID NO. 7  PT
taaactttgggaaggcatgt

SEQ ID NO: 427 - DNA, 20 bp, Cannabis sativa
  misc_feature: Complimentary to position 9246 of SEQ. ID NO. 7  PT
taaaattttaaactttggga

SEQ ID NO: 428 - DNA, 20 bp, Cannabis sativa
  misc_feature: Complimentary to 9250 9776  of SEQ. ID NO. 7 PT
caactaaaattttaaacttt

SEQ ID NO: 429 - DNA, 20 bp, Cannabis sativa
  misc_feature: Complimentary to position 9251 of SEQ. ID NO. 7  PT
gcaactaaaattttaaactt

SEQ ID NO: 430 - DNA, 20 bp, Cannabis sativa
  misc_feature: Complimentary to position 9286 of SEQ. ID NO. 7  PT
actgaatgattatcagattc

SEQ ID NO: 431 - DNA, 20 bp, Cannabis sativa
  misc_feature: Complimentary to position 9289 of SEQ. ID NO. 7  PT
gaatgattatcagattctgg

SEQ ID NO: 432 - DNA, 20 bp, Cannabis sativa
  misc_feature: Complimentary to position 9324 of SEQ. ID NO. 7  PT
aataataataatgtgttaac

SEQ ID NO: 433 - DNA, 20 bp, Cannabis sativa
  misc_feature: Complimentary to position 9379 of SEQ. ID NO. 7  PT
gttgtgtatttttttctttt

SEQ ID NO: 434 - DNA, 20 bp, Cannabis sativa
  misc_feature: Complimentary to position 9420 of SEQ. ID NO. 7  PT
taatattatatttaattaga

SEQ ID NO: 435 - DNA, 20 bp, Cannabis sativa
  misc_feature: Complimentary to position 9421 of SEQ. ID NO. 7  PT
ttaatattatatttaattag

SEQ ID NO: 436 - DNA, 20 bp, Cannabis sativa
  misc_feature: Complimentary to position 9510 of SEQ. ID NO. 7  PT
gagacacacacataccctaa

SEQ ID NO: 437 - DNA, 20 bp, Cannabis sativa
  misc_feature: Complimentary to position 9513 of SEQ. ID NO. 7  PT
aatcgcaaaaaattccatta

SEQ ID NO: 438 - DNA, 20 bp, Cannabis sativa
  misc_feature: Complimentary to position 9514 of SEQ. ID NO. 7  PT
caatcgcaaaaaattccatt

SEQ ID NO: 439 - DNA, 20 bp, Cannabis sativa
  misc_feature: Complimentary to position 9567 of SEQ. ID NO. 7  PT
gttttgtagatgaaaactct

SEQ ID NO: 440 - DNA, 20 bp, Cannabis sativa
  misc_feature: Complimentary to position 9570 of SEQ. ID NO. 7  PT
ttgtagatgaaaactcttgg

SEQ ID NO: 441 - DNA, 20 bp, Cannabis sativa
  misc_feature: Complimentary to position 9585 of SEQ. ID NO. 7  PT
cttggtggagcaatgtttag

SEQ ID NO: 442 - DNA, 20 bp, Cannabis sativa
  misc_feature: Complimentary to position 5986 of SEQ. ID NO. 7  PT
ttggtggagcaatgtttaga

SEQ ID NO: 443 - DNA, 20 bp, Cannabis sativa
  misc_feature: Complimentary to position 9613 of SEQ. ID NO. 7  PT
ttattgtaagagtatttaat

SEQ ID NO: 444 - DNA, 20 bp, Cannabis sativa
  misc_feature: Complimentary to position 9621 of SEQ. ID NO. 7  PT
agagtatttaattggtgttt

SEQ ID NO: 445 - DNA, 20 bp, Cannabis sativa
  misc_feature: Complimentary to position 9622 of SEQ. ID NO. 7  PT
gagtatttaattggtgtttt

SEQ ID NO: 446 - DNA, 20 bp, Cannabis sativa
  misc_feature: Complimentary to position 9623 of SEQ. ID NO. 7  PT
agtatttaattggtgttttg

SEQ ID NO: 447 - DNA, 20 bp, Cannabis sativa
  misc_feature: Complimentary to position 9642 of SEQ. ID NO. 7  PT
ggggtgtcgatataataatg

SEQ ID NO: 448 - DNA, 20 bp, Cannabis sativa
  misc_feature: Complimentary to position 9648 of SEQ. ID NO. 7  PT
tcgatataataatgaggttt

SEQ ID NO: 449 - DNA, 20 bp, Cannabis sativa
  misc_feature: Complimentary to position 9649 of SEQ. ID NO. 7  PT
cgatataataatgaggtttt

SEQ ID NO: 450 - DNA, 20 bp, Cannabis sativa
  misc_feature: Complimentary to position 9664 of SEQ. ID NO. 7  PT
gttttgggattattattgtg

SEQ ID NO: 451 - DNA, 20 bp, Cannabis sativa
  misc_feature: Complimentary to position 9680 of SEQ. ID NO. 7  PT
tgtgaggatttaataaagta

SEQ ID NO: 452 - DNA, 20 bp, Cannabis sativa
  misc_feature: Complimentary to position 9702  of SEQ. ID NO. 7  PT
gtaattagtttgaaatgaaa

SEQ ID NO: 453 - DNA, 20 bp, Cannabis sativa
  misc_feature: Complimentary to position 9731 of SEQ. ID NO. 7  PT
caatcaataataatcttcat

SEQ ID NO: 454 - DNA, 20 bp, Cannabis sativa
  misc_feature: Complimentary to position 9732 of SEQ. ID NO. 7  PT
tcaatcaataataatcttca

SEQ ID NO: 455 - DNA, 20 bp, Cannabis sativa
  misc_feature: Complimentary to position 9765 of SEQ. ID NO. 7  PT
aatattaaaaagaaaaaatg

SEQ ID NO: 456 - DNA, 20 bp, Cannabis sativa
  misc_feature: Complimentary to position 9766 of SEQ. ID NO. 7  PT
atattaaaaagaaaaaatga

SEQ ID NO: 457 - DNA, 20 bp, Cannabis sativa
  misc_feature: Complimentary to position 9775 of SEQ. ID NO. 7  PT
agaaaaaatgagggaaagaa

SEQ ID NO: 458 - DNA, 20 bp, Cannabis sativa
  misc_feature: Complimentary to position 9776  of SEQ. ID NO. 7  PT
gaaaaaatgagggaaagaaa

SEQ ID NO: 459 - DNA, 20 bp, Cannabis sativa
  misc_feature: Complimentary to position 606 of SEQ. ID NO. 10  OLS CM011613.12335391-2338392
catacataatatatatatat

SEQ ID NO: 460 - DNA, 20 bp, Cannabis sativa
  misc_feature: Complimentary to position 694 of SEQ. ID NO. 10  OLS CM011613.12335391-2338392
tatgaatcatcttcgtgctg

SEQ ID NO: 461 - DNA, 20 bp, Cannabis sativa
  misc_feature: Complimentary to position 695 of SEQ. ID NO. 10  OLS CM011613.12335391-2338392
atgaatcatcttcgtgctga

SEQ ID NO: 462 - DNA, 20 bp, Cannabis sativa
  misc_feature: Complimentary to position 700 of SEQ. ID NO. 10  OLS CM011613.12335391-2338392
tcatcttcgtgctgagggtc

SEQ ID NO: 463 - DNA, 20 bp, Cannabis sativa
  misc_feature: Complimentary to position 708 of SEQ. ID NO. 10  OLS CM011613.12335391-2338392
ccgatggcgagaacggaggc

SEQ ID NO: 464 - DNA, 20 bp, Cannabis sativa
  misc_feature: Complimentary to position 712 of SEQ. ID NO. 10  OLS CM011613.12335391-2338392
ggtgccgatggcgagaacgg

SEQ ID NO: 465 - DNA, 20 bp, Cannabis sativa
  misc_feature: Complimentary to position 715 of SEQ. ID NO. 10  OLS CM011613.12335391-2338392
ggcggtgccgatggcgagaa

SEQ ID NO: 466 - DNA, 20 bp, Cannabis sativa
  misc_feature: Complimentary to position 719 of SEQ. ID NO. 10  OLS CM011613.12335391-2338392
ccggcctccgttctcgccat

SEQ ID NO: 467 - DNA, 20 bp, Cannabis sativa
  misc_feature: Complimentary to position 724 of SEQ. ID NO. 10  OLS CM011613.12335391-2338392
ctccggattggcggtgccga

SEQ ID NO: 468 - DNA, 20 bp, Cannabis sativa
  misc_feature: Complimentary to position 733 of SEQ. ID NO. 10  OLS CM011613.12335391-2338392
cgccatcggcaccgccaatc

SEQ ID NO: 469 - DNA, 20 bp, Cannabis sativa
  misc_feature: Complimentary to position 13733, -1, 21 of SEQ. ID NO. 10  OLS CM011613.12335391-2338392
taaaatgttctccggattgg

SEQ ID NO: 470 - DNA, 20 bp, Cannabis sativa
  misc_feature: Complimentary to position 736 of SEQ. ID NO. 10  OLS CM011613.12335391-2338392
tattaaaatgttctccggat

SEQ ID NO: 471 - DNA, 20 bp, Cannabis sativa
  misc_feature: Complimentary to position 741 of SEQ. ID NO. 10  OLS CM011613.12335391-2338392
tcttgtattaaaatgttctc

SEQ ID NO: 472 - DNA, 20 bp, Cannabis sativa
  misc_feature: Complimentary to position 771 of SEQ. ID NO. 10  OLS CM011613.12335391-2338392
gtgacccgaaagtagtagtc

SEQ ID NO: 473 - DNA, 20 bp, Cannabis sativa
  misc_feature: Complimentary to position 777 of SEQ. ID NO. 10  OLS CM011613.12335391-2338392
agtttcctgactactacttt

SEQ ID NO: 474 - DNA, 20 bp, Cannabis sativa
  misc_feature: Complimentary to position 778 of SEQ. ID NO. 10  OLS CM011613.12335391-2338392
gtttcctgactactactttc

SEQ ID NO: 475 - DNA, 20 bp, Cannabis sativa
  misc_feature: Complimentary to position 793 of SEQ. ID NO. 10  OLS CM011613.12335391-2338392
ttgagtcatgtgttcacttt

SEQ ID NO: 476 - DNA, 20 bp, Cannabis sativa
  misc_feature: Complimentary to position 876 of SEQ. ID NO. 10  OLS CM011613.12335391-2338392
catggaaaagtattattagt

SEQ ID NO: 477 - DNA, 20 bp, Cannabis sativa
  misc_feature: Complimentary to position 894 of SEQ. ID NO. 10  OLS CM011613.12335391-2338392
aaatagataatgcttataca

SEQ ID NO: 478 - DNA, 20 bp, Cannabis sativa
  misc_feature: Complimentary to position 917 of SEQ. ID NO. 10  OLS CM011613.12335391-2338392
attatctatttatataataa

SEQ ID NO: 479 - DNA, 20 bp, Cannabis sativa
  misc_feature: Complimentary to position 1000 of SEQ. ID NO. 10  OLS CM011613.12335391-2338392
cttactttatatgtatatgt

SEQ ID NO: 480 - DNA, 20 bp, Cannabis sativa
  misc_feature: Complimentary to position 1019 of SEQ. ID NO. 10  OLS CM011613.12335391-2338392
taggtgacaaaagtatgata

SEQ ID NO: 481 - DNA, 20 bp, Cannabis sativa
  misc_feature: Complimentary to position 1071  of SEQ. ID NO. 10  OLS CM011613.12335391-2338392
tctaaagcaaaacccaagat

SEQ ID NO: 482 - DNA, 20 bp, Cannabis sativa
  misc_feature: Complimentary to position 1072 of SEQ. ID NO. 10  OLS CM011613.12335391-2338392
tctcgtgctccgccaatctt

SEQ ID NO: 483 - DNA, 20 bp, Cannabis sativa
  misc_feature: Complimentary to position 1073 of SEQ. ID NO. 10  OLS CM011613.12335391-2338392
atctcgtgctccgccaatct

SEQ ID NO: 484 - DNA, 20 bp, Cannabis sativa
  misc_feature: Complimentary to position 1074 of SEQ. ID NO. 10  OLS CM011613.12335391-2338392
aaagcaaaacccaagattgg

SEQ ID NO: 485 - DNA, 20 bp, Cannabis sativa
  misc_feature: Complimentary to position 1095 of SEQ. ID NO. 10  OLS CM011613.12335391-2338392
ggagcacgagatgcaaactc

SEQ ID NO: 486 - DNA, 20 bp, Cannabis sativa
  misc_feature: Complimentary to position 1116 of SEQ. ID NO. 10  OLS CM011613.12335391-2338392
ggatgcacgtcaagacatgt

SEQ ID NO: 487 - DNA, 20 bp, Cannabis sativa
  misc_feature: Complimentary to position 1125 of SEQ. ID NO. 10  OLS CM011613.12335391-2338392
tcaagacatgttggtagttg

SEQ ID NO: 488 - DNA, 20 bp, Cannabis sativa
  misc_feature: Complimentary to position 1138 of SEQ. ID NO. 10  OLS CM011613.12335391-2338392
gtagttgaggttccaaaact

SEQ ID NO: 489 - DNA, 20 bp, Cannabis sativa
  misc_feature: Complimentary to position 1139 of SEQ. ID NO. 10  OLS CM011613.12335391-2338392
tagttgaggttccaaaactt

SEQ ID NO: 490 - DNA, 20 bp, Cannabis sativa
  misc_feature: Complimentary to position 1139, -1, of SEQ. ID NO. 10  OLS CM011613.12335391-2338392
caagcatccttcccaagttt

SEQ ID NO: 491 - DNA, 20 bp, Cannabis sativa
  misc_feature: Complimentary to position 1143 of SEQ. ID NO. 10  OLS CM011613.12335391-2338392
tgaggttccaaaacttggga

SEQ ID NO: 492 - DNA, 20 bp, Cannabis sativa
  misc_feature: Complimentary to position 1158 of SEQ. ID NO. 10  OLS CM011613.12335391-2338392
tgggaaggatgcttgtgcaa

SEQ ID NO: 493 - DNA, 20 bp, Cannabis sativa
  misc_feature: Complimentary to position 1170 of SEQ. ID NO. 10  OLS CM011613.12335391-2338392
gggttgaccccattctttga

SEQ ID NO: 494 - DNA, 20 bp, Cannabis sativa
  misc_feature: Complimentary to position 1172 of SEQ. ID NO. 10  OLS CM011613.12335391-2338392
gtgcaaaggccatcaaagaa

SEQ ID NO: 495 - DNA, 20 bp, Cannabis sativa
  misc_feature: Complimentary to position 1173 of SEQ. ID NO. 10  OLS CM011613.12335391-2338392
tgcaaaggccatcaaagaat

SEQ ID NO: 496 - DNA, 20 bp, Cannabis sativa
  misc_feature: Complimentary to position 1174 of SEQ. ID NO. 10  OLS CM011613.12335391-2338392
gcaaaggccatcaaagaatg

SEQ ID NO: 497 - DNA, 20 bp, Cannabis sativa
  misc_feature: Complimentary to position 1190 of SEQ. ID NO. 10  OLS CM011613.12335391-2338392
aaatgagtgattttagactt

SEQ ID NO: 498 - DNA, 20 bp, Cannabis sativa
  misc_feature: Complimentary to position 1191 of SEQ. ID NO. 10  OLS CM011613.12335391-2338392
taaatgagtgattttagact

SEQ ID NO: 499 - DNA, 20 bp, Cannabis sativa
  misc_feature: Complimentary to position 1233 of SEQ. ID NO. 10  OLS CM011613.12335391-2338392
gtctgcaccgggcatgtcag

SEQ ID NO: 500 - DNA, 20 bp, Cannabis sativa
  misc_feature: Complimentary to position 1237 of SEQ. ID NO. 10  OLS CM011613.12335391-2338392
gcatcaaccactgacatgcc

SEQ ID NO: 501 - DNA, 20 bp, Cannabis sativa
  misc_feature: Complimentary to position 1244 of SEQ. ID NO. 10  OLS CM011613.12335391-2338392
gcgcaatggtagtctgcacc

SEQ ID NO: 502 - DNA, 20 bp, Cannabis sativa
  misc_feature: Complimentary to position 1245 of SEQ. ID NO. 10  OLS CM011613.12335391-2338392
agcgcaatggtagtctgcac

SEQ ID NO: 503 - DNA, 20 bp, Cannabis sativa
  misc_feature: Complimentary to position 1258 of SEQ. ID NO. 10  OLS CM011613.12335391-2338392
gtccgagaagcttagcgcaa

SEQ ID NO: 504 - DNA, 20 bp, Cannabis sativa
  misc_feature: Complimentary to position 1267 of SEQ. ID NO. 10  OLS CM011613.12335391-2338392
taccattgcgctaagcttct

SEQ ID NO: 505 - DNA, 20 bp, Cannabis sativa
  misc_feature: Complimentary to position 1286 of SEQ. ID NO. 10  OLS CM011613.12335391-2338392
atcatcacacgcttcactga

SEQ ID NO: 506 - DNA, 20 bp, Cannabis sativa
  misc_feature: Complimentary to position 1287 of SEQ. ID NO. 10  OLS CM011613.12335391-2338392
catcatcacacgcttcactg

SEQ ID NO: 507 - DNA, 20 bp, Cannabis sativa
  misc_feature: Complimentary to position 1309 of SEQ. ID NO. 10  OLS CM011613.12335391-2338392
cgtgtgatgatgtatcaact

SEQ ID NO: 508 - DNA, 20 bp, Cannabis sativa
  misc_feature: Complimentary to position 1318 of SEQ. ID NO. 10  OLS CM011613.12335391-2338392
atgtatcaactaggctgtta

SEQ ID NO: 509 - DNA, 20 bp, Cannabis sativa
  misc_feature: Complimentary to position 1321 of SEQ. ID NO. 10  OLS CM011613.12335391-2338392
tatcaactaggctgttatgg

SEQ ID NO: 510 - DNA, 20 bp, Cannabis sativa
  misc_feature: Complimentary to position 1017 of SEQ. ID NO. 13 AAE1 CM011611.11210973-1228229
attcaaagtgagaaaattgt

SEQ ID NO: 511 - DNA, 20 bp, Cannabis sativa
  misc_feature: Complimentary to position 1036 of SEQ. ID NO. 13 AAE1 CM011611.11210973-1228229
aaccaacaagatcatgagaa

SEQ ID NO: 512 - DNA, 20 bp, Cannabis sativa
  misc_feature: Complimentary to position 1037 of SEQ. ID NO. 13 AAE1 CM011611.11210973-1228229
caaccaacaagatcatgaga

SEQ ID NO: 513 - DNA, 20 bp, Cannabis sativa
  misc_feature: Complimentary to position 1945 of SEQ. ID NO. 13 AAE1 CM011611.11210973-1228229
aacccttctcatgatcttgt

SEQ ID NO: 514 - DNA, 20 bp, Cannabis sativa
  misc_feature: Complimentary to position 1058 of SEQ. ID NO. 13 AAE1 CM011611.11210973-1228229
atcttgttggttgctgttct

SEQ ID NO: 515 - DNA, 20 bp, Cannabis sativa
  misc_feature: Complimentary to position 1073 of SEQ. ID NO. 13 AAE1 CM011611.11210973-1228229
gttctcggaagtgatgaaag

SEQ ID NO: 516 - DNA, 20 bp, Cannabis sativa
  misc_feature: Complimentary to position 1099 of SEQ. ID NO. 13 AAE1 CM011611.11210973-1228229
tgtttggttaattattaaat

SEQ ID NO: 517 - DNA, 20 bp, Cannabis sativa
  misc_feature: Complimentary to position 1115 of SEQ. ID NO. 13 AAE1 CM011611.11210973-1228229
actaattctaatttggtgtt

SEQ ID NO: 518 - DNA, 20 bp, Cannabis sativa
  misc_feature: Complimentary to position 1122 of SEQ. ID NO. 13 AAE1 CM011611.11210973-1228229
attgtatactaattctaatt

SEQ ID NO: 519 - DNA, 20 bp, Cannabis sativa
  misc_feature: Complimentary to position 1157 of SEQ. ID NO. 13 AAE1 CM011611.11210973-1228229
ataagatcatcatcatagtg

SEQ ID NO: 520 - DNA, 20 bp, Cannabis sativa
  misc_feature: Complimentary to position 1168 of SEQ. ID NO. 13 AAE1 CM011611.11210973-1228229
atcatagtgtggaagtgtat

SEQ ID NO: 521 - DNA, 20 bp, Cannabis sativa
  misc_feature: Complimentary to position 1213 of SEQ. ID NO. 13 AAE1 CM011611.11210973-1228229
tttttagtcttgactctcaa

SEQ ID NO: 522 - DNA, 20 bp, Cannabis sativa
  misc_feature: Complimentary to position 1214 of SEQ. ID NO. 13 AAE1 CM011611.11210973-1228229
atttttagtcttgactctca

SEQ ID NO: 523 - DNA, 20 bp, Cannabis sativa
  misc_feature: Complimentary to position 1259 of SEQ. ID NO. 13 AAE1 CM011611.11210973-1228229
aaagttaataatgatgaaat

SEQ ID NO: 524 - DNA, 20 bp, Cannabis sativa
  misc_feature: Complimentary to position 1274 of SEQ. ID NO. 13 AAE1 CM011611.11210973-1228229
gaaattggtaccttgaacag

SEQ ID NO: 525 - DNA, 20 bp, Cannabis sativa
  misc_feature: Complimentary to position 1305 of SEQ. ID NO. 13 AAE1 CM011611.11210973-1228229
ttgaggttatctttcaactt

SEQ ID NO: 526 - DNA, 20 bp, Cannabis sativa
  misc_feature: Complimentary to position 1306 of SEQ. ID NO. 13 AAE1 CM011611.11210973-1228229
attgaggttatctttcaact

SEQ ID NO: 527 - DNA, 20 bp, Cannabis sativa
  misc_feature: Complimentary to position 1322 of SEQ. ID NO. 13 AAE1 CM011611.11210973-1228229
caattgacttaaatcaattg

SEQ ID NO: 528 - DNA, 20 bp, Cannabis sativa
  misc_feature: Complimentary to position 1382 of SEQ. ID NO. 13 AAE1 CM011611.11210973-1228229
aagaaaattactaattgctc

SEQ ID NO: 529 - DNA, 20 bp, Cannabis sativa
  misc_feature: Complimentary to position 1395 of SEQ. ID NO. 13 AAE1 CM011611.11210973-1228229
ggggtgccacctttgggcgg

SEQ ID NO: 530 - DNA, 20 bp, Cannabis sativa
  misc_feature: Complimentary to position 1397 of SEQ. ID NO. 13 AAE1 CM011611.11210973-1228229
tgctcaggtccaccgcccaa

SEQ ID NO: 531 - DNA, 20 bp, Cannabis sativa
  misc_feature: Complimentary to position 1398 of SEQ. ID NO. 13 AAE1 CM011611.11210973-
attggggtgccacctttggg

SEQ ID NO: 532 - DNA, 20 bp, Cannabis sativa
  misc_feature: Complimentary to position 1400 of SEQ. ID NO. 13 AAE1 CM011611.11210973-1228229
tcaggtccaccgcccaaagg

SEQ ID NO: 533 - DNA, 20 bp, Cannabis sativa
  misc_feature: Complimentary to position 1401 of SEQ. ID NO. 13 AAE1 CM011611.11210973-1228229
gctattggggtgccaccttt

SEQ ID NO: 534 - DNA, 20 bp, Cannabis sativa
  misc_feature: Complimentary to position 1402 of SEQ. ID NO. 13 AAE1 CM011611.11210973-1228229
tgctattggggtgccacctt

SEQ ID NO: 535 - DNA, 20 bp, Cannabis sativa
  misc_feature: Complimentary to position 1414 of SEQ. ID NO. 13 AAE1 CM011611.11210973-1228229
tttcgagacaactgctattg

SEQ ID NO: 536 - DNA, 20 bp, Cannabis sativa
  misc_feature: Complimentary to position 1415 of SEQ. ID NO. 13 AAE1 CM011611.11210973-1228229
ttttcgagacaactgctatt

SEQ ID NO: 537 - DNA, 20 bp, Cannabis sativa
  misc_feature: Complimentary to position 1416 of SEQ. ID NO. 13 AAE1 CM011611.11210973-1228229
gttttcgagacaactgctat

SEQ ID NO: 538 - DNA, 20 bp, Cannabis sativa
  misc_feature: Complimentary to position 1555 of SEQ. ID NO. 13 AAE1 CM011611.11210973-1228229
gttacttttgataatttgat

SEQ ID NO: 539 - DNA, 20 bp, Cannabis sativa
  misc_feature: Complimentary to position 7139 of SEQ. ID NO. 13 AAE1 CM011611.11210973-1228229
aatgaatattggaggcatca

SEQ ID NO: 540 - DNA, 20 bp, Cannabis sativa
  misc_feature: Complimentary to position 7147 of SEQ. ID NO. 13 AAE1 CM011611.11210973-1228229
gatgatacaatgaatattgg

SEQ ID NO: 541 - DNA, 20 bp, Cannabis sativa
  misc_feature: Complimentary to position 7150 of SEQ. ID NO. 13 AAE1 CM011611.11210973-1228229
gcagatgatacaatgaatat

SEQ ID NO: 542 - DNA, 20 bp, Cannabis sativa
  misc_feature: Complimentary to position 7177 of SEQ. ID NO. 13 AAE1 CM011611.11210973-1228229
aatggttattatcatgcaca

SEQ ID NO: 543 - DNA, 20 bp, Cannabis sativa
  misc_feature: Complimentary to position 7195 of SEQ. ID NO. 13 AAE1 CM011611.11210973-1228229
atttttgagcttacatctaa

SEQ ID NO: 544 - DNA, 20 bp, Cannabis sativa
  misc_feature: Complimentary to position 7220 of SEQ. ID NO. 13 AAE1 CM011611.11210973-1228229
agaggttttaaggaggcatg

SEQ ID NO: 545 - DNA, 20 bp, Cannabis sativa
  misc_feature: Complimentary to position 7221 of SEQ. ID NO. 13 AAE1 CM011611.11210973-1228229
gagaggttttaaggaggcat

SEQ ID NO: 546 - DNA, 20 bp, Cannabis sativa
  misc_feature: Complimentary to position 7222 of SEQ. ID NO. 13 AAE1 CM011611.11210973-1228229
ggagaggttttaaggaggca

SEQ ID NO: 547 - DNA, 20 bp, Cannabis sativa
  misc_feature: Complimentary to position 7227 of SEQ. ID NO. 13 AAE1 CM011611.11210973-1228229
tgaatggagaggttttaagg

SEQ ID NO: 548 - DNA, 20 bp, Cannabis sativa
  misc_feature: Complimentary to position 7230 of SEQ. ID NO. 13 AAE1 CM011611.11210973-1228229
cattgaatggagaggtttta

SEQ ID NO: 549 - DNA, 20 bp, Cannabis sativa
  misc_feature: Complimentary to position 7238 of SEQ. ID NO. 13 AAE1 CM011611.11210973-1228229
aatgcctacattgaatggag

SEQ ID NO: 550 - DNA, 20 bp, Cannabis sativa
  misc_feature: Complimentary to position 7243 of SEQ. ID NO. 13 AAE1 CM011611.11210973-1228229
aagggaatgcctacattgaa

SEQ ID NO: 551 - DNA, 20 bp, Cannabis sativa
  misc_feature: Complimentary to position 7245 of SEQ. ID NO. 13 AAE1 CM011611.11210973-1228229
aaaacctctccattcaatgt

SEQ ID NO: 552 - DNA, 20 bp, Cannabis sativa
  misc_feature: Complimentary to position 16447 of SEQ. ID NO. 13 AAE1 CM011611.11210973-7261
caccatgatgtttattttaa

SEQ ID NO: 553 - DNA, 20 bp, Cannabis sativa
  misc_feature: Complimentary to position 7262 of SEQ. ID NO. 13 AAE1 CM011611.11210973-1228229
tcaccatgatgtttatttta

SEQ ID NO: 554 - DNA, 20 bp, Cannabis sativa
  misc_feature: Complimentary to position 7270 of SEQ. ID NO. 13 AAE1 CM011611.11210973-1228229
ttcccttaaaataaacatca

SEQ ID NO: 555 - DNA, 20 bp, Cannabis sativa
  misc_feature: Complimentary to position 7288 of SEQ. ID NO. 13 AAE1 CM011611.11210973-1228229
gcatcgaagactctgttgaa

SEQ ID NO: 556 - DNA, 20 bp, Cannabis sativa
  misc_feature: Complimentary to position 7312 of SEQ. ID NO. 13 AAE1 CM011611.11210973-1228229
gcgctcggtccagtcatgtt

SEQ ID NO: 557 - DNA, 20 bp, Cannabis sativa
  misc_feature: Complimentary to position 7314 of SEQ. ID NO. 13 AAE1 CM011611.11210973-1228229
ttcgatgctccaaacatgac

SEQ ID NO: 558 - DNA, 20 bp, Cannabis sativa
  misc_feature: Complimentary to position 7327 of SEQ. ID NO. 13 AAE1 CM011611.11210973-1228229
ggaattggtgaattagcgct

SEQ ID NO: 559 - DNA, 20 bp, Cannabis sativa
  misc_feature: Complimentary to position 7341 of SEQ. ID NO. 13 AAE1 CM011611.11210973-1228229
agcgctaattcaccaattcc

SEQ ID NO: 560 - DNA, 20 bp, Cannabis sativa
  misc_feature: Complimentary to position 7342 of SEQ. ID NO. 13 AAE1 CM011611.11210973-1228229
cctaaaaacaaaccaggaat

SEQ ID NO: 561 - DNA, 20 bp, Cannabis sativa
  misc_feature: Complimentary to position 7348 of SEQ. ID NO. 13 AAE1 CM011611.11210973-1228229
atgcagcctaaaaacaaacc

SEQ ID NO: 562 - DNA, 20 bp, Cannabis sativa
  misc_feature: Complimentary to position 7353 of SEQ. ID NO. 13 AAE1 CM011611.11210973-1228229
ccaattcctggtttgttttt

SEQ ID NO: 563 - DNA, 20 bp, Cannabis sativa
  misc_feature: Complimentary to position 7417 of SEQ. ID NO. 13 AAE1 CM011611.11210973-1228229
aaatgtaacataattttata

SEQ ID NO: 564 - DNA, 20 bp, Cannabis sativa
  misc_feature: Complimentary to position 7418 of SEQ. ID NO. 13 AAE1 CM011611.11210973-1228229
aatgtaacataattttatat

SEQ ID NO: 565 - DNA, 20 bp, Cannabis sativa
  misc_feature: Complimentary to position 7461 of SEQ. ID NO. 13 AAE1 CM011611.11210973-1228229
acttttcaaataagatttga

SEQ ID NO: 566 - DNA, 20 bp, Cannabis sativa
  misc_feature: Complimentary to position 7489 of SEQ. ID NO. 13 AAE1 CM011611.11210973-1228229
aaaatataatttaaaaatat

SEQ ID NO: 567 - DNA, 20 bp, Cannabis sativa
  misc_feature: Complimentary to position 7523 of SEQ. ID NO. 13 AAE1 CM011611.11210973-1228229
ataagtttattaattaccat

SEQ ID NO: 568 - DNA, 20 bp, Cannabis sativa
  misc_feature: Complimentary to position 7528 of SEQ. ID NO. 13 AAE1 CM011611.11210973-1228229
tgacaacaatggttatccaa

SEQ ID NO: 569 - DNA, 20 bp, Cannabis sativa
  misc_feature: Complimentary to position 7539 of SEQ. ID NO. 13 AAE1 CM011611.11210973-1228229
ttatacatacttgacaacaa

SEQ ID NO: 570 - DNA, 20 bp, Cannabis sativa
  misc_feature: Complimentary to position 7569 of SEQ. ID NO. 13 AAE1 CM011611.11210973-1228229
tcatttagttcacaatgtat

SEQ ID NO: 571 - DNA, 20 bp, Cannabis sativa
  misc_feature: Complimentary to position 7570 of SEQ. ID NO. 13 AAE1 CM011611.11210973-1228229
ttcatttagttcacaatgta

SEQ ID NO: 572 - DNA, 20 bp, Cannabis sativa
  misc_feature: Complimentary to position 7620 of SEQ. ID NO. 13 AAE1 CM011611.11210973-1228229
attggtggtgcattttctgc

SEQ ID NO: 573 - DNA, 20 bp, Cannabis sativa
  misc_feature: Complimentary to position 7635 of SEQ. ID NO. 13 AAE1 CM011611.11210973-
tgtggtggcacagaaattgg

SEQ ID NO: 574 - DNA, 20 bp, Cannabis sativa
  misc_feature: Complimentary to position 7638 of SEQ. ID NO. 13 AAE1 CM011611.11210973-1228229
atgtgtggtggcacagaaat

SEQ ID NO: 575 - DNA, 20 bp, Cannabis sativa
  misc_feature: Complimentary to position 7650 of SEQ. ID NO. 13 AAE1 CM011611.11210973-1228229
cctgttatcgaaatgtgtgg

SEQ ID NO: 576 - DNA, 20 bp, Cannabis sativa
  misc_feature: Complimentary to position 7653 of SEQ. ID NO. 13 AAE1 CM011611.11210973-1228229
aagcctgttatcgaaatgtg

SEQ ID NO: 577 - DNA, 20 bp, Cannabis sativa
  misc_feature: Complimentary to position 7661 of SEQ. ID NO. 13 AAE1 CM011611.11210973-1228229
ccaccacacatttcgataac

SEQ ID NO: 578 - DNA, 20 bp, Cannabis sativa
  misc_feature: Complimentary to position 7688 of SEQ. ID NO. 13 AAE1 CM011611.11210973-1228229
atgaatacctatggttgatg

SEQ ID NO: 579 - DNA, 20 bp, Cannabis sativa
  misc_feature: Complimentary to position 7689 of SEQ. ID NO. 13 AAE1 CM011611.11210973-1228229
gatgaatacctatggttgat

SEQ ID NO: 580 - DNA, 20 bp, Cannabis sativa
  misc_feature: Complimentary to position 7690 of SEQ. ID NO. 13 AAE1 CM011611.11210973-1228229
agatgaatacctatggttga

SEQ ID NO: 581 - DNA, 20 bp, Cannabis sativa
  misc_feature: Complimentary to position 7692 of SEQ. ID NO. 13 AAE1 CM011611.11210973-1228229
ttgctctccccatcaaccat

SEQ ID NO: 582 - DNA, 20 bp, Cannabis sativa
  misc_feature: Complimentary to position 7697 of SEQ. ID NO. 13 AAE1 CM011611.11210973-1228229
ctaatgtagatgaataccta

SEQ ID NO: 583 - DNA, 20 bp, Cannabis sativa
  misc_feature: Complimentary to position 7726 of SEQ. ID NO. 13 AAE1 CM011611.11210973-1228229
attagatgcttcaccagaag

SEQ ID NO: 584 - DNA, 20 bp, Cannabis sativa
  misc_feature: Complimentary to position 7728 of SEQ. ID NO. 13 AAE1 CM011611.11210973-1228229
tgtagttgcttttcctcttc

SEQ ID NO: 585 - DNA, 20 bp, Cannabis sativa
  misc_feature: Complimentary to position 7745 of SEQ. ID NO. 13 AAE1 CM011611.11210973-1228229
gaggaaaagcaactacaaga

SEQ ID NO: 586 - DNA, 20 bp, Cannabis sativa
  misc_feature: Complimentary to position 7746 of SEQ. ID NO. 13 AAE1 CM011611.11210973-1228229
aggaaaagcaactacaagaa

SEQ ID NO: 587 - DNA, 20 bp, Cannabis sativa
  misc_feature: Complimentary to position 7780 of SEQ. ID NO. 13 AAE1 CM011611.11210973-1228229
attatattaaacgtattata

SEQ ID NO: 588 - DNA, 20 bp, Cannabis sativa
  misc_feature: Complimentary to position 7828 of SEQ. ID NO. 13 AAE1 CM011611.11210973-1228229
tgtgcatagataagaaatat

SEQ ID NO: 589 - DNA, 20 bp, Cannabis sativa
  misc_feature: Complimentary to position 7853 of SEQ. ID NO. 13 AAE1 CM011611.11210973-1228229
aatatattataattctttac

SEQ ID NO: 590 - DNA, 20 bp, Cannabis sativa
  misc_feature: Complimentary to position 7857 of SEQ. ID NO. 13 AAE1 CM011611.11210973-1228229
tattataattctttaccgga

SEQ ID NO: 591 - DNA, 20 bp, Cannabis sativa
  misc_feature: Complimentary to position 7860 of SEQ. ID NO. 13 AAE1 CM011611.11210973-1228229
tataattctttaccggatgg

SEQ ID NO: 592 - DNA, 20 bp, Cannabis sativa
  misc_feature: Complimentary to position 7861 of SEQ. ID NO. 13 AAE1 CM011611.11210973-1228229
gctatgattggtccaccatc

SEQ ID NO: 593 - DNA, 20 bp, Cannabis sativa
  misc_feature: Complimentary to position 7873 of SEQ. ID NO. 13 AAE1 CM011611.11210973-1228229
attgtgttagtggctatgat

SEQ ID NO: 594 - DNA, 20 bp, Cannabis sativa
  misc_feature: Complimentary to position 7883 of SEQ. ID NO. 13 AAE1 CM011611.11210973-1228229
aaaagtagcaattgtgttag

SEQ ID NO: 595 - DNA, 20 bp, Cannabis sativa
  misc_feature: Complimentary to position 7906 of SEQ. ID NO. 13 AAE1 CM011611.11210973-1228229
tccctagcattgttcgatca

SEQ ID NO: 596 - DNA, 20 bp, Cannabis sativa
  misc_feature: Complimentary to position 7915 of SEQ. ID NO. 13 AAE1 CM011611.11210973-1228229
ttccatgatcgaacaatgct

SEQ ID NO: 597 - DNA, 20 bp, Cannabis sativa
  misc_feature: Complimentary to position 7916 of SEQ. ID NO. 13 AAE1 CM011611.11210973-1228229
tccatgatcgaacaatgcta

SEQ ID NO: 598 - DNA, 20 bp, Cannabis sativa
  misc_feature: Complimentary to position 7929 of SEQ. ID NO. 13 AAE1 CM011611.11210973-1228229
taaagtaacaatgctaggtg

SEQ ID NO: 599 - DNA, 20 bp, Cannabis sativa
  misc_feature: Complimentary to position 7934 of SEQ. ID NO. 13 AAE1 CM011611.11210973-1228229
gatgctaaagtaacaatgct

SEQ ID NO: 600 - DNA, 20 bp, Cannabis sativa
  misc_feature: Complimentary to position 7956 of SEQ. ID NO. 13 AAE1 CM011611.11210973-1228229
atgttttctaatatgtgtgt

SEQ ID NO: 601 - DNA, 20 bp, Cannabis sativa
  misc_feature: Complimentary to position 8037 of SEQ. ID NO. 13 AAE1 CM011611.11210973-1228229
aataaaaaacatgataagtt

SEQ ID NO: 602 - DNA, 20 bp, Cannabis sativa
  misc_feature: Complimentary to position 8086 of SEQ. ID NO. 13 AAE1 CM011611.11210973-1228229
aataaaaatgatgtgattat

SEQ ID NO: 603 - DNA, 20 bp, Cannabis sativa
  misc_feature: Complimentary to position 8114 of SEQ. ID NO. 13 AAE1 CM011611.11210973-1228229
atgtctaattcattatagaa

SEQ ID NO: 604 - DNA, 20 bp, Cannabis sativa
  misc_feature: Complimentary to position 8115 of SEQ. ID NO. 13 AAE1 CM011611.11210973-1228229
aatgtctaattcattataga

SEQ ID NO: 605 - DNA, 20 bp, Cannabis sativa
  misc_feature: Complimentary to position 8199 of SEQ. ID NO. 13 AAE1 CM011611.11210973-1228229
ctaaaaagtttattagttaa

SEQ ID NO: 606 - DNA, 20 bp, Cannabis sativa
  misc_feature: Complimentary to position 8223 of SEQ. ID NO. 13 AAE1 CM011611.11210973-1228229
tagcttcgccaaatttgtgc

SEQ ID NO: 607 - DNA, 20 bp, Cannabis sativa
  misc_feature: Complimentary to position 8226 of SEQ. ID NO. 13 AAE1 CM011611.11210973-1228229
ttagtttacctgcacaaatt

SEQ ID NO: 608 - DNA, 20 bp, Cannabis sativa
  misc_feature: Complimentary to position 8245 of SEQ. ID NO. 13 AAE1 CM011611.11210973-1228229
ttggcgaagctagaaacaag

SEQ ID NO: 609 - DNA, 20 bp, Cannabis sativa
  misc_feature: Complimentary to position 8261 of SEQ. ID NO. 13 AAE1 CM011611.11210973-1228229
gcttctcttgccttgtataa

SEQ ID NO: 610 - DNA, 20 bp, Cannabis sativa
  misc_feature: Complimentary to position 8262 of SEQ. ID NO. 13 AAE1 CM011611.11210973-1228229
aagtggtgatccattataca

SEQ ID NO: 611 - DNA, 20 bp, Cannabis sativa
  misc_feature: Complimentary to position 8282 of SEQ. ID NO. 13 AAE1 CM011611.11210973-1228229
aggcaagagaagccccatta

SEQ ID NO: 612 - DNA, 20 bp, Cannabis sativa
  misc_feature: Complimentary to position 8283 of SEQ. ID NO. 13 AAE1 CM011611.11210973-1228229
ctatgcttcactccttaatg

SEQ ID NO: 613 - DNA, 20 bp, Cannabis sativa
  misc_feature: Complimentary to position 8284 of SEQ. ID NO. 13 AAE1 CM011611.11210973-1228229
tctatgcttcactccttaat

SEQ ID NO: 614 - DNA, 20 bp, Cannabis sativa
  misc_feature: Complimentary to position 8285 of SEQ. ID NO. 13 AAE1 CM011611.11210973-1228229
gtctatgcttcactccttaa

SEQ ID NO: 615 - DNA, 20 bp, Cannabis sativa
  misc_feature: Complimentary to position 8305 of SEQ. ID NO. 13 AAE1 CM011611.11210973-1228229
agtgaagcatagaccagcca

SEQ ID NO: 616 - DNA, 20 bp, Cannabis sativa
  misc_feature: Complimentary to position 8307 of SEQ. ID NO. 13 AAE1 CM011611.11210973-1228229
ttggatgatgggtccttggc

SEQ ID NO: 617 - DNA, 20 bp, Cannabis sativa
  misc_feature: Complimentary to position 8311 of SEQ. ID NO. 13 AAE1 CM011611.11210973-1228229
ttggttggatgatgggtcct

SEQ ID NO: 618 - DNA, 20 bp, Cannabis sativa
  misc_feature: Complimentary to position 8318 of SEQ. ID NO. 13 AAE1 CM011611.11210973-1228229
actaatcttggttggatgat

SEQ ID NO: 619 - DNA, 20 bp, Cannabis sativa
  misc_feature: Complimentary to position 8319 of SEQ. ID NO. 13 AAE1 CM011611.11210973-1228229
cactaatcttggttggatga

SEQ ID NO: 620 - DNA, 20 bp, Cannabis sativa
  misc_feature: Complimentary to position 8326 of SEQ. ID NO. 13 AAE1 CM011611.11210973-1228229
tttggcccactaatcttggt

SEQ ID NO: 621 - DNA, 20 bp, Cannabis sativa
  misc_feature: Complimentary to position 8330 of SEQ. ID NO. 13 AAE1 CM011611.11210973-1228229
attgtttggcccactaatct

SEQ ID NO: 622 - DNA, 20 bp, Cannabis sativa
  misc_feature: Complimentary to position 8331 of SEQ. ID NO. 13 AAE1 CM011611.11210973-1228229
catcatccaaccaagattag

SEQ ID NO: 623 - DNA, 20 bp, Cannabis sativa
  misc_feature: Complimentary to position 8332 of SEQ. ID NO. 13 AAE1 CM011611.11210973-1228229
atcatccaaccaagattagt

SEQ ID NO: 624 - DNA, 20 bp, Cannabis sativa
  misc_feature: Complimentary to position 8344 of SEQ. ID NO. 13 AAE1 CM011611.11210973-1228229
ggaaaggtgatgtcattgtt

SEQ ID NO: 625 - DNA, 20 bp, Cannabis sativa
  misc_feature: Complimentary to position 8360 of SEQ. ID NO. 13 AAE1 CM011611.11210973-1228229
agccatttggacattaggaa

SEQ ID NO: 626 - DNA, 20 bp, Cannabis sativa
  misc_feature: Complimentary to position 8365 of SEQ. ID NO. 13 AAE1 CM011611.11210973-1228229
ggtggagccatttggacatt

SEQ ID NO: 627 - DNA, 20 bp, Cannabis sativa
  misc_feature: Complimentary to position 8369 of SEQ. ID NO. 13 AAE1 CM011611.11210973-1228229
cacctttcctaatgtccaaa

SEQ ID NO: 628 - DNA, 20 bp, Cannabis sativa
  misc_feature: Complimentary to position 8373 of SEQ. ID NO. 13 AAE1 CM011611.11210973-1228229
tgcagatgggtggagccatt

SEQ ID NO: 629 - DNA, 20 bp, Cannabis sativa
  misc_feature: Complimentary to position 8383 of SEQ. ID NO. 13 AAE1 CM011611.11210973-1228229
taaaagcagctgcagatggg

SEQ ID NO: 630 - DNA, 20 bp, Cannabis sativa
  misc_feature: Complimentary to position 8386 of SEQ. ID NO. 13 AAE1 CM011611.11210973-1228229
ctttaaaagcagctgcagat

SEQ ID NO: 631 - DNA, 20 bp, Cannabis sativa
  misc_feature: Complimentary to position 8387 of SEQ. ID NO. 13 AAE1 CM011611.11210973-1228229
cctttaaaagcagctgcaga

SEQ ID NO: 632 - DNA, 20 bp, Cannabis sativa
  misc_feature: Complimentary to position 8398 of SEQ. ID NO. 13 AAE1 CM011611.11210973-1228229
ccatctgcagctgcttttaa

SEQ ID NO: 633 - DNA, 20 bp, Cannabis sativa
  misc_feature: Complimentary to position 8408 of SEQ. ID NO. 13 AAE1 CM011611.11210973-1228229
ctgcttttaaaggagttgct

SEQ ID NO: 634 - DNA, 20 bp, Cannabis sativa
  misc_feature: Complimentary to position 8409 of SEQ. ID NO. 13 AAE1 CM011611.11210973-1228229
tgcttttaaaggagttgctt

SEQ ID NO: 635 - DNA, 20 bp, Cannabis sativa
  misc_feature: Complimentary to position 8416 of SEQ. ID NO. 13 AAE1 CM011611.11210973-1228229
aaaggagttgcttgggtcca

SEQ ID NO: 636 - DNA, 20 bp, Cannabis sativa
  misc_feature: Complimentary to position 8422 of SEQ. ID NO. 13 AAE1 CM011611.11210973-1228229
gggagccaaaggcaattcca

SEQ ID NO: 637 - DNA, 20 bp, Cannabis sativa
  misc_feature: Complimentary to position 8428 of SEQ. ID NO. 13 AAE1 CM011611.11210973-1228229
tgggtccatggaattgcctt

SEQ ID NO: 638 - DNA, 20 bp, Cannabis sativa
  misc_feature: Complimentary to position 8433 of SEQ. ID NO. 13 AAE1 CM011611.11210973-1228229
ttttggtataggggagccaa

SEQ ID NO: 639 - DNA, 20 bp, Cannabis sativa
  misc_feature: Complimentary to position 8442 of SEQ. ID NO. 13 AAE1 CM011611.11210973-1228229
acaatattgttttggtatag

SEQ ID NO: 640 - DNA, 20 bp, Cannabis sativa
  misc_feature: Complimentary to position 8444 of SEQ. ID NO. 13 AAE1 CM011611.11210973-1228229
gacaatattgttttggtata

SEQ ID NO: 641 - DNA, 20 bp, Cannabis sativa
  misc_feature: Complimentary to position 8444 of SEQ. ID NO. 13 AAE1 CM011611.11210973-1228229
agacaatattgttttggtat

SEQ ID NO: 642 - DNA, 20 bp, Cannabis sativa
  misc_feature: Complimentary to position 8450 of SEQ. ID NO. 13 AAE1 CM011611.11210973-1228229
gatggaagacaatattgttt

SEQ ID NO: 643 - DNA, 20 bp, Cannabis sativa
  misc_feature: Complimentary to position 8468 of SEQ. ID NO. 13 AAE1 CM011611.11210973-1228229
atcaatctaaacacttttga

SEQ ID NO: 644 - DNA, 20 bp, Cannabis sativa
  misc_feature: Complimentary to position 8497 of SEQ. ID NO. 13 AAE1 CM011611.11210973-1228229
ttgatgaacaaaactaatta

SEQ ID NO: 645 - DNA, 20 bp, Cannabis sativa
  misc_feature: Complimentary to position 8506 of SEQ. ID NO. 13 AAE1 CM011611.11210973-1228229
aaaactaattaaggatatta

SEQ ID NO: 646 - DNA, 20 bp, Cannabis sativa
  misc_feature: Complimentary to position 8507 of SEQ. ID NO. 13 AAE1 CM011611.11210973-1228229
aaactaattaaggatattaa

SEQ ID NO: 647 - DNA, 20 bp, Cannabis sativa
aactaattaaggatattaag

SEQ ID NO: 648 - DNA, 20 bp, Cannabis sativa
  misc_feature: Complimentary to position 8509 of SEQ. ID NO. 13 AAE1 CM011611.11210973-1228229
actaattaaggatattaagg

SEQ ID NO: 649 - DNA, 20 bp, Cannabis sativa
  misc_feature: Complimentary to position 8529 of SEQ. ID NO. 13 AAE1 CM011611.11210973-1228229
gggctcatgagattttttaa

SEQ ID NO: 650 - DNA, 20 bp, Cannabis sativa
  misc_feature: Complimentary to position 8573 of SEQ. ID NO. 13 AAE1 CM011611.11210973-1228229
attcagtaaataatgatgct

SEQ ID NO: 651 - DNA, 20 bp, Cannabis sativa
  misc_feature: Complimentary to position 8598 of SEQ. ID NO. 13 AAE1 CM011611.11210973-1228229
ttttaagagcgcaaactaaa

SEQ ID NO: 652 - DNA, 20 bp, Cannabis sativa
  misc_feature: Complimentary to position 8648 of SEQ. ID NO. 13 AAE1 CM011611.11210973-1228229
gttttgtttaaagataatag

SEQ ID NO: 653 - DNA, 20 bp, Cannabis sativa
  misc_feature: Complimentary to position 8649 of SEQ. ID NO. 13 AAE1 CM011611.11210973-1228229
ttttgtttaaagataatagc

SEQ ID NO: 654 - DNA, 20 bp, Cannabis sativa
  misc_feature: Complimentary to position 8681 of SEQ. ID NO. 13 AAE1 CM011611.11210973
tcaaaattttatattgtaag

SEQ ID NO: 655 - DNA, 20 bp, Cannabis sativa
  misc_feature: Complimentary to position 8758 of SEQ. ID NO. 13 AAE1 CM011611.11210973-1228229
agacaataagagagtttata

SEQ ID NO: 656 - DNA, 20 bp, Cannabis sativa
  misc_feature: Complimentary to position 8777 of SEQ. ID NO. 13 AAE1 CM011611.11210973-1228229
actctcttattgtcttaaac

SEQ ID NO: 657 - DNA, 20 bp, Cannabis sativa
  misc_feature: Complimentary to position 8778 of SEQ. ID NO. 13 AAE1 CM011611.11210973-1228229
ctctcttattgtcttaaaca

SEQ ID NO: 658 - DNA, 20 bp, Cannabis sativa
  misc_feature: Complimentary to position 8779 of SEQ. ID NO. 13 AAE1 CM011611.11210973-1228229
tctcttattgtcttaaacag

SEQ ID NO: 659 - DNA, 20 bp, Cannabis sativa
  misc_feature: Complimentary to position 8801 of SEQ. ID NO. 13 AAE1 CM011611.11210973-1228229
gatttaatgatcaagaattt

SEQ ID NO: 660 - DNA, 20 bp, Cannabis sativa
  misc_feature: Complimentary to position 8853 of SEQ. ID NO. 13 AAE1 CM011611.11210973-1228229
gaatttattaaaatagcatt

SEQ ID NO: 661 - DNA, 20 bp, Cannabis sativa
  misc_feature: Complimentary to position 8873 of SEQ. ID NO. 13 AAE1 CM011611.11210973-1228229
attttaataaattcaaaaca

SEQ ID NO: 662 - DNA, 20 bp, Cannabis sativa
  misc_feature: Complimentary to position 8876 of SEQ. ID NO. 13 AAE1 CM011611.11210973-1228229
ttaataaattcaaaacatgg

SEQ ID NO: 663 - DNA, 20 bp, Cannabis sativa
  misc_feature: Complimentary to position 8879 of SEQ. ID NO. 13 AAE1 CM011611.11210973-1228229
ataaattcaaaacatggcgg

SEQ ID NO: 664 - DNA, 20 bp, Cannabis sativa
  misc_feature: Complimentary to position 8900 of SEQ. ID NO. 13 AAE1 CM011611.11210973-1228229
ctcttctcatctggaacaac

SEQ ID NO: 665 - DNA, 20 bp, Cannabis sativa
  misc_feature: Complimentary to position 8909 of SEQ. ID NO. 13 AAE1 CM011611.11210973-1228229
acaaacatcctcttctcatc

SEQ ID NO: 666 - DNA, 20 bp, Cannabis sativa
  misc_feature: Complimentary to position 8912 of SEQ. ID NO. 13 AAE1 CM011611.11210973-1228229
ctgttgttccagatgagaag

SEQ ID NO: 667 - DNA, 20 bp, Cannabis sativa
  misc_feature: Complimentary to position 8925 of SEQ. ID NO. 13 AAE1 CM011611.11210973-1228229
tgagaagaggatgtttgtat

SEQ ID NO: 668 - DNA, 20 bp, Cannabis sativa
  misc_feature: Complimentary to position 8934 of SEQ. ID NO. 13 AAE1 CM011611.11210973-1228229
gatgtttgtataggcatcaa

SEQ ID NO: 669 - DNA, 20 bp, Cannabis sativa
  misc_feature: Complimentary to position 8935 of SEQ. ID NO. 13 AAE1 CM011611.11210973-1228229
atgtttgtataggcatcaac

SEQ ID NO: 670 - DNA, 20 bp, Cannabis sativa
  misc_feature: Complimentary to position 8970 of SEQ. ID NO. 13 AAE1 CM011611.11210973-1228229
agtaaattcacaatttctgc

SEQ ID NO: 671 - DNA, 20 bp, Cannabis sativa
  misc_feature: Complimentary to position 8984 of SEQ. ID NO. 13 AAE1 CM011611.11210973-1228229
gggactaatatcttcttgag

SEQ ID NO: 672 - DNA, 20 bp, Cannabis sativa
  misc_feature: Complimentary to position 9004 of SEQ. ID NO. 13 AAE1 CM011611.11210973-1228229
aaaataataagcatatatgt

SEQ ID NO: 673 - DNA, 20 bp, Cannabis sativa
  misc_feature: Complimentary to position 9005 of SEQ. ID NO. 13 AAE1 CM011611.11210973-1228229
caaaataataagcatatatg

SEQ ID NO: 674 - DNA, 20 bp, Cannabis sativa
  misc_feature: Complimentary to position 9017 of SEQ. ID NO. 13 AAE1 CM011611.11210973-1228229
cacatatatgcttattattt

SEQ ID NO: 675 - DNA, 20 bp, Cannabis sativa
  misc_feature: Complimentary to position 9121 of SEQ. ID NO. 13 AAE1 CM011611.11210973-1228229
tttttaattaaaagtaacac

SEQ ID NO: 676 - DNA, 20 bp, Cannabis sativa
  misc_feature: Complimentary to position 9187 of SEQ. ID NO. 13 AAE1 CM011611.11210973-1228229
ttaatttaggagttattttg

SEQ ID NO: 677 - DNA, 20 bp, Cannabis sativa
  misc_feature: Complimentary to position 9188 of SEQ. ID NO. 13 AAE1 CM011611.11210973-1228229
attaatttaggagttatttt

SEQ ID NO: 678 - DNA, 20 bp, Cannabis sativa
  misc_feature: Complimentary to position 9189 of SEQ. ID NO. 13 AAE1 CM011611.11210973-1228229
tattaatttaggagttattt

SEQ ID NO: 679 - DNA, 20 bp, Cannabis sativa
  misc_feature: Complimentary to position 9200 of SEQ. ID NO. 13 AAE1 CM011611.11210973-1228229
aagttgggcattattaattt

SEQ ID NO: 680 - DNA, 20 bp, Cannabis sativa
  misc_feature: Complimentary to position 9215 of SEQ. ID NO. 13 AAE1 CM011611.11210973-1228229
atcaggggttttatcaagtt

SEQ ID NO: 681 - DNA, 20 bp, Cannabis sativa
  misc_feature: Complimentary to position 9216 of SEQ. ID NO. 13 AAE1 CM011611.11210973-1228229
tatcaggggttttatcaagt

SEQ ID NO: 682 - DNA, 20 bp, Cannabis sativa
  misc_feature: Complimentary to position 9230 of SEQ. ID NO. 13 AAE1 CM011611.11210973-1228229
atcacgtttcttattatcag

SEQ ID NO: 683 - DNA, 20 bp, Cannabis sativa
  misc_feature: Complimentary to position 9231 of SEQ. ID NO. 13 AAE1 CM011611.11210973-1228229
tatcacgtttcttattatca

SEQ ID NO: 684 - DNA, 20 bp, Cannabis sativa
  misc_feature: Complimentary to position 9232 of SEQ. ID NO. 13 AAE1 CM011611.11210973-1228229
gtatcacgtttcttattatc

SEQ ID NO: 685 - DNA, 20 bp, Cannabis sativa
  misc_feature: Complimentary to position 9261 of SEQ. ID NO. 13 AAE1 CM011611.11210973-1228229
aaaatacatgtgaatgttag

SEQ ID NO: 686 - DNA, 20 bp, Cannabis sativa
  misc_feature: Complimentary to position 9289 of SEQ. ID NO. 13 AAE1 CM011611.11210973-1228229
tattttagttcaaactccaa

SEQ ID NO: 687 - DNA, 20 bp, Cannabis sativa
  misc_feature: Complimentary to position 9294 of SEQ. ID NO. 13 AAE1 CM011611.11210973-1228229
atatgaacatttggatccat

SEQ ID NO: 688 - DNA, 20 bp, Cannabis sativa
  misc_feature: Complimentary to position 9303 of SEQ. ID NO. 13 AAE1 CM011611.11210973-1228229
tatctatatatatgaacatt

SEQ ID NO: 689 - DNA, 20 bp, Cannabis sativa
  misc_feature: Complimentary to position 9338 of SEQ. ID NO. 13 AAE1 CM011611.11210973-1228229
gtggaatatataaacagtag

SEQ ID NO: 690 - DNA, 20 bp, Cannabis sativa
  misc_feature: Complimentary to position 9357 of SEQ. ID NO. 13 AAE1 CM011611.11210973-1228229
aattcgtatagagattaatg

SEQ ID NO: 691 - DNA, 20 bp, Cannabis sativa
  misc_feature: Complimentary to position 9459 of SEQ. ID NO. 13 AAE1 CM011611.11210973-1228229
gcgtgatggcgatatttctt

SEQ ID NO: 692 - DNA, 20 bp, Cannabis sativa
  misc_feature: Complimentary to position 9460 of SEQ. ID NO. 13 AAE1 CM011611.11210973-1228229
tgcgtgatggcgatatttct

SEQ ID NO: 693 - DNA, 20 bp, Cannabis sativa
  misc_feature: Complimentary to position 9473 of SEQ. ID NO. 13 AAE1 CM011611.11210973-1228229
attggtgcagaattgcgtga

SEQ ID NO: 694 - DNA, 20 bp, Cannabis sativa
  misc_feature: Complimentary to position 9491 of SEQ. ID NO. 13 AAE1 CM011611.11210973-1228229
ccttgtagtggctctaatat

SEQ ID NO: 695 - DNA, 20 bp, Cannabis sativa
  misc_feature: Complimentary to position 9502 of SEQ. ID NO. 13 AAE1 CM011611.11210973-1228229
ccaatattagagccactaca

SEQ ID NO: 696 - DNA, 20 bp, Cannabis sativa
  misc_feature: Complimentary to position 9503 of SEQ. ID NO. 13 AAE1 CM011611.11210973-1228229
gccattgttattccttgtag

SEQ ID NO: 697 - DNA, 20 bp, Cannabis sativa
  misc_feature: Complimentary to position 9513 of SEQ. ID NO. 13 AAE1 CM011611.11210973-1228229
gccactacaaggaataacaa

SEQ ID NO: 698 - DNA, 20 bp, Cannabis sativa
  misc_feature: Complimentary to position 9519 of SEQ. ID NO. 13 AAE1 CM011611.11210973-1228229
acaaggaataacaatggcca

SEQ ID NO: 699 - DNA, 20 bp, Cannabis sativa
  misc_feature: Complimentary to position 9520 of SEQ. ID NO. 13 AAE1 CM011611.11210973-1228229
caaggaataacaatggccat

SEQ ID NO: 700 - DNA, 20 bp, Cannabis sativa
tgtggaagccaagtctccca

SEQ ID NO: 701 - DNA, 20 bp, Cannabis sativa
  misc_feature: Complimentary to position 9528 of SEQ. ID NO. 13 AAE1 CM011611.11210973-1228229
aacaatggccatgggagact

SEQ ID NO: 702 - DNA, 20 bp, Cannabis sativa
  misc_feature: Complimentary to position 9543 of SEQ. ID NO. 13 AAE1 CM011611.11210973-1228229
taaattgtgcagtagagttg

SEQ ID NO: 703 - DNA, 20 bp, Cannabis sativa
  misc_feature: Complimentary to position 9574 of SEQ. ID NO. 13 AAE1 CM011611.11210973-1228229
ttaactctttttttgtgaga

SEQ ID NO: 704 - DNA, 20 bp, Cannabis sativa
  misc_feature: Complimentary to position 16447 of SEQ. ID NO. 13 AAE1 CM011611.11210973-9617
ataatttattattatttgtc

SEQ ID NO: 705 - DNA, 20 bp, Cannabis sativa
  misc_feature: Complimentary to position 13019 of SEQ. ID NO. 13 AAE1 CM011611.11210973-1228229
tgctagtacatgtctatata

SEQ ID NO: 706 - DNA, 20 bp, Cannabis sativa
  misc_feature: Complimentary to position 13060 of SEQ. ID NO. 13 AAE1 CM011611.11210973-1228229
agcaaaagccatttttacac

SEQ ID NO: 707 - DNA, 20 bp, Cannabis sativa
  misc_feature: Complimentary to position 13063 of SEQ. ID NO. 13 AAE1 CM011611.11210973-1228229
tatatatacctgtgtaaaaa

SEQ ID NO: 708 - DNA, 20 bp, Cannabis sativa
  misc_feature: Complimentary to position 13103 of SEQ. ID NO. 13 AAE1 CM011611.11210973-1228229
cgaagtcttgttgatatttc

SEQ ID NO: 709 - DNA, 20 bp, Cannabis sativa
  misc_feature: Complimentary to position 13152 of SEQ. ID NO. 13 AAE1 CM011611.11210973-1228229
tatctagctattgttcttgc

SEQ ID NO: 710 - DNA, 20 bp, Cannabis sativa
  misc_feature: Complimentary to position 13153 of SEQ. ID NO. 13 AAE1 CM011611.11210973-1228229
ctatctagctattgttcttg

SEQ ID NO: 711 - DNA, 20 bp, Cannabis sativa
  misc_feature: Complimentary to position 13180 of SEQ. ID NO. 13 AAE1 CM011611.11210973-1228229
gccaatgcatgtggatgctg

SEQ ID NO: 712 - DNA, 20 bp, Cannabis sativa
  misc_feature: Complimentary to position 13189 of SEQ. ID NO. 13 AAE1 CM011611.11210973-1228229
aattgatatgccaatgcatg

SEQ ID NO: 713 - DNA, 20 bp, Cannabis sativa
  misc_feature: Complimentary to position 13190 of SEQ. ID NO. 13 AAE1 CM011611.11210973-1228229
accacagcatccacatgcat

SEQ ID NO: 714 - DNA, 20 bp, Cannabis sativa
  misc_feature: Complimentary to position 13224 of SEQ. ID NO. 13 AAE1 CM011611.11210973-1228229
gaagaaatgggtttggagaa

SEQ ID NO: 715 - DNA, 20 bp, Cannabis sativa
  misc_feature: Complimentary to position 13225 of SEQ. ID NO. 13 AAE1 CM011611.11210973-1228229
tgaagaaatgggtttggaga

SEQ ID NO: 716 - DNA, 20 bp, Cannabis sativa
  misc_feature: Complimentary to position 13231 of SEQ. ID NO. 13 AAE1 CM011611.11210973-1228229
tgcacttgaagaaatgggtt

SEQ ID NO: 717 - DNA, 20 bp, Cannabis sativa
  misc_feature: Complimentary to position 13236 of SEQ. ID NO. 13 AAE1 CM011611.11210973-1228229
ggttatgcacttgaagaaat

SEQ ID NO: 718 - DNA, 20 bp, Cannabis sativa
  misc_feature: Complimentary to position 13237 of SEQ. ID NO. 13 AAE1 CM011611.11210973-1228229
tggttatgcacttgaagaaa

SEQ ID NO: 719 - DNA, 20 bp, Cannabis sativa
  misc_feature: Complimentary to position 13257 of SEQ. ID NO. 13 AAE1 CM011611.11210973-1228229
tggttatgcacttgaagaaa

SEQ ID NO: 720 - DNA, 20 bp, Cannabis sativa
  misc_feature: Complimentary to position 13265 of SEQ. ID NO. 13 AAE1 CM011611.11210973-1228229
atgtatggtaattaatttga

SEQ ID NO: 721 - DNA, 20 bp, Cannabis sativa
  misc_feature: Complimentary to position 13280 of SEQ. ID NO. 13 AAE1 CM011611.11210973-1228229
ttggatattgaaactatgta

SEQ ID NO: 722 - DNA, 20 bp, Cannabis sativa
  misc_feature: Complimentary to position 13299 of SEQ. ID NO. 13 AAE1 CM011611.11210973-1228229
ttaatatgatatggtttatt

SEQ ID NO: 723 - DNA, 20 bp, Cannabis sativa
  misc_feature: Complimentary to position 13308  of SEQ. ID NO. 13 AAE1 CM011611.11210973-1228229
ttcataaagttaatatgata

SEQ ID NO: 724 - DNA, 20 bp, Cannabis sativa
  misc_feature: Complimentary to position 13386 of SEQ. ID NO. 13 AAE1 CM011611.11210973-1228229
accaattgcgtaaacgtgtt

SEQ ID NO: 725 - DNA, 20 bp, Cannabis sativa
  misc_feature: Complimentary to position 13387 of SEQ. ID NO. 13 AAE1 CM011611.11210973-1228229
gaccaattgcgtaaacgtgt

SEQ ID NO: 726 - DNA, 20 bp, Cannabis sativa
  misc_feature: Complimentary to position 13396 of SEQ. ID NO. 13 AAE1 CM011611.11210973-1228229
acccaacacgtttacgcaat

SEQ ID NO: 727 - DNA, 20 bp, Cannabis sativa
  misc_feature: Complimentary to position 13419 of SEQ. ID NO. 13 AAE1 CM011611.11210973-1228229
tcaagtgtcaatttgtttag

SEQ ID NO: 728 - DNA, 20 bp, Cannabis sativa
  misc_feature: Complimentary to position 13444 of SEQ. ID NO. 13 AAE1 CM011611.11210973-1228229
atgattgtatggcgtgatga

SEQ ID NO: 729 - DNA, 20 bp, Cannabis sativa
  misc_feature: Complimentary to position 13455 of SEQ. ID NO. 13 AAE1 CM011611.11210973-1228229
tgaatgatacaatgattgta

SEQ ID NO: 730 - DNA, 20 bp, Cannabis sativa
  misc_feature: Complimentary to position 13524 of SEQ. ID NO. 13 AAE1 CM011611.11210973-1228229
gctgagttaagataacctcc

SEQ ID NO: 731 - DNA, 20 bp, Cannabis sativa
  misc_feature: Complimentary to position 13528 of SEQ. ID NO. 13 AAE1 CM011611.11210973-1228229
ggtagtgaatggcttccagg

SEQ ID NO: 732 - DNA, 20 bp, Cannabis sativa
  misc_feature: Complimentary to position 13531 of SEQ. ID NO. 13 AAE1 CM011611.11210973-1228229
gggggtagtgaatggcttcc

SEQ ID NO: 733 - DNA, 20 bp, Cannabis sativa
  misc_feature: Complimentary to position 13539 of SEQ. ID NO. 13 AAE1 CM011611.11210973-1228229
ataatccagggggtagtgaa

SEQ ID NO: 734 - DNA, 20 bp, Cannabis sativa
  misc_feature: Complimentary to position 13545 of SEQ. ID NO. 13 AAE1 CM011611.11210973-1228229
ggaagccattcactaccccc

SEQ ID NO: 735 - DNA, 20 bp, Cannabis sativa
  misc_feature: Complimentary to position 13549 of SEQ. ID NO. 13 AAE1 CM011611.11210973-1228229
gatgatattaataatccagg

SEQ ID NO: 736 - DNA, 20 bp, Cannabis sativa
  misc_feature: Complimentary to position 13550 of SEQ. ID NO. 13 AAE1 CM011611.11210973-1228229
tgatgatattaataatccag

SEQ ID NO: 737 - DNA, 20 bp, Cannabis sativa
  misc_feature: Complimentary to position 13551 of SEQ. ID NO. 13 AAE1 CM011611.11210973-1228229
atgatgatattaataatcca

SEQ ID NO: 738 - DNA, 20 bp, Cannabis sativa
  misc_feature: Complimentary to position 13552 of SEQ. ID NO. 13 AAE1 CM011611.11210973-1228229
gatgatgatattaataatcc

SEQ ID NO: 739 - DNA, 20 bp, Cannabis sativa
  misc_feature: Complimentary to position 13584 of SEQ. ID NO. 13 AAE1 CM011611.11210973-1228229
tctctacgcaatatacattc

SEQ ID NO: 740 - DNA, 20 bp, Cannabis sativa
  misc_feature: Complimentary to position 13598 of SEQ. ID NO. 13 AAE1 CM011611.11210973-1228229
gatgaagataagtttttcaa

SEQ ID NO: 741 - DNA, 20 bp, Cannabis sativa
  misc_feature: Complimentary to position 13525 of SEQ. ID NO. 13 AAE1 CM011611.11210973-1228229
gtattggagaacattactaa

SEQ ID NO: 742 - DNA, 20 bp, Cannabis sativa
  misc_feature: Complimentary to position 13641 of SEQ. ID NO. 13 AAE1 CM011611.11210973-1228229
ttatataatgttaggtgtat

SEQ ID NO: 743 - DNA, 20 bp, Cannabis sativa
  misc_feature: Complimentary to position 13649 of SEQ. ID NO. 13 AAE1 CM011611.11210973-1228229
ggttaatattatataatgtt

SEQ ID NO: 744 - DNA, 20 bp, Cannabis sativa
  misc_feature: Complimentary to position 13670 of SEQ. ID NO. 13 AAE1 CM011611.11210973-1228229
tgttaattatttgtaattaa

SEQ ID NO: 745 - DNA, 20 bp, Cannabis sativa
  misc_feature: Complimentary to position 13699 of SEQ. ID NO. 13 AAE1 CM011611.11210973-1228229
tttattattttatttgaggg

SEQ ID NO: 746 - DNA, 20 bp, Cannabis sativa
  misc_feature: Complimentary to position 13702 of SEQ. ID NO. 13 AAE1 CM011611.11210973-1228229
ctatttattattttatttga

SEQ ID NO: 747 - DNA, 20 bp, Cannabis sativa
  misc_feature: Complimentary to position 13703 of SEQ. ID NO. 13 AAE1 CM011611.11210973-1228229
actatttattattttatttg

SEQ ID NO: 748 - DNA, 20 bp, Cannabis sativa
  misc_feature: Complimentary to position 13732 of SEQ. ID NO. 13 AAE1 CM011611.11210973-1228229
atagttattattattacctc

SEQ ID NO: 749 - DNA, 20 bp, Cannabis sativa
  misc_feature: Complimentary to position 13733 of SEQ. ID NO. 13 AAE1 CM011611.11210973-1228229
tagttattattattacctca

SEQ ID NO: 750 - DNA, 20 bp, Cannabis sativa
  misc_feature: Complimentary to position 13737 of SEQ. ID NO. 13 AAE1 CM011611.11210973-1228229
attttctgtaagaaaccctg

SEQ ID NO: 751 - DNA, 20 bp, Cannabis sativa
  misc_feature: Complimentary to position 13754 of SEQ. ID NO. 13 AAE1 CM011611.11210973-1228229
ggtttcttacagaaaattct

SEQ ID NO: 752 - DNA, 20 bp, Cannabis sativa
  misc_feature: Complimentary to position 13776 of SEQ. ID NO. 13 AAE1 CM011611.11210973-1228229
gaaatgagaaaagcttgaaa

SEQ ID NO: 753 - DNA, 20 bp, Cannabis sativa
  misc_feature: Complimentary to position 13777 of SEQ. ID NO. 13 AAE1 CM011611.11210973-1228229
aaatgagaaaagcttgaaat

SEQ ID NO: 754 - DNA, 20 bp, Cannabis sativa
  misc_feature: Complimentary to position 13791 of SEQ. ID NO. 13 AAE1 CM011611.11210973-1228229
gtttttgggagtcaagtata

SEQ ID NO: 755 - DNA, 20 bp, Cannabis sativa
  misc_feature: Complimentary to position 13805 of SEQ. ID NO. 13 AAE1 CM011611.11210973-1228229
aagcgaggaaaagagttttt

SEQ ID NO: 756 - DNA, 20 bp, Cannabis sativa
  misc_feature: Complimentary to position 13806 of SEQ. ID NO. 13 AAE1 CM011611.11210973-1228229
gaagcgaggaaaagagtttt

SEQ ID NO: 757 - DNA, 20 bp, Cannabis sativa
  misc_feature: Complimentary to position 13820 of SEQ. ID NO. 13 AAE1 CM011611.11210973-1228229
ggcgcacttttggagaagcg

SEQ ID NO: 758 - DNA, 20 bp, Cannabis sativa
  misc_feature: Complimentary to position 13830 of SEQ. ID NO. 13 AAE1 CM011611.11210973-1228229
caccaatatgggcgcacttt

SEQ ID NO: 759 - DNA, 20 bp, Cannabis sativa
  misc_feature: Complimentary to position 13839 of SEQ. ID NO. 13 AAE1 CM011611.11210973-1228229
ctccaaaagtgcgcccatat

SEQ ID NO: 760 - DNA, 20 bp, Cannabis sativa
  misc_feature: Complimentary to position 13841 of SEQ. ID NO. 13 AAE1 CM011611.11210973-1228229
aaagtaaagtccaccaatat

SEQ ID NO: 761 - DNA, 20 bp, Cannabis sativa
  misc_feature: Complimentary to position 13842 of SEQ. ID NO. 13 AAE1 CM011611.11210973-1228229
caaaagtgcgcccatattgg

SEQ ID NO: 762 - DNA, 20 bp, Cannabis sativa
  misc_feature: Complimentary to position 13842 of SEQ. ID NO. 13 AAE1 CM011611.11210973-1228229
  misc_feature: Complimentary to position 13842, -1,  of SEQ. ID NO. 13 AAE1 CM011611.11210973-1228229
gaaagtaaagtccaccaata

SEQ ID NO: 763 - DNA, 20 bp, Cannabis sativa
  misc_feature: Complimentary to position 13866 of SEQ. ID NO. 13 AAE1 CM011611.11210973-1386
ttttcttttattatgattca

SEQ ID NO: 764 - DNA, 20 bp, Cannabis sativa
  misc_feature: Complimentary to position 13867 of SEQ. ID NO. 13 AAE1 CM011611.11210973-1228229
tttttcttttattatgattc

SEQ ID NO: 765 - DNA, 20 bp, Cannabis sativa
  misc_feature: Complimentary to position 15978 of SEQ. ID NO. 13 AAE1 CM011611.11210973-1228229
gaaaaaatatatatatagta

SEQ ID NO: 766 - DNA, 20 bp, Cannabis sativa
  misc_feature: Complimentary to position 15995 of SEQ. ID NO. 13 AAE1 CM011611.11210973-1228229
gtaaggaaagagagagatta

SEQ ID NO: 767 - DNA, 20 bp, Cannabis sativa
  misc_feature: Complimentary to position 15996 of SEQ. ID NO. 13 AAE1 CM011611.11210973-1228229
taaggaaagagagagattac

SEQ ID NO: 768 - DNA, 20 bp, Cannabis sativa
  misc_feature: Complimentary to position 16000 of SEQ. ID NO. 13 AAE1 CM011611.11210973-1228229
gaaagagagagattacgggt

SEQ ID NO: 769 - DNA, 20 bp, Cannabis sativa
  misc_feature: Complimentary to position 16001 of SEQ. ID NO. 13 AAE1 CM011611.11210973-1228229
aaagagagagattacgggtc

SEQ ID NO: 770 - DNA, 20 bp, Cannabis sativa
  misc_feature: Complimentary to position 16013 of SEQ. ID NO. 13 AAE1 CM011611.11210973-1228229
tacgggtcgggtatccatgc

SEQ ID NO: 771 - DNA, 20 bp, Cannabis sativa
  misc_feature: Complimentary to position 16016 of SEQ. ID NO. 13 AAE1 CM011611.11210973-1228229
gggtcgggtatccatgcagg

SEQ ID NO: 772 - DNA, 20 bp, Cannabis sativa
  misc_feature: Complimentary to position 16016, -1, of SEQ. ID NO. 13 AAE1 CM011611.11210973-1228229
ttggacccgcccctcctgca

SEQ ID NO: 773 - DNA, 20 bp, Cannabis sativa
  misc_feature: Complimentary to position 16917 of SEQ. ID NO. 13 AAE1 CM011611.11210973-1228229
ggtcgggtatccatgcagga

SEQ ID NO: 774 - DNA, 20 bp, Cannabis sativa
  misc_feature: Complimentary to position 16018 of SEQ. ID NO. 13 AAE1 CM011611.11210973-1228229
gtcgggtatccatgcaggag

SEQ ID NO: 775 - DNA, 20 bp, Cannabis sativa
  misc_feature: Complimentary to position 16021 of SEQ. ID NO. 13 AAE1 CM011611.11210973-1228229
gggtatccatgcaggagggg

SEQ ID NO: 776 - DNA, 20 bp, Cannabis sativa
  misc_feature: Complimentary to position 16022 of SEQ. ID NO. 13 AAE1 CM011611.11210973-1228229
ggtatccatgcaggaggggc

SEQ ID NO: 777 - DNA, 20 bp, Cannabis sativa
  misc_feature: Complimentary to position 16035 of SEQ. ID NO. 13 AAE1 CM011611.11210973-1228229
tatggttgctataaagactt

SEQ ID NO: 778 - DNA, 20 bp, Cannabis sativa
  misc_feature: Complimentary to position 16053 of SEQ. ID NO. 13 AAE1 CM011611.11210973-1228229
ctgcaccagatgctctacta

SEQ ID NO: 779 - DNA, 20 bp, Cannabis sativa
  misc_feature: Complimentary to position 16059 of SEQ. ID NO. 13 AAE1 CM011611.11210973-1228229
agcaaccatagtagagcatc

SEQ ID NO: 780 - DNA, 20 bp, Cannabis sativa
  misc_feature: Complimentary to position 16065 of SEQ. ID NO. 13 AAE1 CM011611.11210973-1228229
catagtagagcatctggtgc

SEQ ID NO: 781 - DNA, 20 bp, Cannabis sativa
  misc_feature: Complimentary to position 16066 of SEQ. ID NO. 13 AAE1 CM011611.11210973-1228229
atagtagagcatctggtgca

SEQ ID NO: 782 - DNA, 20 bp, Cannabis sativa
  misc_feature: Complimentary to position 16072 of SEQ. ID NO. 13 AAE1 CM011611.11210973-1228229
gagcatctggtgcagggaga

SEQ ID NO: 783 - DNA, 20 bp, Cannabis sativa
  misc_feature: Complimentary to position 16073 of SEQ. ID NO. 13 AAE1 CM011611.11210973-1228229
agcatctggtgcagggagaa

SEQ ID NO: 784 - DNA, 20 bp, Cannabis sativa
  misc_feature: Complimentary to position 16074 of SEQ. ID NO. 13 AAE1 CM011611.11210973-1228229
gcatctggtgcagggagaag

SEQ ID NO: 785 - DNA, 20 bp, Cannabis sativa
  misc_feature: Complimentary to position 16077 of SEQ. ID NO. 13 AAE1 CM011611.11210973-1228229
tctggtgcagggagaagggg

SEQ ID NO: 786 - DNA, 20 bp, Cannabis sativa
  misc_feature: Complimentary to position 16082 of SEQ. ID NO. 13 AAE1 CM011611.11210973-1228229
tgcagggagaaggggaggtc

SEQ ID NO: 787 - DNA, 20 bp, Cannabis sativa
  misc_feature: Complimentary to position 16095 of SEQ. ID NO. 13 AAE1 CM011611.11210973-1228229
ggaggtcaggcgagagaata

SEQ ID NO: 788 - DNA, 20 bp, Cannabis sativa
  misc_feature: Complimentary to position 16099 of SEQ. ID NO. 13 AAE1 CM011611.11210973-1228229
gtcaggcgagagaatatggt

SEQ ID NO: 789 - DNA, 20 bp, Cannabis sativa
  misc_feature: Complimentary to position 16119 of SEQ. ID NO. 13 AAE1 CM011611.11210973-1228229
tggcaatattgatccatgtt

SEQ ID NO: 790 - DNA, 20 bp, Cannabis sativa
  misc_feature: Complimentary to position 16120 of SEQ. ID NO. 13 AAE1 CM011611.11210973-1228229
ggcaatattgatccatgttt

SEQ ID NO: 791 - DNA, 20 bp, Cannabis sativa
  misc_feature: Complimentary to position 16121 of SEQ. ID NO. 13 AAE1 CM011611.11210973-1228229
gcaatattgatccatgtttg

SEQ ID NO: 792 - DNA, 20 bp, Cannabis sativa
  misc_feature: Complimentary to position 16121, -1,  of SEQ. ID NO. 13 AAE1 CM011611.11210973-1228229
gcgctgccactccccaaaca

SEQ ID NO: 793 - DNA, 20 bp, Cannabis sativa
  misc_feature: Complimentary to position 16126 of SEQ. ID NO. 13 AAE1 CM011611.11210973-1228229
attgatccatgtttggggag

SEQ ID NO: 794 - DNA, 20 bp, Cannabis sativa
  misc_feature: Complimentary to position 16143 of SEQ. ID NO. 13 AAE1 CM011611.11210973-1228229
gccgagatcgtgtgtaatta

SEQ ID NO: 795 - DNA, 20 bp, Cannabis sativa
  misc_feature: Complimentary to position 16153 of SEQ. ID NO. 13 AAE1 CM011611.11210973-1228229
gccataattacacacgatct

SEQ ID NO: 796 - DNA, 20 bp, Cannabis sativa
  misc_feature: Complimentary to position 16165 of SEQ. ID NO. 13 AAE1 CM011611.11210973-1228229
tgagacactccatggtagac

SEQ ID NO: 797 - DNA, 20 bp, Cannabis sativa
  misc_feature: Complimentary to position 16167 of SEQ. ID NO. 13 AAE1 CM011611.11210973-1228229
cgatctcggccagtctacca

SEQ ID NO: 798 - DNA, 20 bp, Cannabis sativa
  misc_feature: Complimentary to position 16173 of SEQ. ID NO. 13 AAE1 CM011611.11210973-1228229
gaagttgctgagacactcca

SEQ ID NO: 799 - DNA, 20 bp, Cannabis sativa
  misc_feature: Complimentary to position 16188 of SEQ. ID NO. 13 AAE1 CM011611.11210973-1228229
tggagtgtctcagcaacttc

SEQ ID NO: 800 - DNA, 20 bp, Cannabis sativa
  misc_feature: Complimentary to position 16188 of SEQ. ID NO. 13 AAE1 CM011611.11210973-1228229
ggagtgtctcagcaacttca

SEQ ID NO: 801 - DNA, 20 bp, Cannabis sativa
  misc_feature: Complimentary to position 16189 of SEQ. ID NO. 13 AAE1 CM011611.11210973-1228229
gagtgtctcagcaacttcag

SEQ ID NO: 802 - DNA, 20 bp, Cannabis sativa
  misc_feature: Complimentary to position 16200 of SEQ. ID NO. 13 AAE1 CM011611.11210973-1228229
caacttcaggggtgatacct

SEQ ID NO: 803 - DNA, 20 bp, Cannabis sativa
  misc_feature: Complimentary to position 16201of SEQ. ID NO. 13 AAE1 CM011611.11210973-1228229
aacttcaggggtgataccta

SEQ ID NO: 804 - DNA, 20 bp, Cannabis sativa
  misc_feature: Complimentary to position 16206 of SEQ. ID NO. 13 AAE1 CM011611.11210973-1228229
gcctctgacttcatagccct

SEQ ID NO: 805 - DNA, 20 bp, Cannabis sativa
  misc_feature: Complimentary to position 16447 of SEQ. ID NO. 13 AAE1 CM011611.11210973-16216
acctagggctatgaagtcag

SEQ ID NO: 806 - DNA, 20 bp, Cannabis sativa
  misc_feature: Complimentary to position 16228 of SEQ. ID NO. 13 AAE1 CM011611.11210973-1228229
caagtccctggactctgttg

SEQ ID NO: 807 - DNA, 20 bp, Cannabis sativa
  misc_feature: Complimentary to position 16233 of SEQ. ID NO. 13 AAE1 CM011611.11210973-1228229
cagaggccacaacagagtcc

SEQ ID NO: 808 - DNA, 20 bp, Cannabis sativa
  misc_feature: Complimentary to position 16234 of SEQ. ID NO. 13 AAE1 CM011611.11210973-1228229
agaggccacaacagagtcca

SEQ ID NO: 809 - DNA, 20 bp, Cannabis sativa
  misc_feature: Complimentary to position 16240 of SEQ. ID NO. 13 AAE1 CM011611.11210973-1228229
gggtaagaattacaagtccc

SEQ ID NO: 810 - DNA, 20 bp, Cannabis sativa
  misc_feature: Complimentary to position 16260 of SEQ. ID NO. 13 AAE1 CM011611.11210973-1228229
agtttaatagaagtaataat

SEQ ID NO: 811 - DNA, 20 bp, Cannabis sativa
  misc_feature: Complimentary to position 12261of SEQ. ID NO. 13 AAE1 CM011611.11210973-1228229
cagtttaatagaagtaataa

SEQ ID NO: 812 - DNA, 20 bp, Cannabis sativa
  misc_feature: Complimentary to position 16332 of SEQ. ID NO. 13 AAE1 CM011611.11210973-1228229
aatattatatatatttatat

SEQ ID NO: 813 - DNA, 20 bp, Cannabis sativa
  misc_feature: Complimentary to position 16339 of SEQ. ID NO. 13 AAE1 CM011611.11210973-1228229
tatatatttatattggattt

SEQ ID NO: 814 - DNA, 20 bp, Cannabis sativa
  misc_feature: Complimentary to position 16340 of SEQ. ID NO. 13 AAE1 CM011611.11210973-1228229
atatatttatattggatttt

SEQ ID NO: 815 - DNA, 20 bp, Cannabis sativa
  misc_feature: Complimentary to position 16344 of SEQ. ID NO. 13 AAE1 CM011611.11210973-1228229
atttatattggattttggga

SEQ ID NO: 816 - DNA, 20 bp, Cannabis sativa
  misc_feature: Complimentary to position 16345 of SEQ. ID NO. 13 AAE1 CM011611.11210973-1228229
tttatattggattttgggat

SEQ ID NO: 817 - DNA, 20 bp, Cannabis sativa
  misc_feature: Complimentary to position 16398 of SEQ. ID NO. 13 AAE1 CM011611.11210973-1228229
atcaaagacttgctcaataa

SEQ ID NO: 818 - DNA, 20 bp, Cannabis sativa
  misc_feature: Complimentary to position 16404 of SEQ. ID NO. 13 AAE1 CM011611.11210973-1228229
gacttgctcaataatggatt

SEQ ID NO: 819 - DNA, 20 bp, Cannabis sativa
  misc_feature: Complimentary to position 16408 of SEQ. ID NO. 13 AAE1 CM011611.11210973-1228229
tgctcaataatggattaggc

SEQ ID NO: 820 - DNA, 20 bp, Cannabis sativa
  misc_feature: Complimentary to position 16415 of SEQ. ID NO. 13 AAE1 CM011611.11210973-1228229
taatggattaggctggaatt

SEQ ID NO: 821 - DNA, 20 bp, Cannabis sativa
  misc_feature: Complimentary to position 16420 of SEQ. ID NO. 13 AAE1 CM011611.11210973-1228229
gattaggctggaatttggtt

SEQ ID NO: 822 - DNA, 20 bp, Cannabis sativa
  misc_feature: Complimentary to position 16421 of SEQ. ID NO. 13 AAE1 CM011611.11210973-1228229
attaggctggaatttggttt

SEQ ID NO: 823 - DNA, 20 bp, Cannabis sativa
  misc_feature: Complimentary to position 16447 of SEQ. ID NO. 13 AAE1 CM011611.11210973-1228229
tcaaaggcaacacaagtgat

SEQ ID NO: 824 - DNA, 20 bp, Cannabis sativa
  misc_feature: Complimentary to position 1535 of SEQ. ID NO. 4 CBDAS CM011610.121836038-21839672
tgtccacctgcgcaaacagt

SEQ ID NO: 825 - DNA, 20 bp, Cannabis sativa
  misc_feature: Complimentary to position 1555 of SEQ. ID NO. 4 CBDAS CM011610.121836038-21839672
tgcgcaggtggacactttgg

SEQ ID NO: 826 - DNA, 20 bp, Cannabis sativa
  misc_feature: Complimentary to position 1273 of SEQ. ID NO. 13 AAE1 CM011611.11210973-1228229
gaaactaaatcctctgttca

(19) United States
(12) Patent Application Publication
(10) Pub. No.: US 2022/0002742 A1
(43) Pub. Date: Jan. 6, 2022

(54) MODULATION OF CANNABINOID PROFILE IN CANNABIS

(71) Applicant: CANBREED LTD., Givat Chen (IL)

(72) Inventors: Tal SHERMAN, Rehovot (IL); Ido MARGALIT, Gan Yavne (IL)

(73) Assignee: CANBREED LTD., Givat Chen (IL)

(21) Appl. No.: 17/250,651

(22) PCT Filed: Aug. 15, 2019
(86) PCT No.: PCT/IL2019/050920

§ 371 Date: Feb. 16, 2021

Related U.S. Application Data
(60) Provisional application No. 62/719,151, filed on Aug. 17, 2018.

(51) Int. Cl. C12N 15/82; C12N 9/02; A61K 31/05

(52) U.S. Cl. CPC C12N 15/8243; C12Y 121/03007; A61K 31/05; C12N 9/0004

(57) ABSTRACT

Provided is a Cannabis plant with reduced delta-9-tetrahydrocannabinol (THC) content, or reduced cannabidiol (CBD) content, or reduced THC and CBD content. According to a core aspect of the invention, the Cannabis plant comprises at least one targeted genome modification effective in decreasing expression of. at least one Cannabis gene encoding a cannabinoid biosynthesis enzyme selected from the group consisting of Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) and any combination thereof. Further disclose are methods for production of the Cannabis plants and use thereof.

### SEQUENCE LISTING

The Sequence Listing submitted in text format (.txt) filed on Jun. 30, 2021, named “SequenceListing.txt”, created on Jun. 25, 2021 (329 KB), is incorporated herein by reference.

### FIELD OF THE INVENTION

The present invention relates generally to Cannabis plants with altered expression of cannabinoids and/or altered expression of cannabinoid synthesizing enzymes. More specifically, the present disclosure relates to methods for controlling genes associated with cannabinoids synthesis in Cannabis plants.

### BACKGROUND OF THE INVENTION

Cannabis has been bred by many different cultures for various uses such as food, fiber and medicine since the dawn of agricultural societies. In the last few decades, Cannabis breeding has stopped as it became illegal and non-economic to do so. With the recent legislation converting Cannabis back to legality, there is a growing need for the implementation of new and advanced breeding techniques in future Cannabis breeding programs. This will allow speeding up the long process of classical breeding and accelerate reaching new and genetically improved Cannabis varieties for fiber, food and medicine products. Developing and implementing molecular biology tools to support the breeders, will allow creating new traits and tracking the movement of such desired traits across breeders germplasm.

According to some publications, the Cannabis plant chemical profile is composed of at least 483 known chemical compounds, which include cannabinoids, terpenoids, flavonoids, nitrogenous compounds, amino acids, proteins, glycoproteins, enzymes, sugars and related compounds, hydrocarbons, alcohols, aldehydes, ketones, acids, fatty acids, esters, lactones, steroids, terpenes, non-cannabinoid phenols, vitamins, and pigments.

Cannabinoids are of particular interest for research and commercialization. There are at least 113 different cannabinoids isolated from Cannabis, exhibiting varied effects. The classical cannabinoids are concentrated in a viscous resin produced in structures known as glandular trichomes. The most notable cannabinoid is the phytocannabinoid delta 9 tetrahydrocannabinol (THC), the primary psychoactive compound in Cannabis. Cannabidiol (CBD) is another major constituent of the plant.

Other cannabinoids of interest include, Cannabigerol (CBG), Cannabigerolic Acid (CBGA), Cannabinol (CBN), Cannabichromene (CBC), Tetrahydrocannabivarin (THCV), Cannabigerovarin (CBGV), Cannabigerovarinic Acid (CBGVA), tetrahydrocannabinolic acid (THCA), cannabidiolic acid (CBDA), cannabichromene (CBC), cannabicyclol (CBL), cannabivarin (CBV), cannabidivarin (CBDV), cannabichromevarin (CBCV), cannabigerol monomethyl ether (CBGM), cannabielsoin (CBE) and cannabicitran (CBT).

Cannabis plants can exhibit wide variation in the quantity and type of cannabinoids they produce. The mixture of cannabinoids produced by a plant is known as the plant's cannabinoid profile. Selective breeding has been used to control the genetics of plants and modify the cannabinoid profile. For example, strains that are used as fiber (commonly called hemp) are usually bred such that they are low in psychoactive chemicals like THC. Strains used in medicine are often bred for high CBD content, and strains used for recreational purposes are usually bred for high THC content or for a specific chemical balance.

Quantitative analysis of a plant's cannabinoid profile is often determined by analytical methods such as gas chromatography (GC), gas chromatography combined with mass spectrometry (GC/MS) and liquid chromatography (LC) techniques.

A variety of growing and cultivating techniques have been developed for increasing the production of secondary compounds within plants of genus Cannabis. These techniques include outdoor cultivation, indoor cultivation, hydroponics, fertilization, atmospheric manipulation, cloning, crossbreeding etc. There is very limited if any molecular tools supporting or leading the breeding process. Traditional Cannabis breeding is done by mixing breeding material with hope to find the desired traits and phenotypes by random crosses. These methods have allowed the construction of the leading Cannabis varieties on the market today.

As the cultivation of Cannabis intensifies, breeding and farming techniques fail to provide the level of control of cannabinoid production and yield needed. Cannabinoid research is still new and having plants producing modified levels of certain cannabinoids would be advantageous for research and medical purposes. Furthermore, separating and isolating specific molecules of the plant out of hundreds could be challenging and time consuming.

In view of the above there is an unmet and long felt need for non-GMO, advanced breeding of Cannabis plants producing particular amounts of predetermined cannabinoids. In particular, there is a need for Cannabis plants selectively producing predetermined ratios and/or concentrations of cannabinoids for medical use.

### SUMMARY OF THE INVENTION

It is one object of the present invention to disclose a Cannabis plant with reduced delta-9-tetrahydrocannabinol (THC) content, or reduced cannabidiol (CBD) content, or reduced THC and CBD content, wherein said plant comprises at least one targeted genome modification effective in decreasing expression of a at least one Cannabis gene encoding a cannabinoid biosynthesis enzyme selected from the group consisting of Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) and any combination thereof.

It is a further object of the present invention to disclose the Cannabis plant as defined above, wherein said plant comprises reduced THC content, or reduced CBD content, or reduced THC and CBD content relative to a Cannabis plant of a similar genotype or genetic background that does not comprise said targeted genome modification.

It is a further object of the present invention to disclose the Cannabis plant as defined in any of the above, wherein said targeted genome modification is located in the cannabinoid biosynthesis enzyme gene locus.

It is a further object of the present invention to disclose the Cannabis plant as defined in any of the above, wherein said targeted genome modification interrupts or interferes with or down regulate or silence transcription and/or translation of said Cannabis gene encoding said cannabinoid biosynthesis enzyme.

It is a further object of the present invention to disclose the Cannabis plant as defined in any of the above, wherein said plant comprises an endonuclease enzyme targeting a nucleic acid sequence coding for said at least one cannabinoid biosynthesis enzyme.

It is a further object of the present invention to disclose the Cannabis plant as defined in any of the above, wherein said plant does not comprise within its genome exogenous genetic material and said plant is a non-naturally occurring Cannabis plant or cell thereof.

It is a further object of the present invention to disclose the Cannabis plant as defined in any of the above, wherein the nucleotide sequence encoding said at least one cannabinoid biosynthesis enzyme is selected from the group consisting of: CsTHCAS having a genomic nucleotide sequence as set forth in SEQ ID NO:1 or a functional variant thereof, CsCBDAS having a genomic nucleotide sequence as set forth in SEQ ID NO:4 or a functional variant thereof, CsPT having a genomic nucleotide sequence as set forth in SEQ ID NO:7 or a functional variant thereof, CsOLS having a genomic nucleotide sequence as set forth in SEQ ID NO:10 or a functional variant thereof and CsAAE1 having a genomic nucleotide sequence as set forth in SEQ ID NO:13 or a functional variant thereof.

It is a further object of the present invention to disclose the Cannabis plant as defined in any of the above, wherein said functional variant has at least 75% sequence identity to the nucleotide sequence of said cannabinoid biosynthesis enzyme or a codon degenerate nucleotide sequence thereof.

It is a further object of the present invention to disclose the Cannabis plant as defined in any of the above, wherein said plant has decreased expression levels of at least one of CsTHCAS protein having an amino acid sequence with at least 75% identity to the amino acid sequence as set forth in SEQ ID NO:3 or a conservatively substituted amino acid sequence thereof, CsCBDAS protein having an amino acid sequence with at least 75% identity to the amino acid sequence as set forth in SEQ ID NO:6 or a conservatively substituted amino acid sequence thereof, CsPT protein having an amino acid sequence with at least 75% identity to the amino acid sequence as set forth in SEQ ID NO:9 or a conservatively substituted amino acid sequence thereof, CsOLS protein having an amino acid sequence with at least 75% identity to the amino acid sequence as set forth in SEQ ID NO:12 or a conservatively substituted amino acid sequence thereof, and CsAAE1 protein having an amino acid sequence with at least 75% identity to the amino acid sequence as set forth in SEQ ID NO:15 or a conservatively substituted amino acid sequence thereof.

It is a further object of the present invention to disclose the Cannabis plant as defined in any of the above, wherein said Cannabis plant has modulated expression of one or more of the cannabinoids, optionally cannabigerolic acid, cannabigerol, DELTA.9-tetrahydrocannabinolic acid, cannabidiolic acid, cannabichromenic acid, DELTA.9-tetrahydrocannabinol, cannabidiol, cannabichromene, THC, D9-THC, D8-THC, THCA, THCV, D8-THCV, D9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA and cannabidiol.

It is a further object of the present invention to disclose the Cannabis plant as defined in any of the above, wherein said Cannabis plant has reduced expression of one or more of the cannabinoids, optionally cannabigerolic acid, cannabigerol, DELTA.9-tetrahydrocannabinolic acid, cannabidiolic acid, cannabichromenic acid, DELTA.9-tetrahydrocannabinol, cannabidiol, cannabichromene, THC, D9-THC, D8-THC, THCA, THCV, D8-THCV, D9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA and cannabidiol, as compared to a Cannabis plant of a similar genotype or genetic background that does not comprise said targeted genome modification.

It is a further object of the present invention to disclose the Cannabis plant as defined in any of the above, wherein said targeted genome modification is introduced using CRISPR (Clustered Regularly Interspaced Short Palindromic Repeats) and CRISPR-associated (Cas) genes (CRISPR/Cas) system, Transcription activator-like effector nuclease (TALEN), Zinc Finger Nuclease (ZFN), meganuclease or any combination thereof.

It is a further object of the present invention to disclose the Cannabis plant as defined in any of the above, wherein said CRISPR/Cas genes or proteins are selected from the group consisting of Cas3, Cas4, Cas5, Cas5e (or CasD), Cash, Cas6e, Cas6f, Cas7, Cas8a1, Cas8a2, Cas8b, Cas8c, Cas9, Cast 0, Castl Od, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb 1, Csb2, Csb3, Csx17, Csx14, Csx10, Csx16, CsaX, Csx3, Csz1, Csx15, Csf1, Csf2, Csf3, Csf4, and Cu1966.

It is a further object of the present invention to disclose the Cannabis plant as defined in any of the above, wherein said targeted genome modification is introduced via (i) at least one RNA-guided (gRNA) endonuclease or nucleic acid encoding at least one RNA-guided endonuclease, and (ii) at least one guide RNA (gRNA) or DNA encoding at least one guide RNA (gRNA), further wherein each of said at least one gRNA directs said endonuclease to a targeted site located in the genomic sequence of said at least one Cannabis gene encoding a cannabinoid biosynthesis enzyme.

It is a further object of the present invention to disclose the Cannabis plant as defined in any of the above, wherein the gRNA nucleotide sequence targeted to CsTHCAS genomic sequence is as set forth in nucleotide sequence selected from the group consisting of SEQ. ID. NO.: 16-167 and any combination thereof.

It is a further object of the present invention to disclose the Cannabis plant as defined in any of the above, wherein the gRNA nucleotide sequence targeted to CsCBDAS genomic sequence is as set forth in nucleotide sequence selected from the group consisting of SEQ. ID. NO.: 168-304, SEQ. ID. NO.: 824-825, and any combination thereof.

It is a further object of the present invention to disclose the Cannabis plant as defined in any of the above, wherein the gRNA nucleotide sequence targeted to CsPT genomic sequence is as set forth in nucleotide sequence selected from the group consisting of SEQ. ID. NO.: 305-458 and any combination thereof.

It is a further object of the present invention to disclose the Cannabis plant as defined in any of the above, wherein the gRNA nucleotide sequence targeted to CsOLS genomic sequence is as set forth in nucleotide sequence selected from the group consisting of SEQ. ID. NO.: 459-509 and any combination thereof.

It is a further object of the present invention to disclose the Cannabis plant as defined in any of the above, wherein the gRNA nucleotide sequence targeted to CsAAE1 genomic sequence is as set forth in nucleotide sequence selected from the group consisting of SEQ. ID. NO.: 510-823, SEQ. ID. NO.: 826, and any combination thereof.

It is a further object of the present invention to disclose the Cannabis plant as defined in any of the above, wherein said plant is mutated in a gene selected from the group consisting of CsTHCAS, CsSP, CsCBDAS, CsPT, CsOLS, CsAAE1 and any combination thereof.

It is a further object of the present invention to disclose the Cannabis plant as defined in any of the above, wherein said mutated CsTHCAS, CsSP, CsCBDAS, CsPT, CsOLS and/or CsAAE1 gene is a CRISPR/Cas9-induced heritable mutated allele.

It is a further object of the present invention to disclose the Cannabis plant as defined in any of the above, wherein said genome modification is a missense mutation, nonsense mutation, insertion, deletion, indel, substitution or duplication.

It is a further object of the present invention to disclose the Cannabis plant as defined in any of the above, wherein the insertion or the deletion produces a gene comprising a frameshift.

It is a further object of the present invention to disclose the Cannabis plant as defined in any of the above, wherein said plant is homozygous for said at least one mutated gene.

It is a further object of the present invention to disclose the Cannabis plant as defined in any of the above, wherein said mutation is in the coding region of said gene, a mutation in the regulatory region of said gene, a mutation in a gene downstream of said gene in the cannabinoid biosynthesis pathway or an epigenetic factor.

It is a further object of the present invention to disclose the Cannabis plant as defined in any of the above, wherein said mutation is a silencing mutation, a knockdown mutation, a knockout mutation, a loss of function mutation or any combination thereof.

It is a further object of the present invention to disclose the Cannabis plant as defined in any of the above, wherein said genome modification is generated in planta.

It is a further object of the present invention to disclose the Cannabis plant as defined in any of the above wherein said genome modification is generated in planta via introduction of a construct comprising (a) Cas DNA and gRNA sequence selected from the group consisting of SEQ ID NO:16-SEQ ID NO:826 and any combination thereof, or (b) a ribonucleoprotein (RNP) complex comprising Cas protein and gRNA sequence selected from the group consisting of SEQ ID NO:16-826 and any combination thereof.

It is a further object of the present invention to disclose the Cannabis plant as defined in any of the above wherein said gRNA sequence comprises a 3′ NGG Protospacer Adjacent Motif (PAM).

It is a further object of the present invention to disclose the Cannabis plant as defined in any of the above wherein said gRNA is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.

It is a further object of the present invention to disclose the Cannabis plant as defined in any of the above wherein said Cannabis plant is selected from the group of species that includes, but is not limited to, Cannabis sativa (C. sativa), C. indica, C. ruderalis and any hybrid or cultivated variety of the genus Cannabis.

It is a further object of the present invention to disclose a Cannabis plant, plant part or plant cell as defined in any of the above wherein said plant does not comprise a transgene.

It is a further object of the present invention to disclose a plant part, plant cell or plant seed of a plant as defined in any of the above.

It is a further object of the present invention to disclose a tissue culture of regenerable cells, protoplasts or callus obtained from the Cannabis plant as defined in any of the above.

It is a further object of the present invention to disclose the Cannabis plant as defined in any of the above wherein said plant genotype is obtainable by deposit under accession number with NCIMB Aberdeen AB21 9YA, Scotland, UK, or wherein said plant genotype is obtainable by deposit under accession number with ATCC, LGC Standards, Queens Road Teddington Middlesex TW11 OLY UK.

It is a further object of the present invention to disclose the Cannabis plant as defined in any of the above, wherein said Cannabis plant comprises a DNA encoding an antisense RNA or a siRNA effective to suppress expression of CsTHCAS, CsSP, CsCBDAS, CsPT, CsOLS, CsAAE1 and any combination thereof, the DNA operably linked to a heterologous promoter, wherein the Cannabis plant comprises reduced THC content, reduced CBD content, or decreased THC and CBD content relative to a Cannabis plant of a similar genotype that does not comprise the DNA.

It is a further object of the present invention to disclose the Cannabis plant as defined in any of the above, wherein said Cannabis plant has a THC content of up to 30% by weight, particularly between about %0.1 to about 30% by weight, more particularly between about %0.3 to about 30%, even more particularly between about %0.3 to about 10% by weight.

It is a further object of the present invention to disclose the Cannabis plant as defined in any of the above, wherein said Cannabis plant has a CBD content of up to 30% by weight, particularly between about %0.1 to about 30% by weight, more particularly between about %0.3 to about 30%, even more particularly between about %0.3 to about 10% by weight.

It is a further object of the present invention to disclose the Cannabis plant as defined in any of the above, wherein said plant has a THC and/or CBD content of not more than about 0.5% by weight.

It is a further object of the present invention to disclose the Cannabis plant as defined in any of the above, wherein said plant is THC free.

It is a further object of the present invention to disclose the Cannabis plant as defined in any of the above, wherein said plant has at least one targeted genome modification in at least one Cannabis gene encoding cannabinoid precursor synthesis enzyme selected from the group consisting of CsAAE, CsPT and CsOLS.

It is a further object of the present invention to disclose the Cannabis plant as defined in any of the above, wherein said plant exhibits reduced expression of THC, CBD or both relative to a Cannabis plant of a similar genotype or genetic background lacking said targeted genome modification.

It is a further object of the present invention to disclose the Cannabis plant as defined in any of the above, wherein said plant has a THC and/or CBD content of up to 30% by weight, particularly between about %0.1 to about 30% by weight, more particularly between about %0.3 to about 30%, even more particularly between about %0.3 to about 10% by weight.

It is a further object of the present invention to disclose the Cannabis plant as defined in any of the above, wherein said plant has a THC and/or CBD content of not more than about 0.5% by weight.

It is a further object of the present invention to disclose the Cannabis plant as defined in any of the above, wherein said plant has at least one targeted genome modification in at least one Cannabis gene encoding cannabinoid synthesis enzyme selected from the group consisting of CsTHCAS and CsCBDAS.

It is a further object of the present invention to disclose the Cannabis plant as defined in any of the above, wherein said plant has at least one targeted genome modification in CsTHCAS and said plant exhibits reduced expression of THCA or THC, and elevated expression of CBD or CBDA relative to a Cannabis plant of a similar genotype or genetic background lacking said targeted genome modification.

It is a further object of the present invention to disclose the Cannabis plant as defined in any of the above, wherein said plant has a THC and/or THCA content of up to 30% by weight, particularly between about %0.1 to about 30% by weight, more particularly between about %0.3 to about 30%, even more particularly between about %0.3 to about 10% by weight.

It is a further object of the present invention to disclose the Cannabis plant as defined in any of the above, wherein said plant has a THC and/or THCA content of not more than about 0.5% by weight.

It is a further object of the present invention to disclose the Cannabis plant as defined in any of the above, wherein said plant has at least one targeted genome modification in CsCBDAS and said plant exhibits reduced expression of CBDA or CBD, and elevated expression of THC or THCA relative to a Cannabis plant of a similar genotype or genetic background lacking said targeted genome modification.

It is a further object of the present invention to disclose the Cannabis plant as defined in any of the above, wherein said plant has a THC and/or CBD content of up to 30% by weight, particularly between about %0.1 to about 30% by weight, more particularly between about %0.3 to about 30%, even more particularly between about %0.3 to about 10% by weight.

It is a further object of the present invention to disclose the Cannabis plant as defined in any of the above, wherein said plant has a CBD and/or CBDA content of not more than about 0.5% by weight.

It is a further object of the present invention to disclose a Cannabis plant derived product from the plant as defined in any of the above.

It is a further object of the present invention to disclose the Cannabis plant derived product as defined above, comprising a combined cannabidiolic acid and cannabidiol concentration of about 0.3% to about 30% by weight.

It is a further object of the present invention to disclose the Cannabis plant derived product as defined in any of the above, comprising a combined delta-9-tetrahydrocannabinol and tetrahydrocannabinolic acid concentration of between about 0.3% to about 30% by weight.

It is a further object of the present invention to disclose the Cannabis plant derived product as defined in any of the above, comprising Cannabis oil, Cannabis tincture, dried Cannabis flowers, and/or dried Cannabis leaves.

It is a further object of the present invention to disclose the Cannabis plant derived product as defined in any of the above, for medical use.

It is a further object of the present invention to disclose the Cannabis plant derived product as defined in any of the above, formulated for inhalation, oral consumption, sublingual consumption, or topical consumption.

It is a further object of the present invention to disclose a medical composition derived from the plant as defined in any of the above.

It is a further object of the present invention to disclose a method for producing a Cannabis plant with reduced delta-9-tetrahydrocannabinol (THC) content, or reduced cannabidiol (CBD) content, or reduced THC and CBD content, wherein said method comprises steps of introducing into said Cannabis plant genome or a cell thereof at least one targeted genome modification effective in decreasing expression of a at least one Cannabis gene encoding a cannabinoid biosynthesis enzyme selected from the group consisting of tetrahydrocannabinolic acid synthase (THCAS), cannabidiolic acid synthase (CBDAS), aromatic prenyltransferase (PT), olivetol synthase (OLS), acyl-activating enzyme 1 (AAE1) and any combination thereof.

It is a further object of the present invention to disclose the method as defined above, further comprising steps of: (a) constructing an endonuclease enzyme targeting a nucleic acid sequence coding for a cannabinoid biosynthesis enzyme selected from the group consisting of CsTHCAS, CsCBDAS, CsPT, CsOLS, CsAAE1 and any combination thereof; (b) introducing the endonuclease enzyme into the genome of the Cannabis plant of; and (c) decreasing expression of the at least one cannabinoid biosynthesis enzyme within the genome.

It is a further object of the present invention to disclose the method as defined in any of the above, further comprising steps of: (a) introducing into the Cannabis plant or a cell thereof (i) at least one RNA-guided endonuclease or nucleic acid encoding at least one RNA-guided endonuclease, and (ii) at least one guide RNA (gRNA) or DNA encoding at least one gRNA; (b) assaying the Cannabis plant or a cell thereof for an endonuclease-mediated modification in the DNA of said at least one Cannabis cannabinoid biosynthesis enzyme gene locus; and (c) identifying the Cannabis plant, or a cell thereof, or a progeny cell thereof as comprising a modification in said at least one gene locus.

It is a further object of the present invention to disclose the method as defined in any of the above, further comprising steps of culturing the Cannabis plant or cell thereof such that each guide RNA directs an RNA-guided endonuclease to a targeted site in the chromosomal sequence of said at least one Cannabis cannabinoid biosynthesis enzyme, enabling the RNA-guided endonuclease introduce a double-stranded break in the targeted site, and repair of the double-stranded break by a DNA repair process such that the chromosomal sequence is modified, wherein the targeted site is located in the gene locus of the at least one Cannabis cannabinoid biosynthesis enzyme and the chromosomal modification interrupts or interferes with transcription and/or translation of said at least one gene encoding Cannabis cannabinoid biosynthesis enzyme.

It is a further object of the present invention to disclose the method as defined in any of the above, wherein the endonuclease enzyme is a CRISPR/Cas9 system.

It is a further object of the present invention to disclose the method as defined in any of the above, comprising steps of interfering with expression of a cannabinoid biosynthesis enzyme selected from the group consisting of CsTHCAS, CsCBDAS, CsPT, CsOLS, CsAAE1 and any combination thereof.

It is a further object of the present invention to disclose the method as defined in any of the above, wherein said plant comprises reduced THC content, or reduced CBD content, or reduced THC and CBD content relative to a Cannabis plant of a similar genotype or genetic background that does not comprise said targeted genome modification.

It is a further object of the present invention to disclose the method as defined in any of the above, wherein said targeted genome modification is located in the cannabinoid biosynthesis enzyme gene locus.

It is a further object of the present invention to disclose the method as defined in any of the above, wherein said targeted genome modification interrupts or interferes with or down regulate or silence transcription and/or translation of said Cannabis gene encoding said cannabinoid biosynthesis enzyme.

It is a further object of the present invention to disclose the method as defined in any of the above, wherein said plant comprises an endonuclease enzyme targeting a nucleic acid sequence coding for said at least one cannabinoid biosynthesis enzyme.

It is a further object of the present invention to disclose the method as defined in any of the above, wherein said plant does not comprise within its genome exogenous genetic material and said plant is a non-naturally occurring Cannabis plant or cell thereof.

It is a further object of the present invention to disclose the method as defined in any of the above, wherein the nucleotide sequence encoding said at least one cannabinoid biosynthesis enzyme is selected from the group consisting of: CsTHCAS having a genomic nucleotide sequence as set forth in SEQ ID NO:1 or a functional variant thereof, CsCBDAS having a genomic nucleotide sequence as set forth in SEQ ID NO:4 or a functional variant thereof, CsPT having a genomic nucleotide sequence as set forth in SEQ ID NO:7 or a functional variant thereof, CsOLS having a genomic nucleotide sequence as set forth in SEQ ID NO:10 or a functional variant thereof and CsAAE1 having a genomic nucleotide sequence as set forth in SEQ ID NO:13 or a functional variant thereof.

It is a further object of the present invention to disclose the method as defined in any of the above, wherein said functional variant has at least 75% sequence identity to the nucleotide sequence of said cannabinoid biosynthesis enzyme or a codon degenerate nucleotide sequence thereof.

It is a further object of the present invention to disclose the method as defined in any of the above, wherein said plant has decreased expression levels of at least one of CsTHCAS protein having an amino acid sequence with at least 75% identity to the amino acid sequence as set forth in SEQ ID NO:3 or a conservatively substituted amino acid sequence thereof, CsCBDAS protein having an amino acid sequence with at least 75% identity to the amino acid sequence as set forth in SEQ ID NO:6 or a conservatively substituted amino acid sequence thereof, CsPT protein having an amino acid sequence with at least 75% identity to the amino acid sequence as set forth in SEQ ID NO:9 or a conservatively substituted amino acid sequence thereof, CsOLS protein having an amino acid sequence with at least 75% identity to the amino acid sequence as set forth in SEQ ID NO:12 or a conservatively substituted amino acid sequence thereof, and CsAAE1 protein having an amino acid sequence with at least 75% identity to the amino acid sequence as set forth in SEQ ID NO:15 or a conservatively substituted amino acid sequence thereof.

It is a further object of the present invention to disclose the method as defined in any of the above, wherein said Cannabis plant has modulated expression of one or more of the cannabinoids, optionally cannabigerolic acid, cannabigerol, DELTA. 9-tetrahydrocannabinolic acid, cannabidiolic acid, cannabichromenic acid, DELTA. 9-tetrahydrocannabinol, cannabidiol, cannabichromene, THC, D9-THC, D8-THC, THCA, THCV, D8-THCV, D9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA and cannabidiol.

It is a further object of the present invention to disclose the method as defined in any of the above, wherein said Cannabis plant has reduced expression of one or more of the cannabinoids, optionally cannabigerolic acid, cannabigerol, DELTA. 9-tetrahydrocannabinolic acid, cannabidiolic acid, cannabichromenic acid, DELTA. 9-tetrahydrocannabinol, cannabidiol, cannabichromene, THC, D9-THC, D8-THC, THCA, THCV, D8-THCV, D9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA and cannabidiol, as compared to a Cannabis plant of a similar genotype or genetic background that does not comprise said targeted genome modification.

It is a further object of the present invention to disclose the method as defined in any of the above, wherein said targeted genome modification is introduced using CRISPR (Clustered Regularly Interspaced Short Palindromic Repeats) and CRISPR-associated (Cas) genes (CRISPR/Cas) system, Transcription activator-like effector nuclease (TALEN), Zinc Finger Nuclease (ZFN), meganuclease or any combination thereof.

It is a further object of the present invention to disclose the method as defined in any of the above, wherein said CRISPR/Cas genes or proteins are selected from the group consisting of Cas3, Cas4, Cas5, Cas5e (or CasD), Cash, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966.

It is a further object of the present invention to disclose the method as defined in any of the above, wherein said targeted genome modification is introduced via (i) at least one RNA-guided (gRNA) endonuclease or nucleic acid encoding at least one RNA-guided endonuclease, and (ii) at least one guide RNA (gRNA) or DNA encoding at least one guide RNA (gRNA), further wherein each of said at least one gRNA directs said endonuclease to a targeted site located in the genomic sequence of said at least one Cannabis gene encoding a cannabinoid biosynthesis enzyme.

It is a further object of the present invention to disclose the method as defined in any of the above, wherein (a) the gRNA nucleotide sequence targeted to CsTHCAS genomic sequence is as set forth in nucleotide sequence selected from the group consisting of SEQ. ID. NO.: 16-167 and any combination thereof; (b) the gRNA nucleotide sequence targeted to CsCBDAS genomic sequence is as set forth in nucleotide sequence selected from the group consisting of SEQ. ID. NO.: 168-304, SEQ. ID. NO.: 824-825, and any combination thereof; (c) the gRNA nucleotide sequence targeted to CsPT genomic sequence is as set forth in nucleotide sequence selected from the group consisting of SEQ. ID. NO.: 305-458 and any combination thereof; (d) the gRNA nucleotide sequence targeted to CsOLS genomic sequence is as set forth in nucleotide sequence selected from the group consisting of SEQ. ID. NO.: 459-509 and any combination thereof; and (e) the gRNA nucleotide sequence targeted to CsAAE1 genomic sequence is as set forth in nucleotide sequence selected from the group consisting of SEQ. ID. NO.: 510-823, SEQ. ID. NO.: 826, and any combination thereof.

It is a further object of the present invention to disclose the method as defined in any of the above, wherein said plant is mutated in a gene selected from the group consisting of CsTHCAS, CsSP, CsCBDAS, CsPT, CsOLS, CsAAE1 and any combination thereof.

It is a further object of the present invention to disclose the method as defined in any of the above, wherein said at least one genome modification is generated in planta.

It is a further object of the present invention to disclose the method as defined in any of the above, wherein said genome modification is generated in planta via introduction of a construct comprising (a) Cas DNA and gRNA sequence selected from the group consisting of SEQ ID NO:16-SEQ ID NO:826 and any combination thereof, or (b) a ribonucleoprotein (RNP) complex comprising Cas protein and gRNA sequence selected from the group consisting of SEQ ID NO:16-826 and any combination thereof.

It is a further object of the present invention to disclose the method as defined in any of the above, wherein said gRNA is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.

It is a further object of the present invention to disclose a plant part, plant cell or plant seed produced by the method as defined in any of the above.

It is a further object of the present invention to disclose the method as defined in any of the above, wherein said Cannabis plant comprises a DNA encoding an antisense RNA or a siRNA effective to suppress expression of CsTHCAS, CsSP, CsCBDAS, CsPT, CsOLS, CsAAE1 and any combination thereof, the DNA operably linked to a heterologous promoter, wherein the Cannabis plant comprises reduced THC content, reduced CBD content, or decreased THC and CBD content relative to a Cannabis plant of a similar genotype that does not comprise the DNA.

It is a further object of the present invention to disclose a method for producing a medical Cannabis composition, the method comprising: (a) obtaining the Cannabis plant of claim 1; and (b) formulating a medical Cannabis composition from said plant.

It is a further object of the present invention to disclose a method for manipulating a content of one or more cannabinoids in a Cannabis plant, the method comprising down-regulating activity of at least one Cannabis gene encoding a cannabinoid biosynthesis enzyme selected from the group consisting of tetrahydrocannabinolic acid synthase (THCAS), cannabidiolic acid synthase (CBDAS), aromatic prenyltransferase (PT), olivetol synthase (OLS), acyl-activating enzyme 1 (AAE1) and any combination thereof.

It is a further object of the present invention to disclose the method as defined in any of the above, comprises steps of: (a) identifying at least one Cannabis gene locus encoding a cannabinoid biosynthesis enzyme selected from the group consisting of tetrahydrocannabinolic acid synthase (THCAS), cannabidiolic acid synthase (CBDAS), aromatic prenyltransferase (PT), olivetol synthase (OLS), acyl-activating enzyme 1 (AAE1) and any combination thereof; (b) identifying at least one endonuclease recognition sequence in or proximal to the at least one cannabinoid biosynthesis enzyme gene locus; (c) providing at least one guide RNA (gRNA) comprising a nucleotide sequence at least partially complementary to said at least one identified gene locus; (d) introducing into Cannabis plant cells a construct comprising (i) an endonuclease nucleotide sequence operably linked to said gRNA, or (ii) a ribonucleoprotein (RNP) complex comprising an endonuclease protein and said gRNA; (e) assaying the Cannabis plant or a cell thereof for an endonuclease-mediated modification in the DNA of the at least one cannabinoid biosynthesis enzyme gene locus; and (f) identifying the Cannabis plant, a cell thereof, or a progeny cell thereof as comprising a modification in the at least one cannabinoid biosynthesis enzyme gene locus.

It is a further object of the present invention to disclose the method as defined in any of the above, further comprises steps of (a) screening the genome of the transformed Cannabis plant cells for induced targeted mutations in at least one of said cannabinoid biosynthesis enzyme gene locus comprising obtaining a nucleic acid sample from said transformed plant and carrying out nucleic acid amplification and optionally restriction enzyme digestion to detect a mutation in said at least one of said cannabinoid biosynthesis enzyme gene locus; (b) confirming the presence of said genetic mutation in the genome of said plant cells by sequencing said at least one cannabinoid biosynthesis enzyme gene locus; (c) regenerating plants carrying said genetic modification; and (d) screening said regenerated plants for a plant with modified cannabinoid content.

It is a further object of the present invention to disclose the method as defined in any of the above, wherein the endonuclease is expressed transiently or stably in the Cannabis plant.

It is a further object of the present invention to disclose an isolated nucleotide sequence having at least 75% sequence identity to a nucleotide sequence selected from the group consisting of SEQ ID NO:1, SEQ ID NO:4, SEQ ID NO:7, SEQ ID NO:10, and SEQ ID NO:13.

It is a further object of the present invention to disclose an isolated nucleotide sequence having at least 75% sequence identity to a nucleotide sequence selected from the group consisting of SEQ ID NO:2, SEQ ID NO:5, SEQ ID NO:8, SEQ ID NO:11 and SEQ ID NO:14.

It is a further object of the present invention to disclose an isolated amino acid sequence having at least 75% sequence identity to an amino acid sequence selected from the group consisting of SEQ ID NO:3, SEQ ID NO:6, SEQ ID NO:9, SEQ ID NO:12 and SEQ ID NO:15.

It is a further object of the present invention to disclose an isolated nucleotide sequence having at least 75% sequence identity to a nucleotide sequence as set forth in SEQ ID NO:16-826.

It is a further object of the present invention to disclose a vector, construct or expression system or cassette comprising the nucleic acid sequence as defined in any one of claims 90-93.

It is a further object of the present invention to disclose use of a nucleotide sequence as set forth in at least one of SEQ ID NO:16-826 and any combination thereof for down regulation of at least one Cannabis cannabinoid biosynthesis enzyme gene selected from the group consisting of Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) and any combination thereof.

It is a further object of the present invention to disclose use of a nucleotide sequence as set forth in at least one of SEQ ID NO:16-167 and any combination thereof for targeted genome modification of Cannabis tetrahydrocannabinolic acid synthase (CsTHCAS) gene.

It is a further object of the present invention to disclose use of a nucleotide sequence as set forth in at least one of SEQ ID NO:168-304, SEQ ID NO:824-825, and any combination thereof for targeted genome modification of Cannabis cannabidiolic acid synthase (CsCBDAS).

It is a further object of the present invention to disclose use of a nucleotide sequence as set forth in at least one of SEQ ID NO:305-458 and any combination thereof for targeted genome modification of Cannabis aromatic prenyltransferase (CsPT).

It is a further object of the present invention to disclose use of a nucleotide sequence as set forth in at least one of SEQ ID NO:459-509 and any combination thereof for targeted genome modification of Cannabis olivetol synthase (CsOLS).

It is a further object of the present invention to disclose use of a nucleotide sequence as set forth in at least one of SEQ ID NO:510-823, SEQ ID NO:826, and any combination thereof for targeted genome modification of Cannabis acyl-activating enzyme 1 (CsAAE1).

### BRIEF DESCRIPTION OF THE DRAWINGS

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature of an embodiment of the disclosure in a figure may be used to reference the same given feature in other embodiments. Dimensions of features shown in the figures are chosen for convenience and clarity of presentation and are not necessarily shown to scale.

The invention, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 is schematically presenting CRISPR/Cas9 mode of action as depicted by Xie, Kabin, and Yinong Yang. “RNA-guided genome editing in plants using a CRISPRtCas system.” Molecular plant 6.6 (2013): 1975-1983;

FIG. 2 is schematically illustrating the cannabinoid biosynthesis pathway as depicted by the C. sativa (Cannabis) Genome Browser internet site;

FIG. 3 is photographically presenting staining of Cannabis plants after transient GUS transformation of (A) axillary buds (B) leaf (C) calli, and (D) cotyledons;

FIG. 4 is presenting regenerated transformed Cannabis tissue;

FIG. 5 is photographically presenting PCR detection of Cas9 DNA in shoots of Cannabis plants transformed using biolistics; and

FIG. 6 is illustrating in vitro cleavage activity of CRISPR/Cas9; (A) a scheme of genomic area targeted for editing, and (B) a gel showing digestion of PCR amplicon containing RNP complex of Cas9 and gene specific gRNA.

It will be appreciated that, for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

The present invention discloses manipulation of the biosynthesis pathways of a Cannabis plant of genus Cannabis. Accordingly, Cannabis plants of the present invention having a modified therapeutic component(s) profile may be useful in the production of medical Cannabis and/or may also be useful in the production of specific components or therapeutic formulations derived therefrom.

According to other main aspects of the present invention, THC free (e,g, hemp) plants for seeds, fiber and/or medical use are produced.

According to a main embodiment, the present invention provides a Cannabis plant with reduced delta-9-tetrahydrocannabinol (THC) content, or reduced cannabidiol (CBD) content, or reduced THC and CBD content, wherein said plant comprises at least one targeted genome modification effective in decreasing expression of a at least one Cannabis gene encoding a cannabinoid biosynthesis enzyme selected from the group consisting of Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) and any combination thereof.

According to one embodiment of the present invention, genes encoding cannabinoid precursor synthesis enzymes, namely AAE, PT and OLS are down regulated using targeted genome modification (e.g. gene editing techniques as inter alia presented). These enzymes are responsible for the production of a main cannabinoid precursor cannabigerolic acid (CBGA).

The final steps catalysing the synthesis of major active cannabinoids, namely, cannabichromenic acid (CBCA), cannabidiolic acid (CBDA) and Δ9-tetrahydrocannabinolic acid (THCA), are performed by oxidocyclases, namely, CBCA synthase (CBCAS), CBDA synthase (CBDAS) and THCA synthase (THCAS).

According to a further embodiment of the present invention, cannabinoid biosynthesis enzymes namely cannabidiolic acid (CBDA) synthase (CBDAS) and/or 49-tetrahydrocannabinolic acid (THCA) synthase (THCAS) are down regulated using targeted genome modification (e.g. gene editing techniques as inter alia presented). As a result, the production of the major cannabinoids CBDA (converted into CBD by decarboxylation) and/or THCA (converted into THC by decarboxylation) is significantly reduced and/or totally abolished.

Thus, according to one embodiment, targeted genome modification of one or more of the herein identified Cannabis genes encoding cannabinoid precursor synthesis enzymes, namely, CsAAE, CsPT and CsOLS, negatively affects the production of the cannabinoid precursor CBGA. As a result Cannabis plants with reduced content, or free of, THCA (and/or THC) and reduced content or free of CBDA (and/or CBD) are provided by the present invention.

According to a further embodiment of the present invention, targeted genome modification of the herein identified Cannabis gene encoding cannabinoid synthesis enzyme CsTHCAS results in reduced or no production of THCA and thus Cannabis plants with reduced content, or free of, THCA and/or THC are provided by the present invention.

According to a further embodiment of the present invention, targeted genome modification of the herein identified Cannabis gene encoding cannabinoid synthesis enzyme CsCBDAS results in reduced or no production of CBDA and thus Cannabis plants with reduced content, or free of, CBDA and/or CBD are provided by the present invention.

Breeding Cannabis plants is currently mostly done by small Cannabis growers. There is very limited if any molecular tools supporting or leading the breeding process. Traditional Cannabis breeding is done by mixing breeding material with hope to find the desired traits and phenotypes in random crosses. These methods have allowed the construction of the leading Cannabis varieties on the market today. During the last few decades, most of the breeding was focused on the psychoactive phytochemicals of the Cannabis plant. These phytochemicals, known as cannabinoids, are the compounds responsible for the medical attributes of the Cannabis plant.

As the medical Cannabis pharmaceutical industry is focusing on developing new cannabinoid based drugs, and these are mostly extracted from the Cannabis plant, there is a growing need for Cannabis plants bred for producing high levels of specific cannabinoids. In addition, there is a need for advanced breeding programs for food and fiber (Hemp) as well.

The present invention is aimed at enhancing cannabinoid breeding capabilities by using advanced molecular genome editing technologies in order to maximize the plants' phyto-chemical molecules production potential.

According to a further aspect of the present invention, a method or a tool is provided that enables the regulation in planta or the production of specific cannabinoid molecules.

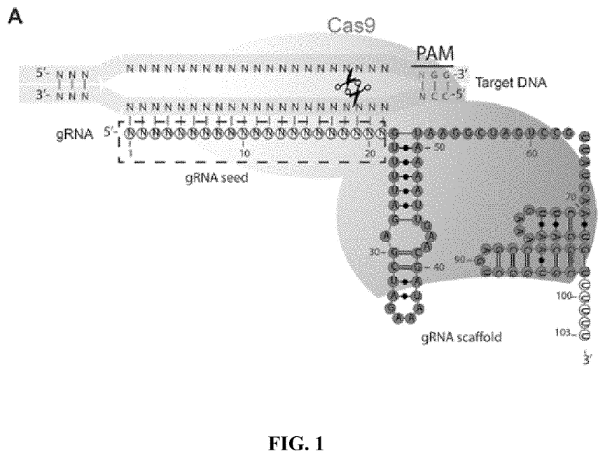

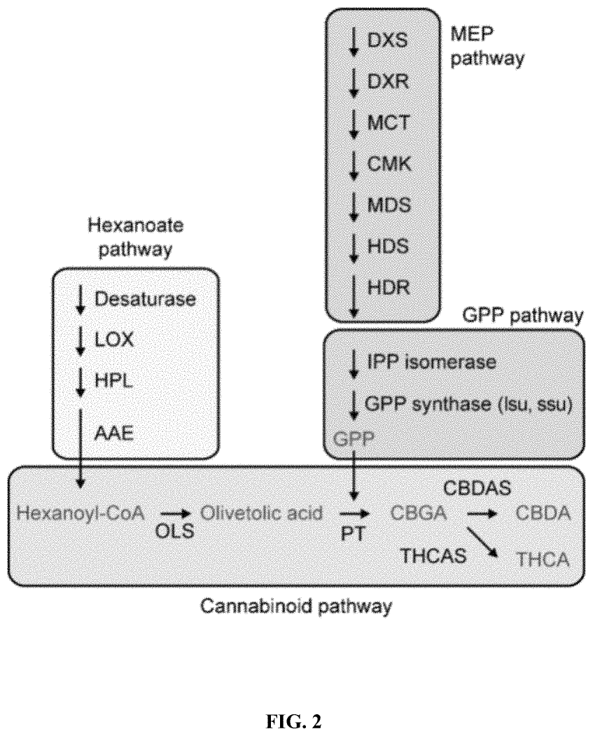

It is further within the scope of the present invention to provide means and methods for in planta modification of specific genes that relate to and/or control the cannabinoid biosynthesis pathways (as indicated in FIG. 2). More specifically, but not limited to, the present invention achieves the use of the CRISPR/Cas technology (see FIG. 1), such as, but not limited to Cas9 or Cpf1, in order to generate knockout alleles of the genes depicted in FIG. 2, rendering the enzymes inactive thereby controlling in planta the production of the resulting cannabinoid products depicted in FIG. 2.

According to some embodiments of the present invention, the above in planta modification can be based on alternative gene silencing technologies such as Zinc Finger Nucleases (ZFN's), Transcription activator-like effector nucleases (TALEN's), RNA silencing, amiRNA or any other gene silencing technique known in the art.

According to some other embodiments of the present invention, DNA introduction into the plant cells can be done by Agrobacterium infiltration, viral based plasmids for virus induced gene silencing (VIGS) and by mechanical insertion of DNA (PEG, gene gun etc).

According to further aspects of the present invention, it is possible to directly insert the Cas9 protein together with a gRNA in order to bypass the need for in vivo transcription and translation of the Cas9+gRNA plasmid in planta in order to achieve the same desired outcome.

It is a core aspect of the present invention that the above CRISPR/Cas system allows the modification of specific DNA sequences. This is achieved by combining the Cas nuclease (Cas9, Cpf1 or the like) with a guide RNA molecule (gRNA). The gRNA is designed such that it should be complementary to a specific DNA sequence targeted for editing in the plant genome and which guides the Cas nuclease to a specific nucleotide sequence (see FIG. 1). Gene specific gRNA's are cloned into the same plasmid as the Cas gene and this plasmid is inserted into plant cells. Insertion of this plasmid DNA can be done, but not limited to, by different delivery systems biological and or mechanical.

Without wishing to be bound by theory, according to further specific aspects of the present invention, upon reaching the specific DNA sequence, the Cas9 nuclease cleaves both DNA strands to create double stranded breaks leaving blunt ends. This cleavage site is then repaired by the cellular non homologous end joining DNA repair mechanism resulting in insertions or deletions which eventually creates a mutation around the cleavage site. The deletion form of the mutation consists of at least 1 base pair deletion. As a result of this base pair deletion the gene coding sequence is disrupted and the translation of the encoded protein is compromised either by a premature stop codon or disruption of a functional or structural property of the protein.

It is further within the scope that by introducing a gRNA with homology to a specific site of a gene described in FIG. 2, and sub cloning this gRNA into a plasmid containing the Cas9 gene, and upon insertion of the described plasmid into the plant cells, site specific mutations are generated in the genes herein described (delta-9-tetrahydrocannabinol (THC) content, or reduced cannabidiol (CBD) content, or reduced THC and CBD content, wherein said plant comprises a targeted genome modification effective in decreasing expression of a at least one Cannabis gene encoding a cannabinoid biosynthesis enzyme selected from the group consisting of Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) and any combination thereof). Thus effectively creating non-active proteins in the cannabinoid biosynthesis pathway, resulting in inactivation of their enzymatic activity. As a result, the present disclosure enables altering cannabinoid content in the genome edited plant. This alteration of cannabinoid content can result in a plant with significantly reduced synthesis of the molecules depicted in FIG. 2 and/or of one or more cannabinoids produced by these enzymes.

It is herein acknowledged that as the pharma industry is interested in extracting the cannabinoids from the Cannabis plant, individual Cannabis plants or strains or varieties containing modulated levels of such cannabinoids can be developed, tailored to the specific needs of the pharma industry thereby increasing the cost effectiveness and attractiveness of this crop.

The solution proposed by the current invention is using genome editing such as the CRISPR/Cas system in order to create cultivated Cannabis plants with modulated levels or ratios of cannabinoids. More specifically alternation of specific cannabinoids, i.e. THC and CBD is achieved by using genome editing techniques to reduce the expression of enzymes in the cannabinoid biosynthesis pathway.

Breeding using genome editing allows a precise and significantly shorter breeding process in order to achieve these goals with a much higher success rate. Thus genome editing, has the potential to generate improved varieties faster and at a lower cost.

In order to generate a reproducible product, Cannabis growers are currently using vegetative propagation (cloning or tissue culture). However, in conventional agricultural, genetic stability of field crops and vegetables is maintained by using F1 hybrid seeds. These hybrids are generated by crossing homozygous parental lines.

The next step for the Cannabis industry is the adoption and use of hybrid seeds for propagation, which is common practice in the conventional seed industry (from field crops to vegetables). This will allow growing and supplying high quality and reproducible raw material for the pharmaceutical industry.

The current invention discloses the generation of non GMO Cannabis plants with manipulated and controlled cannabinoid content, using the genome editing technology, e.g., the CRISPR/Cas9 highly precise tool. The generated mutations can be introduced into elite or locally adapted Cannabis lines rapidly, with relatively minimal effort and investment.

Genome editing is an efficient and useful tool for increasing crop productivity traits, and there is particular interest in advancing manipulation of genes controlling cannabinoids biosynthesis in Cannabis species, to produce strains which are adapted to specific therapeutic or regulatory needs.

Genome-editing technologies, such as the clustered regularly interspaced short palindromic repeats (CRISPR)-CRISPR-associated protein-9 nuclease (Cas9) (CRISPR-Cas9) provide opportunities to address these deficiencies, with the aims of increasing quality and yield.

A major obstacle for CRISPR-Cas9 plant genome editing is lack of efficient tissue culture and transformation methodologies. The present invention achieves these aims and surprisingly provides transformed and regenerated Cannabis plants with modified desirable cannabinoids content.

To that end, guide RNAs (gRNAs) were designed for each of the target genes herein identified in Cannabis to induce mutations in at least one cannabinoid biosynthesis enzyme including Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) and any combination thereof.

The present invention shows that Cannabis plants which contain genome editing events with at least one of the CsAAE1, CsOLS, CsPT genes or any combination thereof, express not more than 0.5% THC (or THCA) and CBD (or THCA) by weight. In specific embodiments, such plants express less than 0.5%, preferably less than 0.4%, less than 0.3%, less than 0.2%, less than 0.1% or 0% THC and CBD by weight (e.g. by dry weight).

It is further within the scope that Cannabis plants which contain genome editing events with at least one of the CsAAE1, CsOLS, CsPT genes or any combination thereof, express not more than 0.5% THC (or THCA) and/or CBD (or THCA) by weight. In specific embodiments, such plants express less than 0.5%, preferably less than 0.4%, less than 0.3%, less than 0.2%, less than 0.1% or 0% THC and/or CBD by weight (e.g. by dry weight).

The present invention further shows that Cannabis plants containing genome editing events within the CsTHCAS gene express higher levels of CBD (or CBDA) compared to non-edited plants. In a further embodiment, the CsTHCAS edited plants contain very low levels of THCA (or THC), preferably not more than 0.5% by weight.

The present invention further shows that plants containing genome editing events within the CsCBDAS gene express higher levels of THC (or THCA) as compared to non-edited plants. In a further embodiment, the CsCBDAS edited plants contain very low levels of CBDA (or CBD), preferably not more than 0.5% by weight.

It is a further aspect of the present invention to provide the Cannabis plant as defined in any of the above, wherein said plant has a THC and/or CBD content of up to 30% by weight, particularly between about %0.1 to about 30% by weight, more particularly between about %0.3 to about 30%, even more particularly between about %0.3 to about 10% by weight.

As used herein the term “about” denotes ±25% of the defined amount or measure or value.

As used herein the term “similar” denotes a correspondence or resemblance range of about ±20%, particularly ±15%, more particularly about ±10% and even more particularly about ±5%.

As used herein the term “corresponding” generally means similar, analogous, like, alike, akin, parallel, identical, resembling or comparable. In further aspects it means having or participating in the same relationship (such as type or species, kind, degree, position, correspondence, or function). It further means related or accompanying. In some embodiments of the present invention it refers to plants of the same Cannabis species or strain or variety or to sibling plant, or one or more individuals having one or both parents in common.

A “plant” as used herein refers to any plant at any stage of development, particularly a seed plant. The term “plant” includes the whole plant or any parts or derivatives thereof, such as plant cells, seeds, plant protoplasts, plant cell tissue culture from which tomato plants can be regenerated, plant callus or calli, meristematic cells, microspores, embryos, immature embryos, pollen, ovules, anthers, fruit, flowers, leaves, cotyledons, pistil, seeds, seed coat, roots, root tips and the like.

The term “plant cell” used herein refers to a structural and physiological unit of a plant, comprising a protoplast and a cell wall. The plant cell may be in a form of an isolated single cell or a cultured cell, or as a part of higher organized unit such as, for example, plant tissue, a plant organ, or a whole plant.

The term “plant cell culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits, pollen, egg cells, zygotes, seeds, seed coat, cuttings, cell or tissue cultures, or any other part or product of a plant or a combination thereof.

A “plant organ” as used herein means a distinct and visibly structured and differentiated part of a plant such as a root, stem, leaf, flower, flower bud, or embryo.

The term “Plant tissue” as used herein means a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “progeny” or “progenies” refers in a non limiting manner to offspring or descendant plants. According to certain embodiments, the term “progeny” or “progenies” refers to plants developed or grown or produced from the disclosed or deposited seeds as detailed inter alia. The grown plants preferably have the desired traits of the disclosed or deposited seeds, i.e. loss of function mutation in at least one CsSP gene or at least one CsSP5G gene.

The term “Cannabis” refers hereinafter to a genus of flowering plants in the family Cannabaceae. Cannabis is an annual, dioecious, flowering herb that includes, but is not limited to three different species, Cannabis sativa, Cannabis indica and Cannabis ruderalis. The term also refers to hemp. Cannabis plants produce a group of chemicals called cannabinoids. Cannabinoids, terpenoids, and other compounds are secreted by glandular trichomes that occur most abundantly on the floral calyxes and bracts of female Cannabis plants.

The term “nonpsychoactive” refers hereinafter to products or compositions or elements or components of Cannabis not significantly affecting the mind or mental processes.

The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in Cannabis and some other plants), and synthetic cannabinoids.

The main cannabinoids are concentrated in a viscous resin produced in structures known as glandular trichomes. Up until now, at least 113 different cannabinoids have been isolated from the Cannabis plant. The main classes of cannabinoids from Cannabis are THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), CBT (cannabicitran) and any combination thereof.

The best studied cannabinoids include tetrahydrocannabinol (THC), cannabidiol (CBD) and cannabinol (CBN).

Reference is now made to Tetrahydrocannabinol (THC), the primary psychoactive component of the Cannabis plant. Delta-9-tetrahydrocannabinol (Δ9-THC, THC) and delta-8-tetrahydrocannabinol (Δ8-THC), through intracellular CB1 activation, induce anandamide and 2-arachidonoylglycerol synthesis produced naturally in the body and brain. These cannabinoids produce the effects associated with Cannabis by binding to the CB1 cannabinoid receptors in the brain.

Tetrahydrocannabinolic acid (THCA, 2-COOH-THC; conjugate base tetrahydrocannabinolate) is a precursor of tetrahydrocannabinol (THC), the active component of cannabis. THCA is found in variable quantities in fresh, undried cannabis, but is progressively decarboxylated to THC with drying, and especially under intense heating such as when cannabis is smoked or cooked into cannabis edibles. In the context of the present invention, the term THC also refers to THCA and vice versa.

Reference is now made to Cannabidiol (CBD) which is considered as non-psychotropic. Cannabidiol has little affinity for CB1 and CB2 receptors but acts as an indirect antagonist of cannabinoid agonists. It is further acknowledged herein that it is an antagonist at the putative cannabinoid receptor, GPR55, a GPCR expressed in the caudate nucleus and putamen. Cannabidiol has also been shown to act as a 5-HT1A receptor agonist. Cannabis produces CBD-carboxylic acid through the same metabolic pathway as THC, until the next to last step, where CBDA synthase performs catalysis instead of THCA synthase. CBDA is converted into CBD by decarboxylation. In the context of the present invention, the term CBD also refers to CBDA and vice versa.

CBD shares a precursor with THC and is the main cannabinoid in CBD-dominant Cannabis strains.

In the context of the current invention, enzymes within the biosynthetic pathway of THC and CBD, especially AAE, OLS, PT, CBDAS and THCAS (depicted in FIG. 2), are down regulated to control and the content of CBD and/or THC in the Cannabis plant.

Reference is now made to FIG. 2 schematically illustrating the proposed pathway leading to the major cannabinoids Δ 9-tetrahydrocannabinolic acid (THCA) and cannabidiolic acid (CBDA), which decarboxylate to yield Δ 9-tetrahydrocannabinol (THC) and cannabidiol (CBD), respectively. The biosynthesis of THC and CBD in Cannabis follows a similar pathway.

Cannabigerolic acid (CBGA), the precursor to all natural cannabinoids, is cyclized into tetrahydrocannabinolic acid (THCA) and cannabidiolic acid (CBDA) by THCA and CBDA synthase (THCAS and CBDAS in FIG. 2), respectively. The final products of THC and CBD are formed via decarboxylation of these acidic forms. Structurally, there is an important difference between these major cannabinoids. Where THC contains a cyclic ring, CBD contains a hydroxyl group. This seemingly small difference in molecular structure may give the two compounds their different pharmacological properties.

A more specific and detailed description of the biosynthetic pathway of cannabinoids in the Cannabis plant follows below:

The isoprenoid and prenyl precursors for cannabigerolic acid (CBGA), are provided by the hexanoate and 2-C-methyl-D-erythritol 4-phosphate (MEP) pathways, respectively. Geranyl diphosphate (GPP), is a key intermediate metabolite and building block for both cannabinoid and terpenoid biosynthesis. The seven-step mevalonate (MVA) pathway converts pyruvate and glyceraldehyde-3-phosphate (G-3-P) into isopentenyl diphosphate (IPP) and dimethylallyl diphosphate (DMAPP). Key catalytic enzymes controlling flux through this pathway include the first two steps, 1-deoxy-D-xylulose 5-phosphate synthase (DXS) and 1-deoxy-D-xylulose 5-phosphate reductase (DXR). In the six-step MEP pathway, three units of acetyl coenzyme A (CoA) are converted to IPP, which is isomerized with DMAPP by IPP isomerase. The enzyme catalysing the synthesis of MEV, 3-hydroxy-3-methylglutaryl-CoA reductase (HMGR), is considered to control flux through this pathway. The number of consecutive condensations of the five-carbon monomer isopentenyl diphosphate (IPP) to its isomer, dimethylallyl diphosphate (DMAPP) is indicated by 1x, 2x, 3x. Longer-chain isoprenoids, GPP, farnesyl diphosphate (FPP) and geranyl geranyl diphosphate (GGPP), are the products of IPP and DMAPP condensation catalysed by GPP synthase, FPP synthase and GGPP synthase, respectively. GPP, FPP and geranyl-geranyl diphosphate (GGPP) are the precursors for mono-, sequi-, and di-terpines, respectively. The final steps catalysing the synthesis of major active cannabinoids, cannabichromenic acid (CBCA), cannabidiolic acid (CBDA) and Δ9-tetrahydrocannabinolic acid (THCA), are oxidocyclases, CBCA synthase (CBCAS), CBDA synthase (CBDAS) and THCA synthase (THCAS).

In the current invention, AAE, refers to acyl-activating enzyme; CBD: cannabidiol; CYP76F39, α/β-santalene monooxygenase; GPP synthase small subunit; OLS, olivetol synthase; P450: haemoprotein cytochrome P450; PT, prenyltransferase; STS, santalene synthase; TS, gamma-terpinene synthase; and TXS, taxadiene synthase.

As used herein the term “genetic modification” or “genome modification” refers hereinafter to genetic manipulation or modulation, which is the direct manipulation of an organism's genes using biotechnology. It also refers to a set of technologies used to change the genetic makeup of cells, including the transfer of genes within and across species, targeted mutagenesis and genome editing technologies to produce improved organisms. According to main embodiments of the present invention, modified Cannabis plants with altered cannabinoid content traits are generated using genome editing mechanism. This technique enables to achieve in planta modification of specific genes that control the biosynthesis of main cannabinoids, namely, THC and/or CBD in the Cannabis plant.

The term “genome editing”, or “genome/genetic modification” or “genome engineering” generally refers hereinafter to a type of genetic engineering in which DNA is inserted, deleted, modified or replaced in the genome of a living organism. Unlike previous genetic engineering techniques that randomly insert genetic material into a host genome, genome editing targets the insertions to site specific locations.

It is within the scope of the present invention that the common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’). Families of engineered nucleases used by the current invention include, but are not limited to: meganucleases, zinc finger nucleases (ZFNs), transcription activator-like effector-based nucleases (TALEN), and the clustered regularly interspaced short palindromic repeats (CRISPR/Cas9) system.

Reference is now made to exemplary genome editing terms used by the current disclosure:

### Genome Editing Glossary

Cas=CRISPR-associated genes

Cas9, Csn1=a CRISPR-associated protein containing two nuclcease domains, that is programmed by small RNAs to cleave DNA

crRNA=CRISPR RNA

dCAS9=nuclease-deficient Cas9

DSB=Double-Stranded Break

gRNA=guide RNA

HDR=Homology-Directed Repair

HNH=an endonuclease domain named for characteristic histidine and asparagine residues

Indel=insertion and/or deletion

NHEJ=Non-Homologous End Joining

PAM=Protospacer-Adjacent Motif

RuvC=an endonuclease domain named for an E. coli protein involved to DNA repair

sgRNA=single guide RNA

tracrRNA, trRNA=trans-activating crRNA

TALEN=Transcription-Activator Like Effector Nuclease

ZFN=Zinc-Finger Nuclease

According to specific aspects of the present invention, the CRISPR (Clustered Regularly Interspaced Short Palindromic Repeats) and CRISPR-associated (Cas) genes are used for the first time for generating genome modification in targeted genes in the Cannabis plant. It is herein acknowledged that the functions of CRISPR (Clustered Regularly Interspaced Short Palindromic Repeats) and CRISPR-associated (Cas) genes are essential in adaptive immunity in select bacteria and archaea, enabling the organisms to respond to and eliminate invading genetic material. These repeats were initially discovered in the 1980s in E. coli. Without wishing to be bound by theory, reference is now made to a type of CRISPR mechanism, in which invading DNA from viruses or plasmids is cut into small fragments and incorporated into a CRISPR locus comprising a series of short repeats (around 20 bps). The loci are transcribed, and transcripts are then processed to generate small RNAs (crRNA, namely CRISPR RNA), which are used to guide effector endonucleases that target invading DNA based on sequence complementarity.

According to further aspects of the invention, Cas protein, such as Cas9 (also known as Csn1) is required for gene silencing. Cas9 participates in the processing of crRNAs, and is responsible for the destruction of the target DNA. Cas9's function in both of these steps relies on the presence of two nuclease domains, a RuvC-like nuclease domain located at the amino terminus and a HNH-like nuclease domain that resides in the mid-region of the protein. To achieve site-specific DNA recognition and cleavage, Cas9 is complexed with both a crRNA and a separate trans-activating crRNA (tracrRNA or trRNA), that is partially complementary to the crRNA. The tracrRNA is required for crRNA maturation from a primary transcript encoding multiple pre-crRNAs. This occurs in the presence of RNase III and Cas9.

Without wishing to be bound by theory, it is herein acknowledged that during the destruction of target DNA, the HNH and RuvC-like nuclease domains cut both DNA strands, generating double-stranded breaks (DSBs) at sites defined by a 20-nucleotide target sequence within an associated crRNA transcript. The HNH domain cleaves the complementary strand, while the RuvC domain cleaves the noncomplementary strand.

It is further noted that the double-stranded endonuclease activity of Cas9 also requires that a short conserved sequence, (2-5 nts) known as protospacer-associated motif (PAM), follows immediately 3′- of the crRNA complementary sequence.

According to further aspects of the invention, a two-component system may be used by the current invention, combining trRNA and crRNA into a single synthetic single guide RNA (sgRNA) for guiding targeted gene alterations.

It is further within the scope that Cas9 nuclease variants include wild-type Cas9, Cas9D10A and nuclease-deficient Cas9 (dCas9).

Reference is now made to FIG. 1 schematically presenting an example of CRISPR/Cas9 mechanism of action as depicted by Xie, Kabin, and Yinong Yang. “RNA guided genome editing in plants using a CRISPR-Cas system.” Molecular plant 6.6 (2013): 1975-1983. As shown in this figure, the Cas9 endonuclease forms a complex with a chimeric RNA (called guide RNA or gRNA), replacing the crRNA-transcrRNA heteroduplex, and the gRNA could be programmed to target specific sites. The gRNA-Cas9 should comprise at least 15-base-pairing (gRNA seed region) without mismatch between the 5′-end of engineered gRNA and targeted genomic site, and an NGG motif (called protospacer-adjacent motif or PAM) that follows the base-pairing region in the complementary strand of the targeted DNA.

The term “meganucleases” as used herein refers hereinafter to endodeoxyribonucleases characterized by a large recognition site (double-stranded DNA sequences of 12 to 40 base pairs); as a result this site generally occurs only once in any given genome. Meganucleases are therefore considered to be the most specific naturally occurring restriction enzymes.

The term “protospacer adjacent motif” or “PAM” as used herein refers hereinafter to a 2-6 base pair DNA sequence immediately following the DNA sequence targeted by the Cas9 nuclease in the CRISPR bacterial adaptive immune system. PAM is a component of the invading virus or plasmid, but is not a component of the bacterial CRISPR locus. PAM is an essential targeting component which distinguishes bacterial self from non-self DNA, thereby preventing the CRISPR locus from being targeted and destroyed by nuclease.

The term “Next-generation sequencing” or “NGS” as used herein refers hereinafter to massively, parallel, high-throughput or deep sequencing technology platforms that perform sequencing of millions of small fragments of DNA in parallel. Bioinformatics analyses are used to piece together these fragments by mapping the individual reads to the reference genome.

The term “gene knockdown” as used herein refers hereinafter to an experimental technique by which the expression of one or more of an organism's genes is reduced. The reduction can occur through genetic modification, i.e. targeted genome editing or by treatment with a reagent such as a short DNA or RNA oligonucleotide that has a sequence complementary to either gene or an mRNA transcript. The reduced expression can be at the level of RNA or at the level of protein. It is within the scope of the present invention that the term gene knockdown also refers to a loss of function mutation and/or gene knockout mutation in which an organism's genes is made inoperative or nonfunctional.

The term “gene silencing” as used herein refers hereinafter to the regulation of gene expression in a cell to prevent the expression of a certain gene. Gene silencing can occur during either transcription or translation. In certain aspects of the invention, gene silencing is considered to have a similar meaning as gene knockdown. When genes are silenced, their expression is reduced. In contrast, when genes are knocked out, they are completely not expressed. Gene silencing may be considered a gene knockdown mechanism since the methods used to silence genes, such as RNAi, CRISPR, or siRNA, generally reduce the expression of a gene by at least 70% but do not completely eliminate it.

The term “loss of function mutation” as used herein refers to a type of mutation in which the altered gene product lacks the function of the wild-type gene. A synonyms of the term included within the scope of the present invention is null mutation.

The term “microRNAs” or “miRNAs” refers hereinafter to small non-coding RNAs that have been found in most of the eukaryotic organisms. They are involved in the regulation of gene expression at the post-transcriptional level in a sequence specific manner. MiRNAs are produced from their precursors by Dicer-dependent small RNA biogenesis pathway. MiRNAs are candidates for studying gene function using different RNA-based gene silencing techniques. For example, artificial miRNAs (amiRNAs) targeting one or several genes of interest is a potential tool in functional genomics.

The term “in planta” means in the context of the present invention within the plant or plant cells. More specifically, it means introducing CRISPR/Cas complex into plant material comprising a tissue culture of several cells, a whole plant, or into a single plant cell, without introducing a foreign gene or a mutated gene. It also used to describe conditions present in a non-laboratory environment (e.g. in vivo).

The term “genotype” or “genetic background” refers hereinafter to the genetic constitution of a cell or organism. An individual's genotype includes the specific alleles, for one or more genetic marker loci, present in the individual's haplotype. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest. Thus, in some embodiments a genotype comprises a summary of one or more alleles present within an individual at one or more genetic loci. In some embodiments, a genotype is expressed in terms of a haplotype. It further refers to any inbreeding group, including taxonomic subgroups such as subspecies, taxonomically subordinate to species and superordinate to a race or subrace and marked by a pre-determined profile of latent factors of hereditary traits.

The term “orthologue” as used herein refers hereinafter to one of two or more homologous gene sequences found in different species.

The term “functional variant” or “functional variant of a nucleic acid or amino acid sequence” as used herein, for example with reference to SEQ ID NOs: 1, 4 or 7 refers to a variant of a sequence or part of a sequence which retains the biological function of the full non-variant allele and hence has the activity of the expressed gene or protein. A functional variant also comprises a variant of the gene of interest encoding a polypeptide which has sequence alterations that do not affect function of the resulting protein, for example, in non-conserved residues. Also encompassed is a variant that is substantially identical, i.e. has only some sequence variations, for example, in non-conserved residues, to the wild type nucleic acid or amino acid sequences of the alleles as shown herein, and is biologically active.

The term “variety” or “cultivar” used herein means a group of similar plants that by structural features and performance can be identified from other varieties within the same species.

The term “allele” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those mat are identical or associated with a single gene or multiple genes or their products or even a gene disrupting or controlled by a genetic factor contributing to the phenotype represented by the locus. According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele.

As used herein, the term “locus” (loci plural) means a specific place or places or region or a site on a chromosome where for example a gene or genetic marker element or factor is found. In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

Conversely, as used herein, the term “heterozygous” means a genetic condition or configuration existing when two different or unlike alleles reside at a specific locus, but are positioned individually on corresponding pairs of homologous chromosomes in the cell of a diploid organism.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance.

Proteins may have a sequence motif and/or a structural motif, a motif formed by the three-dimensional arrangement of amino acids which may not be adjacent.

As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds.

According to other aspects of the invention, a “modified” or a “mutant” plant is a plant that has been altered compared to the naturally occurring wild type (WT) plant. Specifically, the endogenous nucleic acid sequences of one or more of the Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1), and any combination thereof, homologs in Cannabis have been altered compared to wild type sequences using mutagenesis and/or genome editing methods as described herein. This causes inactivation of at least one of these endogenous genes and thus disables their function in production of THC and/or CBD, depending on the gene or combination of genes down regulated.

Such plants have an altered cannabinoid profile which may be suitable for treatment of different medical conditions or diseases. Therefore, the cannabinoid profile is affected by the presence of at least one mutated endogenous cannabinoid biosynthesis enzyme gene in the Cannabis plant genome which has been specifically targeted using genome editing technique.

As used herein, the term “cannabinoid biosynthesis enzyme” refers to a protein acting as a catalyst for producing one or more cannabinoids in a plant of genus Cannabis.

Examples of cannabinoid biosynthesis enzymes within the context of this disclosure include, but are not limited to: tetrahydrocannabinolic acid synthase (THCAS), cannabidiolic acid synthase (CBDAS), aromatic prenyltransferase (PT), olivetol synthase (OLS), acyl-activating enzyme 1 (AAE1), polyketide synthase (PKS), olivetolic acid cyclase (OAC), tetraketide synthase (TKS), type III PKS, chalcone synthase (CHS), prenyltransferase, CBCA synthase, GPP synthase, FPP synthase, Limonene synthase, aromatic prenyltransferase, and geranylphosphate: olivetolate geranyltrasferase.

Disclosed herein, is a method of controlling cannabinoid synthesis in a plant of genus Cannabis. In some embodiments, the method comprising: Manipulating expression of a gene coding for a cannabinoid biosynthesis enzyme selected from the group consisting of Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) and any combination thereof.

As used herein, the term “controlling” refers to directing, governing, steering, and/or manipulating, specifically reducing, decreasing or down regulating or silencing the amount of a cannabinoid or cannabinoids produced in a plant of genus Cannabis. In one embodiment, controlling comprises modifying a plant of genus Cannabis to produce an unnaturally occurring concentration of a first cannabinoid. In one embodiment, controlling comprises modifying a plant of genus Cannabis to produce an unnaturally occurring ratio of a first cannabinoid. In one embodiment, controlling comprises modifying a plant of genus Cannabis to produce an unnaturally occurring concentration of a second cannabinoid. In one embodiment, controlling comprises modifying a plant of genus Cannabis to produce an unnaturally occurring ratio of a second cannabinoid.

As used herein, the term “expression of a gene” refers to a plant's ability to utilize information from genetic material for producing functional gene products. Within the context of this disclosure, expression is meant to encompass the plant's ability to produce proteins, such as enzymes, and various other molecules from the plant's genetic material. In one embodiment, the plant expresses mutated or modified cannabinoid biosynthesis enzymes for cannabinoid biosynthesis. In one embodiment it refers to transcription (RNA) or translation (protein) levels of gene expression.

As used herein, the term “manipulating expression of a gene” refers to intentionally changing the genome of a plant of genus Cannabis to control the expression of certain features.

In one embodiment, the plant's genome is manipulated to express less CBDA synthase.

In one embodiment, the plant's genome is manipulated to express less THCA synthase.

In one embodiment, the plant's genome is manipulated to express less aromatic prenyltransferase (PT).

In one embodiment, the plant's genome is manipulated to express less olivetol synthase (OLS).

In one embodiment, the plant's genome is manipulated to express less acyl-activating enzyme 1 (AAE1).

According to a further embodiment, the plant's genome is manipulated to express less of any combination of the above mentioned cannabinoid biosynthesis enzymes.

As used herein, the term “coding” refers to storing genetic information and accessing the genetic information for producing functional gene products.

According to further aspects of the present invention, the altered THC and/or CBD content trait is not conferred by the presence of transgenes expressed in Cannabis.

Cannabis plants of the invention are modified plants compared to wild type plants which comprise and express at least one mutant Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) and any combination thereof allele.

Main aspects of the invention involve targeted mutagenesis methods, specifically genome editing, and exclude embodiments that are solely based on generating plants by traditional breeding methods.

Described are polynucleotides as well as methods for modifying metabolite biosynthesis pathways in Cannabis plants and/or Cannabis plant cells, Cannabis plants and/or plant cells exhibiting modified metabolite biosynthesis pathways. In particular, described are methods for modifying production of THC and/or CBD in Cannabis plants by modulating the expression and/or activity of at least one of Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) and any combination thereof and Cannabis plants having modified expression and/or activity of at least one of these genes/proteins.

Accordingly, in certain embodiments, the present invention provides methods of downregulating production of THC and/or CBD. In particular embodiments, there is provided methods of downregulating expression and/or activity THCA synthase and/or CBDA synthase.

Also provided are plants and/or plant cells having modified production of THC and/or CBD. In certain embodiments, there are provided Cannabis plants and/or cells having down-regulated expression of and/or activity of at least one of Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) and any combination thereof.

Down regulation of key steps in metabolic pathway re-directs intermediates and energy to alternative metabolic pathways and results in increased production and accumulation or reduced production and elimination of other end products. THC and other Cannabis metabolites share a biosynthetic pathway; that cannabigerolic acid is a precursor of THC, CBD and Cannabichromene. In particular, THCA synthase catalyzes the production of delta-9-tetrahydrocannabinolic acid from cannabigerolic acid; delta-9-tetrahydrocannabinolic undergoes thermal conversion to form THC. CBDA synthase catalyzes the production of cannabidiolic acid from cannabigerolic acid; cannabidiolic acid undergoes thermal conversion to CBD. CBCA synthase catalyzes the production of cannabichromenic acid from cannabigerolic acid; cannabichromenic acid undergoes thermal conversion to cannabichromene.

A reduction in the production of THC, CBD, or Cannabichromene will enhance production of the remaining metabolites in this shared pathway. For example, production of CBD and/or Cannabichromene is enhanced by inhibiting production of THC. THC production may be inhibited by inhibiting expression and/or activity of tetrahydrocannabinolic acid (THCA) synthase enzyme.

Described are certain embodiments of enhancing production of one or more secondary metabolites by downregulation of the production of one or more metabolites having a shared biosynthetic pathway. Certain embodiments provide methods of enhancing production of one or more secondary metabolites that share steps and intermediates in the THC and/or CBD biosynthetic pathway by downregulation of THC and/or CBD production. In specific embodiments, there are provided methods of enhancing production of CBD and/or Cannabichromene by inhibiting production of THC. In other specific embodiments, there are provided methods of enhancing production of THC by inhibiting production of CBD.

In other specific embodiments, both the production of CBD and THC is inhibited by targeting at least one of the herein identified genes CsAAE1 (SEQ ID NO:13), or CsOLS (SEQ ID NO:10), or CsPT (SEQ ID NO:7) or any combination thereof.

In other specific embodiments, the production of CBD is inhibited (and THC is induced or not affected) by targeting the herein identified gene CsCBDAS (SEQ ID NO:4).

In other specific embodiments, the production of THC is inhibited (and CBD is induced or not affected) by targeting the herein identified gene CsTHCAS (SEQ ID NO:1).

Certain embodiments provide methods of enhancing production of one or more secondary metabolites which share steps and intermediates in the THC biosynthetic pathway by downregulation of expression and/or activity of CsTHCA synthase (SEQ ID NO:1).

In specific embodiments, there are provided methods of enhancing production of CBD and/or Cannabichromene by downregulation of expression and/or activity of THCA synthase.

Also provided are plants and plant cells having modified production of one or more metabolites having a shared biosynthetic pathway. In certain embodiments, there are provided Cannabis plants and cells enhanced production of one or more secondary metabolites and downregulation of one or more other metabolites having a shared biosynthetic pathway. In certain embodiments, there are provided Cannabis plants and cells having enhanced production of one or more secondary metabolites and downregulation of one or more other metabolites in the THC and or CBD biosynthetic pathway. In certain embodiments, there are provided Cannabis plants and cells having enhanced production of one or more secondary metabolites in the THC biosynthetic pathway and downregulated THC production. In specific embodiments, there are provided Cannabis plants and cells having enhanced production of CBD and/or Cannabichromene and downregulated THC production.

In specific embodiments, there are provided Cannabis plants and/or cells having enhanced production of CBD and/or Cannabichromene and downregulated expression and/or activity of THCA synthase.

The loss of function mutation may be a deletion or insertion (“indels”) with reference the wild type allele sequence. The deletion may comprise 1-20 or more nucleotides, for example 1, 2, 3, 4, 5, 6, 7, 8, 9, 10, 1, 12, 13, 14, 15, 16, 17, 18 or 20 nucleotides or more in one or more strand. The insertion may comprise 1-20 or more nucleotides, for example 1, 2, 3, 4, 5, 6, 7, 8, 9, 10, 1, 12, 13, 14, 15, 16, 17, 18 or 20 or more nucleotides in one or more strand.

The plant of the invention includes plants wherein the plant is heterozygous for the each of the mutations. In other embodiment however, the plant is homozygous for the mutations. Progeny that is also homozygous can be generated from these plants according to methods known in the art.

It is further within the scope that variants of a particular nucleotide or amino acid sequence according to the various aspects of the invention will have at least about 50%-99%, for example at least 75%, for example at least 85%, 86%, 87%, 88%, 89%, 90%, 92%, 94%, 95%, 96%, 97%, 98% or 99% or more sequence identity to that particular non-variant nucleotide sequence of the Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) allele as shown in SEQ ID NO 1, 4, 7, 10 or 13; and/or SEQ ID NO 2, 5, 8, 11 or 14, respectively. Sequence alignment programs to determine sequence identity are well known in the art.

Also, the various aspects of the invention encompass not only a Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) nucleic acid sequence or amino acid sequence, but also fragments thereof. By “fragment” is intended a portion of the nucleotide sequence or a portion of the amino acid sequence and hence of the protein encoded thereby. Fragments of a nucleotide sequence may encode protein fragments that retain the biological activity of the native protein, in this case cannabinoid biosynthesis enzymes.

According to further embodiments of the present invention, DNA introduction into the plant cells can be done by Agrobacterium infiltration, virus based plasmids for delivery of the genome editing molecules and mechanical insertion of DNA (PEG mediated DNA transformation, biolistics, etc.).

In addition, it is within the scope of the present invention that the Cas9 protein is directly inserted together with a gRNA (ribonucleoprotein-RNP's) in order to bypass the need for in vivo transcription and translation of the Cas9+gRNA plasmid in planta to achieve gene editing.

It is within the scope of the present invention that the usage of CRISPR/Cas system for the generation of Cannabis plants with at least one improved domestication trait, allows the modification of predetermined specific DNA sequences without introducing foreign DNA into the genome by GMO techniques. According to one embodiment of the present invention, this is achieved by combining the Cas nuclease (e.g. Cas9, Cpf1 and the like) with a predefined guide RNA molecule (gRNA). The gRNA is complementary to a specific DNA sequence targeted for editing in the plant genome and which guides the Cas nuclease to a specific nucleotide sequence (for example see FIG. 1). The predefined gene specific gRNA's are cloned into the same plasmid as the Cas gene and this plasmid is inserted into plant cells. Insertion of the aforementioned plasmid DNA can be done, but not limited to, using different delivery systems, biological and/or mechanical, e.g. Agrobacterium infiltration, virus based plasmids for delivery of the genome editing molecules and mechanical insertion of DNA (PEG mediated DNA transformation, biolistics, etc.).

It is further within the scope of the present invention that upon reaching the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which eventually create a mutation at the cleavage site. For example, it is acknowledged that a deletion form of the mutation consists of at least 1 base pair deletion. As a result of this base pair deletion the gene coding sequence is disrupted and the translation of the encoded protein is compromised either by a premature stop codon or disruption of a functional or structural property of the protein. Thus DNA is cut by the Cas9 protein and re-assembled by the cell's DNA repair mechanism.

It is further within the scope that manipulation of cannabinoid biosynthesis enzymes in Cannabis plants is herein achieved by generating gRNA with homology to a specific site of predetermined genes in the Cannabis genome i.e. Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) genes, sub cloning this gRNA into a plasmid containing the Cas9 gene, and insertion of the plasmid into the Cannabis plant cells. In this way site specific mutations in the aforementioned genes are generated thus effectively creating non-active molecules, resulting in loss of function of at least one of the enzymes, reduced content of THC, CBD or both of the cannabinoids in the genome edited plant.

In order to understand the invention and to see how it may be implemented in practice, a plurality of preferred embodiments will now be described, by way of non-limiting example only, with reference to the following examples.

### Example 1

### Production of Cannabis Plants with Modulated Cannabinoid Expression

Production of Cannabis lines with mutated Cannabis tetrahydrocannabinolic acid synthase (CsTHCAS) or Cannabis cannabidiolic acid synthase (CsCBDAS) or Cannabis aromatic prenyltransferase (CsPT) or Cannabis olivetol synthase (CsOLS) or Cannabis acyl-activating enzyme 1 (CsAAE1) gene or any combination thereof may be achieved by at least one of the following breeding/cultivation schemes:

Scheme 1:
line stabilization by self pollination
Generation of F6 parental lines
Genome editing of parental lines
Crossing edited parental lines to generate an F1 hybrid plant

Scheme 2:
Identifying genes/alleles of interest
Designing gRNA
Transformation of plants with Cas9+gRNA constructs
Screening and identifying editing events
Genome editing of parental lines

It is noted that line stabilization may be performed by the following:
Induction of male flowering on female (XX) plants
Self pollination

According to some embodiments of the present invention, line stabilization requires about 6 self-crossing (6 generations) and done through a single seed descent (SSD) approach.

F1 hybrid seed production: Novel hybrids are produced by crosses between different Cannabis strains.

According to a further aspect of the current invention, shortening line stabilization is performed by Doubled Haploids (DH). More specifically, the CRISPR-Cas9 system is transformed into microspores to achieve DH homozygous parental lines. A doubled haploid (DH) is a genotype formed when haploid cells undergo chromosome doubling. Artificial production of doubled haploids is important in plant breeding. It is herein acknowledged that conventional inbreeding procedures take about six generations to achieve approximately complete homozygosity, whereas doubled haploidy achieves it in one generation.

It is within the scope of the current invention that genetic markers specific for Cannabis are developed and provided by the current invention:
Sex markers—molecular markers are used for identification and selection of female vs male plants in the herein disclosed breeding program
Genotyping markers—germplasm used in the current invention is genotyped using molecular markers, in order to allow a more efficient breeding process and identification of the Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS) or Cannabis acyl-activating enzyme 1 (CsAAE1) editing event.

It is further within the scope of the current invention that allele and genetic variation is analysed for the Cannabis strains used.

Reference is now made to optional stages that have been used for the production of mutated Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) Cannabis plants by genome editing:

Stage 1: Identifying Cannabis sativa (C. sativa), C. indica and C. ruderalis tetrahydrocannabinolic acid synthase (THCAS), cannabidiolic acid synthase (CBDAS), aromatic prenyltransferase (PT), olivetol synthase (OLS), acyl-activating enzyme 1 (AAE1) orthologues/homologs.

The following homologs have herein been identified in Cannabis sativa (C. sativa), C. indica and C. ruderalis, namely Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS) and Cannabis acyl-activating enzyme 1 (CsAAE1). These homologous genes have been sequenced and mapped.

CsTHCAS has been mapped to CM011610.1:22243181-22246809 and has a genomic sequence as set forth in SEQ ID NO:1. The CsTHCAS gene has a coding sequence as set forth in SEQ ID NO:2 and it encodes an amino acid sequence as set forth in SEQ ID NO:3.

CsCBDAS has been mapped to CM011610.1:21836038-21839672 and has a genomic sequence as set forth in SEQ ID NO:4. The CsCBDAS gene has a coding sequence as set forth in SEQ ID NO:5 and it encodes an amino acid sequence as set forth in SEQ ID NO:6.

CsPT has been mapped to CM011614.1:1184501-1186728 and has a genomic sequence as set forth in SEQ ID NO:7. The CsPT gene has a coding sequence as set forth in SEQ ID NO:8 and it encodes an amino acid sequence as set forth in SEQ ID NO:9.

CsOLS has been mapped to CM011613.1:2335391-2338392 and has a genomic sequence as set forth in SEQ ID NO:10. The CsOLS gene has a coding sequence as set forth in SEQ ID NO:11 and it encodes an amino acid sequence as set forth in SEQ ID NO:12.

CsAAE1 has been mapped to CM011611.1:1210973-1228229 and has a genomic sequence as set forth in SEQ ID NO:13. The CsAAElgene has a coding sequence as set forth in SEQ ID NO:14 and it encodes an amino acid sequence as set forth in SEQ ID NO:15.

Stage 2: Designing and synthesizing gRNA molecules corresponding to the sequence targeted for editing, i.e. sequences of each of the genes Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1). It is noted that the editing event is preferably targeted to a unique restriction site sequence to allow easier screening for plants carrying an editing event within their genome.

According to some aspects of the invention, the nucleotide sequence of the gRNAs should be completely compatible with the genomic sequence of the target gene. Therefore, for example, suitable gRNA molecules should be constructed for different Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) homologues of different Cannabis strains.

Reference is now made to Tables 1-5 presenting gRNA molecules targeted for silencing Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1), respectively. The term ‘PAM’ refers hereinafter to Protospacer Adjacent Motif, which is a 2-6 base pair DNA sequence immediately following the DNA sequence targeted by the Cas9 nuclease in the CRISPR bacterial adaptive immune system.

TABLE 1
gRNA sequences targeted for CsTHCAS

| Position | | | | | | |
|---|---|---|---|---|---|---|
| on SEQ. | | | | Specificity | Efficiency | SEQ. |
| ID. NO.: 1 | Strand | Sequence | PAM | Score | Score | ID NO |
| 919 | -1 | TATAACTTTATATTGGAGCG | GGG | 91.68505 | 59.10928 | 16 |
| 920 | -1 | CTATAACTTTATATTGGAGC | GGG | 81.97792 | 39.46084 | 17 |
| 921 | -1 | TCTATAACTTTATATTGGAG | CGG | 59.73169 | 58.95429 | 18 |
| 926 | -1 | TCCTTTCTATAACTTTATAT | TGG | 56.2663 | 28.95317 | 19 |
| 936 | 1 | TCCAATATAAAGTTATAGAA | AGG | 52.27911 | 43.64749 | 20 |
| 1013 | 1 | ACTACTCAACATTCTCCTTT | AGG | 78.87146 | 29.78268 | 21 |
| 1017 | -1 | AATTTTGTAAACAAACCTAA | AGG | 47.82242 | 50.8107 | 22 |
| 1085 | -1 | CAATTTAGGAAATTTTCTTG | AGG | 53.52986 | 57.27972 | 23 |
| 1099 | -1 | TATATTGGGAGAAGCAATTT | AGG | 72.42583 | 23.48898 | 24 |
| 1113 | -1 | TGGATTGTTATGAATATATT | GGG | 39.7547 | 29.09505 | 25 |
| 1114 | -1 | CTGGATTGTTATGAATATAT | TGG | 62.7209 | 37.8461 | 26 |
| 1133 | -1 | TATACGAGTTTTAGATTTGC | TGG | 72.07547 | 31.53966 | 27 |
| 1168 | -1 | TCAGGACAGACATATACAAT | TGG | 85.35755 | 47.27478 | 28 |
| 1186 | -1 | GATTTTGTATTGTCAAATTC | AGG | 60.33738 | 31.33693 | 29 |
| 1218 | -1 | TGGTTTTGGGGTTGTATCAG | AGG | 87.42544 | 54.0448 | 30 |
| 1230 | -1 | GACAATAACGAGTGGTTTTG | GGG | 52.23624 | 53.9614 | 31 |
| 1231 | -1 | TGACAATAACGAGTGGTTTT | GGG | 75.404 | 22.91377 | 32 |
| 1232 | -1 | GTGACAATAACGAGTGGTTT | TGG | 84.75157 | 30.78067 | 33 |
| 1238 | -1 | GAAGGAGTGACAATAACGAG | TGG | 81.66494 | 66.45396 | 34 |
| 1256 | -1 | TGGATATGGGAGACATTTGA | AGG | 79.3898 | 47.59851 | 35 |
| 1269 | -1 | TAGAATAGTGGCTTGGATAT | GGG | 90.41741 | 42.20222 | 36 |
| 1270 | -1 | ATAGAATAGTGGCTTGGATA | TGG | 86.00061 | 40.03718 | 37 |
| 1276 | -1 | TGGAGCATAGAATAGTGGCT | TGG | 91.91503 | 51.2944 | 38 |
| 1281 | -1 | TTTCTTGGAGCATAGAATAG | TGG | 71.98696 | 51.24085 | 39 |
| 1296 | -1 | AATCTGCAAGCCAACTTTCT | TGG | 83.889 | 23.52241 | 40 |
| 1297 | 1 | ATTCTATGCTCCAAGAAAGT | TGG | 76.79961 | 44.57193 | 41 |
| 1321 | 1 | TTGCAGATTCGAACTCGAAG | CGG | 91.27714 | 68.55563 | 42 |
| 1324 | 1 | CAGATTCGAACTCGAAGCGG | TGG | 95.1427 | 59.66476 | 43 |
| 1336 | -1 | AGGACAAACCCTCAGCATCA | TGG | 91.96485 | 64.63997 | 44 |
| 1338 | 1 | AAGCGGTGGCCATGATGCTG | AGG | 89.20633 | 53.30037 | 45 |
| 1339 | 1 | AGCGGTGGCCATGATGCTGA | GGG | 92.84794 | 63.02093 | 46 |
| 1356 | -1 | AAATGGGACTTGAGATGTGT | AGG | 82.02117 | 58.19267 | 47 |
| 1372 | -1 | TCAAGTCTACTATAACAAAT | GGG | 66.95566 | 44.7251 | 48 |
| 1373 | -1 | CTCAAGTCTACTATAACAAA | TGG | 79.05133 | 34.85902 | 49 |
| 1398 | 1 | AGACTTGAGAAACATGCATT | CGG | 78.16459 | 54.20818 | 50 |
| 1429 | -1 | CGGCTTCAACCCACGCAGTT | TGG | 99.57277 | 40.06864 | 51 |
| 1430 | 1 | ATATTCGTAGCCAAACTGCG | TGG | 92.73046 | 65.82128 | 52 |
| 1431 | 1 | TATTCGTAGCCAAACTGCGT | GGG | 97.53917 | 62.7453 | 53 |
| 1441 | 1 | CAAACTGCGTGGGTTGAAGC | CGG | 95.87721 | 52.5429 | 54 |
| 1449 | -1 | AACTTCTCCAAGGGTAGCTC | CGG | 72.36938 | 46.01821 | 55 |
| 1453 | 1 | GTTGAAGCCGGAGCTACCCT | TGG | 97.29983 | 54.79341 | 56 |
| 1458 | -1 | CCAATAATAAACTTCTCCAA | GGG | 74.16963 | 63.94759 | 57 |
| 1459 | -1 | TCCAATAATAAACTTCTCCA | AGG | 73.52665 | 52.58996 | 58 |
| 1469 | 1 | CCCTTGGAGAAGTTTATTAT | TGG | 83.24938 | 16.35292 | 59 |
| 1504 | 1 | AATGAGAATCTTAGTTTTCC | TGG | 66.81185 | 30.01853 | 60 |
| 1507 | 1 | GAGAATCTTAGTTTTCCTGG | TGG | 85.44233 | 66.58972 | 61 |
| 1508 | 1 | AGAATCTTAGTTTTCCTGGT | GGG | 81.70394 | 50.34528 | 62 |
| 1511 | -1 | ACAGTAGGGCAATACCCACC | AGG | 96.88637 | 64.82422 | 63 |
| 1525 | 1 | GGTGGGTATTGCCCTACTGT | TGG | 78.50407 | 53.56142 | 64 |
| 1525 | -1 | GTCCACCTACGCCAACAGTA | GGG | 89.72665 | 55.22571 | 65 |
| 1526 | -1 | TGTCCACCTACGCCAACAGT | AGG | 89.39852 | 57.68998 | 66 |
| 1531 | 1 | TATTGCCCTACTGTTGGCGT | AGG | 94.7426 | 55.96514 | 67 |
| 1534 | 1 | TGCCCTACTGTTGGCGTAGG | TGG | 95.81918 | 46.27569 | 68 |
| 1546 | 1 | GGCGTAGGTGGACACTTTAG | TGG | 66.85409 | 46.38521 | 69 |
| 1549 | 1 | GTAGGTGGACACTTTAGTGG | AGG | 13.8355 | 69.31429 | 70 |
| 1552 | 1 | GGTGGACACTTTAGTGGAGG | AGG | 16.66937 | 53.37905 | 71 |
| 1558 | 1 | CACTTTAGTGGAGGAGGCTA | TGG | 45.06996 | 40.83788 | 72 |
| 1579 | 1 | GGAGCATTAATGCGAAATTA | TGG | 83.83888 | 31.63254 | 73 |
| 1591 | -1 | CAATGATATTATCAGCTGCG | AGG | 87.7846 | 69.05545 | 74 |
| 1627 | 1 | GCACACTTAGTCAATGTTGA | TGG | 81.81927 | 49.79998 | 75 |
| 1653 | 1 | AGTTCTAGATCGAAAATCCA | TGG | 82.25517 | 53.08044 | 76 |
| 1654 | 1 | GTTCTAGATCGAAAATCCAT | GGG | 87.53577 | 49.36309 | 77 |
| 1655 | 1 | TTCTAGATCGAAAATCCATG | GGG | 80.87126 | 69.96673 | 78 |
| 1656 | 1 | TCTAGATCGAAAATCCATGG | GGG | 87.02608 | 75.88737 | 79 |
| 1659 | -1 | CCAAAATAGATCTTCCCCCA | TGG | 52.2372 | 64.79896 | 80 |
| 1670 | 1 | CCATGGGGGAAGATCTATTT | TGG | 40.87315 | 10.50835 | 81 |
| 1671 | 1 | CATGGGGGAAGATCTATTTT | GGG | 45.38645 | 26.29582 | 82 |
| 1681 | 1 | GATCTATTTTGGGCTATACG | TGG | 92.9509 | 65.38681 | 83 |
| 1684 | 1 | CTATTTTGGGCTATACGTGG | TGG | 93.89012 | 56.74158 | 84 |
| 1687 | 1 | TTTTGGGCTATACGTGGTGG | TGG | 89.67677 | 47.46932 | 85 |
| 1690 | 1 | TGGGCTATACGTGGTGGTGG | AGG | 89.86004 | 51.37554 | 86 |
| 1702 | 1 | GGTGGTGGAGGTGAAAACTT | TGG | 82.68444 | 59.30357 | 87 |
| 1718 | 1 | ACTTTGGAATCATTGCAGCG | TGG | 94.22981 | 66.30813 | 88 |
| 1731 | 1 | TGCAGCGTGGAAAATTAGAC | TGG | 88.9567 | 50.21323 | 89 |
| 1748 | 1 | GACTGGTTGCTGTCCCATCA | AGG | 98.25535 | 54.41897 | 90 |
| 1749 | 1 | ACTGGTTGCTGTCCCATCAA | GGG | 90.45162 | 53.09475 | 91 |
| 1750 | -1 | TGAATATAGTAGCCCTTGAT | GGG | 89.11324 | 51.54889 | 92 |
| 1751 | -1 | CTGAATATAGTAGCCCTTGA | TGG | 94.41378 | 50.50505 | 93 |
| 1772 | 1 | CTACTATATTCAGTGTTAAA | AGG | 79.2234 | 36.92709 | 94 |
| 1779 | 1 | ATTCAGTGTTAAAAGGAATA | TGG | 65.11384 | 40.20542 | 95 |
| 1789 | 1 | AAAAGGAATATGGAGATACA | TGG | 63.91743 | 56.49626 | 96 |
| 1790 | 1 | AAAGGAATATGGAGATACAT | GGG | 68.15381 | 55.96289 | 97 |
| 1814 | 1 | TTGTCAAGTTATTTAACAAA | TGG | 47.11745 | 34.12702 | 98 |
| 1874 | 1 | TCATGACTCACTTCATAACC | AGG | 35.67077 | 53.19672 | 99 |
| 1881 | -1 | TTGATTATCTATAATATTCC | TGG | 75.11494 | 33.49257 | 100 |
| 1894 | 1 | AGGAATATTATAGATAATCA | AGG | 61.99209 | 56.41876 | 101 |
| 1918 | 1 | AAGAATAAGACTACAGTACA | CGG | 22.03371 | 69.66059 | 102 |
| 1942 | 1 | TACTTCTCTTGCATTTTCCA | TGG | 70.76593 | 46.22116 | 103 |
| 1945 | 1 | TTCTCTTGCATTTTCCATGG | TGG | 64.23993 | 47.59533 | 104 |
| 1948 | -1 | CTAGACTATCCACTCCACCA | TGG | 94.11237 | 66.93721 | 105 |
| 1950 | 1 | TTGCATTTTCCATGGTGGAG | TGG | 86.29578 | 54.03153 | 106 |
| 1992 | 1 | GAACAAGAGCTTTCCTGAGT | TGG | 94.74542 | 48.70955 | 107 |
| 1993 | 1 | AACAAGAGCTTTCCTGAGTT | GGG | 91.66248 | 41.21334 | 108 |
| 1994 | -1 | GTTTTTTTAATACCCAACTC | AGG | 81.54763 | 48.23303 | 109 |
| 2027 | 1 | CTGATTGCAAAGAATTGAGC | TGG | 91.63062 | 45.41598 | 110 |
| 2049 | -1 | TACAACACCACTGTAGAAGA | TGG | 87.1194 | 55.28908 | 111 |
| 2053 | 1 | GATACTACCATCTTCTACAG | TGG | 85.64352 | 61.29416 | 112 |
| 2088 | 1 | TAACACTACTAATTTTCAAA | AGG | 52.892 | 37.63415 | 113 |
| 2113 | 1 | ATTTTGCTTGATAGATCAGC | TGG | 89.54269 | 45.49052 | 114 |
| 2114 | 1 | TTTTGCTTGATAGATCAGCT | GGG | 83.06148 | 69.66798 | 115 |
| 2168 | -1 | ACAATTGCAGTTTCTGGAAT | TGG | 83.65549 | 27.85148 | 116 |
| 2174 | -1 | ATTTTGACAATTGCAGTTTC | TGG | 71.89285 | 13.8671 | 117 |
| 2190 | 1 | AACTGCAATTGTCAAAATTT | TGG | 51.26105 | 21.65345 | 118 |
| 2215 | 1 | AAATTGTATGAAGAAGATGT | AGG | 50.42741 | 60.89104 | 119 |
| 2221 | 1 | TATGAAGAAGATGTAGGAGT | TGG | 74.0566 | 40.67797 | 120 |
| 2245 | 1 | GTGTATGTATTGTACCCTTA | CGG | 87.47868 | 52.95626 | 121 |
| 2248 | 1 | TATGTATTGTACCCTTACGG | TGG | 88.53147 | 70.67558 | 122 |
| 2248 | -1 | TGTCCATTATACCACCGTAA | GGG | 98.58031 | 65.38253 | 123 |
| 2249 | -1 | TTGTCCATTATACCACCGTA | AGG | 96.99197 | 52.80478 | 124 |
| 2256 | 1 | GTACCCTTACGGTGGTATAA | TGG | 86.96972 | 36.4762 | 125 |
| 2291 | -1 | ATTCCAGCTCGATGAGGGAA | AGG | 85.04932 | 58.09939 | 126 |
| 2296 | -1 | ACATGATTCCAGCTCGATGA | GGG | 95.76248 | 60.07786 | 127 |
| 2297 | -1 | TACATGATTCCAGCTCGATG | AGG | 96.20958 | 66.08229 | 128 |
| 2299 | 1 | ATTCCTTTCCCTCATCGAGC | TGG | 72.65637 | 45.46834 | 129 |
| 2318 | 1 | CTGGAATCATGTACGAAGTT | TGG | 91.44916 | 38.23198 | 130 |
| 2333 | 1 | AAGTTTGGTACGCAGCTACC | TGG | 97.19351 | 52.48753 | 131 |
| 2334 | 1 | AGTTTGGTACGCAGCTACCT | GGG | 99.34096 | 54.73117 | 132 |
| 2340 | -1 | ATTATCTTCTTGCTTCTCCC | AGG | 65.57768 | 47.7251 | 133 |
| 2369 | 1 | ATAATGAAAAGCATATAAAC | TGG | 54.90991 | 38.48962 | 134 |
| 2370 | 1 | TAATGAAAAGCATATAAACT | GGG | 52.19307 | 58.36084 | 135 |
| 2408 | -1 | CTTGGATTTTGGGACACATA | AGG | 86.23392 | 45.11168 | 136 |
| 2418 | -1 | ATACGCCATTCTTGGATTTT | GGG | 83.33913 | 19.22763 | 137 |
| 2419 | -1 | GATACGCCATTCTTGGATTT | TGG | 84.63939 | 21.34065 | 138 |
| 2424 | 1 | TGTGTCCCAAAATCCAAGAA | TGG | 72.13536 | 54.39396 | 139 |
| 2426 | -1 | TAATTGAGATACGCCATTCT | TGG | 94.47859 | 25.9349 | 140 |
| 2441 | 1 | GAATGGCGTATCTCAATTAT | AGG | 87.03451 | 33.66134 | 141 |
| 2442 | 1 | AATGGCGTATCTCAATTATA | GGG | 83.63841 | 42.44805 | 142 |
| 2455 | 1 | AATTATAGGGACCTTGATTT | AGG | 51.02913 | 28.76282 | 143 |
| 2455 | -1 | GATCAGTTTTTCCTAAATCA | AGG | 81.50991 | 49.62644 | 144 |
| 2477 | -1 | GTGTAATTATTAGGACTCTT | GGG | 63.03567 | 37.12308 | 145 |
| 2478 | -1 | GGTGTAATTATTAGGACTCT | TGG | 87.51322 | 36.60593 | 146 |
| 2486 | -1 | CGTGCTTGGGTGTAATTATT | AGG | 89.37979 | 20.5205 | 147 |
| 2499 | -1 | TTCACCCCAGATACGTGCTT | GGG | 96.64962 | 52.4944 | 148 |
| 2500 | -1 | TTTCACCCCAGATACGTGCT | TGG | 98.66657 | 52.02383 | 149 |
| 2504 | 1 | ATTACACCCAAGCACGTATC | TGG | 74.79545 | 34.14523 | 150 |
| 2505 | 1 | TTACACCCAAGCACGTATCT | GGG | 95.37111 | 41.9996 | 151 |
| 2506 | 1 | TACACCCAAGCACGTATCTG | GGG | 98.75694 | 62.87462 | 152 |
| 2521 | 1 | ATCTGGGGTGAAAAGTACTT | TGG | 87.2143 | 49.68151 | 153 |
| 2547 | 1 | AAACTTTGACAAGTTAGTTA | AGG | 62.31057 | 39.88018 | 154 |
| 2565 | -1 | AAAATTATTGGGATCAACTT | TGG | 58.80076 | 33.4693 | 155 |
| 2576 | -1 | TCGTTTCTAAAAAAATTATT | GGG | 48.06619 | 22.76069 | 156 |
| 2577 | -1 | CTCGTTTCTAAAAAAATTAT | TGG | 55.23725 | 14.78939 | 157 |
| 2608 | -1 | GACGTCGTGGCGGAAGAGGT | GGG | 98.09602 | 65.65899 | 158 |
| 2609 | -1 | TGACGTCGTGGCGGAAGAGG | TGG | 91.81254 | 50.45154 | 159 |
| 2612 | -1 | TAATGACGTCGTGGCGGAAG | AGG | 98.07448 | 47.29841 | 160 |
| 2618 | -1 | AATAATTAATGACGTCGTGG | CGG | 89.39021 | 64.62174 | 161 |
| 2621 | -1 | AAAAATAATTAATGACGTCG | TGG | 78.57945 | 59.53964 | 162 |
| 2684 | -1 | ATATATAACAGATACATGTA | TGG | 62.7441 | 56.45533 | 163 |
| 2719 | -1 | CTTAGGAGCATACATAGTAC | AGG | 83.60953 | 55.16134 | 164 |
| 2736 | -1 | TACTGTAGATGTTCATACTT | AGG | 72.89098 | 47.9896 | 165 |
| 2776 | 1 | TGTAGACATCATAAGATATA | TGG | 61.18482 | 42.96127 | 166 |
| 2807 | 1 | AAATTATCTTTCTTATTTAA | TGG | 40.09022 | 10.00087 | 167 |

TABLE 2
gRNA sequences targeted for CsCBDAS
Position

| on SEQ. | | | | Efficiency | SEQ. |
|---|---|---|---|---|---|
| ID. NO.: 4 | Strand | Sequence | PAM | Score | ID NO. |
| 927 | -1 | AGCTTTATATATTGGAGCAG | GGG | 54.68597 | 168 |
| 928 | -1 | TAGCTTTATATATTGGAGCA | GGG | 56.13836 | 169 |
| 929 | -1 | ATAGCTTTATATATTGGAGC | AGG | 39.33975 | 170 |
| 935 | -1 | CTATTTATAGCTTTATATAT | TGG | 24.05235 | 171 |
| 947 | 1 | CAATATATAAAGCTATAAAT | AGG | 27.93541 | 172 |
| 971 | -1 | TAATGAATTTTGAATTACTA | TGG | 34.40115 | 173 |
| 1022 | 1 | AGTACTCAACATTCTCCTTT | TGG | 25.84144 | 174 |
| 1026 | -1 | TATCTTGCAAACAAACCAAA | AGG | 54.35998 | 175 |
| 1075 | -1 | GAGGATTAGCAATGGAAGTT | TGG | 35.85374 | 176 |
| 1083 | -1 | GTTTTCTCGAGGATTAGCAA | TGG | 61.84745 | 177 |
| 1094 | -1 | CATTTAAGGAAGTTTTCTCG | AGG | 62.59449 | 178 |
| 1108 | -1 | TATATTGCGAGAAGCATTTA | AGG | 22.98833 | 179 |
| 1133 | -1 | TTTAGATTTGTTGCATTATT | GGG | 18.30136 | 180 |
| 1134 | -1 | TTTTAGATTTGTTGCATTAT | TGG | 22.12177 | 181 |
| 1177 | -1 | TTAGGACAGACATATACAAT | GGG | 50.41844 | 182 |
| 1178 | -1 | TTTAGGACAGACATATACAA | TGG | 52.3325 | 183 |
| 1195 | -1 | GATTGTGTATTGTCGAATTT | AGG | 29.00652 | 184 |
| 1239 | -1 | GACGATAACAAGTGGTTTTG | GGG | 53.7312 | 185 |
| 1240 | -1 | TGACGATAACAAGTGGTTTT | GGG | 22.17034 | 186 |
| 1241 | -1 | GTGACGATAACAAGTGGTTT | TGG | 32.43789 | 187 |
| 1247 | -1 | GAAGGAGTGACGATAACAAG | TGG | 66.33896 | 188 |
| 1265 | -1 | TGGATATGAGAGACATGTGA | AGG | 69.66263 | 189 |
| 1279 | 1 | TCACATGTCTCTCATATCCA | AGG | 60.60093 | 190 |
| 1285 | -1 | TGGAGCATAGAATAGTGCCT | TGG | 51.2944 | 191 |
| 1305 | -1 | AATCTGCAAGCCAACTTTCT | TGG | 23.52241 | 192 |
| 1306 | 1 | ATTCTATGCTCCAAGAAAGT | TGG | 44.57193 | 193 |
| 1330 | 1 | TTGCAGATTCGAACTCGAAG | TGG | 63.85956 | 194 |
| 1333 | 1 | CAGATTCGAACTCGAAGTGG | TGG | 60.21029 | 195 |
| 1347 | 1 | AAGTGGTGGTCATGATTCTG | AGG | 61.14171 | 196 |
| 1348 | 1 | AGTGGTGGTCATGATTCTGA | GGG | 66.1321 | 197 |
| 1365 | -1 | AAATGGGACTTGAGATATGT | AGG | 48.94352 | 198 |
| 1381 | -1 | TCAAGTCTACTATAACAAAT | GGG | 44.7251 | 199 |
| 1382 | -1 | CTCAAGTCTACTATAACAAA | TGG | 34.85902 | 200 |
| 1438 | -1 | CGGCTTCAACCCATGCAGTT | TGG | 41.98925 | 201 |
| 1439 | 1 | ATGTTCATAGCCAAACTGCA | TGG | 53.04758 | 202 |
| 1440 | 1 | TGTTCATAGCCAAACTGCAT | GGG | 62.17149 | 203 |
| 1450 | 1 | CAAACTGCATGGGTTGAAGC | CGG | 57.17967 | 204 |
| 1458 | -1 | AACTTCTCCAAGGGTAGCTC | CGG | 46.01821 | 205 |
| 1462 | 1 | GTTGAAGCCGGAGCTACCCT | TGG | 56.02393 | 206 |
| 1467 | -1 | CCAATAATAAACTTCTCCAA | GGG | 65.85225 | 207 |
| 1468 | -1 | CCCAATAATAAACTTCTCCA | AGG | 53.53392 | 208 |
| 1478 | 1 | CCCTTGGAGAAGTTTATTAT | TGG | 20.92657 | 209 |
| 1479 | 1 | CCTTGGAGAAGTTTATTATT | GGG | 22.0283 | 210 |
| 1509 | 1 | GAAAAATGAGAGTCTTAGTT | TGG | 32.53304 | 211 |
| 1516 | 1 | GAGAGTCTTAGTTTGGCTGC | TGG | 40.11193 | 212 |
| 1517 | 1 | AGAGTCTTAGTTTGGCTGCT | GGG | 53.68777 | 213 |
| 1534 | -1 | GTCCACCTGCGCAAACAGTA | GGG | 51.52452 | 214 |
| 1540 | 1 | TATTGCCCTACTGTTTGCGC | AGG | 51.06289 | 215 |
| 1543 | 1 | TGCCCTACTGTTTGCGCAGG | TGG | 42.48452 | 216 |
| 1552 | 1 | GTTTGCGCAGGTGGACACTT | TGG | 56.37796 | 217 |
| 1558 | 1 | GCAGGTGGACACTTTGGTGG | AGG | 55.26903 | 218 |
| 1561 | 1 | GGTGGACACTTTGGTGGAGG | AGG | 53.00302 | 219 |
| 1567 | 1 | CACTTTGGTGGAGGAGGCTA | TGG | 43.62549 | 220 |
| 1580 | -1 | AGGCCATAGCTTCTCATCAA | TGG | 51.67311 | 221 |
| 1588 | 1 | GGACCATTGATGAGAAGCTA | TGG | 51.46816 | 222 |
| 1596 | 1 | GATGAGAAGCTATGGCCTCG | CGG | 72.19046 | 223 |
| 1600 | -1 | CAATGATATTATCAGCCGCG | AGG | 70.84796 | 224 |
| 1636 | 1 | GCACACTTAGTCAACGTTCA | TGG | 51.20136 | 225 |
| 1662 | 1 | AGTGCTAGATCGAAAATCTA | TGG | 38.12486 | 226 |
| 1663 | 1 | GTGCTAGATCGAAAATCTAT | GGG | 38.40821 | 227 |
| 1664 | 1 | TGCTAGATCGAAAATCTATG | GGG | 57.41791 | 228 |
| 1665 | 1 | GCTAGATCGAAAATCTATGG | GGG | 68.96928 | 229 |
| 1679 | 1 | CTATGGGGGAAGATCTCTTT | TGG | 21.51399 | 230 |
| 1680 | 1 | TATGGGGGAAGATCTCTTTT | GGG | 27.66662 | 231 |
| 1690 | 1 | GATCTCTTTTGGGCTTTACG | TGG | 59.22284 | 232 |
| 1693 | 1 | CTCTTTTGGGCTTTACGTGG | TGG | 46.97883 | 233 |
| 1696 | 1 | TTTTGGGCTTTACGTGGTGG | TGG | 42.65142 | 234 |
| 1711 | 1 | GGTGGTGGAGCAGAAAGCTT | CGG | 56.63543 | 235 |
| 1727 | 1 | GCTTCGGAATCATTGTAGCA | TGG | 58.92273 | 236 |
| 1740 | 1 | TGTAGCATGGAAAATTAGAC | TGG | 47.49685 | 237 |
| 1759 | -1 | CACTAAACATAGTAGACTTT | GGG | 33.44838 | 238 |
| 1760 | -1 | ACACTAAACATAGTAGACTT | TGG | 43.66107 | 239 |
| 1785 | 1 | GTTTAGTGTTAAAAAGATCA | TGG | 50.38465 | 240 |
| 1820 | 1 | TTGTCAAGTTAGTTAACAAA | TGG | 43.40152 | 241 |
| 1880 | 1 | TCATGACTCACTTCATAACT | AGG | 50.92123 | 242 |
| 1900 | 1 | AGGAACATTACAGATAATCA | AGG | 55.24406 | 243 |
| 1901 | 1 | GGAACATTACAGATAATCAA | GGG | 59.25054 | 244 |
| 1948 | 1 | TACTTCTCTTCAGTTTTCCT | TGG | 33.58233 | 245 |
| 1951 | 1 | TTCTCTTCAGTTTTCCTTGG | TGG | 33.88507 | 246 |
| 1954 | -1 | CTAGACTATCCACTCCACCA | AGG | 70.9998 | 247 |
| 1956 | 1 | TTCAGTTTTCCTTGGTGGAG | TGG | 49.36462 | 248 |
| 1998 | 1 | GAACAAGAGTTTTCCTGAGT | TGG | 44.24263 | 249 |
| 1999 | 1 | AACAAGAGTTTTCCTGAGTT | GGG | 38.37804 | 250 |
| 2000 | -1 | GTTTTTTTAATACCCAACTC | AGG | 47.70664 | 251 |
| 2013 | 1 | TGAGTTGGGTATTAAAAAAA | CGG | 35.28865 | 252 |
| 2033 | 1 | CGGATTGCAGACAATTGAGC | TGG | 51.29451 | 253 |
| 2059 | 1 | GATACTATCATCTTCTATAG | TGG | 50.42687 | 254 |
| 2094 | 1 | CGACACTGATAATTTTAACA | AGG | 47.46003 | 255 |
| 2119 | 1 | ATTTTGCTTGATAGATCCGC | TGG | 45.52498 | 256 |
| 2120 | 1 | TTTTGCTTGATAGATCCGCT | GGG | 66.15602 | 257 |
| 2124 | -1 | GAAAGCACCGTTCTGCCCAG | CGG | 74.32675 | 258 |
| 2128 | 1 | GATAGATCCGCTGGGCAGAA | CGG | 60.09095 | 259 |
| 2174 | -1 | ACAAATACAGATTCTGGAAT | TGG | 29.64316 | 260 |
| 2180 | -1 | ATTTGGACAAATACAGATTC | TGG | 32.93366 | 261 |
| 2196 | 1 | ATCTGTATTTGTCCAAATTT | TGG | 19.19094 | 262 |
| 2197 | -1 | CATATAATTTTTCCAAAATT | TGG | 22.7704 | 263 |
| 2221 | 1 | AAATTATATGAAGAAGATAT | AGG | 48.31475 | 264 |
| 2227 | 1 | TATGAAGAAGATATAGGAGC | TGG | 37.66845 | 265 |
| 2228 | 1 | ATGAAGAAGATATAGGAGCT | GGG | 55.50564 | 266 |
| 2251 | 1 | ATGTATGCGTTGTACCCTTA | CGG | 51.83563 | 267 |
| 2254 | 1 | TATGCGTTGTACCCTTACGG | TGG | 70.89895 | 268 |
| 2254 | -1 | CATCCATTATACCACCGTAA | GGG | 65.50071 | 269 |
| 2255 | -1 | TCATCCATTATACCACCGTA | AGG | 52.60015 | 270 |
| 2262 | 1 | GTACCCTTACGGTGGTATAA | TGG | 37.81553 | 271 |
| 2297 | -1 | ATTCCAGCTCGATGAGGGAA | TGG | 52.19017 | 272 |
| 2302 | -1 | ACAAGATTCCAGCTCGATGA | GGG | 58.64874 | 273 |
| 2303 | -1 | TACAAGATTCCAGCTCGATG | AGG | 64.02127 | 274 |
| 2305 | 1 | ATTCCATTCCCTCATCGAGC | TGG | 52.09796 | 275 |
| 2324 | 1 | CTGGAATCTTGTATGAGTTA | TGG | 42.0067 | 276 |
| 2339 | 1 | AGTTATGGTACATATGTAGC | TGG | 42.23778 | 277 |
| 2340 | 1 | GTTATGGTACATATGTAGCT | GGG | 57.33278 | 278 |
| 2375 | 1 | ATAACGAAAAGCATCTAAAC | TGG | 36.44159 | 279 |
| 2414 | -1 | CTTGGATTTTGGGACACATA | AGG | 45.61377 | 280 |
| 2424 | -1 | ATATGCCAATCTTGGATTTT | GGG | 20.28758 | 281 |
| 2425 | -1 | GATATGCCAATCTTGGATTT | TGG | 27.04467 | 282 |
| 2430 | 1 | TGTGTCCCAAAATCCAAGAT | TGG | 48.79389 | 283 |
| 2432 | -1 | TAATTGAGATATGCCAATCT | TGG | 37.73585 | 284 |
| 2461 | 1 | AATTATAGAGACCTTGATAT | AGG | 48.98627 | 285 |
| 2461 | -1 | GATCATTTATTCCTATATCA | AGG | 43.92608 | 286 |
| 2483 | -1 | GTGTAATTATTTGGATTCTT | GGG | 35.25388 | 287 |
| 2484 | -1 | TGTGTAATTATTTGGATTCT | TGG | 32.83863 | 288 |
| 2492 | -1 | CGTGCTTGTGTGTAATTATT | TGG | 14.08447 | 289 |
| 2510 | 1 | ATTACACACAAGCACGTATT | TGG | 23.81028 | 290 |
| 2511 | 1 | TTACACACAAGCACGTATTT | GGG | 22.17912 | 291 |
| 2512 | 1 | TACACACAAGCACGTATTTG | GGG | 52.47749 | 292 |
| 2527 | 1 | ATTTGGGGTGAGAAGTATTT | TGG | 23.04503 | 293 |
| 2543 | 1 | ATTTTGGTAAAAATTTTGAC | AGG | 51.19317 | 294 |
| 2565 | 1 | GCTAGTAAAAGTGAAAACCC | TGG | 70.67066 | 295 |
| 2571 | -1 | AAAATTATTGGGATCAACCA | GGG | 60.21659 | 296 |
| 2572 | -1 | AAAAATTATTGGGATCAACC | AGG | 51.83397 | 297 |
| 2582 | -1 | TCGTTTCTAAAAAAATTATT | GGG | 23.03526 | 298 |
| 2583 | -1 | TTCGTTTCTAAAAAAATTAT | TGG | 16.97799 | 299 |
| 2614 | -1 | GATGATGCCGTGGAAGAGGT | GGG | 70.41151 | 300 |
| 2615 | -1 | TGATGATGCCGTGGAAGAGG | TGG | 55.81165 | 301 |
| 2618 | 1 | AAAGCATCCCACCTCTTCCA | CGG | 62.29392 | 302 |
| 2618 | -1 | TAATGATGATGCCGTGGAAG | AGG | 53.28713 | 303 |
| 2624 | -1 | GATCATTAATGATGATGCCG | TGG | 59.64588 | 304 |
| 1535 | -1 | TGTCCACCTGCGCAAACAGT | AGG | 50.51317 | 824 |
| 1555 | -1 | TGCGCAGGTGGACACTTTGG | TGG | 53.04254 | 825 |

TABLE 3
gRNA sequences targeted for CsPT

| Position | | | | | | |
|---|---|---|---|---|---|---|
| on SEQ. | | | | Specificity | Efficiency | SEQ. |
| ID. NO.: 7 | Strand | Sequence | PAM | Score | Score | ID NO. |
| 2533 | 1 | CTATAAATAATATAATGTGT | TGG | 50.15504 | 52.66784 | 305 |
| 2548 | -1 | TCATATAATTAAGACACATT | AGG | 58.08987 | 39.51504 | 306 |
| 2605 | -1 | GAAGATTTTACGAGTTCATG | TGG | 78.97558 | 61.36868 | 307 |
| 2630 | -1 | GGTTGTACAATGCAGGAAGC | AGG | 94.76609 | 53.10248 | 308 |
| 2637 | -1 | TATATTGGGTTGTACAATGC | AGG | 89.85941 | 51.19467 | 309 |
| 2651 | -1 | ATCTGATTATATTTTATATT | GGG | 39.36258 | 19.27719 | 310 |
| 2652 | -1 | CATCTGATTATATTTTATAT | TGG | 30.46803 | 22.76394 | 311 |
| 2717 | 1 | TTTTGTATGTACCAATAAAG | AGG | 62.64158 | 47.70007 | 312 |
| 2717 | -1 | TGTGTTATTATCCTCTTTAT | TGG | 59.87869 | 13.1344 | 313 |
| 2736 | 1 | GAGGATAATAACACAATTAA | TGG | 53.00849 | 32.95598 | 314 |
| 2758 | 1 | GTAGTCATTTTGAGTATTAC | CGG | 65.22014 | 38.84988 | 315 |
| 2759 | 1 | TAGTCATTTTGAGTATTACC | GGG | 71.968 | 55.98069 | 316 |
| 2766 | -1 | TGCGTTAACAAATTACGACC | CGG | 95.86365 | 58.36396 | 317 |
| 2886 | 1 | ATAGATAATTTAATTCAAAA | TGG | 40.48669 | 36.67217 | 318 |
| 2895 | 1 | TTAATTCAAAATGGCAAACC | TGG | 77.80096 | 45.1845 | 319 |
| 2898 | 1 | ATTCAAAATGGCAAACCTGG | AGG | 80.97824 | 62.29508 | 320 |
| 2902 | -1 | AGCATTTTGGTTAATCCTCC | AGG | 91.75956 | 45.33793 | 321 |
| 2915 | -1 | TTCATGCAATCTTAGCATTT | TGG | 58.15822 | 22.94305 | 322 |
| 2962 | -1 | ATCATTTCTTATTTAATTAC | AGG | 45.18918 | 19.61169 | 323 |
| 2988 | 1 | GAAATGATGAGAAGAAATGA | TGG | 34.5444 | 57.85824 | 324 |
| 2997 | 1 | AGAAGAAATGATGGCAAACC | TGG | 78.33764 | 53.71781 | 325 |
| 3004 | -1 | TAAGAATTTATTAAAAAACC | AGG | 51.75369 | 55.62077 | 326 |
| 3817 | 1 | TATAGATGAGATGAGATACC | TGG | 85.29248 | 42.90625 | 327 |
| 3818 | 1 | ATAGATGAGATGAGATACCT | GGG | 81.64461 | 53.0755 | 328 |
| 3819 | 1 | TAGATGAGATGAGATACCTG | GGG | 90.29784 | 74.15808 | 329 |
| 3824 | -1 | TGCTGGGATTATCTGGCCCC | AGG | 99.76501 | 49.69658 | 330 |
| 3831 | -1 | CTATACTTGCTGGGATTATC | TGG | 91.47874 | 32.32128 | 331 |
| 3840 | -1 | ATGTGGCTGCTATACTTGCT | GGG | 78.11018 | 54.0404 | 332 |
| 3841 | -1 | TATGTGGCTGCTATACTTGC | TGG | 89.74351 | 29.36968 | 333 |
| 3854 | 1 | AGCAAGTATAGCAGCCACAT | AGG | 88.70643 | 65.60028 | 334 |
| 3857 | -1 | AATTGTTCTCCTATCCTATG | TGG | 83.08341 | 61.11113 | 335 |
| 3859 | 1 | GTATAGCAGCCACATAGGAT | AGG | 88.86311 | 52.55338 | 336 |
| 3880 | -1 | AACTTGACATTATTTTGTTC | TGG | 60.92679 | 30.32567 | 337 |
| 3896 | 1 | ACAAAATAATGTCAAGTTTC | TGG | 60.06363 | 20.93761 | 338 |
| 3910 | -1 | TCAACGTTGGCAAGTAAATA | TGG | 58.30165 | 30.24285 | 339 |
| 3923 | -1 | AAGATTTGGCATATCAACGT | TGG | 83.63482 | 54.17247 | 340 |
| 3937 | -1 | TATATATTTCTTTCAAGATT | TGG | 43.05887 | 26.00648 | 341 |
| 3994 | 1 | AATTATTTAAACTAATTATA | AGG | 27.59395 | 24.43668 | 342 |
| 4038 | -1 | AAAGATGCTTCAGACGTTGA | AGG | 87.29059 | 53.27521 | 343 |
| 4069 | -1 | TAAATCAATGGGTGCAGCTT | TGG | 87.80614 | 30.65775 | 344 |
| 4080 | -1 | CTAGCATTTATTAAATCAAT | GGG | 56.10289 | 36.01011 | 345 |
| 4081 | -1 | GCTAGCATTTATTAAATCAA | TGG | 77.76418 | 42.71614 | 346 |
| 4095 | 1 | TTGATTTAATAAATGCTAGC | AGG | 80.47522 | 49.30411 | 347 |
| 4109 | 1 | GCTAGCAGGAAAGTAAAAGA | AGG | 82.20668 | 59.95433 | 348 |
| 4121 | -1 | ATTTCAATTTGATTATTTTC | AGG | 42.72452 | 19.67918 | 349 |
| 4166 | 1 | ATACAATCAAATTAAAATAC | AGG | 41.34514 | 46.2972 | 350 |
| 4167 | 1 | TACAATCAAATTAAAATACA | GGG | 38.73103 | 56.21882 | 351 |
| 4186 | 1 | AGGGAAATCGTTTATGTTAT | TGG | 77.90025 | 22.30105 | 352 |
| 4224 | -1 | GAAACACGTATTTTAGAGAT | TGG | 86.63062 | 41.49631 | 353 |
| 4473 | -1 | ACCACTTAAGAATTTTCTTT | TGG | 55.0929 | 24.80139 | 354 |
| 4483 | 1 | GCCAAAAGAAAATTCTTAAG | TGG | 64.12831 | 50.01367 | 355 |
| 4515 | -1 | CAAGATATACTATATAATAT | AGG | 44.93458 | 38.54408 | 356 |
| 4527 | 1 | CTATATTATATAGTATATCT | TGG | 56.9374 | 31.81188 | 357 |
| 4528 | 1 | TATATTATATAGTATATCTT | GGG | 49.01302 | 47.17769 | 358 |
| 4573 | -1 | CCTACCATTTGAGTTGAGGT | GGG | 84.17055 | 57.97852 | 359 |
| 4574 | -1 | TCCTACCATTTGAGTTGAGG | TGG | 72.81109 | 48.59222 | 360 |
| 4577 | -1 | TTGTCCTACCATTTGAGTTG | AGG | 79.45986 | 49.37752 | 361 |
| 4580 | 1 | ATTACCCACCTCAACTCAAA | TGG | 78.76399 | 43.71813 | 362 |
| 4584 | 1 | CCCACCTCAACTCAAATGGT | AGG | 88.70205 | 65.11965 | 363 |
| 4603 | 1 | TAGGACAAGAGCTGCTCTGC | TGG | 96.15743 | 52.64779 | 364 |
| 4635 | 1 | ATGTGAAATTTGTAATAATA | TGG | 46.50782 | 21.55043 | 365 |
| 4636 | 1 | TGTGAAATTTGTAATAATAT | GGG | 44.42897 | 39.03579 | 366 |
| 4648 | -1 | TTTTTTTAATCTGCTTGCAC | AGG | 81.28024 | 42.68662 | 367 |
| 4763 | 1 | TACTCTTATAGTAACCAGAG | AGG | 91.51612 | 71.60058 | 368 |
| 4766 | -1 | ATAGTAATTAGTTACCTCTC | TGG | 88.7712 | 40.73368 | 369 |
| 4898 | -1 | TTATATAAAAATATATTCGT | TGG | 52.69209 | 44.66746 | 370 |
| 4915 | 1 | AATATATTTTTATATAAATA | TGG | 25.28327 | 23.82603 | 371 |
| 4916 | 1 | ATATATTTTTATATAAATAT | GGG | 20.87203 | 33.8773 | 372 |
| 4989 | 1 | AATTATCTCATCTAACTAAA | TGG | 61.01435 | 39.06579 | 373 |
| 5099 | 1 | TGTTAGTAAAGTAAAATACC | AGG | 75.01818 | 60.50068 | 374 |
| 5106 | -1 | TGCATTTCTTCTCAATTTCC | TGG | 55.3698 | 27.57883 | 375 |
| 5121 | 1 | GAAATTGAGAAGAAATGCAG | TGG | 66.10407 | 61.792 | 376 |
| 5125 | 1 | TTGAGAAGAAATGCAGTGGA | AGG | 82.89142 | 54.57658 | 377 |
| 5146 | 1 | GGATTTTGCTTCCATCTAAA | TGG | 80.36516 | 33.72734 | 378 |
| 5146 | -1 | ATTCTGTTCCACCATTTAGA | TGG | 81.59639 | 41.29933 | 379 |
| 5149 | 1 | TTTTGCTTCCATCTAAATGG | TGG | 73.22228 | 58.83392 | 380 |
| 5177 | -1 | TACTGTTTTGGTATTTTTGG | TGG | 52.22343 | 46.50211 | 381 |
| 5180 | -1 | GGCTACTGTTTTGGTATTTT | TGG | 57.1959 | 14.32438 | 382 |
| 5189 | -1 | TATATATTTGGCTACTGTTT | TGG | 70.57808 | 21.40652 | 383 |
| 5201 | -1 | GGTGGACCACTCTATATATT | TGG | 88.25064 | 28.7384 | 384 |
| 5206 | 1 | CAGTAGCCAAATATATAGAG | TGG | 80.8101 | 56.01321 | 385 |
| 5219 | -1 | ATAACTATAAAAATGAAGGG | TGG | 58.67371 | 66.9197 | 386 |
| 5222 | -1 | ATAATAACTATAAAAATGAA | GGG | 32.04873 | 55.75115 | 387 |
| 5223 | -1 | GATAATAACTATAAAAATGA | AGG | 44.11535 | 43.57819 | 388 |
| 5249 | -1 | AGCATAATTGTGGCACTGTT | TGG | 91.98088 | 35.4872 | 389 |
| 5259 | -1 | TTGGATTATGAGCATAATTG | TGG | 56.2773 | 59.04583 | 390 |
| 5278 | -1 | AAATATCAGTAAACACAGCT | TGG | 83.11907 | 51.55396 | 391 |
| 5301 | -1 | TGATCTACCACTAGCTTCAG | GGG | 89.84025 | 61.37871 | 392 |
| 5302 | -1 | CTGATCTACCACTAGCTTCA | GGG | 92.57816 | 49.17639 | 393 |
| 5303 | -1 | CCTGATCTACCACTAGCTTC | AGG | 91.25108 | 35.18826 | 394 |
| 5305 | 1 | GATATTTCCCCTGAAGCTAG | TGG | 85.99172 | 60.18662 | 395 |
| 5314 | 1 | CCTGAAGCTAGTGGTAGATC | AGG | 92.20769 | 47.61895 | 396 |
| 5403 | 1 | TACAAAAAATTGTTGTAGCT | AGG | 60.78952 | 43.73403 | 397 |
| 5404 | 1 | ACAAAAAATTGTTGTAGCTA | GGG | 62.02877 | 48.6504 | 398 |
| 5484 | -1 | TGTGGGTCATAAATAAAGTT | GGG | 63.20534 | 39.71502 | 399 |
| 5485 | -1 | TTGTGGGTCATAAATAAAGT | TGG | 76.86574 | 49.65417 | 400 |
| 5501 | -1 | ATAACAATTATAATTTTTGT | GGG | 35.20761 | 38.75031 | 401 |
| 5502 | -1 | GATAACAATTATAATTTTTG | TGG | 40.09979 | 38.6558 | 402 |
| 5524 | -1 | TATGCTATTGAACTTCTAGT | AGG | 73.91656 | 50.40142 | 403 |
| 5565 | -1 | TAATTTGAGTTAATAATTTT | AGG | 35.14236 | 12.79093 | 404 |
| 5737 | -1 | TTTACGATCTTCACATTGAC | AGG | 86.84326 | 43.60669 | 405 |
| 5760 | 1 | AAGATCGTAAATCTGATTGA | TGG | 79.39694 | 53.97011 | 406 |
| 5802 | -1 | CAAGGCATTCTTTTTTTTGG | TGG | 78.28594 | 36.74021 | 407 |
| 5805 | -1 | GTTCAAGGCATTCTTTTTTT | TGG | 77.61678 | 15.8281 | 408 |
| 5820 | -1 | AAGTTGGTCTCTGATGTTCA | AGG | 78.39446 | 48.10956 | 409 |
| 5836 | -1 | ATAACACAAATTTAATAAGT | TGG | 49.51096 | 36.82689 | 410 |
| 5872 | -1 | GGGAGAGCAGTGGATTGTTT | GGG | 90.26781 | 39.34907 | 411 |
| 5873 | -1 | TGGGAGAGCAGTGGATTGTT | TGG | 88.36253 | 32.36556 | 412 |
| 5882 | -1 | ATTTTGGTATGGGAGAGCAG | TGG | 94.63991 | 58.35153 | 413 |
| 5892 | -1 | ATTTGTTTTAATTTTGGTAT | GGG | 41.95799 | 36.82488 | 414 |
| 5893 | -1 | TATTTGTTTTAATTTTGGTA | TGG | 44.60475 | 38.88369 | 415 |
| 5898 | -1 | ATATATATTTGTTTTAATTT | TGG | 27.84114 | 26.40511 | 416 |
| 5979 | -1 | TTAGATTTGAGAGTTAAATG | TGG | 55.46527 | 69.10913 | 417 |
| 5998 | 1 | AACTCTCAAATCTAAATTTT | TGG | 45.16858 | 7.464133 | 418 |
| 5999 | 1 | ACTCTCAAATCTAAATTTTT | GGG | 45.01203 | 12.96086 | 419 |
| 6000 | 1 | CTCTCAAATCTAAATTTTTG | GGG | 47.56245 | 39.64619 | 420 |
| 6033 | 1 | TTAATCTTTTTTGTTATTTA | AGG | 39.86712 | 15.52071 | 421 |
| 6036 | 1 | ATCTTTTTTGTTATTTAAGG | TGG | 61.57787 | 43.97947 | 422 |
| 9091 | -1 | ATACTTTCTGTGCAAAAATA | TGG | 63.71265 | 24.27214 | 423 |
| 9190 | -1 | TAGCATTTACTTCATGCGCT | TGG | 88.85683 | 40.57145 | 424 |
| 9214 | 1 | GTAAATGCTATGATTGTATA | TGG | 67.17232 | 42.70364 | 425 |
| 9238 | -1 | TAAACTTTGGGAAGGCATGT | TGG | 82.83401 | 50.01002 | 426 |
| 9246 | -1 | TAAAATTTTAAACTTTGGGA | AGG | 42.81657 | 55.00889 | 427 |
| 9250 | -1 | CAACTAAAATTTTAAACTTT | GGG | 34.37113 | 28.08549 | 428 |
| 9251 | -1 | GCAACTAAAATTTTAAACTT | TGG | 48.74808 | 29.25676 | 429 |
| 9286 | 1 | ACTGAATGATTATCAGATTC | TGG | 81.6358 | 34.3321 | 430 |
| 9289 | 1 | GAATGATTATCAGATTCTGG | AGG | 77.96775 | 65.74172 | 431 |
| 9324 | -1 | AATAATAATAATGTGTTAAC | AGG | 53.34883 | 37.14458 | 432 |
| 9379 | 1 | GTTGTGTATTTTTTTCTTTT | AGG | 44.5762 | 18.62322 | 433 |
| 9420 | -1 | TAATATTATATTTAATTAGA | GGG | 39.10845 | 36.29759 | 434 |
| 9421 | -1 | TTAATATTATATTTAATTAG | AGG | 31.68565 | 39.80062 | 435 |
| 9510 | 1 | GAGACACACACATACCCTAA | TGG | 86.59004 | 50.40612 | 436 |
| 9513 | -1 | AATCGCAAAAAATTCCATTA | GGG | 57.75175 | 49.45903 | 437 |
| 9514 | -1 | CAATCGCAAAAAATTCCATT | AGG | 59.61224 | 37.96305 | 438 |
| 9567 | 1 | GTTTTGTAGATGAAAACTCT | TGG | 57.02108 | 52.35675 | 439 |
| 9570 | 1 | TTGTAGATGAAAACTCTTGG | TGG | 76.57239 | 57.2048 | 440 |
| 9585 | 1 | CTTGGTGGAGCAATGTTTAG | AGG | 81.85424 | 43.12889 | 441 |
| 9586 | 1 | TTGGTGGAGCAATGTTTAGA | GGG | 81.38342 | 49.63563 | 442 |
| 9613 | 1 | TTATTGTAAGAGTATTTAAT | TGG | 42.08458 | 25.1192 | 443 |
| 9621 | 1 | AGAGTATTTAATTGGTGTTT | TGG | 28.15366 | 29.48476 | 444 |
| 9622 | 1 | GAGTATTTAATTGGTGTTTT | GGG | 59.62235 | 18.36114 | 445 |
| 9623 | 1 | AGTATTTAATTGGTGTTTTG | GGG | 51.98403 | 49.93003 | 446 |
| 9642 | 1 | GGGGTGTCGATATAATAATG | AGG | 74.06144 | 54.09539 | 447 |
| 9648 | 1 | TCGATATAATAATGAGGTTT | TGG | 66.02342 | 29.35407 | 448 |
| 9649 | 1 | CGATATAATAATGAGGTTTT | GGG | 54.71808 | 18.42158 | 449 |
| 9664 | 1 | GTTTTGGGATTATTATTGTG | AGG | 54.73719 | 60.27909 | 450 |
| 9680 | 1 | TGTGAGGATTTAATAAAGTA | TGG | 62.9832 | 45.48923 | 451 |
| 9702 | 1 | GTAATTAGTTTGAAATGAAA | AGG | 42.78812 | 38.59876 | 452 |
| 9731 | -1 | CAATCAATAATAATCTTCAT | GGG | 51.22321 | 46.33554 | 453 |
| 9732 | -1 | TCAATCAATAATAATCTTCA | TGG | 52.54212 | 42.7126 | 454 |
| 9765 | 1 | AATATTAAAAAGAAAAAATG | AGG | 32.91192 | 50.40054 | 455 |
| 9766 | 1 | ATATTAAAAAGAAAAAATGA | GGG | 31.58425 | 59.29349 | 456 |
| 9775 | 1 | AGAAAAAATGAGGGAAAGAA | AGG | 37.46114 | 45.55358 | 457 |
| 9776 | 1 | GAAAAAATGAGGGAAAGAAA | GGG | 19.94773 | 54.89242 | 458 |

TABLE 4
gRNA sequences targeted for CsOLS

| Position on | | | | | | |
|---|---|---|---|---|---|---|
| SEQ. ID. | | | | Specificity | Efficiency | SEQ. |
| NO.: 10 | Strand | Sequence | PAM | Score | Score | ID NO. |
| 606 | 1 | CATACATAATATATATATAT | AGG | 38.80015 | 37.5899 | 459 |
| 694 | 1 | TATGAATCATCTTCGTGCTG | AGG | 92.51688 | 55.65201 | 460 |
| 695 | 1 | ATGAATCATCTTCGTGCTGA | GGG | 92.26269 | 55.16668 | 461 |
| 700 | 1 | TCATCTTCGTGCTGAGGGTC | CGG | 97.38454 | 45.93456 | 462 |
| 708 | -1 | CCGATGGCGAGAACGGAGGC | CGG | 99.23283 | 53.34917 | 463 |
| 712 | -1 | GGTGCCGATGGCGAGAACGG | AGG | 99.09248 | 64.53414 | 464 |
| 715 | -1 | GGCGGTGCCGATGGCGAGAA | CGG | 98.67263 | 50.70203 | 465 |
| 719 | 1 | CCGGCCTCCGTTCTCGCCAT | CGG | 99.67834 | 47.54908 | 466 |
| 724 | -1 | CTCCGGATTGGCGGTGCCGA | TGG | 99.54478 | 39.05761 | 467 |
| 733 | 1 | CGCCATCGGCACCGCCAATC | CGG | 99.93027 | 36.80185 | 468 |
| 733 | -1 | TAAAATGTTCTCCGGATTGG | CGG | 90.17454 | 51.19588 | 469 |
| 736 | -1 | TATTAAAATGTTCTCCGGAT | TGG | 88.80068 | 45.44233 | 470 |
| 741 | -1 | TCTTGTATTAAAATGTTCTC | CGG | 73.04651 | 45.0436 | 471 |
| 771 | -1 | GTGACCCGAAAGTAGTAGTC | AGG | 96.00665 | 45.73853 | 472 |
| 777 | 1 | AGTTTCCTGACTACTACTTT | CGG | 89.23006 | 34.52546 | 473 |
| 778 | 1 | GTTTCCTGACTACTACTTTC | GGG | 92.15777 | 29.58151 | 474 |
| 793 | -1 | TTGAGTCATGTGTTCACTTT | TGG | 73.05001 | 32.95869 | 475 |
| 876 | -1 | CATGGAAAAGTATTATTAGT | TGG | 56.37872 | 42.87624 | 476 |
| 894 | -1 | AAATAGATAATGCTTATACA | TGG | 65.92159 | 54.09676 | 477 |
| 917 | 1 | ATTATCTATTTATATAATAA | AGG | 38.73228 | 37.78037 | 478 |
| 1000 | 1 | CTTACTTTATATGTATATGT | AGG | 48.96629 | 45.21245 | 479 |
| 1019 | 1 | TAGGTGACAAAAGTATGATA | AGG | 63.42832 | 45.85195 | 480 |
| 1071 | 1 | TCTAAAGCAAAACCCAAGAT | TGG | 65.43717 | 53.59795 | 481 |
| 1072 | -1 | TCTCGTGCTCCGCCAATCTT | GGG | 95.9415 | 42.77202 | 482 |
| 1073 | -1 | ATCTCGTGCTCCGCCAATCT | TGG | 97.96263 | 28.38723 | 483 |
| 1074 | 1 | AAAGCAAAACCCAAGATTGG | CGG | 51.53179 | 58.86478 | 484 |
| 1095 | 1 | GGAGCACGAGATGCAAACTC | TGG | 94.77341 | 54.21811 | 485 |
| 1116 | 1 | GGATGCACGTCAAGACATGT | TGG | 94.27103 | 64.06157 | 486 |
| 1125 | 1 | TCAAGACATGTTGGTAGTTG | AGG | 77.53627 | 54.5857 | 487 |
| 1138 | 1 | GTAGTTGAGGTTCCAAAACT | TGG | 70.93462 | 44.35067 | 488 |
| 1139 | 1 | TAGTTGAGGTTCCAAAACTT | GGG | 68.9903 | 55.5133 | 489 |
| 1139 | -1 | CAAGCATCCTTCCCAAGTTT | TGG | 89.31912 | 22.9458 | 490 |
| 1143 | 1 | TGAGGTTCCAAAACTTGGGA | AGG | 81.7532 | 56.98226 | 491 |
| 1158 | 1 | TGGGAAGGATGCTTGTGCAA | AGG | 86.76519 | 63.49574 | 492 |
| 1170 | -1 | GGGTTGACCCCATTCTTTGA | TGG | 82.91197 | 29.12733 | 493 |
| 1172 | 1 | GTGCAAAGGCCATCAAAGAA | TGG | 72.44945 | 46.30634 | 494 |
| 1173 | 1 | TGCAAAGGCCATCAAAGAAT | GGG | 70.64931 | 38.21922 | 495 |
| 1174 | 1 | GCAAAGGCCATCAAAGAATG | GGG | 78.23762 | 71.1964 | 496 |
| 1190 | -1 | AAATGAGTGATTTTAGACTT | GGG | 54.17939 | 50.10922 | 497 |
| 1191 | -1 | TAAATGAGTGATTTTAGACT | TGG | 64.67096 | 46.53421 | 498 |
| 1233 | -1 | GTCTGCACCGGGCATGTCAG | TGG | 97.80392 | 53.96746 | 499 |
| 1237 | 1 | GCATCAACCACTGACATGCC | CGG | 92.69975 | 59.55302 | 500 |
| 1244 | -1 | GCGCAATGGTAGTCTGCACC | GGG | 99.19901 | 57.07458 | 501 |
| 1245 | -1 | AGCGCAATGGTAGTCTGCAC | CGG | 98.52043 | 48.84276 | 502 |
| 1258 | -1 | GTCCGAGAAGCTTAGCGCAA | TGG | 94.35214 | 55.9059 | 503 |
| 1267 | 1 | TACCATTGCGCTAAGCTTCT | CGG | 91.29961 | 37.63117 | 504 |
| 1286 | -1 | ATCATCACACGCTTCACTGA | GGG | 89.91737 | 65.13509 | 505 |
| 1287 | -1 | CATCATCACACGCTTCACTG | AGG | 96.68067 | 65.997 | 506 |
| 1309 | 1 | CGTGTGATGATGTATCAACT | AGG | 84.90515 | 64.72637 | 507 |
| 1318 | 1 | ATGTATCAACTAGGCTGTTA | TGG | 89.65638 | 40.36473 | 508 |
| 1321 | 1 | TATCAACTAGGCTGTTATGG | TGG | 89.40848 | 68.01826 | 509 |

TABLE 5
gRNA sequences targeted for CsAAE1

| Position on | | | | | | |
|---|---|---|---|---|---|---|
| SEQ. ID. | | | | Specificity | Efficiency | SEQ. |
| NO.: 13 | Strand | Sequence | PAM | Score | Score | ID No. |
| 1017 | 1 | ATTCAAAGTGAGAAAATTGT | TGG | 46.82883 | 42.08966 | 510 |
| 1036 | -1 | AACCAACAAGATCATGAGAA | GGG | 65.50328 | 65.57937 | 511 |
| 1037 | -1 | CAACCAACAAGATCATGAGA | AGG | 69.62583 | 61.96231 | 512 |
| 1045 | 1 | AACCCTTCTCATGATCTTGT | TGG | 26.15799 | 41.67638 | 513 |
| 1058 | 1 | ATCTTGTTGGTTGCTGTTCT | CGG | 83.86054 | 29.09087 | 514 |
| 1073 | 1 | GTTCTCGGAAGTGATGAAAG | AGG | 79.35978 | 74.00854 | 515 |
| 1099 | -1 | TGTTTGGTTAATTATTAAAT | AGG | 33.15963 | 19.05195 | 516 |
| 1115 | -1 | ACTAATTCTAATTTGGTGTT | TGG | 59.84418 | 32.05943 | 517 |
| 1122 | -1 | ATTGTATACTAATTCTAATT | TGG | 54.12193 | 21.41009 | 518 |
| 1157 | 1 | ATAAGATCATCATCATAGTG | TGG | 66.94162 | 60.62696 | 519 |
| 1168 | 1 | ATCATAGTGTGGAAGTGTAT | AGG | 73.55537 | 49.07951 | 520 |
| 1213 | -1 | TTTTTAGTCTTGACTCTCAA | GGG | 77.37359 | 51.59533 | 521 |
| 1214 | -1 | ATTTTTAGTCTTGACTCTCA | AGG | 74.23792 | 43.89766 | 522 |
| 1259 | 1 | AAAGTTAATAATGATGAAAT | TGG | 35.52392 | 37.97787 | 523 |
| 1274 | 1 | GAAATTGGTACCTTGAACAG | AGG | 81.08644 | 61.31573 | 524 |
| 1305 | -1 | TTGAGGTTATCTTTCAACTT | GGG | 64.77667 | 37.47441 | 525 |
| 1306 | -1 | ATTGAGGTTATCTTTCAACT | TGG | 77.67685 | 49.4408 | 526 |
| 1322 | -1 | CAATTGACTTAAATCAATTG | AGG | 58.54721 | 51.56257 | 527 |
| 1382 | 1 | AAGAAAATTACTAATTGCTC | AGG | 70.79317 | 49.81138 | 528 |
| 1395 | -1 | GGGGTGCCACCTTTGGGCGG | TGG | 98.94197 | 45.7826 | 529 |
| 1397 | 1 | TGCTCAGGTCCACCGCCCAA | AGG | 99.42523 | 59.45534 | 530 |
| 1398 | -1 | ATTGGGGTGCCACCTTTGGG | CGG | 95.01333 | 60.80505 | 531 |
| 1400 | 1 | TCAGGTCCACCGCCCAAAGG | TGG | 97.56412 | 60.82488 | 532 |
| 1401 | -1 | GCTATTGGGGTGCCACCTTT | GGG | 95.35281 | 30.579 | 533 |
| 1402 | -1 | TGCTATTGGGGTGCCACCTT | TGG | 97.59112 | 22.5998 | 534 |
| 1414 | -1 | TTTCGAGACAACTGCTATTG | GGG | 95.02736 | 53.10779 | 535 |
| 1415 | -1 | TTTTCGAGACAACTGCTATT | GGG | 91.01659 | 36.67599 | 536 |
| 1416 | -1 | GTTTTCGAGACAACTGCTAT | TGG | 93.27798 | 46.45164 | 537 |
| 1555 | -1 | GTTACTTTTGATAATTTGAT | AGG | 45.16312 | 42.37079 | 538 |
| 7139 | -1 | AATGAATATTGGAGGCATCA | AGG | 69.38955 | 59.92139 | 539 |
| 7147 | -1 | GATGATACAATGAATATTGG | AGG | 65.15451 | 59.16492 | 540 |
| 7150 | -1 | GCAGATGATACAATGAATAT | TGG | 70.32188 | 47.43563 | 541 |
| 7177 | -1 | AATGGTTATTATCATGCACA | TGG | 69.87898 | 60.70248 | 542 |
| 7195 | -1 | ATTTTTGAGCTTACATCTAA | TGG | 68.8764 | 35.50075 | 543 |
| 7220 | -1 | AGAGGTTTTAAGGAGGCATG | GGG | 90.60318 | 66.45911 | 544 |
| 7221 | -1 | GAGAGGTTTTAAGGAGGCAT | GGG | 82.79088 | 50.05129 | 545 |
| 7222 | -1 | GGAGAGGTTTTAAGGAGGCA | TGG | 80.93082 | 48.72577 | 546 |
| 7227 | -1 | TGAATGGAGAGGTTTTAAGG | AGG | 74.09145 | 69.25707 | 547 |
| 7230 | -1 | CATTGAATGGAGAGGTTTTA | AGG | 69.65457 | 16.77595 | 548 |
| 7238 | -1 | AATGCCTACATTGAATGGAG | AGG | 85.66528 | 65.63677 | 549 |
| 7243 | -1 | AAGGGAATGCCTACATTGAA | TGG | 89.92094 | 39.76854 | 550 |
| 7245 | 1 | AAAACCTCTCCATTCAATGT | AGG | 79.28148 | 56.68625 | 551 |
| 7261 | -1 | CACCATGATGTTTATTTTAA | GGG | 58.97317 | 9.801739 | 552 |
| 7262 | -1 | TCACCATGATGTTTATTTTA | AGG | 58.8467 | 15.41337 | 553 |
| 7270 | 1 | TTCCCTTAAAATAAACATCA | TGG | 61.84936 | 57.29224 | 554 |
| 7288 | -1 | GCATCGAAGACTCTGTTGAA | TGG | 91.56656 | 52.95186 | 555 |
| 7312 | -1 | GCGCTCGGTCCAGTCATGTT | TGG | 94.43272 | 39.68016 | 556 |
| 7314 | 1 | TTCGATGCTCCAAACATGAC | TGG | 86.8283 | 47.62248 | 557 |
| 7327 | -1 | GGAATTGGTGAATTAGCGCT | CGG | 96.74799 | 68.37405 | 558 |
| 7341 | 1 | AGCGCTAATTCACCAATTCC | TGG | 93.7153 | 36.16436 | 559 |
| 7342 | -1 | CCTAAAAACAAACCAGGAAT | TGG | 86.34378 | 41.08083 | 560 |
| 7348 | -1 | ATGCAGCCTAAAAACAAACC | AGG | 90.91529 | 63.74234 | 561 |
| 7353 | 1 | CCAATTCCTGGTTTGTTTTT | AGG | 66.41673 | 7.967407 | 562 |
| 7417 | 1 | AAATGTAACATAATTTTATA | TGG | 30.24509 | 21.5658 | 563 |
| 7418 | 1 | AATGTAACATAATTTTATAT | GGG | 36.43879 | 34.67268 | 564 |
| 7461 | -1 | ACTTTTCAAATAAGATTTGA | TGG | 49.31759 | 24.4411 | 565 |
| 7489 | -1 | AAAATATAATTTAAAAATAT | TGG | 23.50641 | 25.15222 | 566 |
| 7523 | 1 | ATAAGTTTATTAATTACCAT | TGG | 43.96007 | 52.6669 | 567 |
| 7528 | -1 | TGACAACAATGGTTATCCAA | TGG | 81.46885 | 63.06849 | 568 |
| 7539 | -1 | TTATACATACTTGACAACAA | TGG | 72.70705 | 49.88993 | 569 |
| 7569 | -1 | TCATTTAGTTCACAATGTAT | GGG | 69.87154 | 43.07873 | 570 |
| 7570 | -1 | TTCATTTAGTTCACAATGTA | TGG | 64.11188 | 34.27846 | 571 |
| 7620 | -1 | ATTGGTGGTGCATTTTCTGC | TGG | 75.70066 | 41.54012 | 572 |
| 7635 | -1 | TGTGGTGGCACAGAAATTGG | TGG | 86.97385 | 62.37008 | 573 |
| 7638 | -1 | ATGTGTGGTGGCACAGAAAT | TGG | 91.18051 | 35.67967 | 574 |
| 7650 | -1 | CCTGTTATCGAAATGTGTGG | TGG | 86.4401 | 69.73002 | 575 |
| 7653 | -1 | AAGCCTGTTATCGAAATGTG | TGG | 86.07817 | 69.79364 | 576 |
| 7661 | 1 | CCACCACACATTTCGATAAC | AGG | 91.98911 | 34.09153 | 577 |
| 7688 | -1 | ATGAATACCTATGGTTGATG | GGG | 79.66939 | 61.20548 | 578 |
| 7689 | -1 | GATGAATACCTATGGTTGAT | GGG | 77.02024 | 47.22441 | 579 |
| 7690 | -1 | AGATGAATACCTATGGTTGA | TGG | 76.43674 | 51.54312 | 580 |
| 7692 | 1 | TTGCTCTCCCCATCAACCAT | AGG | 89.17718 | 57.68331 | 581 |
| 7697 | -1 | CTAATGTAGATGAATACCTA | TGG | 79.03258 | 52.66036 | 582 |
| 7726 | 1 | ATTAGATGCTTCACCAGAAG | AGG | 76.7559 | 48.05163 | 583 |
| 7728 | -1 | TGTAGTTGCTTTTCCTCTTC | TGG | 70.77754 | 18.10598 | 584 |
| 7745 | 1 | GAGGAAAAGCAACTACAAGA | AGG | 77.08372 | 61.08762 | 585 |
| 7746 | 1 | AGGAAAAGCAACTACAAGAA | GGG | 59.40687 | 56.12144 | 586 |
| 7780 | -1 | ATTATATTAAACGTATTATA | TGG | 58.83333 | 26.89437 | 587 |
| 7828 | 1 | TGTGCATAGATAAGAAATAT | TGG | 49.90268 | 36.74205 | 588 |
| 7853 | 1 | AATATATTATAATTCTTTAC | CGG | 49.5763 | 28.18052 | 589 |
| 7857 | 1 | TATTATAATTCTTTACCGGA | TGG | 78.22362 | 52.20352 | 590 |
| 7860 | 1 | TATAATTCTTTACCGGATGG | TGG | 93.78137 | 54.29303 | 591 |
| 7861 | -1 | GCTATGATTGGTCCACCATC | CGG | 82.3879 | 55.49635 | 592 |
| 7873 | -1 | ATTGTGTTAGTGGCTATGAT | TGG | 85.22929 | 56.76141 | 593 |
| 7883 | -1 | AAAAGTAGCAATTGTGTTAG | TGG | 71.54968 | 45.76116 | 594 |
| 7906 | -1 | TCCCTAGCATTGTTCGATCA | TGG | 95.58084 | 52.55436 | 595 |
| 7915 | 1 | TTCCATGATCGAACAATGCT | AGG | 89.83435 | 51.35939 | 596 |
| 7916 | 1 | TCCATGATCGAACAATGCTA | GGG | 83.34292 | 59.40933 | 597 |
| 7929 | -1 | TAAAGTAACAATGCTAGGTG | TGG | 86.0951 | 63.10284 | 598 |
| 7934 | -1 | GATGCTAAAGTAACAATGCT | AGG | 68.92833 | 59.04437 | 599 |
| 7956 | -1 | ATGTTTTCTAATATGTGTGT | AGG | 35.74139 | 53.28168 | 600 |
| 8037 | -1 | AATAAAAAACATGATAAGTT | CGG | 49.74918 | 45.14228 | 601 |
| 8086 | 1 | AATAAAAATGATGTGATTAT | TGG | 45.03911 | 43.8586 | 602 |
| 8114 | -1 | ATGTCTAATTCATTATAGAA | GGG | 64.65734 | 51.67938 | 603 |
| 8115 | -1 | AATGTCTAATTCATTATAGA | AGG | 55.7369 | 45.68541 | 604 |
| 8199 | 1 | CTAAAAAGTTTATTAGTTAA | TGG | 50.73953 | 27.333 | 605 |
| 8223 | -1 | TAGCTTCGCCAAATTTGTGC | AGG | 88.30821 | 49.16688 | 606 |
| 8226 | 1 | TTAGTTTACCTGCACAAATT | TGG | 74.90104 | 37.25004 | 607 |
| 8245 | 1 | TTGGCGAAGCTAGAAACAAG | TGG | 82.9657 | 68.53364 | 608 |
| 8261 | -1 | GCTTCTCTTGCCTTGTATAA | TGG | 81.45785 | 36.91329 | 609 |
| 8262 | 1 | AAGTGGTGATCCATTATACA | AGG | 80.45179 | 62.09832 | 610 |
| 8282 | 1 | AGGCAAGAGAAGCCCCATTA | AGG | 93.10259 | 42.00278 | 611 |
| 8283 | -1 | CTATGCTTCACTCCTTAATG | GGG | 92.90881 | 51.33474 | 612 |
| 8284 | -1 | TCTATGCTTCACTCCTTAAT | GGG | 80.41893 | 33.5345 | 613 |
| 8285 | -1 | GTCTATGCTTCACTCCTTAA | TGG | 92.13485 | 27.46679 | 614 |
| 8305 | 1 | AGTGAAGCATAGACCAGCCA | AGG | 93.2408 | 55.91368 | 615 |
| 8307 | -1 | TTGGATGATGGGTCCTTGGC | TGG | 89.87415 | 37.38458 | 616 |
| 8311 | -1 | TTGGTTGGATGATGGGTCCT | TGG | 90.64716 | 46.50603 | 617 |
| 8318 | -1 | ACTAATCTTGGTTGGATGAT | GGG | 55.88848 | 43.22222 | 618 |
| 8319 | -1 | CACTAATCTTGGTTGGATGA | TGG | 64.35474 | 40.68855 | 619 |
| 8326 | -1 | TTTGGCCCACTAATCTTGGT | TGG | 63.95374 | 49.8337 | 620 |
| 8330 | -1 | ATTGTTTGGCCCACTAATCT | TGG | 83.91578 | 42.92642 | 621 |
| 8331 | 1 | CATCATCCAACCAAGATTAG | TGG | 45.46742 | 53.20986 | 622 |
| 8332 | 1 | ATCATCCAACCAAGATTAGT | GGG | 44.6141 | 53.44581 | 623 |
| 8344 | -1 | GGAAAGGTGATGTCATTGTT | TGG | 78.65278 | 38.65702 | 624 |
| 8360 | -1 | AGCCATTTGGACATTAGGAA | AGG | 84.2141 | 58.69325 | 625 |
| 8365 | -1 | GGTGGAGCCATTTGGACATT | AGG | 86.64479 | 40.00615 | 626 |
| 8369 | 1 | CACCTTTCCTAATGTCCAAA | TGG | 75.0476 | 45.46945 | 627 |
| 8373 | -1 | TGCAGATGGGTGGAGCCATT | TGG | 93.51908 | 44.95053 | 628 |
| 8383 | -1 | TAAAAGCAGCTGCAGATGGG | TGG | 93.4923 | 62.38047 | 629 |
| 8386 | -1 | CTTTAAAAGCAGCTGCAGAT | GGG | 88.26499 | 50.81653 | 630 |
| 8387 | -1 | CCTTTAAAAGCAGCTGCAGA | TGG | 86.2835 | 54.0118 | 631 |
| 8398 | 1 | CCATCTGCAGCTGCTTTTAA | AGG | 87.21073 | 15.05639 | 632 |
| 8408 | 1 | CTGCTTTTAAAGGAGTTGCT | TGG | 88.7393 | 43.96243 | 633 |
| 8409 | 1 | TGCTTTTAAAGGAGTTGCTT | GGG | 70.87059 | 39.59579 | 634 |
| 8416 | 1 | AAAGGAGTTGCTTGGGTCCA | TGG | 92.13848 | 46.59847 | 635 |
| 8422 | -1 | GGGAGCCAAAGGCAATTCCA | TGG | 82.72992 | 60.08041 | 636 |
| 8428 | 1 | TGGGTCCATGGAATTGCCTT | TGG | 83.84223 | 36.75466 | 637 |
| 8433 | -1 | TTTTGGTATAGGGGAGCCAA | AGG | 62.72308 | 58.65204 | 638 |
| 8442 | -1 | ACAATATTGTTTTGGTATAG | GGG | 70.04203 | 44.96968 | 639 |
| 8443 | -1 | GACAATATTGTTTTGGTATA | GGG | 65.06063 | 24.34171 | 640 |
| 8444 | -1 | AGACAATATTGTTTTGGTAT | AGG | 60.62704 | 36.77509 | 641 |
| 8450 | -1 | GATGGAAGACAATATTGTTT | TGG | 49.88722 | 31.0149 | 642 |
| 8468 | -1 | ATCAATCTAAACACTTTTGA | TGG | 65.25066 | 22.9316 | 643 |
| 8497 | 1 | TTGATGAACAAAACTAATTA | AGG | 60.26021 | 31.42456 | 644 |
| 8506 | 1 | AAAACTAATTAAGGATATTA | AGG | 28.77328 | 23.99012 | 645 |
| 8507 | 1 | AAACTAATTAAGGATATTAA | GGG | 47.82816 | 35.89387 | 646 |
| 8508 | 1 | AACTAATTAAGGATATTAAG | GGG | 56.45894 | 52.91201 | 647 |
| 8509 | 1 | ACTAATTAAGGATATTAAGG | GGG | 66.78309 | 63.26044 | 648 |
| 8529 | 1 | GGGCTCATGAGATTTTTTAA | CGG | 72.44404 | 35.38053 | 649 |
| 8573 | -1 | ATTCAGTAAATAATGATGCT | TGG | 74.0009 | 56.25971 | 650 |
| 8598 | -1 | TTTTAAGAGCGCAAACTAAA | AGG | 69.75899 | 34.44909 | 651 |
| 8648 | 1 | GTTTTGTTTAAAGATAATAG | CGG | 63.36248 | 63.28955 | 652 |
| 8649 | 1 | TTTTGTTTAAAGATAATAGC | GGG | 66.03681 | 52.89056 | 653 |
| 8681 | 1 | TCAAAATTTTATATTGTAAG | CGG | 40.99317 | 51.22546 | 654 |
| 8758 | -1 | AGACAATAAGAGAGTTTATA | CGG | 63.10426 | 33.70903 | 655 |
| 8777 | 1 | ACTCTCTTATTGTCTTAAAC | AGG | 82.59379 | 27.0699 | 656 |
| 8778 | 1 | CTCTCTTATTGTCTTAAACA | GGG | 76.21161 | 46.02143 | 657 |
| 8779 | 1 | TCTCTTATTGTCTTAAACAG | GGG | 81.69869 | 60.34407 | 658 |
| 8801 | -1 | GATTTAATGATCAAGAATTT | AGG | 52.45697 | 36.47312 | 659 |
| 8853 | -1 | GAATTTATTAAAATAGCATT | TGG | 58.39351 | 33.17128 | 660 |
| 8873 | 1 | ATTTTAATAAATTCAAAACA | TGG | 42.10593 | 49.13089 | 661 |
| 8876 | 1 | TTAATAAATTCAAAACATGG | CGG | 51.71372 | 68.38405 | 662 |
| 8879 | 1 | ATAAATTCAAAACATGGCGG | TGG | 87.21197 | 46.77449 | 663 |
| 8900 | -1 | CTCTTCTCATCTGGAACAAC | AGG | 83.95127 | 38.49164 | 664 |
| 8909 | -1 | ACAAACATCCTCTTCTCATC | TGG | 82.47663 | 39.35798 | 665 |
| 8912 | 1 | CTGTTGTTCCAGATGAGAAG | AGG | 82.81433 | 53.17806 | 666 |
| 8925 | 1 | TGAGAAGAGGATGTTTGTAT | AGG | 75.94583 | 50.65718 | 667 |
| 8934 | 1 | GATGTTTGTATAGGCATCAA | CGG | 83.23553 | 48.99018 | 668 |
| 8935 | 1 | ATGTTTGTATAGGCATCAAC | GGG | 71.89291 | 50.59686 | 669 |
| 8970 | 1 | AGTAAATTCACAATTTCTGC | AGG | 72.90705 | 51.93162 | 670 |
| 8984 | -1 | GGGACTAATATCTTCTTGAG | TGG | 81.64895 | 66.64607 | 671 |
| 9004 | -1 | AAAATAATAAGCATATATGT | GGG | 46.06348 | 54.81391 | 672 |
| 9005 | -1 | CAAAATAATAAGCATATATG | TGG | 50.75457 | 48.01509 | 673 |
| 9017 | 1 | CACATATATGCTTATTATTT | TGG | 49.76422 | 8.576781 | 674 |
| 9121 | -1 | TTTTTAATTAAAAGTAACAC | AGG | 60.1073 | 53.94147 | 675 |
| 9187 | -1 | TTAATTTAGGAGTTATTTTG | GGG | 44.33331 | 53.49833 | 676 |
| 9188 | -1 | ATTAATTTAGGAGTTATTTT | GGG | 41.75312 | 20.1865 | 677 |
| 9189 | -1 | TATTAATTTAGGAGTTATTT | TGG | 46.05579 | 9.863159 | 678 |
| 9200 | -1 | AAGTTGGGCATTATTAATTT | AGG | 64.26833 | 29.09929 | 679 |
| 9215 | -1 | ATCAGGGGTTTTATCAAGTT | GGG | 73.93607 | 33.45243 | 680 |
| 9216 | -1 | TATCAGGGGTTTTATCAAGT | TGG | 84.29187 | 44.06854 | 681 |
| 9230 | -1 | ATCACGTTTCTTATTATCAG | GGG | 80.66179 | 58.62752 | 682 |
| 9231 | -1 | TATCACGTTTCTTATTATCA | GGG | 82.42224 | 47.4101 | 683 |
| 9232 | -1 | GTATCACGTTTCTTATTATC | AGG | 58.04329 | 29.70134 | 684 |
| 9261 | -1 | AAAATACATGTGAATGTTAG | TGG | 69.1364 | 51.00635 | 685 |
| 9289 | 1 | TATTTTAGTTCAAACTCCAA | TGG | 60.62746 | 52.04271 | 686 |
| 9294 | -1 | ATATGAACATTTGGATCCAT | TGG | 46.78738 | 41.8429 | 687 |
| 9303 | -1 | TATCTATATATATGAACATT | TGG | 54.10978 | 35.78905 | 688 |
| 9338 | -1 | GTGGAATATATAAACAGTAG | AGG | 80.1669 | 56.2241 | 689 |
| 9357 | -1 | AATTCGTATAGAGATTAATG | TGG | 73.95175 | 62.50764 | 690 |
| 9459 | -1 | GCGTGATGGCGATATTTCTT | GGG | 86.54713 | 37.70124 | 691 |
| 9460 | -1 | TGCGTGATGGCGATATTTCT | TGG | 91.24965 | 32.52202 | 692 |
| 9473 | -1 | ATTGGTGCAGAATTGCGTGA | TGG | 89.64882 | 66.39713 | 693 |
| 9491 | -1 | CCTTGTAGTGGCTCTAATAT | TGG | 83.67391 | 26.74643 | 694 |
| 9502 | 1 | CCAATATTAGAGCCACTACA | AGG | 88.94997 | 62.54443 | 695 |
| 9503 | -1 | GCCATTGTTATTCCTTGTAG | TGG | 86.75924 | 41.11451 | 696 |
| 9513 | 1 | GCCACTACAAGGAATAACAA | TGG | 78.65214 | 62.36806 | 697 |
| 9519 | 1 | ACAAGGAATAACAATGGCCA | TGG | 84.70821 | 54.69464 | 698 |
| 9520 | 1 | CAAGGAATAACAATGGCCAT | GGG | 88.76204 | 62.99032 | 699 |
| 9525 | -1 | TGTGGAAGCCAAGTCTCCCA | TGG | 96.83523 | 60.94207 | 700 |
| 9528 | 1 | AACAATGGCCATGGGAGACT | TGG | 91.04348 | 37.2658 | 701 |
| 9543 | -1 | TAAATTGTGCAGTAGAGTTG | TGG | 74.79858 | 59.21973 | 702 |
| 9574 | 1 | TTAACTCTTTTTTTGTGAGA | TGG | 67.00048 | 39.91071 | 703 |
| 9617 | 1 | ATAATTTATTATTATTTGTC | AGG | 34.22417 | 35.90263 | 704 |
| 13019 | 1 | TGCTAGTACATGTCTATATA | AGG | 51.98406 | 29.87248 | 705 |
| 13060 | -1 | AGCAAAAGCCATTTTTACAC | AGG | 81.1356 | 53.00551 | 706 |
| 13063 | 1 | TATATATACCTGTGTAAAAA | TGG | 60.80392 | 35.0674 | 707 |
| 13103 | 1 | CGAAGTCTTGTTGATATTTC | AGG | 57.63384 | 20.64668 | 708 |
| 13152 | -1 | TATCTAGCTATTGTTCTTGC | GGG | 77.9103 | 33.48401 | 709 |
| 13153 | -1 | CTATCTAGCTATTGTTCTTG | CGG | 74.55985 | 43.94593 | 710 |
| 13180 | -1 | GCCAATGCATGTGGATGCTG | TGG | 92.72347 | 60.88652 | 711 |
| 13189 | -1 | AATTGATATGCCAATGCATG | TGG | 77.53646 | 64.53936 | 712 |
| 13190 | 1 | ACCACAGCATCCACATGCAT | TGG | 81.43998 | 59.13625 | 713 |
| 13224 | -1 | GAAGAAATGGGTTTGGAGAA | GGG | 49.22523 | 49.88879 | 714 |
| 13225 | -1 | TGAAGAAATGGGTTTGGAGA | AGG | 69.09616 | 48.87133 | 715 |
| 13231 | -1 | TGCACTTGAAGAAATGGGTT | TGG | 71.57783 | 32.7508 | 716 |
| 13236 | -1 | GGTTATGCACTTGAAGAAAT | GGG | 58.12192 | 38.87319 | 717 |
| 13237 | -1 | TGGTTATGCACTTGAAGAAA | TGG | 67.04056 | 33.35969 | 718 |
| 13257 | -1 | TAATTAATTTGATGGTTAGT | TGG | 56.6831 | 44.59873 | 719 |
| 13265 | -1 | ATGTATGGTAATTAATTTGA | TGG | 46.32665 | 36.88262 | 720 |
| 13280 | -1 | TTGGATATTGAAACTATGTA | TGG | 64.43349 | 55.56421 | 721 |
| 13299 | -1 | TTAATATGATATGGTTTATT | TGG | 51.05981 | 16.59851 | 722 |
| 13308 | -1 | TTCATAAAGTTAATATGATA | TGG | 45.86946 | 38.61687 | 723 |
| 13386 | -1 | ACCAATTGCGTAAACGTGTT | GGG | 92.64873 | 38.15694 | 724 |
| 13387 | -1 | GACCAATTGCGTAAACGTGT | TGG | 94.58325 | 51.95048 | 725 |
| 13396 | 1 | ACCCAACACGTTTACGCAAT | TGG | 94.35879 | 35.38154 | 726 |
| 13419 | 1 | TCAAGTGTCAATTTGTTTAG | AGG | 60.27782 | 38.1724 | 727 |
| 13444 | -1 | ATGATTGTATGGCGTGATGA | AGG | 93.06641 | 56.01275 | 728 |
| 13455 | -1 | TGAATGATACAATGATTGTA | TGG | 65.25489 | 43.79748 | 729 |
| 13524 | 1 | GCTGAGTTAAGATAACCTCC | TGG | 95.01444 | 53.02965 | 730 |
| 13528 | -1 | GGTAGTGAATGGCTTCCAGG | AGG | 97.82923 | 69.29733 | 731 |
| 13531 | -1 | GGGGGTAGTGAATGGCTTCC | AGG | 97.10675 | 35.56817 | 732 |
| 13539 | -1 | ATAATCCAGGGGGTAGTGAA | TGG | 91.39645 | 59.2292 | 733 |
| 13545 | 1 | GGAAGCCATTCACTACCCCC | TGG | 99.45732 | 52.97305 | 734 |
| 13549 | -1 | GATGATATTAATAATCCAGG | GGG | 87.72204 | 72.36028 | 735 |
| 13550 | -1 | TGATGATATTAATAATCCAG | GGG | 70.74045 | 64.58516 | 736 |
| 13551 | -1 | ATGATGATATTAATAATCCA | GGG | 56.34847 | 64.43657 | 737 |
| 13552 | -1 | GATGATGATATTAATAATCC | AGG | 64.68399 | 47.30268 | 738 |
| 13584 | 1 | TCTCTACGCAATATACATTC | TGG | 85.48205 | 37.55777 | 739 |
| 13598 | -1 | GATGAAGATAAGTTTTTCAA | AGG | 34.9946 | 46.22336 | 740 |
| 13625 | -1 | GTATTGGAGAACATTACTAA | TGG | 81.61714 | 54.192 | 741 |
| 13641 | -1 | TTATATAATGTTAGGTGTAT | TGG | 50.0441 | 37.07111 | 742 |
| 13649 | -1 | GGTTAATATTATATAATGTT | AGG | 46.14634 | 41.29752 | 743 |
| 13670 | -1 | TGTTAATTATTTGTAATTAA | TGG | 30.06382 | 19.30784 | 744 |
| 13699 | -1 | TTTATTATTTTATTTGAGGG | TGG | 56.87973 | 54.11921 | 745 |
| 13702 | -1 | CTATTTATTATTTTATTTGA | GGG | 22.86306 | 26.95378 | 746 |
| 13703 | -1 | ACTATTTATTATTTTATTTG | AGG | 24.37755 | 35.02855 | 747 |
| 13732 | 1 | ATAGTTATTATTATTACCTC | AGG | 69.1903 | 42.38884 | 748 |
| 13733 | 1 | TAGTTATTATTATTACCTCA | GGG | 66.22878 | 58.43041 | 749 |
| 13737 | -1 | ATTTTCTGTAAGAAACCCTG | AGG | 86.29961 | 70.70242 | 750 |
| 13754 | 1 | GGTTTCTTACAGAAAATTCT | TGG | 66.82242 | 29.73772 | 751 |
| 13776 | 1 | GAAATGAGAAAAGCTTGAAA | TGG | 53.70676 | 37.62262 | 752 |
| 13777 | 1 | AAATGAGAAAAGCTTGAAAT | GGG | 61.18077 | 43.36214 | 753 |
| 13791 | -1 | GTTTTTGGGAGTCAAGTATA | AGG | 79.38507 | 47.93209 | 754 |
| 13805 | -1 | AAGCGAGGAAAAGAGTTTTT | GGG | 66.05009 | 27.99152 | 755 |
| 13806 | -1 | GAAGCGAGGAAAAGAGTTTT | TGG | 65.56542 | 30.89956 | 756 |
| 13820 | -1 | GGCGCACTTTTGGAGAAGCG | AGG | 98.3174 | 63.90309 | 757 |
| 13830 | -1 | CACCAATATGGGCGCACTTT | TGG | 96.32144 | 22.47601 | 758 |
| 13839 | 1 | CTCCAAAAGTGCGCCCATAT | TGG | 94.31583 | 41.35959 | 759 |
| 13841 | -1 | AAAGTAAAGTCCACCAATAT | GGG | 68.75283 | 36.52054 | 760 |
| 13842 | 1 | CAAAAGTGCGCCCATATTGG | TGG | 97.30856 | 48.92306 | 761 |
| 13842 | -1 | GAAAGTAAAGTCCACCAATA | TGG | 84.33286 | 38.41171 | 762 |
| 13866 | -1 | TTTTCTTTTATTATGATTCA | GGG | 39.04863 | 43.80485 | 763 |
| 13867 | -1 | TTTTTCTTTTATTATGATTC | AGG | 29.8602 | 27.80478 | 764 |
| 15978 | 1 | GAAAAAATATATATATAGTA | AGG | 36.13766 | 41.8761 | 765 |
| 15995 | 1 | GTAAGGAAAGAGAGAGATTA | CGG | 54.88255 | 36.8303 | 766 |
| 15996 | 1 | TAAGGAAAGAGAGAGATTAC | GGG | 57.53794 | 51.49059 | 767 |
| 16000 | 1 | GAAAGAGAGAGATTACGGGT | CGG | 86.61929 | 64.70693 | 768 |
| 16001 | 1 | AAAGAGAGAGATTACGGGTC | GGG | 93.1411 | 48.83463 | 769 |
| 16013 | 1 | TACGGGTCGGGTATCCATGC | AGG | 98.86045 | 52.97598 | 770 |
| 16016 | 1 | GGGTCGGGTATCCATGCAGG | AGG | 97.58735 | 53.65399 | 771 |
| 16016 | -1 | TTGGACCCGCCCCTCCTGCA | TGG | 96.38923 | 49.42741 | 772 |
| 16017 | 1 | GGTCGGGTATCCATGCAGGA | GGG | 99.45455 | 58.97398 | 773 |
| 16018 | 1 | GTCGGGTATCCATGCAGGAG | GGG | 98.23654 | 50.30444 | 774 |
| 16021 | 1 | GGGTATCCATGCAGGAGGGG | CGG | 94.19216 | 43.31877 | 775 |
| 16022 | 1 | GGTATCCATGCAGGAGGGGC | GGG | 98.70541 | 48.06551 | 776 |
| 16035 | -1 | TATGGTTGCTATAAAGACTT | TGG | 72.61024 | 46.52942 | 777 |
| 16053 | -1 | CTGCACCAGATGCTCTACTA | TGG | 93.10997 | 49.44754 | 778 |
| 16059 | 1 | AGCAACCATAGTAGAGCATC | TGG | 93.28253 | 48.85983 | 779 |
| 16065 | 1 | CATAGTAGAGCATCTGGTGC | AGG | 93.11563 | 41.82045 | 780 |
| 16066 | 1 | ATAGTAGAGCATCTGGTGCA | GGG | 83.82199 | 65.30317 | 781 |
| 16072 | 1 | GAGCATCTGGTGCAGGGAGA | AGG | 95.16992 | 42.4774 | 782 |
| 16073 | 1 | AGCATCTGGTGCAGGGAGAA | GGG | 92.19717 | 45.18153 | 783 |
| 16074 | 1 | GCATCTGGTGCAGGGAGAAG | GGG | 81.91369 | 46.14286 | 784 |
| 16077 | 1 | TCTGGTGCAGGGAGAAGGGG | AGG | 92.48414 | 60.72469 | 785 |
| 16082 | 1 | TGCAGGGAGAAGGGGAGGTC | AGG | 93.10184 | 46.08437 | 786 |
| 16095 | 1 | GGAGGTCAGGCGAGAGAATA | TGG | 93.54221 | 39.68623 | 787 |
| 16099 | 1 | GTCAGGCGAGAGAATATGGT | TGG | 92.48836 | 56.48815 | 788 |
| 16119 | 1 | TGGCAATATTGATCCATGTT | TGG | 82.02049 | 32.56259 | 789 |
| 16120 | 1 | GGCAATATTGATCCATGTTT | GGG | 77.55255 | 27.19259 | 790 |
| 16121 | 1 | GCAATATTGATCCATGTTTG | GGG | 74.7566 | 63.12063 | 791 |
| 16121 | -1 | GCGCTGCCACTCCCCAAACA | TGG | 96.37777 | 52.90008 | 792 |
| 16126 | 1 | ATTGATCCATGTTTGGGGAG | TGG | 87.10662 | 48.37676 | 793 |
| 16143 | -1 | GCCGAGATCGTGTGTAATTA | TGG | 97.83461 | 28.59523 | 794 |
| 16153 | 1 | GCCATAATTACACACGATCT | CGG | 96.59886 | 55.77195 | 795 |
| 16165 | -1 | TGAGACACTCCATGGTAGAC | TGG | 91.79216 | 52.52207 | 796 |
| 16167 | 1 | CGATCTCGGCCAGTCTACCA | TGG | 100 | 52.39825 | 797 |
| 16173 | -1 | GAAGTTGCTGAGACACTCCA | TGG | 95.9579 | 67.21232 | 798 |
| 16187 | 1 | TGGAGTGTCTCAGCAACTTC | AGG | 90.0358 | 32.39625 | 799 |
| 16188 | 1 | GGAGTGTCTCAGCAACTTCA | GGG | 93.36081 | 51.14577 | 800 |
| 16189 | 1 | GAGTGTCTCAGCAACTTCAG | GGG | 89.51798 | 72.6193 | 801 |
| 16200 | 1 | CAACTTCAGGGGTGATACCT | AGG | 97.10935 | 43.25481 | 802 |
| 16201 | 1 | AACTTCAGGGGTGATACCTA | GGG | 90.00002 | 51.96256 | 803 |
| 16206 | -1 | GCCTCTGACTTCATAGCCCT | AGG | 93.23889 | 55.05182 | 804 |
| 16216 | 1 | ACCTAGGGCTATGAAGTCAG | AGG | 96.23264 | 65.86427 | 805 |
| 16228 | -1 | CAAGTCCCTGGACTCTGTTG | TGG | 96.47011 | 52.94814 | 806 |
| 16233 | 1 | CAGAGGCCACAACAGAGTCC | AGG | 98.55521 | 53.21894 | 807 |
| 16234 | 1 | AGAGGCCACAACAGAGTCCA | GGG | 94.01286 | 58.98263 | 808 |
| 16240 | -1 | GGGTAAGAATTACAAGTCCC | TGG | 90.97415 | 50.38745 | 809 |
| 16260 | -1 | AGTTTAATAGAAGTAATAAT | GGG | 46.88021 | 37.37628 | 810 |
| 16261 | -1 | CAGTTTAATAGAAGTAATAA | TGG | 62.668 | 33.52716 | 811 |
| 16332 | 1 | AATATTATATATATTTATAT | TGG | 27.51617 | 28.97861 | 812 |
| 16339 | 1 | TATATATTTATATTGGATTT | TGG | 45.60089 | 18.21184 | 813 |
| 16340 | 1 | ATATATTTATATTGGATTTT | GGG | 41.67556 | 15.57417 | 814 |
| 16344 | 1 | ATTTATATTGGATTTTGGGA | TGG | 54.07712 | 37.86541 | 815 |
| 16345 | 1 | TTTATATTGGATTTTGGGAT | GGG | 52.6776 | 44.14789 | 816 |
| 16398 | 1 | ATCAAAGACTTGCTCAATAA | TGG | 65.21274 | 25.05297 | 817 |
| 16404 | 1 | GACTTGCTCAATAATGGATT | AGG | 79.79469 | 45.48502 | 818 |
| 16408 | 1 | TGCTCAATAATGGATTAGGC | TGG | 82.83171 | 48.09015 | 819 |
| 16415 | 1 | TAATGGATTAGGCTGGAATT | TGG | 87.54181 | 34.74462 | 820 |
| 16420 | 1 | GATTAGGCTGGAATTTGGTT | TGG | 73.47545 | 37.68495 | 821 |
| 16421 | 1 | ATTAGGCTGGAATTTGGTTT | GGG | 61.27861 | 39.60131 | 822 |
| 16447 | -1 | TCAAAGGCAACACAAGTGAT | AGG | 89.10229 | 58.40638 | 823 |
| 1273 | -1 | GAAACTAAATCCTCTGTTCA | AGG | 90.64196 | 43.40519 | 826 |

Reference is made to Table 6 presenting a summary of the sequences within the scope of the current invention.

TABLE 6
Summary of sequences within the scope of the present invention

| Sequence type | CsTHCAS | CsCBDAS | CsPT | CsOLS | CsAAE1 |
|---|---|---|---|---|---|
| Genomic | SEQ ID NO: 1 | SEQ ID NO: 4 | SEQ ID NO: 7 | SEQ ID NO: 10 | SEQ ID NO: 13 |
| sequence | | | | | |
| Coding sequence | SEQ ID NO: 2 | SEQ ID NO: 5 | SEQ ID NO: 8 | SEQ ID NO: 11 | SEQ ID NO: 14 |
| (CDS) | | | | | |
| Amino acid | SEQ ID NO: 3 | SEQ ID NO: 6 | SEQ ID NO: 9 | SEQ ID NO: 12 | SEQ ID NO: 15 |
| sequence | | | | | |
| gRNA sequence | SEQ ID NO: 16- | SEQ ID NO: 168-304 & | SEQ ID NO: 305- | SEQ ID NO: 459- | SEQ ID NO: 510-823 & |
| | SEQ ID NO: 167 | SEQ ID NO: 824-825 | SEQ ID NO: 458 | SEQ ID NO: 509 | SEQ ID NO: 826 |
| | (Table 1) | (Table 2) | (Table 3) | (Table 4) | (Table 5) |

The above gRNA molecules have been cloned into suitable vectors and their sequence has been verified. In addition different Cas9 versions have been analyzed for optimal compatibility between the Cas9 protein activity and the gRNA molecule in the Cannabis plant.

The efficiency of the designed gRNA molecules have been validated by transiently transforming Cannabis tissue culture. A plasmid carrying a gRNA sequence together with the Cas9 gene has been transformed into Cannabis protoplasts. The protoplast cells have been grown for a short period of time and then were analyzed for existence of genome editing events. The positive constructs have been subjected to the herein established stable transformation protocol into Cannabis plant tissue for producing genome edited Cannabis plants in Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) genes.

Stage 3: Transforming Cannabis plants using Agrobacterium or biolistics (gene gun) methods. For Agrobacterium and bioloistics, a DNA plasmid carrying (Cas9+gene specific gRNA) can be used. A vector containing a selection marker, Cas9 gene and relevant gene specific gRNA's is constructed. For biolistics, Ribonucleoprotein (RNP) complexes carrying (Cas9 protein+gene specific gRNA) are used. RNP complexes are created by mixing the Cas9 protein with relevant gene specific gRNA's.

According to some embodiments of the present invention, transformation of various Cannabis tissues was performed using particle bombardment of:
DNA vectors
Ribonucleoprotein complex (RNP's)

According to further embodiments of the present invention, transformation of various Cannabis tissues was performed using Agrobacterium (Agrobacterium tumefaciens) by:
Regeneration-based transformation
Floral-dip transformation
Seedling transformation

Transformation efficiency by A. tumefaciens has been compared to the bombardment method by transient GUS transformation experiment. After transformation, GUS staining of the transformants has been performed.

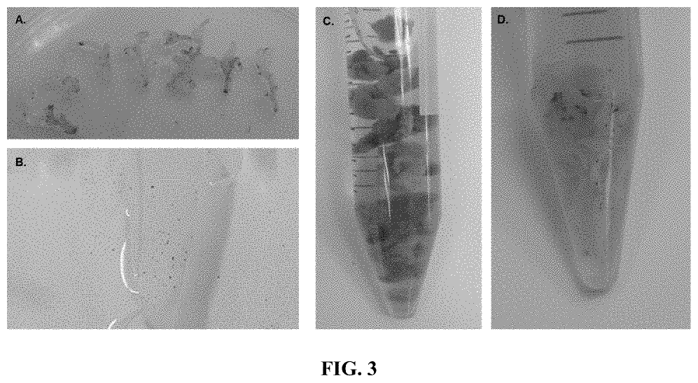

Reference is now made to FIG. 3 photographically presenting GUS staining after transient transformation of the following Cannabis tissues (A) axillary buds (B) leaf (C) calli, and (D) cotyledons. FIG. 3 demonstrates that various Cannabis tissues have been successfully transiently transformed using biolistics system. Transformation has been performed into calli, leaves, axillary buds and cotyledons of Cannabis.

According to further embodiments of the present invention, additional transformation tools were used in Cannabis, including, but not limited to:
Protoplast PEG transformation
Extend RNP use
Directed editing screening using fluorescent tags
Electroporation

Stage 4: Regeneration in tissue-culture. When transforming DNA constructs into the plant, antibiotics is used for selection of positive transformed plants. An improved regeneration protocol was herein established for the Cannabis plant.

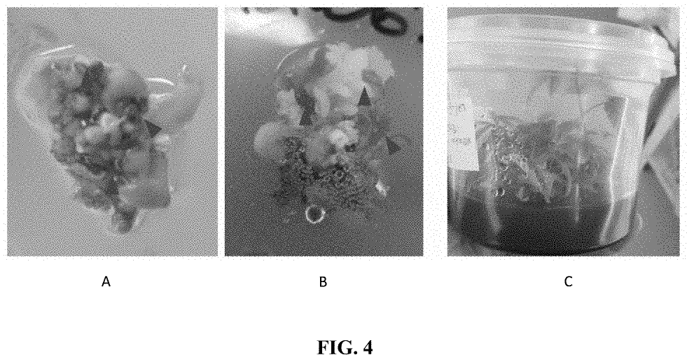

Reference is now made to FIG. 4A-C presenting regeneration of Cannabis tissue. In this figure, arrows indicate new meristem emergence.

Stage 5: Selection of positive transformants. Once regenerated plants appear in tissue culture, DNA is extracted from leaf sample of the transformed plant and PCR is performed using primers flanking the edited region. PCR products are then digested with enzymes recognizing the restriction site near the original gRNA sequence. If editing event occurred, the restriction site will be disrupted and the PCR product will not be cleaved. No editing event will result in a cleaved PCR product.

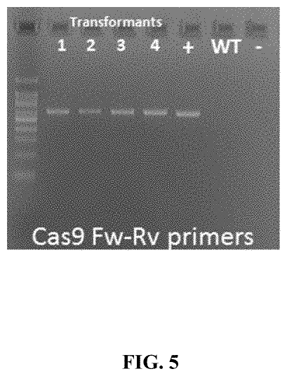

Reference is now made to FIG. 5 showing PCR detection of Cas9 DNA in shoots of transformed Cannabis plants. DNA extracted from shoots of plants transformed with Cas9 using biolistics. This figure shows that three weeks post transformation, Cas9 DNA was detected in shoots of transformed plants.

Screening for CRISPR/Cas9 gene editing events has been performed by at least one of the following analysis methods:
Restriction Fragment Length Polymorphism (RFLP)
Next Generation Sequencing (NGS)
PCR fragment analysis
Fluorescent-tag based screening
High resolution melting curve analysis (HRMA)

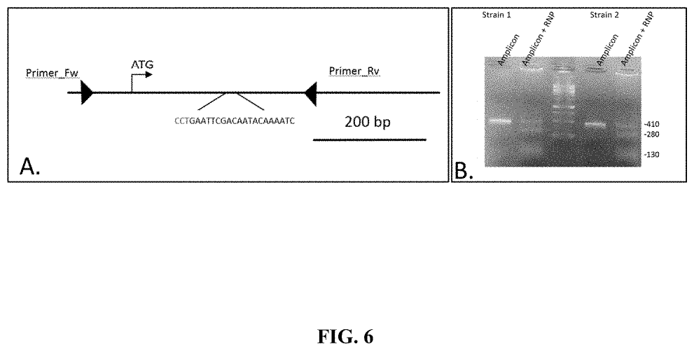

Reference is now made to FIG. 6 presenting results of in vitro analysis of CRISPR/Cas9 cleavage activity. FIG. 6A schematically shows the genomic area targeted for editing (PAM is marked in red) and amplified by the reverse and forward designed primers FIG. 6B photographically presents a gel showing successful digestion of the resulted PCR amplicon containing the gene specific gRNA sequence, by RNP complex containing Cas9. The analysis included the following steps:
1) Amplicon was isolated from two exemplified Cannabis strains by primers flanking the sequence of the gene of interest targeted by the predesigned sgRNA.
2) RNP complex was incubated with the isolated amplicon.
3) The reaction mix was then loaded on agarose gel to evaluate Cas9 cleavage activity at the target site.

Stage 6: Selection of transformed Cannabis plants presenting reduced expression of at least one of Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) as described above. It is within the scope that different gRNA promoters were tested in order to maximize editing efficiency.

Various embodiments have been presented. Each of these embodiments may of course include features from other embodiments presented, and embodiments not specifically described may include various features described herein.

1. A Cannabis plant with reduced delta-9-tetrahydrocannabinol (THC) content, or reduced cannabidiol (CBD) content, or reduced THC and CBD content, wherein said plant comprises at least one mutated gene encoding a cannabinoid biosynthesis enzyme selected from the group consisting of Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) and any combination thereof, further wherein said mutation is introduced by targeted genome modification using at least one RNA-guided (gRNA) endonuclease selected from the group consisting of:
a. gRNA targeted to CsTHCAS genomic sequence, said gRNA having a nucleotide sequence selected from the group consisting of SEQ ID NO: 16-167 and any combination thereof;
b. gRNA targeted to CsCBDAS genomic sequence, said gRNA having a nucleotide sequence selected from the group consisting of SEQ ID NO: 168-304, 824-825 and any combination thereof;
c. gRNA targeted to CsPT genomic sequence, said gRNA having a nucleotide sequence selected from the group consisting of SEQ ID NO: 305-458 and any combination thereof;
d. gRNA targeted to CsOLS genomic sequence, said gRNA having a nucleotide sequence selected from the group consisting of SEQ ID NO: 459-509 and any combination thereof; and
e. gRNA targeted to CsAAE1 genomic sequence, said gRNA having a nucleotide sequence selected from the group consisting of SEQ ID NO: 510-823, 826 and any combination thereof.

2. (canceled)

3. (canceled)

4. (canceled)

5. (canceled)

6. (canceled)

7. The Cannabis plant according to claim 1, wherein the genomic sequence encoding said at least one cannabinoid biosynthesis enzyme is selected from the group consisting of: CsTHCAS having a genomic nucleotide sequence as set forth in SEQ ID NO:1 or a functional variant thereof, CsCBDAS having a genomic nucleotide sequence as set forth in SEQ ID NO:4 or a functional variant thereof, CsPT having a genomic nucleotide sequence as set forth in SEQ ID NO:7 or a functional variant thereof, CsOLS having a genomic nucleotide sequence as set forth in SEQ ID NO:10 or a functional variant thereof and CsAAE1 having a genomic nucleotide sequence as set forth in SEQ ID NO:13 or a functional variant thereof.

8. The Cannabis plant according to claim 7, wherein said functional variant has at least 75% sequence identity to the nucleotide sequence of said cannabinoid biosynthesis enzyme or a codon degenerate nucleotide sequence thereof.

9. The Cannabis plant according to claim 1, wherein said plant has decreased expression levels of at least one of CsTHCAS protein having an amino acid sequence with at least 75% identity to the amino acid sequence as set forth in SEQ ID NO:3 or a conservatively substituted amino acid sequence thereof, CsCBDAS protein having an amino acid sequence with at least 75% identity to the amino acid sequence as set forth in SEQ ID NO:6 or a conservatively substituted amino acid sequence thereof, CsPT protein having an amino acid sequence with at least 75% identity to the amino acid sequence as set forth in SEQ ID NO:9 or a conservatively substituted amino acid sequence thereof, CsOLS protein having an amino acid sequence with at least 75% identity to the amino acid sequence as set forth in SEQ ID NO:12 or a conservatively substituted amino acid sequence thereof, and CsAAE1 protein having an amino acid sequence with at least 75% identity to the amino acid sequence as set forth in SEQ ID NO:15 or a conservatively substituted amino acid sequence thereof.

10. The Cannabis plant according to claim 1, wherein said Cannabis plant has modulated expression of one or more of the cannabinoids, optionally cannabigerolic acid, cannabigerol, DELTA.9-tetrahydrocannabinolic acid, cannabidiolic acid, cannabichromenic acid, DELTA.9-tetrahydrocannabinol, cannabidiol, cannabichromene, THC, D9-THC, D8-THC, THCA, THCV, D8-THCV, D9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA and cannabidiol.

11. (canceled)

12. (canceled)

13. (canceled)

14. (canceled)

15. (canceled)

16. (canceled)

17. (canceled)

18. The Cannabis plant of claim 1, wherein at least one of the following holds true: (a) said plant is homozygous for said at least one mutated gene; (b) said plant genotype is obtainable by deposit under accession number with NCIMB Aberdeen AB21 9YA, Scotland, UK; or (c) said Cannabis plant comprises a DNA encoding an antisense RNA or a siRNA effective to suppress expression of CsTHCAS, CsSP, CsCBDAS, CsPT, CsOLS, CsAAE1 and any combination thereof, the DNA operably linked to a heterologous promoter, wherein the Cannabis plant comprises reduced THC content, reduced CBD content, or decreased THC and CBD content relative to a Cannabis plant of a similar genotype that does not comprise the DNA.

19. (canceled)

20. (canceled)

21. (canceled)

22. (canceled)

23. (canceled)

24. (canceled)

25. (canceled)

26. (canceled)

27. A plant part, plant cell or plant seed, tissue culture of regenerable cells, protoplasts or callus of a plant according to claim 1.

28. (canceled)

29. (canceled)

30. (canceled)

31. The Cannabis plant according to claim 1, wherein said Cannabis plant has at least one of the following characteristics: (a) a THC content of up to 30% by weight, particularly between about 0.1% to about 30% by weight, more particularly between about 0.3% to about 30%, even more particularly between about 0.3% to about 10% by weight; (b) a CBD content of up to 30% by weight, particularly between about 0.1% to about 30% by weight, more particularly between about 0.3% to about 30%, even more particularly between about 0.3% to about 10% by weight; (c) a THC and/or CBD content of not more than about 0.5% by weight; or (d) said plant is THC free.

32. (canceled)

33. (canceled)

34. (canceled)

35. (canceled)

36. The Cannabis plant according to claim 1, wherein said plant has at least one targeted genome modification in at least one Cannabis gene encoding cannabinoid precursor synthesis enzyme selected from the group consisting of CsAAE, CsPT and CsOLS and said plant exhibits at least one of the following characteristics: (a) reduced expression of THC, CBD or both relative to a Cannabis plant of a similar genotype or genetic background lacking said targeted genome modification; (b) a THC and/or CBD content of up to 30% by weight, particularly between about 0.1% to about 30% by weight, more particularly between about 0.3% to about 30%, even more particularly between about 0.3% to about 10% by weight; or (c) a THC and/or CBD content of not more than about 0.5% by weight.

37. (canceled)

38. (canceled)

39. (canceled)

40. (canceled)

41. The Cannabis plant according to claim 1, wherein said plant has at least one targeted genome modification in CsTHCAS and said plant exhibits reduced expression of THCA or THC, and elevated expression of CBD or CBDA relative to a Cannabis plant of a similar genotype or genetic background lacking said targeted genome modification, and wherein said plant has at least one of the following characteristics: (a) a THC and/or THCA content of up to 30% by weight, particularly between about 0.1% to about 30% by weight, more particularly between about 0.3% to about 30%, even more particularly between about 0.3% to about 10% by weight; or (b) a THC and/or THCA content of not more than about 0.5% by weight.

42. (canceled)

43. (canceled)

44. The Cannabis plant according to claim 1, wherein said plant has at least one targeted genome modification in CsCBDAS and said plant exhibits reduced expression of CBDA or CBD, and elevated expression of THC or THCA relative to a Cannabis plant of a similar genotype or genetic background lacking said targeted genome modification, and wherein said plant has at least one of the following characteristics: (a) a CBD and/or CBDA content of up to 30% by weight, particularly between about 0.1% to about 30% by weight, more particularly between about 0.3% to about 30%, even more particularly between about 0.3% to about 10% by weight; or (b) a CBD and/or CBDA content of not more than about 0.5% by weight.

45. (canceled)

46. A Cannabis plant derived product or a derived medical composition from the plant of claim 1.

47. The Cannabis plant derived product of claim 46, comprising at least one of the following characteristics: (a) a combined cannabidiolic acid and cannabidiol concentration of about 0.3% to about 30% by weight; (b) a combined delta-9-tetrahydrocannabinol and tetrahydrocannabinolic acid concentration of between about 0.3% to about 30% by weight; (c) Cannabis oil, Cannabis tincture, dried Cannabis flowers, and/or dried Cannabis leaves; (d) utilized as a medicament; or (e) formulated for inhalation, oral consumption, sublingual consumption, or topical consumption.

48. (canceled)

49. (canceled)

50. (canceled)

51. (canceled)

52. (canceled)

53. A method for producing a Cannabis plant according to claim 1, wherein said method comprises steps of introducing into the genome of a Cannabis plant or a cell thereof at least one targeted genome modification effective in decreasing expression of a at least one Cannabis gene encoding a cannabinoid biosynthesis enzyme selected from the group consisting of tetrahydrocannabinolic acid synthase (THCAS), cannabidiolic acid synthase (CBDAS), aromatic prenyltransferase (PT), olivetol synthase (OLS), acyl-activating enzyme 1 (AAE1) and any combination thereof, said introducing of at least one targeted genome modification comprises steps of introducing into said Cannabis plant or a cell thereof at least one RNA-guided (gRNA) endonuclease selected from the group consisting of:
a. gRNA targeted to CsTHCAS genomic sequence, said gRNA having a nucleotide sequence selected from the group consisting of SEQ ID NO: 16-167 and any combination thereof;
b. gRNA targeted to CsCBDAS genomic sequence, said gRNA having a nucleotide sequence selected from the group consisting of SEQ ID NO: 168-304, 824-825 and any combination thereof;
c. gRNA targeted to CsPT genomic sequence, said gRNA having a nucleotide sequence selected from the group consisting of SEQ ID NO: 305-458 and any combination thereof;
d. gRNA targeted to CsOLS genomic sequence, said gRNA having a nucleotide sequence selected from the group consisting of SEQ ID NO: 459-509 and any combination thereof; and
e. gRNA targeted to CsAAE1 genomic sequence, said gRNA having a nucleotide sequence selected from the group consisting of SEQ ID NO: 510-823, 826 and any combination thereof.

54. (canceled)

55. (canceled)

56. (canceled)

57. (canceled)

58. (canceled)

59. (canceled)

60. (canceled)

61. (canceled)

62. (canceled)

63. (canceled)

64. The method according to claim 53, wherein at least one of the following holds true: (a) the nucleotide sequence encoding said at least one cannabinoid biosynthesis enzyme is selected from the group consisting of: CsTHCAS having a genomic nucleotide sequence as set forth in SEQ ID NO:1 or a functional variant thereof, CsCBDAS having a genomic nucleotide sequence as set forth in SEQ ID NO:4 or a functional variant thereof, CsPT having a genomic nucleotide sequence as set forth in SEQ ID NO:7 or a functional variant thereof, CsOLS having a genomic nucleotide sequence as set forth in SEQ ID NO:10 or a functional variant thereof and CsAAE1 having a genomic nucleotide sequence as set forth in SEQ ID NO:13 or a functional variant thereof; or (b) said plant has decreased expression levels of at least one of CsTHCAS protein having an amino acid sequence with at least 75% identity to the amino acid sequence as set forth in SEQ ID NO:3 or a conservatively substituted amino acid sequence thereof, CsCBDAS protein having an amino acid sequence with at least 75% identity to the amino acid sequence as set forth in SEQ ID NO:6 or a conservatively substituted amino acid sequence thereof, CsPT protein having an amino acid sequence with at least 75% identity to the amino acid sequence as set forth in SEQ ID NO:9 or a conservatively substituted amino acid sequence thereof, CsOLS protein having an amino acid sequence with at least 75% identity to the amino acid sequence as set forth in SEQ ID NO:12 or a conservatively substituted amino acid sequence thereof, and CsAAE1 protein having an amino acid sequence with at least 75% identity to the amino acid sequence as set forth in SEQ ID NO:15 or a conservatively substituted amino acid sequence thereof.

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. A plant part, plant cell or plant seed produced by the method according to claim 53.

77. (canceled)

78. A method for producing a medical Cannabis composition, the method comprising:
a. obtaining the Cannabis plant of claim 1; and
b. formulating a medical Cannabis composition from said plant.

79. A method for manipulating a content of one or more cannabinoids in a Cannabis plant, the method comprising down-regulating activity of at least one Cannabis gene encoding a cannabinoid biosynthesis enzyme selected from the group consisting of tetrahydrocannabinolic acid synthase (THCAS) having a genomic nucleotide sequence with at least 75% sequence identity to a sequence as set forth in SEQ ID NO:1, cannabidiolic acid synthase (CBDAS) having a genomic nucleotide sequence with at least 75% sequence identity to a sequence as set forth in SEQ ID NO:4, aromatic prenyltransferase (PT) having a genomic nucleotide sequence with at least 75% sequence identity to a sequence as set forth in SEQ ID NO:7, olivetol synthase (OLS) having a genomic nucleotide sequence with at least 75% sequence identity to a sequence as set forth in SEQ ID NO:10, acyl-activating enzyme 1 (AAE1) having a genomic nucleotide sequence with at least 75% sequence identity to a sequence as set forth in SEQ ID NO:13 and any combination thereof, said down regulating comprising introducing a loss of function mutation into said nucleic acid sequence encoding said at least one cannabinoid biosynthesis enzyme using targeted genome modification.

80. (canceled)

81. (canceled)

82. (canceled)

83. An isolated nucleotide sequence having at least 75% sequence identity to a nucleotide sequence selected from the group consisting of SEQ ID NO:1, SEQ ID NO:2 SEQ ID NO:4, SEQ ID NO:5, SEQ ID NO:7, SEQ ID NO:8, SEQ ID NO:10, SEQ ID NO:11, SEQ ID NO:13, SEQ ID NO:14 and SEQ ID NO:16-826; and/or an isolated amino acid sequence having at least 75% sequence identity to an amino acid sequence selected from the group consisting of SEQ ID NO:3, SEQ ID NO:6, SEQ ID NO:9, SEQ ID NO:12 and SEQ ID NO:15; and/or a vector, construct or expression system or cassette comprising nucleic acid sequence having at least 75% sequence identity to a nucleotide sequence selected from the group consisting of SEQ ID NO:1, SEQ ID NO:2, SEQ ID NO:4, SEQ ID NO:5, SEQ ID NO:7, SEQ ID NO:8, SEQ ID NO:10, SEQ ID NO:11, SEQ ID NO:13, SEQ ID NO:14 and SEQ ID NO:16-826.

84. (canceled)

85. (canceled)

86. (canceled)

87. (canceled)

88. A method for down regulation of at least one Cannabis cannabinoid biosynthesis enzyme gene selected from the group consisting of Cannabis tetrahydrocannabinolic acid synthase (CsTHCAS), Cannabis cannabidiolic acid synthase (CsCBDAS), Cannabis aromatic prenyltransferase (CsPT), Cannabis olivetol synthase (CsOLS), Cannabis acyl-activating enzyme 1 (CsAAE1) and any combination thereof, which comprises utilizing the nucleotide sequence as set forth in at least one of SEQ ID NO:16-826 and any combination thereof said utilizing comprising introducing a loss of function mutation into said at least one gene encoding said at least one cannabinoid biosynthesis enzyme using targeted genome modification.

89. (canceled)

90. (canceled)

91. (canceled)

92. (canceled)

93.-100. (canceled)
